(19) United States
(12) Patent Application Publication
(10) Pub. No.: US 2026/0028630 A1
(43) Pub. Date: Jan. 29, 2026

(54) HYBRID OLIGONUCLEOTIDES

(71) Applicant: ENTRADA THERAPEUTICS, INC., Boston, MA (US)

(72) Inventors: Ireneusz NOWAK, Allston, MA (US); Ziqing QIAN, Lexington, MA (US); Mahboubeh KHEIRABADI, Natick, MA (US)

(21) Appl. No.: 18/993,485

(22) PCT Filed: Jul. 13, 2023
(86) PCT No.: PCT/US2023/070154

§ 371 Date: Jan. 10, 2025

Related U.S. Application Data
(60) Provisional application No. 63/389,775, filed on Jul. 15, 2022.
(60) Provisional application No. 63/389,870, filed on Jul. 16, 2022.
(60) Provisional application No. 63/498,633, filed on Apr. 27, 2023.

(51) Int. Cl. C12N 15/113

(52) U.S. Cl. CPC C12N 15/1137; C12N 2310/11; C12N 2310/14; C12N 2310/321; C12N 2310/322; C12N 2310/3233; C12N 2310/341; C12N 2310/51

(57) ABSTRACT

Provided herein are methods for making hybrid oligonucleotides, hybrid oligonucleotides, and compounds and compositions comprising hybrid oligonucleotides.

### CROSS-REFERENCE TO RELATED APPLICATIONS

This application claims priority to U.S. provisional patent application No. 63/389,775, which was filed on Jul. 15, 2022, U.S. provisional patent application No. 63/389,870, which was filed on Jul. 16, 2022, and U.S. provisional patent application No. 63/498,633, which was filed on Apr. 27, 2023, the disclosures of each of which are hereby incorporated by reference in their entireties.

### BACKGROUND

Recognition of specific RNA target nucleic acid sequences via complementary base pairing oligonucleotides has been investigated as a method for treating a variety of diseases and disorders. Both single and double stranded oligonucleotides with diverse chemical modifications have been designed to modulate gene expression, for example, through antisense mechanisms, such as RNA interference (RNAi), gapmers, or steric block.

Two classes of internucleotidic linkages dominate today in clinically significant oligonucleotides: linkages with a charged backbone such as phosphodiesters and phosphorothioates (PS) (Stec et al., J. Am. Chem. Soc. 106 (20), 1984, 6077-6079), and neutral linkages such as phosphordiamidate linkages, for example, of morpholino oligonucleotide analogs in which the ribose sugar of the nucleobase is replaced with a morpholino ring (PMOs). PMOs are widely used as steric block oligonucleotides that do not require RNAse H activity, including, for example, as splice-modulating oligonucleotides.

Gapmers are oligonucleotide constructs that include a central gap region of approximately 8-12 nucleotides that support RNAse H activity and two flanking regions of approximately 1-5 modified nucleotides (also referred to as “wings”) that provide stability and increase affinity towards RNA. Typically, a gapmer includes phosphorothioate (PS) linkages along the entire length of the oligonucleotide. The nucleotides in the central gap region typically include phosphorothioate (PS) linked DNA nucleotides and the flanking wings typically include sugar modifications such as 2′-O-methylation (2′-OMe), 2′-O-methoxyethyl (2′-MOE), or linked nucleic acid (LNA), linked by phosphorothioate (PS) linkages. In addition to supporting RNAse H activity, it is believed that the gap region facilitates transfection by a dynamic covalent interaction with membrane proteins (Laurent et al., Angew. Chem. Int. Ed. 2021, 60, 1-6). It is believed that 8 or 9 continuous PS linkages in the gap region are needed for RNAse H activity (Shen et al., (2014) Nucleic Acids Res., 42, 8648-8662; Brown et al., (1994) J. Biol. Chem., 269, 26801-26805) and 10-13 anionic PS linkages facilitate electrostatic and hydrophobic interactions with amino acid side chains, for example, for protein binding (Hyjek-Skladanowska, et al., (2020). J. Am. Chem. Soc., 142, 7456-7468). However, there is growing evidence that PS engages in unfavorable interactions with cell proteins that can result in toxicity.

Selective chemical substitutions of nucleotides in the gap region have been investigated to improve the therapeutic index and reduce toxicity of gapmer oligonucleotides. Because many proteins favor binding to the 5′ wing (flank) of the gapmer, it has been found that hydrophilic or neutral linkages at positions 2-4 from the 5′ end of the gap region tend to reduce toxicity. Other designs that have been investigated introduce unique chemistries to the flanking regions of gapmers, for example, charged N3′—P5′ or neutral guanidinium (PN) linkages, which improve pharmacology and gene silencing (Kupryushkin et al., Phosphoryl Guanidines: A New Type of Nucleic Acid Analogues. Acta Nat. 2014, 6, 116-118; Gryaznov et al., Nucleic Acids Res., 24: 1508-1514, 1996).

Use of neutral linkages, for example, methylphosphonates, has also been reported, but was found to have reduced solubility in aqueous solutions, and was associated with destabilization of helix and synthetic challenges (Guo et al., Phosphorus, Sulfur and Silicon 1999, Vol 144-146, 363-366).

Recently, mixed-backbone gapmers have been proposed to produce less toxic antisense oligonucleotides (ASOs) without compromising activity in the central nervous system (CNS). A prodrug concept based on charge neutralizing phosphotriester modifications has also been proposed (Meade et al., Nat Biotechnol. 2014 32(12), 1256-1261).

Despite the individual successes of morpholino and DNA-PS containing oligonucleotides, chemical synthesis of oligonucleotide hybrids that incorporate both morpholino and DNA-PS subunits has been hampered by the synthetic strategy that is currently used to generate PMOs (Heera et al., J. Am. Chem. Soc., 142, 38:16240 (2020)). PMOs are typically prepared in a 6′ to 3′ direction on a polystyrene resin (Paul and Marvin, J. Am. Chem. Soc., 138:15663 (2016)), which is not compatible with standard methods for the chemical synthesis of natural DNA/RNA or most analogues, which are prepared in a stepwise 3′ to 5′ direction. (FIG. 1). Additionally, PMO synthesis typically employs P(V) chemistry while DNA or RNA synthesis employs P(III) phosphoramidite chemistry. For these and other reasons, PMO and DNA syntheses have been considered incompatible.

Recent attempts at the synthesis of PMO-ASO chimeras include thiophosphoroamidate (also referred to as phosphorothioamidate) morpholinos (TMOs)—oligonucleotides that combine PS charged linkages and morpholino rings in the backbone. Synthesis of these oligonucleotides is accomplished in a 3′ to 5′ direction by sequential addition of morpholino 3′-phosphorodiamidite P(III) nucleotides (Langner, et al., J. Am. Chem. Soc. 2020, 142(38), 16240-16253). Caruthers reports an antisense oligonucleotide that can specifically hybridize to a target region in an exon of the human dystrophin gene to induce exon skipping that is 8 to 50 nucleotides in length, comprising a thiomorpholino nucleotide (TMO) comprising at least 8 to 10 consecutive nucleotides complementary to a target region in the human dystrophin gene exon, wherein the TMO comprises a morpholino subunit, wherein the morpholino nitrogen of the morpholino subunit is linked by a thiophosphate-containing internucleotidic linkage to a 5′ exocyclic carbon of an adjacent nucleotide, or the 6′-exocyclic carbon of an adjacent morpholino subunit, or a TMO/DNA chimera, wherein and at least one nucleotide base comprises a base other than uracil (FIGS. 2 and 3).

There remains a need for a robust and flexible synthetic route for generating chimeras that contain morpholino nucleotides and other therapeutically relevant modifications, for example, for use in new therapeutic drugs with improved biological attributes.

### SUMMARY

The disclosure provides methods of making a hybrid oligonucleotide comprising assembling P(III) and P(V) nucleotide nucleotides in a 6′ to 3′ or 5′ to 3′ direction on a support, and employing PMO 6′-phosphoramidite P(I) or phosphoramidate P(V) as the first nucleotide from the support.

The disclosure provides a hybrid oligonucleotide synthesized by the method of the disclosure.

The disclosure also provides a hybrid oligonucleotide comprising:
(i) a first nucleotide sequence comprising a first 5′ or 6′ end and a first 3′ end, wherein the first 3′ end comprises a terminal 3′ nucleic acid residue that is a morpholino nucleotide analog;
(ii) a second nucleotide sequence comprising a second 5′ end and a second 3′ end, wherein the second 5′ end comprises a terminal 5′ nucleic acid residue that is a deoxyribonucleotide or analog thereof, or a ribonucleotide or analog thereof; and
(iii) at least one phosphrothioamidate linkage that links the terminal 3′ nucleic acid residue of the first nucleotide sequence and the terminal 5′ nucleic acid residue of the second nucleotide sequence.

The disclosure also provides a hybrid oligonucleotide comprising:
(i) at least one P(III) morpholino nucleotide analog;
(ii) at least one P(V) morpholino nucleotide analog;
(iii) at least one P(III) ribonucleotide, at least one P(III) deoxyribonucleotide analog, or a combination thereof;
(iv) at least one phosphorodiamidate linkage

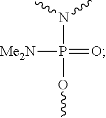

and
(v) at least one phosphorothioamidite

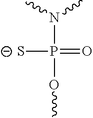

linkage.

In embodiments, each 5′ nucleotide adjacent to each P(V) morpholino nucleotide analog is a P(III) or P(V) morpholino nucleotide analog.

In embodiments, the hybrid oligonucleotide comprises two or more consecutive P(V) morpholino nucleotide analogs linked through phosphorodiamidate linkages.

In embodiments, any P(V) morpholino nucleotide analog is linked to any downstream nucleotide that is not a P(V) morpholino nucleotide analog through a phosphorothioamidate linkage.

The disclosure also relates to compound comprising: the hybrid oligonucleotide of the disclosure, and a cyclic peptide of formula (I):

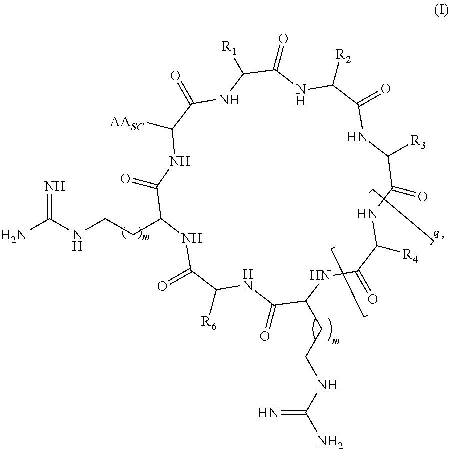

or a protonated form thereof,
wherein:
R₁, R₂, and R₃ are each independently H or an aromatic or heteroaromatic side chain of an amino acid;
R₄ and R₆ are independently H or an amino acid side chain;
AA_{SC} is an amino acid side chain;
q is 1, 2, 3 or 4; and
each m is independently an integer from 0-3.

The disclosure also relates to a compound comprising: the hybrid oligonucleotide of the disclosure, and an EEV of Formula (B):

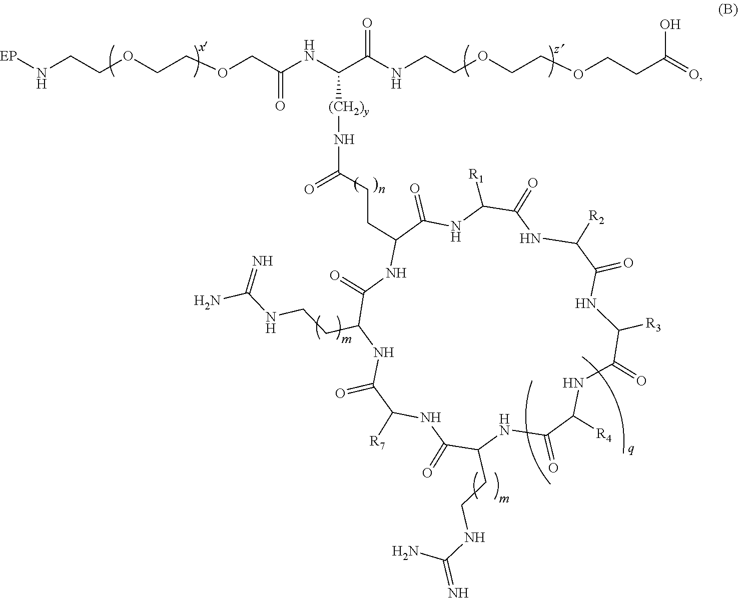

or a protonated form thereof,
wherein:
R₁, R₂, and R₃ are each independently H or an aromatic or heteroaromatic side chain of an amino acid;
R₄ and R₇ are independently H or an amino acid side chain;
EP is an exocyclic peptide as defined herein;
each m is independently an integer from 0-3;
n is an integer from 0-2:
x′ is an integer from 1-20;
y is an integer from 1-5;
q is 1-4; and
z′ is an integer from 1-23.

The disclosure further relates to compositions comprising the compounds of the disclosure and method of treating a disease with such compounds and compositions.

### BRIEF DESCRIPTION OF THE FIGURES

FIG. 1A-IB. A schematic comparing: (A) a typical gapmer structure comprising 5′ and 3′ ribonucleic acid (RNA) flanking regions in which the RNA monomer units are linked through phosphorothioate (PS) linkages (RNA-PS) and a deoxyribonucleotide (DNA) containing center gap region in which the DNA monomer units are linked through phosphorothioate (PS) linkages (DNA-PS); and (B) a hybrid gapmer oligonucleotide as described herein comprising 5′ and 3′ phosphorodiamidate morpholino oligomer (PMO) flanking regions and a DNA containing center gap region in which the DNA monomer units are linked through phosphorothioate (PS) linkages (DNA-PS).

FIG. 2. A table comparing properties of hybrid oligonucleotides having a PMO-ASO backbone to oligonucleotides having a PMO-only backbone or PS-only backbone.

FIG. 3. A schematic comparing the common direction of synthesis for a phosphorodiamidate morpholino oligonucleotide (PMO) (6′ to 3′) to the typical direction of synthesis for deoxyribonucleotide-containing antisense oligonucleotide (ASO) (3′ to 5′).

FIG. 4. A schematic showing the reversed synthesis direction for ASO (5′ to 3′) according to the method described herein.

FIG. 5A-5I. Graphic representation of sample oligonucleotides. (A) types of monomers: P(V) morpholino; P(III) morpholino; reverse DNA P(III), and reverse 2′ modified RNA P(III) ribonucleotides (e.g., reverse 2′-O-methyl (2′-OMe) P(III) or 2′-O-methoxyethyl (2′-MOE) P(l) ribonucleotides) with 6′ or 5′ phosphoro linking groups; (B) types of internucleotidic linkages: phosphorodiamidate; phosphorothioamidate; and phosphothioate; (C) hybrid gapmer oligonucleotide with 5′ and 3′ flanks comprising reverse RNA P(III) nucleotide monomers and a central gap region comprising reverse DNA P(III) nucleotide monomers linked by phosphorothioate linkages: (D) a traditional PMO oligonucleotide comprising P(V) morpholino nucleotide analogs linked via phosphorodiamidate linkages; (E) hybrid gapmer oligonucleotide with 6′ and 3′ flanks comprising P(V) morpholino nucleotide analogs linked by phosphorodiamidate linkages; a central gap region comprising reverse DNA P(III) nucleotides linked by phosphorothioate linkages, wherein the 5′ (6′) terminal nucleotide in each of the 5′ and 3′ flanks is a P(III) morpholino nucleotide analog and the 5′ flank comprises of a series of reverse morpholino P(III) nucleotides linked by phosphorothioamidate linkages; (F) hybrid gapmer oligonucleotide with 6′ and 3′ flanks comprising P(V) morpholino nucleotide analogs linked by phosphorodiamidate linkages; a central gap region comprising reverse DNA P(III) nucleotides linked by phosphorothioate linkages, wherein the 5′ (6′) terminal nucleotide in each of the 5′ and 3′ flanks is a P(III) morpholino nucleotide analog and the 5′ flank is linked to the gap region by a phosphorothioamidate linkage; (G) hybrid gapmer oligonucleotide with 5′ flank comprising P(V) morpholino nucleotide analogs linked by phosphorodiamidate linkages in which the 5′ terminal nucleotide is a P(III) morpholino nucleotide analog; a central gap region comprising reverse DNA P(III) nucleotides linked by phosphorothioate linkages, wherein the 5′ flank is linked to the gap region by a phosphorothioamidate phosphorothioamidate linkage, and a 3′ flank comprising reverse RNA P(III) nucleotides linked by phosphorothioate linkages; (III) hybrid oligonucleotide comprising P(III) morpholino linked by a phosphorodiamidate to a series of P(V) morpholino linked by phophorodiamidate linkages, and a series of reverse RNA P(III) nucleotides linked by phosphorothioate linkages, wherein the upstream RNA (III) nucleotide is linked to the downstream P(V) morpholino by a phosphorothioamidate linkage; (I) hybrid oligonucleotide comprising P(l) morpholino linked by a phosphorodiamidate to a series of P(V) morpholino linked by phophorodiamidate linkages, and a series of reverse morpholino P(III) nucleotides linked by phosphorothioamidate linkages, wherein the upstream morpholino (III) nucleotide is linked to the downstream P(V) morpholino by a phosphorothioamidate linkage.

FIG. 6A-6F. Schematic of sample oligonucleotides. (A) types of monomers: P(V) morpholino; P(III) morpholino; reverse DNA P(III), reverse 2′ modified RNA P(III) (e.g., reverse 2′-O-Methyl (2′-OMe) P(III), reverse 2′-O-Methoxyethyl (2′-MOE) P(III) and reverse 2′-Flouro (2′-F) P(III) ribonucleotides) with 6′ or 5′ phosphoro linking groups; (B) types of internucleotidic linkages: phosphorodiamidate; phosphorothioamidate phosphorothioamidate; phosphorothioate and phosphodiester; (C) hybrid oligonucleotide comprising two or more regions comprising P(V) morpholino nucleotide analogs linked through phosphorodiamidate linkages wherein each region independently comprises a 5′ end and a 3′ end, in which the terminal 5′ (6′) nucleotide of each region is a P(III) morpholino nucleotide analog (referred to herein as a “lynchpin” nucleotide) and the 3′ end of each upstream region is linked to the 5′ end of the adjacent downstream region via a phosphorothioamidate linkage; (D) a hybrid gapmer oligonucleotide as in (C) including one or more regions comprising reverse RNA P(III) nucleotides linked by phosphorothioate linkages, wherein the 5′ terminal end of the RNA P(III) region is linked to the upstream P(V) morpholino region through a P(III) morpholino nucleotide analog (“lynchpin”) that is linked to the upstream P(V) morpholino region and to the downstream RNA P(MII) region through phosphorothioamidate linkage; (E) and (F) are examples of hybrid oligonucleotides comprising one or more P(V) morpholino nucleotide analogs, one or more P(III) morpholino nucleotide analogs, or combinations thereof; and one or more DNA or RNA nucleotide analogs selected from: reverse DNA P(III), reverse 2′ modified RNA P(III), or combinations thereof, wherein the adjacent 5′ nucleotide to each P(V) morpholino nucleotide analog is a P(III) or P(V) morpholino nucleotide analog, wherein two or more consecutive P(V) morpholino nucleotide analogs are linked through phosphorodiamidate linkages, wherein any P(V) morpholino nucleotide analog is linked to a downstream nucleotide that is not a P(V) morpholino nucleotide analog through a phosphorothioamidate linkage.

FIG. 7A-7C. Graphic representation of a siRNA duplex. (A) types of monomers; P(V) morpholino; P(III) morpholino; reverse DNA P(III), reverse 2′ modified RNA P(III) (e.g., reverse 2′-O-Methyl (2′-OMe) P(III), reverse 2′-O-Methoxyethyl (2′-MOE) P(III) and reverse 2′-Flouro 10 (2′-F) P(III) ribonucleotides) with 6′ or 5′ phosphoro linking groups; (B) types of internucleotidic linkages: phosphorodiamidate; phosphorothioamidate; phosphothioate; (C) Schematic of a siRNA duplex in which the sense and antisense strands are hybrid oligonucleotides as described herein.

FIG. 8. One of conventional siRNA designs.

FIG. 9. Introduction of PMO to siRNA strands at terminal or internal positions.

FIG. 10. Structures of triphenylmethyl or “trityl” (Tr) and dimethoxytriphenylmethyl or “dimethoxytrityl” (DMT) protected P(III) moropholino nucleotide analogs (A, C, G, and T) that can be included in hybrid oligonucleotides as described herein.

FIG. 11. Structures of dimethoxytrityl (DMT) protected reverse DNA P(III) nucleotide analogs (A, C, G and T) that can be included in hybrid oligonucleotides as described herein.

FIG. 12. Structures of dimethoxytrityl (DMT) protected 2′ modified (2′-F, 2-′O-Me, 2′-OMOE) reverse RNA P(III) nucleotide analogs (A, C, G and T) that can be included in hybrid oligonucleotides as described herein.

FIG. 13. Structures of dimethoxytrityl (DMT) protected 2′ modified reverse RNA P(III) nucleotide analogs with methylated C and U bases that can be included in hybrid oligonucleotides as described herein.

FIG. 14. Trityl (Tr) protected P(V) morpholino nucleobase analogs with modified G bases that can be included in hybrid oligonucleotides as described herein.

FIG. 15. Trityl (Tr) protected P(V) morpholino nucleobase analogs (G, T, A, C) that can be included in hybrid oligonucleotides as described herein.

FIG. 16. Examples of solid supports that are typically used in synthesis of PMOs (A and B) and charged oligonucleotides C.

FIG. 17A-17F. Structures of monomers that can be used in hybrid oligonucleotides: (A) P(V) morpholino nucleotide analog; (B) P(III) morpholino nucleotide analog; (C) reverse DNA P(III) deoxyribonucleotide; (D) reverse 2′ modified RNA P(III) ribonucleotide (e.g., reverse 2′-O-methyl (2′-OMe) P(III) or 2′-O-methoxyethyl (2′-MOE) P(III) ribonucleotides); (E) reverse P(III) locked nucleic acid (LNA); and (F) reverse 2′-fluoro P(III) ribonucleotide.

FIG. 18. A schematic of a method for processing and purifying a hybrid oligonucleotide.

FIG. 19. A schematic showing an example of a hybrid gapmer oligonucleotide on solid support with PMO containing 6′ and 3′ flanking regions and a central DNA-PS gap region. Regions synthesized by hand as well as automated fashion are marked above the sequence.

FIG. 20. A graph showing the mRNA expression levels of DMPK after 15 μM dose of PMO/ASO hybrid oligonucleotides generated in a first library compared to a positive, a negative control, and no treatment (NT). Oligonucleotides were delivered by electroporation.

FIG. 21. A graph showing the mRNA expression levels of DMPK after dosing with 20 mer ASO/PMO hybrid oligonucleotides at 15 μM Oligonucleotides were delivered by electroporation.

FIG. 22. A graph showing DMPK expression in HeLa cells after lipofectamine promoted uptake of PMO/ASO hybrid oligonucleotides at 100 nM.

FIG. 23. A graph showing DMPK expression in HeLa cells after lipofectamine promoted uptake of PMO/ASO hybrid oligonucleotides at 100 nM.

FIG. 24. A graph showing DMPK expression in HeLa cells after free uptake of PMO/ASO hybrid oligonucleotides at 100 μM.

FIG. 25. A graph showing DMPK expression in HeLa cells after treatment with PMO/ASO hybrid oligonucleotides, ASO gapmers and Endosomal escape vehicle (EEV) conjugated gapmers (lipofectamine and free uptake).

FIG. 26. A graph showing exon skipping efficacy by RT-PCR of mixed backbone EGFP PMOs oligonucleotides:

Oligo 49:
5′-GCTATTACCTTAACCCAG-3′,
Oligo 50:
5′-GCTATTACCTTAACCCAG-3′,
Oligo 51:
5′-GCTATTACCTTAACCCAG-3′,
Oligo 52:
5′-GCTATTACCTTAACCCAG-3′,
Oligo 53
5′-GCTATTACCTTAACCCAG-3′,
and
control sequences:
Oligo 48:
5′-GCTATTACCTTAACCCAG-3′
and
Oligo 54:
5′-GCTATTACCTTAACCCAG-3′;
where ATCG indicate PMO P(V),
ATCG indicates 2′-OMe ribonucleotides

Oligo 50 shows 66% exon skipping and Oligo 53 shows 8% exon skipping, controls show 37-40% exon skipping. Nucleofection was used to deliver oligonucleotides to HeLa eGFP cells.

FIG. 27. A graph showing viability for Hela and HepG2 cells exposed to conventional gapmer (Oligo 14): 5′-ACAGACAATAAATACCGAGG-3′, PMO gapmer (Oligo 17): 6′-ACAGACAATAAATACCGAGG-3′, and PMO gapmer (Oligo 55): 6′-ACAGACAATAATACCGAGG-3′, where ATCG indicate PMO, ATCG indicates DNA, bold underlined 2′-OMOE RNA. PMO gapmer (Oligo 17) does not show any toxicity up to 1 mM concentration. Conventional gapmer (Oligo 14) shows toxicity above 100 mM in both cell lines.

FIG. 28. A gel showing RNase H digestion activity when a fluorogenic RNA is duplexed with a ASO-PMO hybrid oligonucleotide as compared to a conventional gapmer control and PMO control.

FIG. 29. A gel showing concentration dependence of RNase H digestion activity when a fluorogenic RNA is duplexed with ASO-PMO hybrid oligonucleotides Oligo 11 (5′-ACAGACAATAAATACCGAGG-3′) and Oligo 55 (6′-ACAGACAATAAATACCGAGG-3′) as compared to a conventional gapmer control.

FIG. 30. A gel showing RNase H digestion activity when a fluorogenic RNA is contacted with different ASO-PMO hybrid oligonucleotides.

FIG. 31. HPLC trace results (IEX-HPLC) of a snake venom phosphodiesterase (SVDPE) assay using a control gapmer (0053).

FIG. 32. HPLC traces showing that ASO-PMO hybrid oligonucleotides are stable in the presence of snake venom phosphodiesterase (SVDPE). Oligo 3: 5′-ACAGACAATAAATACGAGG-3′; Oligo 6: 5′-ACAGACAATAAATACCGAGG-3′ (ATCG indicate PMO, ATCG indicates DNA, underlined 2′-OMe RNA, bold underlined 2′-OMOE RNA).

FIG. 33. HPLC traces showing that PMO-ASO (Oligo 21) is stable in phosphodiesterase U digestion.

FIG. 34. HPLC traces showing that phosphodiesterase II digestion with control oligonucleotides having a phosphodiester (PO) and phosphothioate (PS) backbones.

FIG. 35. HPLC trace results (IEX-HPLC) of a snake venom phosphodiesterase (SVDPE) assay using a control gapmer with a phosphorothioate (PS) backbone (Oligo 37).

FIG. 36. A schematic comparing backbones of thiomorpholino oligonucleotides (TMO) and their DNA hybrids to oligonucleotides in the current invention.

FIG. 37. Structures of P(III) PMO suitable for use in transitioning between ASO and PMO monomeric subunits.

FIG. 38. A graph showing exon skipping efficacy by RT-PCR of mixed backbone EGFP PMOs with P-S linkage; Oligo 55: 5′-GCTATTACCTTAAC\vec{CA}CG-3′, Oligo 56: 5′-GCTATTACCTTAACCCAG-3′, morpholino control (Oligo 57) 5′-GCTATTACCTTAACCCAG-3′ and their conjugates with EEV-3 (Ac-PKKKRKV-PEG₂—K(cyclo[FGFGRGRQ])-PEG₁₂—OH): Oligo 55-EEV3 and Oligo 56-EEV3 and Oligo 57-EEV3; where ATCG indicate PMO P(V), ATCG indicates PMO P(III), Oligo 55 shows higher efficacy than Oligo 56. Addition of EEV3 to PMOs seems to improve the efficacy as observed for Oligo 55-EEV3 and Oligo 56-EEV3. HeLa eGFP cells were treated (free-uptake) with the 50 μM formulations in a 24 well plate for a duration of 24 hrs, n=3. Cell density of 0.1E+06 cells/well.

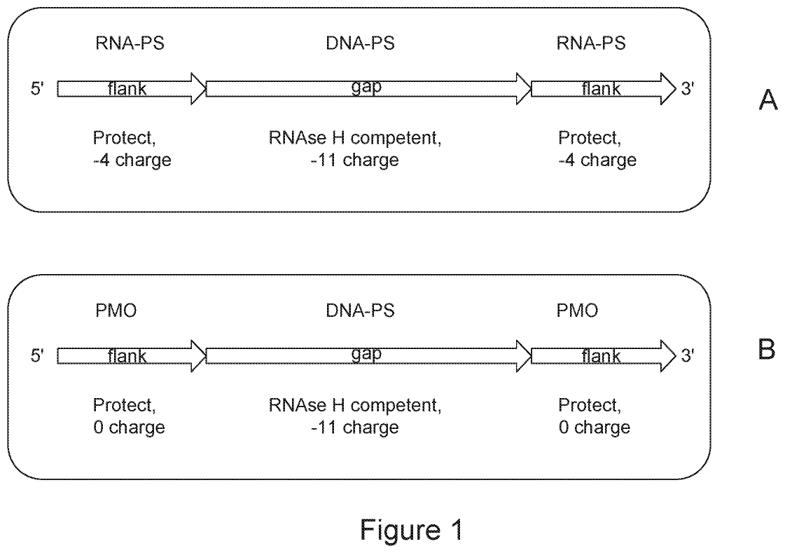

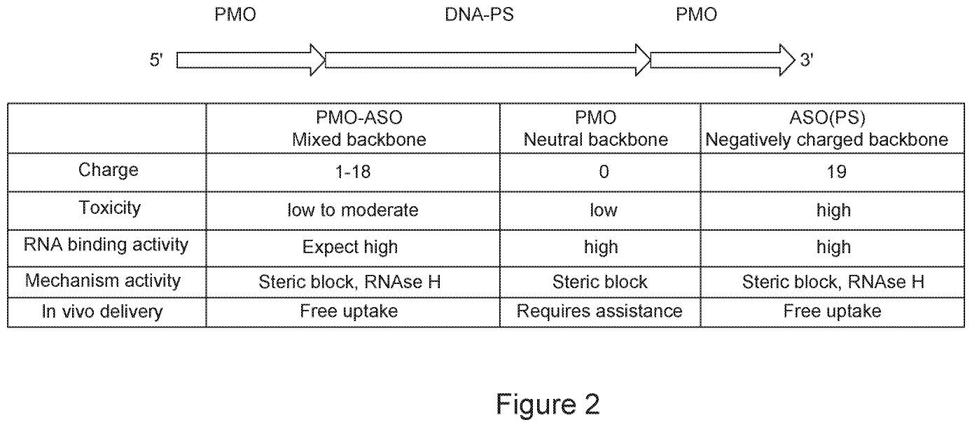

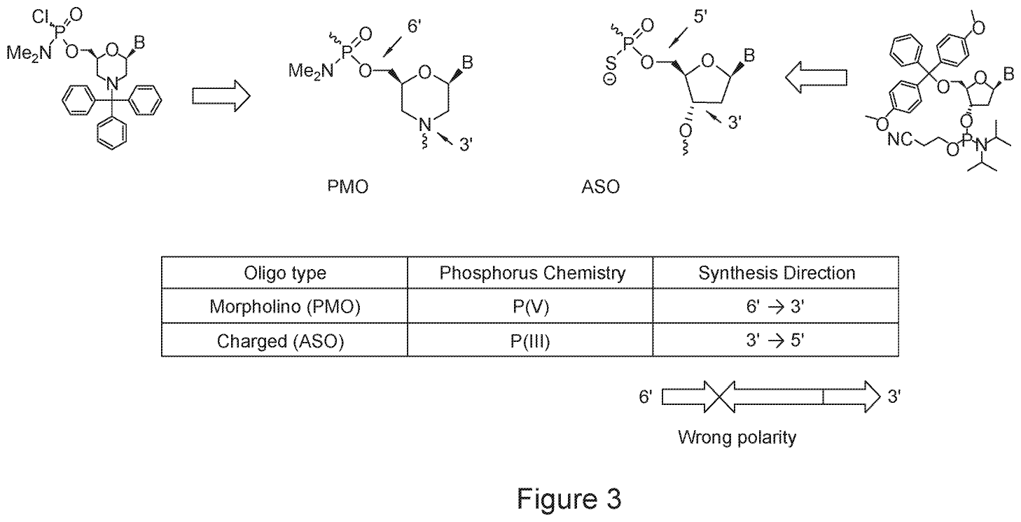

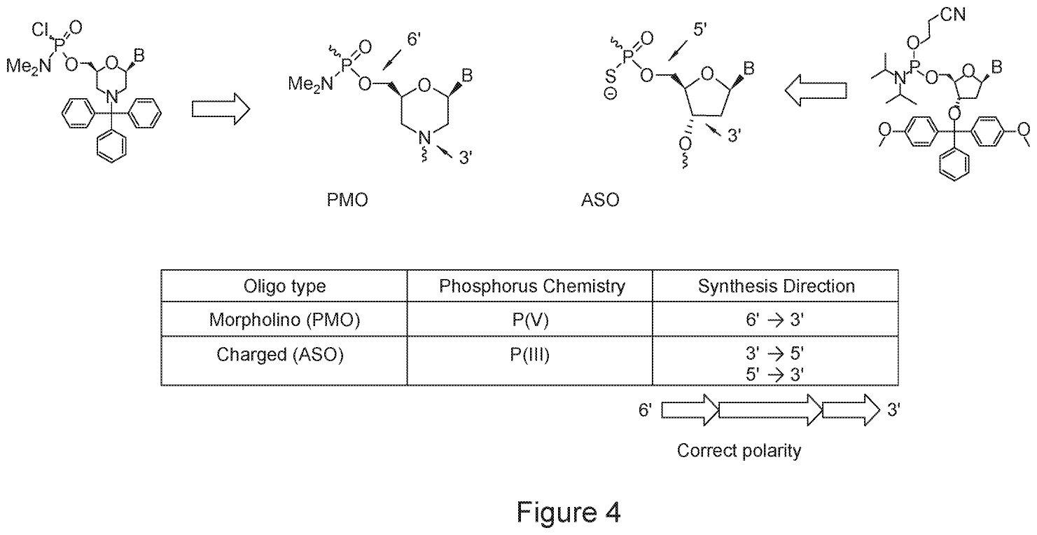

### DETAILED DESCRIPTION

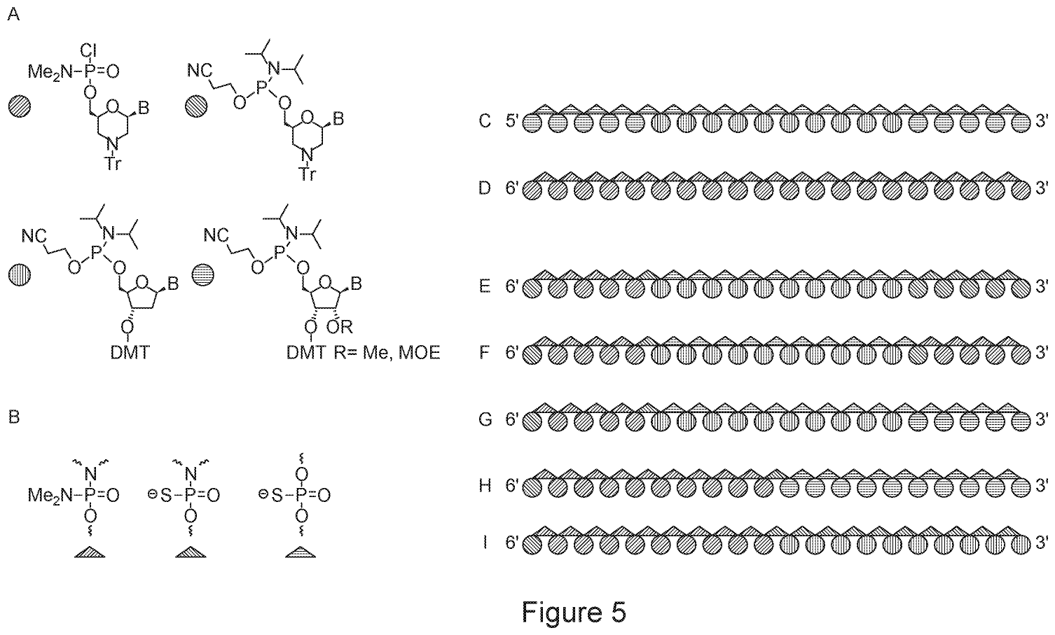

While not wishing to be bound by theory, it is believed that the disclosed hybrid oligonucleotides have advantages, for example, relative to phosphorothioate (PS) only oligonucleotides or phosphorodiamidate (PMO) only oligonucleotides, including but not limited to, decreased toxicity, increased uptake of free (unformulated) oligonucleotide, for example, as a result of the decrease in charge of the hybrid oligonucleotides and stabilization of the oligonucleotide ends from enzyme degradation. The properties of hybrid oligonucleotides with PMOs as compared to oligonucleotides with a phosphorothioate (PS) only backbone or phosphoro morpholino oligonucleotides (PMOs) is shown in FIG. 5. The hybrid oligonucleotides are useful for treating a variety of genetic diseases, including, but not limited to, cancer, immune diseases or disorder, and neuromuscular diseases or disorders

### Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

As used herein “oligonucleoside” refers to an oligonucleotide in which the internucleotidic linkages do not contain a phosphorus atom.

As used herein, the term “oligonucleotide” refers to an oligomeric compound comprising a plurality of linked nucleotides or nucleosides. The linkages in the “oligonucleotide” may or may not contain a phosphorous atom. In embodiments, oligonucleotides are composed of natural and/or modified nucleobases, sugars, and covalent internucleotidic linkages. In embodiments, the oligonucleotide comprises one or more P(V) morpholino nucleotide analogs; one or more P(III) morpholino nucleotide analogs; and one or more deoxyribonucleotide (DNA) or ribonucleotide (RNA) analogs, including, but not limited to: DNA P(III) nucleotides, reverse DNA P(III) nucleotides, RNA P(III) nucleotides, reverse RNA P(III) nucleotides, P(V) morpholino nucleotide analogs, P(III) morpholino nucleotide analogs, or combinations thereof. In embodiments, the RNA nucleotide is a 2′ modified RNA nucleotide. In embodiments, the oligonucleotide comprises one or more of phosphorus containing internucleotidic linkages selected from: phosphoroamidate, phosphorodiamidate, phosphorothioamidate, phosphorothioate, phosphodiester, phosphotriesters, methylphosphonates and combinations thereof. In embodiments, the oligonucleotide comprises one or more non-phosphorus containing internucleoside linkages selected from: methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); N,N′-dimethylhydrazine (—CH₂—N(CH₃)—N(CH)—), and combinations thereof. In embodiments, the oligonucleotide comprises one or more phosphorous containing internucleotidic linkages and one or more non-phosphorous containing internucleotidic linkages.

As used herein, the term “2′-modified” nucleotide refers to a nucleotide in which the ribose sugar comprises a substituent at the 2′ position other than H or OH. 2′-modified monomers, include, but are not limited to, BNA's and monomers (e.g., nucleosides and nucleotides) with 2′-substituents, such as allyl, amino, azido, thio, O-allyl, O—C₁-C₁₀ alkyl, —OCF₃, O—(CH₂)₂O—CH₃, 2′—O(CH₂)₂SCH₃, O(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. Examples of 2′ modified monomers include, but are not limited to, 2′-O-methyl (2′-OMe) P(III) RNA, reverse 2′-O-methyl (2′-OMe) P(III) RNA, 2′-O-methoxyethyl (2′-OMOE) P(III) RNA, reverse 2′-O-methoxyethyl (2′-OMOE) P(III) RNA, 2′-fluoro (2′-F) P(III) RNA, reverse 2′-fluoro (2′-F) P(III) RNA, 2′-O-ethyl (cET) P(III) nucleotides, reverse 2′O-Ethyl (cET) P(III) nucleotides, or combinations thereof.

As used herein the term “hybrid oligonucleotide” refers to an oligonucleotide that contains at least one nucleotide, nucleoside, internucleotidic linkage, or a combination thereof that is(are) different compared to at least one other nucleotide(s), nucleoside(s) or internucleotidic linkage(s) within the same oligonucleotide. In embodiments, the hybrid oligonucleotide includes a different nucleoside in an isolated position. In embodiments, the hybrid oligonucleotide includes different nucleosides that are grouped together in regions. In embodiments, the grouped nucleosides define a particular motif. As used herein, the term “mixed-backbone oligonucleotide” refers to an oligonucleotide wherein at least one internucleotidic linkage is different from at least one other internucleotidic linkage of the oligonucleotide.

As used herein, the term “gapmer” refers to a hybrid oligonucleotide that includes a central gap region that supports RNAse H activity and upstream and downstream flanking regions (also referred to as “wings”). In embodiments, the upstream and downstream flanking regions increase oligonucleotide stability and affinity of the gapmer for its target nucleic acid sequence. A “stereorandom gapmer” is a gapmer that possesses a mixture of (R) or (S) configurations at each of its stereocenters. In some embodiments a stereorandom gapmer is a product from elongation reactions with morpholino or deoxyribonucleoside monomers. A “stereodefined gapmer” is a gapmer that possesses (R) or (S) stereochemical configurations at each of its stereocenters, wherein the configurations are controlled. A stereodefined gapmer may be a product from streospecific elongation reactions with stereopure morpholino or deoxyribonucleoside monomers, wherein the phosphorus stereochemistry of the gapmer is controlled as a sequence of defined stereochemical (R) or (S) configurations.

The terms “miniPEG”, “PEG2” and “AEEA” are used interchangeably herein to refer to 2-[2-[2-aminoethoxy]ethoxy]acetic acid.

A “PMO-gapmer” is a gapmer including flank regions comprising morpholino monomers linked to each other by phosphorodiamidate bonds. Flank regions can also be referred to as wing regions.

“Stereorandom” when referring to a reaction means that a reaction has been conducted without preference for a resulting stereochemistry.

“R” and “S” are terms describing isomers are descriptors of the stereochemical configuration at asymmetrically substituted atoms, including but not limited to; carbon, sulfur, phosphorus and quaternary nitrogen. The designation of asymmetrically substituted atoms as “R” or “S” is done by application of the Cahn-Ingold-Prelog priority rules, as are well known to those skilled in the art, and described in the International Union of Pure and Applied Chemistry (TUPAC) Rules for the Nomenclature of Organic Chemistry. Section E, Stereochemistry.

As used herein, the terms “upstream” and “downstream” refer to relative positions in an oligonucleotide. Naturally occurring oligonucleotides have a 5′ end and a 3′ end, named for the carbon position on the 5-membered deoxyribose (or ribose) ring. By convention, upstream refers to a relative position towards the 5′ end of the oligonucleotide and downstream refers to a relative position towards the 3′ end of the oligonucleotide. For oligonucleotides that include nucleotides comprising a 6-membered ring, such as a morpholino nucleotide analog, the “upstream” end of the oligonucleotide is referred to interchangeably herein as either the 6′ end or the 5′ end. The 3′ end of oligonucleotides that include morpholino ring is the downstream end.

As used herein, the term “nucleobase” refers to portion of a nucleoside or nucleotide that is capable of hydrogen bonding to a nucleobase of another nucleic acid. A nucleobase can comprise any atom or group of atoms capable of hydrogen bonding. In embodiments, the nucleobase is a nitrogenous base. A natural nucleobase is a nucleobase that is unmodified from its naturally occurring form found in RNA or DNA and include, for example, cytosine (C), guanine (G), adenine (A), thymine (T) (in DNA) or uracil (U) (in RNA). Modified DNA nucleobases include 5-methylcytidine (5mC). Modified RNA nucleobases include, but are not limited to, pseudouridine (Ψ), dihydrouridine (D), inosine (I), ribothymidine (rT) and 7-methylguanosine (m7G).

As used herein, the term “nucleoside” refers to a molecule comprising a nucleobase and a sugar but lacking a phosphate group. Nucleosides include, but are not limited to, natural nucleosides, abasic nucleosides, modified nucleosides, and nucleosides having mimetic bases and/or sugar groups. A “natural nucleoside” or “unmodified nucleoside” is a nucleoside comprising a natural nucleobase and a natural sugar. Natural nucleosides include RNA and DNA nucleosides.

As used herein, the term “nucleotide” refers to a molecule comprising a nucleobase, a sugar, and an internucleotidic linkage. In embodiments the internucleotidic linkage comprises a phosphate group. Nucleotides may be modified with any of a variety of substituents, and can include, for example, a modified nucleobase, a modified sugar, a modified phosphate group, or a combination thereof. Modified sugars are sugar moieties that are modified from its naturally occurring form found in RNA (2′—OH) or DNA (2′-H) and include, but are not limited to a ribose sugar having a 2′ modification and nucleotides in which the sugar moiety is replaced with an analog such as a morpholine ring. In embodiments, a phosphate group can be linked to the 2′, 3′ or 5′ hydroxyl moiety of a sugar. In embodiments, when the modified nucleoside includes a six membered ring, such as a morpholino ring, the phosphate group can be linked to the 2′, 3′ or 6′ hydroxyl moiety. A modified nucleotide can be referred to herein as a “nucleotide analog”, e.g., a modified deoxyribonucleotide can be referred to as deoxyribonucleotide analog and a modified ribonucleotide can be referred to as a ribonucleotide analog.

As used herein, the term “heterocyclic base moiety” refers to a nucleobase comprising a heterocycle.

As used herein “internucleotidic linkage” refers to a covalent linkage between adjacent nucleosides. Naturally occurring DNA and RNA include 3′ to 5′ phosphodiester (PO) internucleotidic linkages. Modified internucleotidic linkages, compared to natural phosphodiester linkages, can be used to alter, for example, increase nuclease resistance of the oligonucleotide. The two main classes of internucleoside linking groups are defined by the presence or absence of a phosphorus atom. Representative phosphorus containing internucleotidic linkages include, but are not limited to, phosphodiesters, phosphotriesters, methylphosphonates, phosphorothioamidate phosphoroamidate, phosphorothioamidates, and phosphorothioates. Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). In embodiments, the non-naturally occurring internucleotidic linkages include, but are not limited to, phosphorodiamidate, phosphorothioamidate, phosphothioate (PS) and phosphoryl guanidine linkages.

“Morpholino” or “morpholino nucleotide analog” refers to a modified nucleotide analog that includes a base-pairing nucleobase (Pi), for example, a puridine or pyrimidine base-pairing nucleobase, in which the sugar molecule of the nucleotide is replaced with a morpholine ring as shown below:

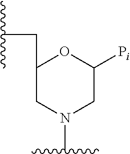

A “morpholino oligonucleotide” refers to a polymeric molecule that is capable of hydrogen bonding to a target nucleic acid sequence, wherein the polymer contains at least one morpholino nucleotide analog that is coupled to an adjacent nucleotide or nucleotide analog through the nitrogen in the morpholine ring. In embodiments, the morpholino oligomer comprises morpholino nucleotide analogs that are linked by (thio)phosphorodiamidate linkages, in which the morpholino nitrogen of one subunit is linked to a 5′ or 6′ carbon of an adjacent nucleotide or nucleotide analog. Morpholino oligomers are described, for example, in U.S. Pat. Nos. 5,698,685, 5,217,866, 5,142,047, 5,034,506, 5,166,315, 5,185,444, 5,521,063, and 5,506,337, each of which are each incorporated by reference herein in their entireties.

A “phosphorodiamidate” group comprises phosphorus having two attached oxygen atoms and two attached nitrogen atoms, and herein may also refer to phosphorus having one attached oxygen atom and three attached nitrogen atoms. In intersubunit linkages (e.g., between a flank region and a gapmer) of the oligomers described herein, one nitrogen is typically pendant to the backbone chain, and the second nitrogen is the ring nitrogen in a morpholino ring structure, as shown in formula II below. Alternatively, or in addition, a nitrogen may be present at the 5′-exocyclic carbon, as shown in formulas III and IV below.

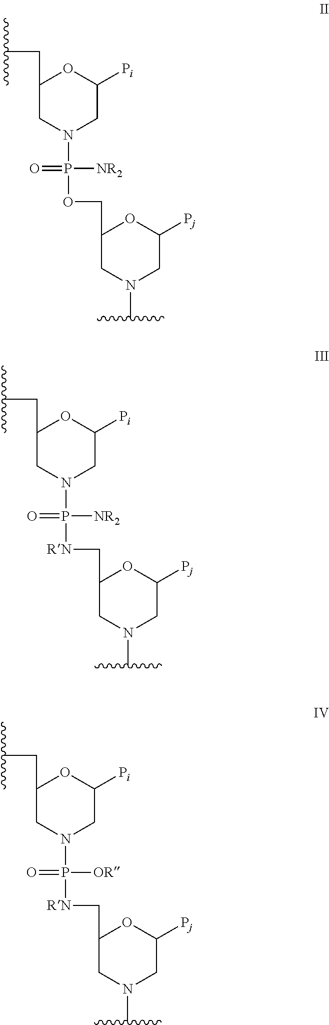

In a thiophosphorodiamidate linkage, one oxygen atom, typically an oxygen pendant to the backbone in the oligomers described herein, is replaced with sulfur.

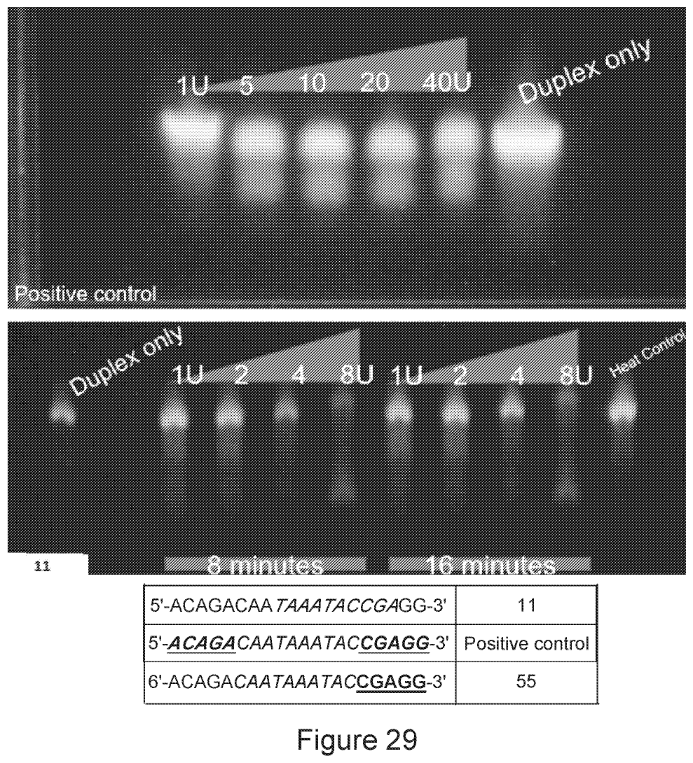

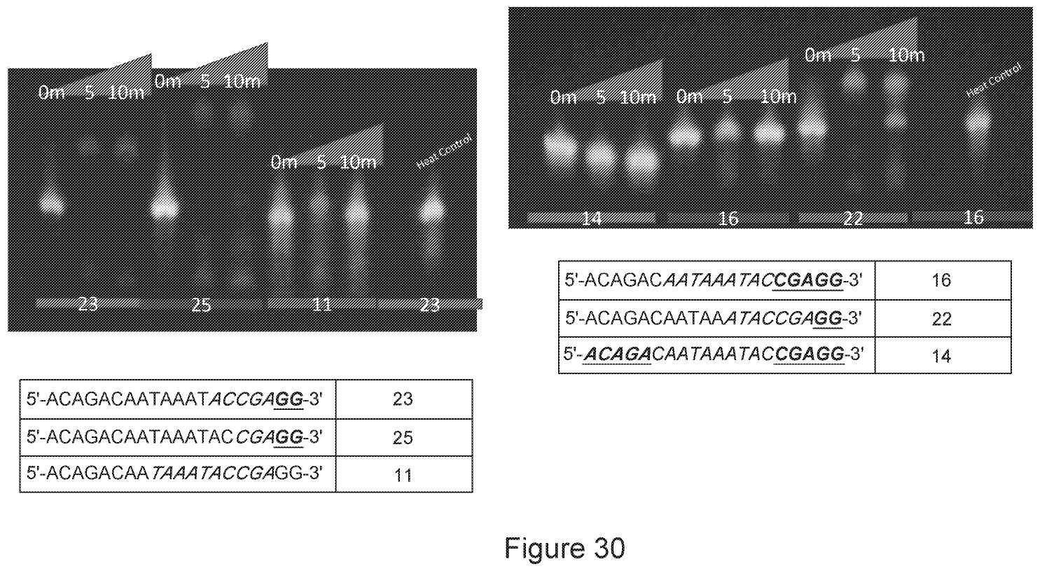

As used herein, a “P(V)” nucleotide is a nucleotide in which the phosphorous group has five bonds. Examples of P(V) nucleotides include, but are not limited to P(V) PMO. See, for example, FIGS. 29, 30. P(V) nucleotides include nucleotides in which the phosphorous group is attached to the 5′ or 6′ carbon of the sugar (or sugar analog) and also nucleotides in which the phosphorous group is attached to the 3′ carbon of the sugar (or sugar analog). P(V) nucleotide in which the phosphorous group is attached to the 3′ carbon of the sugar (or sugar analog) is referred to herein as a “reversed P(V) nucleotide.”

As used herein, a “P(III)” nucleotide is a nucleotide in which the phosphorous group has 3 bonds. Examples of P(III) nucleotides include, but are not limited to: P(III) PMO (FIGS. 29,30), P(III) DNA, and P(III) RNA (FIGS. 29,31). P(III) nucleotides include nucleotides in which the phosphorous group is attached to the 5′ or 6′ carbon of the sugar (or sugar analog) and also nucleotides in which the phosphorous group is attached to the 3′ carbon of the sugar (or sugar analog). P(III) nucleotide in which the phosphorous group is attached to the 3′ carbon of the sugar (or sugar analog) is referred to herein as a “reversed P(III) nucleotide.”

As used herein the term “mimetic” refers to groups that are substituted for a sugar, a nucleobase, and/or internucleotidic linkage in an oligonucleotide. Generally, a mimetic is used in place of the sugar or sugar-internucleotidic linkage combination, and the nucleobase is maintained for hybridization to a selected target. Representative examples of a sugar mimetic include, but are not limited to, cyclohexenyl or morpholino. Representative examples of a mimetic for a sugar-internucleotidic linkage combination include, but are not limited to, peptide nucleic acids (PNA) and morpholino groups linked by uncharged achiral linkages. In some instances, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

As used herein, the term “bicyclic nucleoside” or “BNA” refers to a nucleoside in which the furanose portion of the nucleoside includes a bridge connecting two atoms on the furanose ring, thereby forming a bicyclic ring system. BNAs include, but are not limited to, α-L-LNA, β-D-LNA, ENA, Oxyamino BNA (2′-O—N(CH₃)—CH₂-4′) and Aminooxy BNA (2′-N(CH)—O—CH₂-4′).

As used herein, the term “4′ to 2′ bicyclic nucleoside” refers to a BNA wherein the bridge connecting two atoms of the furanose ring bridges the 4′ carbon atom and the 2′ carbon atom of the furanose ring, thereby forming a bicyclic ring system.

As used herein, a “locked nucleic acid” or “LNA” refers to a nucleotide modified such that the 2′-hydroxyl group of the ribosyl sugar ring is linked to the 4′ carbon atom of the ribosyl sugar ring via a methylene groups, thereby forming a 2′-C,4′-C-oxymethylene linkage. LNAs include, but are not limited to, α-L-LNA, and β-D-LNA.

A “solid-phase-supported morpholino subunit” refers to the first or any subsequent morpholino subunit monomer incorporated into a morpholino oligomer by solid-phase stepwise synthesis as described herein. The subunit is attached to the solid support, or to a growing oligomer chain on the solid support, via its 5′ (or 6′) exocyclic carbon. “Base-protected” refers to protection of the base-pairing groups, e.g., purine or pyrimidine bases, on the morpholino subunits with protecting groups suitable to prevent reaction or interference of the base-pairing groups during stepwise oligomer synthesis.

As used herein, the term “nucleobase complementarity” refers to the ability of a nucleobase to base pair with another nucleobase. For example, in DNA, adenine (A) is complementary to thymine (T) and, in RNA, adenine (A) is complementary to uracil (U). In embodiments, complementary nucleobase refers to a nucleobase of an oligonucleotide that is capable of base pairing with a nucleobase of its target nucleic acid. For example, if a nucleobase at a certain position of an oligonucleotide is capable of hydrogen bonding with a nucleobase at a certain position of a target nucleic acid, then the position of hydrogen bonding between the oligonucleotide and the target nucleic acid is considered to be complementary at that nucleobase pair.

As used herein, the term “non-complementary nucleobase” refers to a pair of nucleobases that do not form hydrogen bonds with one another or otherwise support hybridization.

As used herein, the term “complementary” refers to the capacity of an oligomeric compound to hybridize to another oligomeric compound or nucleic acid through nucleobase complementarity. In embodiments, an oligonucleotide and its target are complementary to each other when a sufficient number of corresponding positions in each molecule are occupied by nucleobases that can bond with each other to allow stable association between the oligonucleotide and the target. One skilled in the art recognizes that the inclusion of mismatches is possible without eliminating the ability of the oligomeric compounds to remain in association. Therefore, described herein are oligonucleotides that may comprise up to about 20% nucleotides that are mismatched (i.e., are not nucleobase complementary to the corresponding nucleotides of the target). In embodiments, the oligonucleotides contain no more than about 15%, no more than about 10%, no more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% nucleobase complementary to a target nucleic acid.

As used herein, “hybridization” refers to the pairing of complementary oligomeric compounds (e.g., an oligonucleotide and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine (A) is nucleobase complementary to the natural nucleobases thymidine (T) and uracil (U) which pair through the formation of hydrogen bonds. The natural base guanine (G) is nucleobase complementary to the natural bases cytosine (C) and 5-methyl cytosine (5mC). Hybridization can occur under varying circumstances.

As used herein, the term “specifically hybridizes” refers to the ability of an oligomeric compound to hybridize to one nucleic acid sequence with substantially greater affinity than it hybridizes to another nucleic acid sequence. In embodiments, an oligonucleotide specifically hybridizes to more than one target site. In embodiments, an oligonucleotide specifically hybridizes with its target under stringent hybridization conditions.

As used herein, the term “sequence identity”, when used in the context of two oligonucleotide, refers to the percentage of residues between the two sequences that are the same and in the same relative position. As such, one sequence has a certain percentage of sequence identity compared to another sequence. For sequence comparison, typically one sequence acts as a reference sequence, to which test sequences are compared. Those of ordinary skill in the art will appreciate that two sequences are generally considered to be “substantially identical” if they contain identical residues in corresponding positions. In embodiments, the sequence identity may be determined using the Needleman-Wunsch algorithm (Needleman and Wunsch, 1970, J. Mol. Biol. 48. 443-453) as implemented in the Needle program of the EMBOSS package (EMBOSS: The European Molecular Biology Open Software Suite, Rice et al., 2000, Trends Genet. 16: 276-277), in the version that exists as of the date of filing. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows. (Identical Residues×100)/(Length of Alignment−Total Number of Gaps in Alignment).

In other embodiments, sequence identity can also be determined using the Smith-Waterman algorithm, in the version that exists as of the date of filing.

As is well known in this art, sequences may be compared using any of a variety of algorithms, including those available in commercial computer programs such as BLASTN, BLASTP, gapped BLAST, and PSI-BLAST, in existence as of the date of filing. Exemplary such programs are described in Altschul, et al., Basic local alignment search tool, J. Mol. Biol., 215(3): 403-410, 1990: Altschul, et al., Methods in Enzymology: Altschul, et al., “Gapped BLAST and PSI-BLAST: a new generation of protein database search programs”, Nucleic Acids Res. 25:3389-3402, 1997; Baxevanis, et al., Bioinformatics A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology.

The terms “pre-mRNA” and “primary transcript” as used herein refer to a newly synthesized eukaryotic mRNA molecule directly after DNA transcription. A pre-mRNA is typically capped with a 5′ cap, modified with a 3′ poly-A tail, and spliced to produce a mature mRNA sequence.

As used herein, the terms “target nucleic acid” and “target nucleic acid sequence” refer to the nucleic acid sequence to which the hybrid oligonucleotide binds or hybridizes. Target nucleic acids include, but are not limited to, RNA (including, but not limited to mRNA, including pre-mRNA, mature mRNA, or portions thereof), genomic DNA, cDNA derived from such RNA, as well as non-translated RNA, such as miRNA. In embodiments, a target nucleic acid can be a cellular gene (or mRNA transcribed from such gene) whose expression is associated with a particular disorder or disease state, or a nucleic acid molecule from an infectious agent.

As used herein, the terms “splicing” and “processing” refer to the modification of a pre-mRNA following transcription, in which introns are removed and exons are joined. Splicing occurs in a series of reactions that are catalyzed by a large RNA-protein complex composed of five small nuclear ribonucleoproteins (snRNPs) referred to as a spliceosome. Within an intron, a 3′ splice site, a 5′ splice site, and a branch site are required for splicing. The RNA components of snRNPs interact with the intron and may be involved in catalysis

The “target pre-mRNA” is the pre-mRNA comprising the target nucleic acid sequence to which the oligonucleotide hybridizes.

The “mature target mRNA” is the mRNA sequence resulting from splicing of the target pre-mRNA sequence. In embodiments, the mature target mRNA encodes a functional protein. In embodiments, the mature target mRNA does not encode a functional protein. In embodiments, the mature target mRNA retains one or more intron sequences.

The term “target gene” refers to the gene that encodes the target pre-mRNA.

The “target protein” refers to the amino acid sequence encoded by the target mRNA. In embodiments, the target protein may not be a functional protein.

As used herein, the term “expression” refers to all the functions and steps by which a gene's coded information is converted into structures present and operating in a cell. Such structures include, but are not limited to the products of transcription and translation, such as proteins.

“Wild type target protein” refers to a native, functional protein isomer produced by a wild type, “normal,” or unmutated version of the target gene.

A “re-spliced target protein”, as used herein, refers to the protein encoded by the mRNA resulting from the splicing of the target pre-mRNA to which the oligonucleotide hybridizes. Re-spliced target protein may be identical to a wild type target protein, may be homologous to a wild type target protein, may be a functional variant of a wild type target protein, or may be an active fragment of a wild type target protein.

As used herein, the term “modulation” refers to a perturbation of function or activity when compared to the level of the function or activity prior to modulation. For example, modulation includes the change, either an increase (stimulation or induction) or a decrease (inhibition or reduction) in gene expression.

The terms “inhibit”, “inhibiting” or “inhibition” refer to a decrease in an activity, expression, function or other biological parameter and can include, but does not require complete ablation of the activity, expression, function or other biological parameter. Inhibition can include, for example, at least about a 10% reduction in the activity, response, condition, or disease as compared to a control. In embodiments, expression, activity or function of a gene or protein is decreased by a statistically significant amount. In embodiments, activity or function is decreased by at least about 10%, about 20%, about 30%, about 40%, about 50%, and up to about 60%, about 70%, about 80%, about 90% or about 100%.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred to.

As used herein, “treat,” “treating,” “treatment” and variants thereof, refers to any administration of the disclosed compounds that partially or completely alleviates, ameliorates, relieves, inhibits, delays onset of, reduces severity of, and/or reduces incidence of one or more symptoms or features of a disease as described herein. In reference to a patient, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to an amount of the disclosed compound and/or composition that is sufficient to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and/or animals without excessive toxicity, irritation, allergic response, or other problems or complications commensurate with a reasonable benefit/risk ratio.

As used herein, the term “cap structure” or “terminal cap moiety” refers to chemical modifications, which have been incorporated at either end of an oligonucleotide.

As used herein, “subject” refers to an individual to be treated and can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

### Antisense Oligonucleotide (ASO) Mechanism of Action

In embodiments, the hybrid oligonucleotide is a single stranded oligonucleotide. In embodiments, the hybrid oligonucleotide is a double stranded oligonucleotide duplex comprising a sense and an antisense strand. In embodiments, the sense and antisense strands of the double stranded oligonucleotide duplex are the same length (i.e., there is no overhang on either end of the duplex). In embodiments, the sense and antisense strands of the double stranded oligonucleotide duplex are not the same length (i.e., there is an overhang at one or more of the 3′ end of the sense strand, the 5′ end of the sense strand, the 3′ end of the antisense strand and the 5′ end of the antisense strand).

In embodiments, the hybrid oligonucleotide is complementary to a target nucleic acid sequence. In embodiments, the hybrid oligonucleotide has a sequence that is complementary to a target nucleic acid sequence. In embodiments, the hybrid oligonucleotide is fully complementary to the target nucleic acid sequence. In embodiments, the hybrid oligonucleotide is not fully complementary to the target nucleic acid sequence (i.e., the hybrid oligonucleotide sequence may contain one or more mismatches). The target nucleic acid sequence can be a coding sequence or a non-coding sequence. In embodiments, the target nucleic acid sequence comprises DNA. In embodiments, the target nucleic acid sequence comprises chromosomal DNA. In embodiments, the target nucleic acid sequence comprises RNA. In embodiments, the target nucleic acid sequence is in a mRNA. In embodiments, the target nucleic acid sequence is in a pre-mRNA. In embodiments, the target nucleic acid sequence is in a mature mRNA. In embodiments, the hybrid oligonucleotide hybridizes to the target nucleic acid sequence by Watson-Crick base pairing.

In embodiments, hybridization of the hybrid oligonucleotide to its target nucleic acid sequence modulates one or more aspects of protein transcription, translation, and expression. In embodiments, hybridization of the hybrid oligonucleotide to its target nucleic acid sequence blocks access to the target nucleic acid sequence by cellular machinery, for example, splicing factors. In embodiments, hybridization of the hybrid oligonucleotide to its target nucleic acid sequence results in degradation of the target oligonucleotide, for example, degradation of an mRNA transcript via RNase H. Other mechanisms are known and are reviewed in by Agrawal, S. (1996) “Antisense Oligonucleotides: towards clinical trials.” Trends Biotechnol. 14(10):376-387.

In embodiments, hybridization of the hybrid oligonucleotide to a target nucleic acid sequence suppresses expression of the target protein. In embodiments, the hybridization of the hybrid oligonucleotide to a target nucleic acid sequence downregulates expression of one or more wild type target protein isomers. In embodiments, the hybridization of the hybrid oligonucleotide to a target nucleic acid sequence upregulates expression of the target protein. In embodiments, the hybridization of the hybrid oligonucleotide to a target nucleic acid sequence increases expression of one or more wild type target protein isomers.

In embodiments, the hybrid oligonucleotide stabilizes a target oligonucleotide, for example, a target mRNA. In embodiments, the hybrid oligonucleotide increases the half-life of the target oligonucleotide. In embodiments, the hybrid oligonucleotide increases the half-life of a target mRNA. In embodiments, the hybrid oligonucleotide increases expression of the protein product of a target mRNA.

In embodiments, a hybrid oligonucleotide is an antisense oligonucleotide (ASO). In embodiments, the hybrid oligonucleotide is a steric block oligonucleotide, a gapmer, a splice switching oligonucleotide, an exon skipping oligonucleotide, microRNA (miRNA), antagomir, aptamer, ribozyme, immunostimulatory oligonucleotide, decoy oligonucleotide, miRNA mimic, miRNA inhibitor, or U1 adaptor. In embodiments, the hybrid oligonucleotide is a sense strand of an siRNA. In embodiments, the hybrid oligonucleotide is an antisense strand of an siRNA.

The efficacy of the hybrid oligonucleotide may be assessed by evaluating any detectable and/or measurable activity attributable to the hybridization of the hybrid oligonucleotide to its target nucleic acid. Detection, measuring or both, may be direct or indirect. In embodiments, activity is assessed by detecting, measuring, or both, the amount of target mRNA. In embodiments, activity is assessed by detecting, measuring, or both, the amount of re-spliced mRNA. In embodiments, activity is assessed by detecting, measuring, or both, the amount of target protein. In embodiments, activity is assessed by detecting, measuring, or both, the amount of a target protein isomer.

### Seric Block

In embodiments, the hybrid oligonucleotide is a steric-blocking antisense oligonucleotide (ASO). In embodiments, transcription, translation, or expression of a target protein is modulated through steric blocking. See, e.g., Scharner and Aznarez (2020) “Clinical Applications of Single-Stranded Oligonucleotides: Current Landscape of Approved and In-Development Therapeutics.” Mol. Therapy. 29(2):540-554. Steric-blocking ASOs are short, synthetic, single-stranded oligonucleotides that range from about 8 to about 50, about 15 to about 30, about 20 to about 30, or about 15, 16, 17, 18, 19, 20, 21, 22, 23, 24, 25, 26, 27, 28, 29, or 30 nucleotides in length. Unlike gapmers, steric-blocking ASOs do not elicit RNase-H mediated degradation of a target nucleic acid sequence (e.g., target mRNA). As used herein “steric block” oligonucleotide refers to an oligonucleotide that binds to a target nucleic acid sequence via Watson and Crick base-pairing and hinders the binding of trans-activating factors, for example, small nuclear RNA (snRNA), microRNA (miRNA), or RNA-binding proteins, to the target oligonucleotide or prevents formation of RNA secondary structures.

### Splice Switching

In embodiments, the hybrid oligonucleotide is a splice-switching oligonucleotide (also referred to as an exon skipping oligonucleotide). Splice-switching oligonucleotides are short, synthetic, single-stranded oligonucleotides that range from about 8 to about 50, about 15 to about 30, about 20 to about 30, or about 15, 16, 17, 18, 19, 20, 21, 22, 23, 24, 25, 26, 27, 28, 29, or 30 nucleotides in length. Splice switching oligonucleotides are a type of steric blocking oligonucleotide that base-pair with a pre-mRNA and disrupt the normal splicing repertoire of the transcript by blocking the RNA-RNA base-pairing or protein-RNA binding interactions that occur between components of the splicing machinery and the pre-mRNA. In embodiments, the splice switching oligonucleotide does not elicit RNase-H mediated degradation of a target nucleic acid sequence (e.g., target mRNA).

Because splicing of pre-mRNA is required for the proper expression of most protein-coding genes, targeting the process offers a means to manipulate protein production from a gene. Splicing modulation can be used to treat diseases caused by mutations that lead to disruption of normal splicing or when interfering with the normal splicing process of a gene transcript may be therapeutic. Such antisense oligonucleotides offer an effective and specific way to target and alter splicing in a therapeutic manner. See, e.g., Havens and Hastings (2016) “Splice-switching antisense oligonucleotides as therapeutic drugs.” Nucleic Acids Res. 44(14):6549-6563.

In embodiments, hybridization of the hybrid oligonucleotide to a target nucleic acid sequence within a target pre-mRNA modulates one or more aspects of pre-mRNA splicing. In embodiments, the hybridization of the hybrid oligonucleotide causes one or more exons to be skipped (sometimes called exon skipping). In embodiments, hybridization results in increased or decreased expression or activity of a target protein and/or a downstream protein that is regulated by the target gene. In embodiments, the hybridization of the hybrid oligonucleotide to a target mRNA induces alternative splicing that leads to the addition or deletion of nucleotides in a target transcript. In embodiments, the hybridization induces alternative splicing that leads to the addition or deletion of nucleotides within a single exon of a target transcript. In embodiments, the hybridization induces alternative splicing that leads to the deletion of nucleotides within a single exon of a target transcript. In embodiments, deletion of nucleotides within a single exon results in the translation of truncated protein. In embodiments, the truncated protein is less toxic to the cells than the untruncated protein.

As used herein, modulation of splicing refers to altering the processing of a pre-mRNA transcript such that the spliced mRNA molecule contains either a different combination of exons as a result of exon skipping or exon inclusion, a deletion in one or more exons, or the deletion or addition of a sequence not normally found in the spliced mRNA (e.g., an intron sequence). In embodiments, hybridization of the hybrid oligonucleotide to a target pre-mRNA restores native splicing to a mutated pre-mRNA sequence. In embodiments, hybridization results in alternative splicing of the target pre-mRNA. In embodiments, hybridization results in exon inclusion or exon skipping of one or more exons. In embodiments, the skipped exon sequence comprises a frameshift mutation, a nonsense mutation, or a missense mutation. In embodiments, the skipped exon sequence comprises a nucleic acid deletion, substitution, or insertion. In embodiments, the skipped exon itself does not comprise a sequence mutation, but a neighboring intron comprises a mutation leading to a frameshift mutation or a nonsense mutation. In embodiments, hybridization of the hybrid oligonucleotide to a target nucleic acid sequence within a target pre-mRNA prevents inclusion of an intron sequence in the mature mRNA molecule. In embodiments, hybridization results in preferential expression of a wild-type target protein isomer. In embodiments, hybridization results in expression of a re-spliced target protein comprising an active fragment of a wild-type target protein.

In embodiments, the re-spliced target protein can rescue one or more phenotypes or symptoms of a disease associated with the transcription and translation of the target gene. In embodiments, the re-spliced target protein can rescue one or more phenotypes or symptoms of a disease associated with the expression of the target protein. In embodiments, the re-spliced target protein is an active fragment of a wild-type target protein. In embodiments, the re-spliced target protein functions in a substantially similar manner to the wild-type target protein. In embodiments, the re-spliced target protein allows the cell to function substantially similar to a similar cell which expresses a wild-type target protein. In embodiments, the re-spliced target protein does not cure the disease associated with the target gene or with the target protein, but ameliorates one or more symptoms of the disease.

In embodiments, the re-spliced target protein may have one or more properties that are improved relative to the target protein. In embodiments, the re-spliced target protein may have one or more properties that are improved relative to a wild type target protein. In embodiments, the enzymatic activity or stability may be enhanced by promoting different splicing of the target pre-mRNA. In embodiments, the re-spliced target protein may have a sequence identical or substantially similar to a wild type target protein isomer having improved properties compared to another wild type target protein isomer.

In embodiments, hybridization of the hybrid oligonucleotide to an mRNA target generates an mRNA that encodes a truncated protein and/or a nonfunctional protein, for example, by the introduction of a frameshift mutation that results in a premature termination codon. In embodiments, hybridization results in an mRNA that encodes a truncated protein and/or a nonfunctional protein through alternative splicing. In embodiments, the hybrid oligonucleotide triggers degradation of the target transcript, for example, through nonsense mediated decay. In embodiments, hybridization of the hybrid oligonucleotide to the mRNA target generates an alternate mRNA isoform that has beneficial properties.

In embodiments, the hybrid oligonucleotide induces the addition or deletion of one or more nucleotides in a resulting processed transcript, such as a mRNA. If the number of nucleotides added or removed from the open reading is divisible by three to produce a whole number, the resultant transcript may be translated into a functioning or non-functioning protein having more or less amino acids than a counterpart protein expressed from a transcript but otherwise has the same amino acid sequence, other than the added or deleted amino acids, as a protein expressed from a transcript that did not have the nucleotides added or removed. If the number of nucleotides added or removed from the open reading frame is not divisible by three to produce a whole number, the open reading frame of the resulting processed transcript, such as an mRNA, is shifted. For example, the number of nucleotides added or deleted to induce a such a “frameshift” alteration may be 1, 2, 4, 5, 7, 8, 10, 11, 13, 14, 16, 17, 19, 20, 22, 23, etc. Due to the triplet nature of the genetic code, the addition or deletion of a number of nucleotides that is not divisible by three, shifts the reading frame of the resulting processed transcript, such as an mRNA, downstream of frameshift. The shifted reading frame may result in nonsense mediated decay, may result in a premature stop codon within the nonsense downstream of the frameshift, and/or may result in expression of a protein having a completely different sequence of amino acids downstream of the frameshift.

In embodiments, hybridization of the hybrid oligonucleotide induces introduction of a premature termination codon (PTC) into the open reading frame. As used herein, “premature termination codon” is a stop codon in phase with the translational start codon and located upstream of the physiological stop codon that is in phase with the translation start codon. A target transcript having a PTC may be destabilized and degraded through various mechanisms including nonsense mediated decay.

In embodiments, the hybrid oligonucleotide induces exon skipping of an exon within a target transcript where the exon has a has a number of nucleotides not divisible by three. In embodiments, the hybrid oligonucleotide induces exon skipping of an exon that has a number of nucleotides not divisible by three resulting in a preliminary termination codon (PTC) within the target transcript. In embodiments, the hybrid oligonucleotide induces exon skipping of an exon that has a number of nucleotides not divisible by three resulting in a PTCT within the target transcript which leads to nonsense mediated decay of the target transcript. In embodiments, inducing nonsense mediated decay of a target transcript results in a decreased concentration of the target transcript. In embodiments, inducing nonsense mediated decay of a target transcript results in a decreased concentration of the target protein encoded by the target transcript. In embodiments, inducing nonsense mediated decay of a target transcript results in increased and/or decreased levels of proteins of downstream genes regulated by the target gene.

### Gapmer

In embodiments, the hybrid oligonucleotide is a gapmer. Gapmers are short, synthetic, single-stranded, oligonucleotides that range from about 10 to about 30, about 20 to about 30, or about 15 to about 20, or about 10, 11, 12, 13, 14, 15, 16, 17, 18, 19, 20, 21, 22, 23, 24, 25, 26, 27, 28, 29 or 30 nucleotides in length and include a central “gap” region, flanked by a 3′ wing (also referred to herein as a 3′ flank) and a 5′ wing (also referred to herein as a 5′ flank) that form a contiguous sequence of monomer subunits. The central “gap” region generally includes from about 5 to about 20, or about 8 to about 10, or about 5, 6, 7, 8, 9, 10, 11, 12, 13, 14, 15, 16, 17, 18, 19 or 20 deoxyribonucleotides or deoxyribonucleotide analogs. The 3′ and 5′ flanks generally include from about 2 to about 20, about 2 to about 10, or about 4 to about 6 nucleotides.

In embodiments, the gapmer binds to a target RNA sequence in a sequence-specific manner by Watson and Crick base-pairing to form a double-stranded RNA-DNA duplex that mimics endogenous RNA-DNA hybrids that occur naturally, for example, during DNA replication, and are recognized and degraded via RNase H-mediated cleavage of the RNA strand which results in degradation of the RNA.

In embodiments, the sugar groups of 5′ flank and the 3′ flank are different than the sugar groups of the central gap region. In embodiments, the sugar group of each monomer subunit within the 5′ flank are the same. In embodiments, the sugar group of each monomer subunit within the 3′ flank are the same. In embodiments, the sugar group of each monomer subunit within the central gap are the same. In embodiments, not all of the the sugar groups for the monomer subunits within the 5′ flank are the same. In embodiments, not all of the sugar groups for the monomer subunits within the 3′ flank are the same. In embodiments, not all of the sugar groups for the monomer subunits within the central gap are the same.

In embodiments, one or more nucleotides in the 3′ flank, the 5′ flank, the gap region or combinations thereof include one or more modifications to increase nuclease resistance of the oligonucleotide, to reduce immunogenicity of the oligonucleotide, to increase binding affinity, or a combination thereof. In embodiments, one or more nucleotides in the 5′ flank, the 3′ flank, or both include a modification in the sugar ring of a DNA or RNA nucleotide. In embodiments, the 5′ flank, the 3′ flank, the central “gap” or a combination thereof include modifications to one ore more internucleotidic linkages. In embodiments, the gapmer can include modifications to one or more internucleotidic linkages to increase stability, improve plasma protein binding, or a combination thereof.

### RNA Interference Nucleic Acids

In embodiments, the hybrid oligonucleotide is a RNA interference (RNAi) molecule or a small interfering RNA (siRNA) molecule.

Small interfering RNAs (siRNAs) are oligonucleotide duplexes having a sense strand (also referred to as a passenger strand) and an antisense strand (also referred to as a guide strand) that can associate with a cytoplasmic multi-protein complex known as RNAi-induced silencing complex (RISC) and thereby mediate the degradation of homologous mRNA transcripts. As such, siRNA can be designed to knock down protein expression.

In embodiments, the siRNA compound is made up of a single molecule that includes a duplexed region, formed by intra-strand pairing, for example, a hairpin or pan-handle structure.

MicroRNA (miRNA)

In embodiments, the hybrid oligonucleotide is a microRNA (“miRNA”) mimic. As used herein, the term “miRNA mimic” refers to synthetic oligonucleotide that imitates the gene silencing ability of miRNA, for example, by entering the RNAi pathway and regulating gene expression. As used herein, microRNAs (miRNAs) are small, single stranded molecules that target the 5′ or 3′ untranslated (UTR) to control gene expression, for example, by translational repression or degradation of mRNA. In embodiments, the miRNA mimic interacts with the 3′UTR of a target mRNA to suppress expression. In embodiments, the miRNA mimic interacts with the 5′UTR of a target mRNA to suppress expression.

In embodiments, the miRNA mimic includes from about 15 to about 30 nucleotides, or about 17 to about 25 nucleotides, or about 15, about 16, 17, 18, 19, 20, 21, 22, 23, 24, 25, 26, 27, 28, 28 or 30 nucleotides

miRNA Inhibitor

In embodiments, the hybrid oligonucleotide is a miRNA inhibitor (also referred to as “antimir” “microRNA inhibitor”, “miR inhibitor”, or “miRNA inhibitor”). As used herein, a “miRNA inhibitor” is an oligonucleotide that interferes with miRNA activity. In embodiments, the miRNA inhibitor includes one or more sequences that the reverse complement of the mature miRNA that are chemically modified to prevent RISC-induced cleavage, enhance binding affinity, provide resistance to nucleolytic degradation, or a combination thereof. In embodiments, the miRNA inhibitor binds to maturemiRNA and sequester the the endogenous miRNA, making it unavailable for normal function.

### Antagomirs

In embodiments, the hybrid oligonucleotide is an antagomir. Antagomirs are RNA-like oligonucleotides that harbor various modifications for RNAse protection and pharmacologic properties, such as enhanced tissue and cellular uptake. In embodiments, antagomirs silence endogenous miRNAs by forming duplexes comprising the antagomir and endogenous miRNA, thereby preventing miRNA-induced gene silencing.

### Supermir

In embodiments, the hybrid oligonucleotide is a supermir. A supermir refers to a single stranded, double stranded or partially double stranded oligomer or polymer of ribonucleic acid (RNA) or deoxyribonucleic acid (DNA) or both, or modifications thereof, which has a nucleotide sequence that is substantially identical to a miRNA and that is antisense with respect to its target. In embodiments, the supermir includes oligonucleotides composed of naturally-occurring and non-naturally-occurring nucleobases, sugars internucleotidic linkages, or combinations thereof. In embodiments, the supermir is substantially single-stranded, e.g., less than about 50% (e.g., less than about 40%, about 30%, about 20%, about 10%, or about 5%) of the supermir is duplexed with itself. In embodiments, the supermir includes a hairpin segment, for example, at the 3′ end, that can self hybridize and form a duplex region, for example, a duplex region of at least about 1, about 2, about 3, or about 4 and less than about 8, about 7, about 6, or nucleotides.

### Immunostimulatory Oligonucleotides

In embodiments, the hybrid oligonucleotide is an immunostimulatory oligonucleotide. Immunostimulatory oligonucleotides are single or double stranded oligonucleotides that are capable of inducing an immune response when administered to a subject. In embodiments, the immunostimulatory oligonucleotides includes a palindrome that forms a hairpin secondary structure or a CpG motifs.

In embodiments, the immunostimulatory oligonucleotide comprises at least one CpG dinucleotide. The oligonucleotide or CpG dinucleotide may be unmethylated or methylated. In another embodiment, the immunostimulatory oligonucleotide comprises at least one CpG dinucleotide having a methylated cytosine. In embodiments, the immunostimulatory oligonucleotide comprises a single CpG dinucleotide, wherein the cytosine in said CpG dinucleotide is methylated. In embodiments, the immunostimulatory oligonucleotide comprises at least two CpG dinucleotides, wherein at least one cytosine in the CpG dinucleotides is methylated. In embodiments, each cytosine in the CpG dinucleotides is methylated. In embodiments, the immunostimulatory oligonucleotide comprises a plurality of CpG dinucleotides, wherein at least one of said CpG dinucleotides comprises a methylated cytosine.

### Decoy Oligonucleotides

In embodiments, the hybrid oligonucleotide is a decoy oligonucleotide. Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to upregulate genes that are suppressed by the binding of a transcription factor.

### U1 Adaptor

In embodiments, the hybrid oligonucleotide is a U1 adaptor. U1 adaptors inhibit polyA sites and are bifunctional oligonucleotides with a target domain complementary to a site in the target gene's terminal exon and a ‘U1 domain’ that binds to the U1 smaller nuclear RNA component of the U1 snRNP (Goraczniak, et al., 2008, Nature Biotechnology, 27(3), 257-263, which is expressly incorporated by reference herein, in its entirety). U1 snRNP is a ribonucleoprotein complex that functions primarily to direct early steps in spliceosome formation by binding to the pre-mRNA exon-intron boundary (Brown and Simpson, 1998, Annu Rev Plant Physiol Plant Mol Biol 49.77-95). Nucleotides 2-11 of the 5′end of U1 snRNA base pair bind with the 5′ss of the pre mRNA. In one embodiment, oligonucleotides of the invention are U1 adaptors. In one embodiment, the U1 adaptor can be administered in combination with at least one other iRNA agent.

### Hybridization Site

The hybridization site of the hybrid oligonucleotide will vary depending on the target nucleic acid sequence, and/or the disease being treated. In embodiments, the target nucleic acid sequence is an RNA sequence. In embodiments, the target nucleic acid sequence is in a target mRNA. In embodiments, the target nucleic acid sequence is in a target pre-mRNA. In embodiments, the target nucleic acid sequence is a DNA sequence. In embodiments, the target nucleic acid sequence is a genomic DNA sequence.

### Splice Site

In embodiments, the hybrid oligonucleotide hybridizes to and sterically blocks access to a splice site (e.g., a splice acceptor or a splice donor site), or at least a portion of a splicing element (SE) and/or a cis-acting splicing regulatory element (SRE), thereby redirecting splicing to a cryptic or de novo splice site. In embodiments, the hybrid oligonucleotide is targeted to a splicing enhancer sequence (e.g., ESE an/or ISE) or splicing silencer sequence (e.g., ESS and/or ISS) to prevent binding of trans-acting regulatory splicing factors at the target site and effectively block or promote splicing. In embodiments, the hybrid oligonucleotide can be designed to base-pair across the base of a splicing regulatory stem loop to strengthen the stem-loop structure.

### Nucleotide Repeats

In embodiments, the hybrid oligonucleotide targets a nucleotide repeat (e.g., trinucleotide repeat expansions, pentanucleotide repeat expansions, or hexanucleotide repeat expansions) in a target nucleic acid sequence, or targets a sequence that has a nucleotide repeat. In embodiments, the hybrid oligonucleotide blocks expansion of a nucleotide repeat. In embodiments, the hybrid oligonucleotide blocks transcription of a nucleotide repeat.

In embodiments, the hybrid oligonucleotide is complementary to a target nucleic acid sequence that has a trinucleotide repeat expansion. In embodiments, the hybrid oligonucleotide hybridizes to the trinucleotide repeat expansion of a target nucleic acid sequence. In embodiments, the hybrid oligonucleotide is complementary to about 5 to 10 trinucleotide repeats in the target nucleic acid sequence. In embodiments, the hybrid oligonucleotide is complementary to from 5 to 15 trinucleotide repeats in the target nucleic acid sequence, or about 5, about 6, 7, 8, 9, 10, 11, 12, 13, 14, or 15 trinucleotide repeats in the target nucleic acid sequence.

In embodiments, the hybrid oligonucleotide is complementary to trinucleotide repeats, such as a CAG repeat, a CGG repeat, a GCC repeat, a GAA repeat, or a CUG repeat. In embodiments, the target nucleic acid sequence comprises at least 5, at least 10, 15, 20, 25, 30, 35, 40, 45, 50, 60, 70, 80, 90, 100, 150, 200, 300, 400, 500, 600, 700, 800, 900, 1000 or 2000 trinucleotide repeats (e.g., CAG, CGG, GCC, GAA, or CUG repeats).

In embodiments, the hybrid oligonucleotide degrades trinucleotide repeats. In embodiments, after binding of a hybrid oligonucleotide to a target nucleic acid sequence, the target nucleic acid sequence is degraded by RNase H.

### Polyadenylation Signal

In embodiments, the hybrid oligonucleotide targets a polyadenylation signal (PAS) of a target. In embodiments, the hybrid oligonucleotide inhibits polyadenylation of a gene transcript.

In embodiments, the hybrid oligonucleotide interacts with (e.g., binds to) a polyadenylation sequence element (PSE), including, but not limited to, a polyadenylation signal (PAS), a cleavage stie (CS), and a GU-rich downstream element (DSE). In embodiments, cases, the polyadenylation sequence elements includes one or more of an auxiliary upstream element (USE), a G-rich sequence (GRS) auxiliary downstream element (AUX DSE), and/or a sequence downstream of a core U-rich element (URE) (See, e.g., Chen and Wilusz (1998) Nuc. Acid. Rec. 1998 26(12):2891-2898).

In embodiments, the PAS is an adenosine-rich hexamer sequence that includes a canonical AATAAA hexamer or a variant differing by a single nucleotide (e.g., AAUAAA, AUUAAA, UAUAAA, AGUAAA, AAGAAA, AAUAUA, AAUACA, CAUAAA, GAUAAA, CAUAAA, GAUAAA, AAUGAA, UUUAAA, ACUAAA, AAUAGA, AAAAAG, AAAACA, GGGGCU; Marsollier et al. Int. J. Mol. Sci., (2018), 19, 1347, doi:10.3390/ijms19051347; Beaudoing, et al. Genome Res. (2000), 10, 1001-1010; and Tian, B. et al., Nucleic Acids Res. (2005), 33, 201-212). The hexamer sequences occur with varying frequencies, with AAUAAA and AUUAAA being the most frequent (see, Ibid). The PAS is typically found upstream of the CS. The hexamer sequence of the PAS serves as the binding site for a cleavage and polyadenylation specific factor (CPSF). The PAS can also be determined by the presence of other auxiliary elements, such as upstream U-rich elements (USE) (see, Tian et al., Nuc. Acid. Res. (2005), 33(1):201-212 and Neve et al. (2017) RNA Biology, 2017, 14(7):865-890).

The DSE is a U-rich or U/G-rich element that serves as the binding site for a cleavage stimulatory factor (CstF). The DSE is typically found downstream of the CS. The DSE may be followed by a stretch of three or more uracil bases present downstream of the CS, often within 20 to 40 nucleotides of the CS. In mammals, CA and UA are the most frequent dinucleotides that precede the cleavage site (CS), although the actual cleavage site is known to be heterogeneous.

CPSF and CstF, two multi-subunit complexes, cooperate with each other and two additional factors (cleavage factors I and II) to cleave the mRNA sequence. Poly(A) polymerase (PAP), a single-subunit enzyme is also involved in cleavage of most pre-mRNAs, as is RNA polymerase I. CPSF and PAP together with a poly(A) binding protein II and cleavage stimulating factor (CstF) are involved in the addition of the poly(A) tail (Takagaki and Manley, Mol Cell Biol. (2000), 20(5):1515-1525).

Methods for identifying polyadenylation sequence elements are known and can include but are not limited to, for example, the methodologies described by: Tian et al., Nuc. Acid. Res. (2005) 33(0):201-212; Beaudoing, et al., Genome Res. (2000), 10, 1001-1010; Marsollier et al., Int. J. Mol. Sci. (2018), 19, 1347, doi:10.3390/ijms19051347; Chen, Molec. Therapy (2016), 24(8) 1405-1411; Venkataraman et al. Genes and Dev. (2005) 19:1315-1327; Nourse et al. Biomolecules (2000), 10(915) doi:10.3390/biom10060915; and Vickers et al. Nucleic Acids Research (2001) 29(6) 1293-1299.

### Methods of Making

The disclosure relates to method of synthesizing hybrid oligonucleotides and cleaving them from solid support. The solid support can be a Universal solid supports. P(V) PMO nucleotides can be used with other resins typically employed in PMO synthesis.

The synthesis is carried out from 6′ or 5′ end towards 3′ end and involves PMO 6′-chlorophosphoroamidates P(V), PMO 6′-β-cyanoethylamidites P(M), and reversed 5′-β-cyanoethylamidites P(III) based on 2′-deoxyribose, 2′-substituted ribose, LNA or other nucleotides.

The transition between 6′ PMO flank and the gap is accomplished by reacting 5′ phosphoramidite with N3 of the morpholino ring in the presence of activating agent and sulfurization. The result is negatively charged N3′>P5′ thiophosphoramidate linker.

The transition between the gap and the 3′ PMO flank is accomplished by coupling the PMO 6′-β-cyanoethylamidite P(III) with 3′ hydroxyl group in the presence of activating agent and sulfurization or oxidation. That results in a formation of phosphorothio PS or phoshodiester PO linker.

If the PMO is introduced later in the sequence the labile protecting group on the nucleobase should be avoided. Adenosine and cytidine protecting group can be N-benzoyl, and guanosine protecting group can be N-isobutyryl.

The phosphate protecting group can be O-cyanoethyl, O-methyl, O-ethyl, O-benzyl, O-allyl.

If the PMO is introduced later in the sequence the Cap A contains iso-butyric anhydride, pivalic anhydride or benzoic anhydride but not acetic anhydride.

The method of making an oligonucleotide comprising assembling P(III) and P(V) nucleotide building blocks in a 6′ to 3′ or 5′ to 3′ direction on a support, and employing PMO 6′-phosphoramidite P(III) or phosphoramidate P(V) as the first nucleotide from the support.

The disclosure further relates to methods of making an oligonucleotide comprising assembling P(III) and P(V) nucleotide building blocks in a 6′ to 3′ or 5′ to 3′ direction on a support, and employing PMO 6′-phosphoramidite P(III) or phosphoramidate P(V) as the second nucleotide from the support, where the first nucleotide is reversed DNA (PI).

The disclosure also relates to a method of making an oligonucleotide comprising: a first nucleotide sequence comprising a first 5′ or 6′ end and a first 3′ end, wherein the terminal 3′ nucleic acid residue of the first nucleotide sequence is a morpholino nucleotide analog;
(ii) a second nucleotide sequence comprising a second 5′ or 6′ end and a second 3′ end, wherein the terminal 5′ nucleic acid residue of the second nucleotide sequence is a deoxyribonucleotide or analog thereof, or a ribonucleotide or analog thereof; and
(iii) at least one phosphrothioamidate linkage that links the terminal 3′ nucleic acid residue of the first nucleotide sequence and the terminal 5′ nucleic acid residue of the second nucleotide sequence.

In embodiments, the support can be a polystyrene resin.

In embodiments, the PMO 6′-phosphoramidite P(UM) can be the first nucleotide from the support. PMO 6′-phosphoramidate P(V) can be the first nucleotide from the support.

In embodiments, the oligonucleotide prepared by the method can comprise phosphorothioester, phosphorodiester linkages and/or phosphorodiamidate linkages and at least one phosphorothioamidate linkage. In embodiments, the oligonucleotide prepared by the method can comprise phosphorothioester, phosphorodiester linkages and/or phosphorodiamidate linkages and one phosphorothioamidate linkage.

In embodiments, the oligonucleotide prepared by the method can comprise a phosphorodiamidate linkage and at least one phosphorothioamidate linkage (FIG. 5). In embodiments, the oligonucleotide prepared by the method only has phosphorodiamidate and phosphorothioamidate linkages. In embodiments, the oligonucleotide prepared by the method can comprise phosphorodiamidate and phosphorothioester linkages and at least one phosphorothioamidate linkage.

In embodiments, a phosphorothioester can link two reversed DNA amidite. In embodiments, a phosphorothioester can link two reversed RNA amidite. In embodiments, a phosphorothioester can link link a reversed DNA amidite and a reversed DNA amidite. In embodiments, a phosphorothioester links a reversed DNA amidite and a PMO (III).

In embodiments, a phosphorodiamidate can link a PMO(III) and a PMO (V). In embodiments, a phosphorodiamidate can link two PMO(V).

In some embodiments, a phosphorothioamidate can link a PMO (V) to a PMO(III). In some embodiments, a phosphorothioamidate can link a PMO(III) to a a reversed DNA P(III).

In some embodiments, a phosphodiester can link a reversed RNA and a reversed DNA. In some embodiments, a phosphodiester can link two reversed RNA.

In embodiments a deoxyribonucleotide analogs, two or more ribonucleotide analogs can precede the PMO.

The method can comprise coupling a first reverse amidite to a secondary amino group of a morpholino ring to form a phosphorothioamidate linkage.

Hence the method provides adding (PIII) and P(V) building blocks, wherein the building blocks are PMO, reversed DNA, reversed RNA and reversed LNA.

In embodiments, the method can further comprise adding at least one P(III) building block. In embodiments, the method can further comprise adding at least two P(III) building blocks. In embodiments, the building blocks can be added consecutively or can be added as a chain of building blocks.

In embodiments, the P(III) building blocks can be consecutive.

In embodiments, the method can further comprise adding at least one P(V) building block.

In embodiments, the method can comprise adding at least two P(V) building blocks. The P(V) building blocks can be consecutive. In embodiments, at least two P(III) building blocks can be followed by at least two P(V) building blocks. In embodiments, at least two P(V) building blocks can be followed by at least two P(III) building blocks. In embodiments, at least one P(III) building block and one P(V) building block can alternate to form a P(III)-P(V)-P(III) or P(V)-P(III)-P(V) motif. In embodiments, the P(III) building blocks can be the same. In embodiments, the P(III) building blocks can be different. In embodiments, the P(V) building blocks can be the same. In embodiments, the P(V) building blocks can be different. In embodiments, the building blocks can be added consecutively or can be added as a group of building blocks.

In embodiments, the method can comprise deprotecting the 3′-terminal secondary amino group of a first DNA monomer. The method can further comprise neutralizing the deprotected 3′-terminal secondary amino group of a first DNA monomer

In embodiments, the method can comprise further adding one or more additional DNA nucleotides to the first DNA nucleotide, wherein two or more DNA nucleotides are linked via a phosphorothioate (PS) linkage

In embodiments, the method can further comprise adding a second PMO 6′-phosphoramidite P(III) to a 3′ terminal DNA nucleotide of the hybrid oligonucleotide.

In embodiments, the method can provide an oligonucleotide that can comprise a gapmer comprising:
(i) a 6′ flank comprising two or more morpholino nucleotide analogs, or a 5′ flank comprising two or more ribonucleotides or ribonucleotide analogs;
(ii) a gap region comprising 5 or more deoxyribonucleotides or deoxyribonucleotide analogs; and
(iii) a 3′ flank comprising two or more morpholino nucleotide analogs, or a 3′ flank comprising two or more ribonucleotides or ribonucleotide analogs.

In embodiments, the methods can further comprise extending the oligonucleotide by adding one or more additional DNA monomers to the first DNA monomer that are linked through a phosphorothioate bond (DNA-PS).

In embodiments, the methods can further comprise adding PMO 6′-phosphoramidite P(III) to the 3′ end of the oligonucleotide.

In embodiments, the 3′-PMO wing can be added if all Cytidine based nucleotides in the 5′ flank and the gap can be N-benzoyl protected and Cap A based on iso-butyric, pivalic or benzoic anhydride instead of acetic anhydride.

In embodiments, the methods of the disclosure provide means to synthesized the compounds disclosed herein.

In embodiments, the synthesis of the oligonucleotides can be automated. In embodiments, the synthesis of the oligonucleotides can be done manually. In embodiments, the synthesis of the oligonucleotides can be done via a combination of automation and manual synthesis. In embodiments, the synthesis of the oligonucleotides can be on solid support. In embodiments, the synthesis of the oligonucleotides can be in solution phase. In embodiments, the synthesis of the oligonucleotides can be a combination of synthesis on solid support and in solid phase.

In embodiments, the oligonucleotides are assembled by stepwise coupling of individual monomers. In embodiments, the oligonucleotides are assembled by by coupling of linked monomers to an individual monomer. In embodiments, the oligonucleotides are assembled by by coupling of a first set linked monomers to a second set of linked monomer. Any method of coupling reaction for the synthesis of oligonucleotide can be employed in the disclosed methods.

### Hybrid Oligonucleotide Design

Design of the hybrid oligonucleotide will depend upon the sequence being targeted. One of skill in the art can design, synthesize, and screen compounds of different nucleobase sequences to identify a sequence that results in the desired activity. In embodiments, the hybrid oligonucleotide includes one or more modified nucleotides, one or more modified internucleotidic linkages, or a combination thereof. In embodiments, the hybrid oligonucleotide can be, but is not limited to, a gapmer, splice switching oligonucleotide, sense strand of siRNA, antisense strand of siRNA, deoxyribozyme, steric block antisense oligonucleotide (ASO), a DNAzyme, RNAzyme, aptamer, immunomodulatory oligonucleotide, or antagomir.

In embodiments, the hybrid oligonucleotide includes one or more modified nucleotides. In embodiments, all of the nucleotides in the hybrid oligonucleotide are modified. In embodiments, one or more nucleotides comprise a modified nucleobase. In embodiments, one or more nucleotides comprise a modified sugar. In embodiments, the hybrid oligonucleotide includes one or more modified internucleotidic linkages.

In embodiments, the hybrid oligonucleotide comprises: (i) at least one P(III) morpholino nucleotide analog; (ii) at least one P(V) morpholino nucleotide analog; (iii) at least one P(III) ribonucleotide, at least one P(III) deoxyribonucleotide analog, or a combination thereof; (iv) at least one phosphorodiamidate linkage; and (iv) at least one phosphorothioamidite linkage. In embodiments, each upstream nucleotide adjacent to each P(V) morpholino nucleotide analog is a P(III) or P(V) morpholino nucleotide analog. In embodiments, the hybrid oligonucleotide comprises 2 or more consecutive P(V) morpholino nucleotide analogs linked through phosphorodiamidate linkages. In embodiments, the hybrid oligonucleotide comprises from 2 to 20, 3 to 10 or 4 to 6 consecutive P(V) morpholino nucleotide analogs linked through phosphorodiamidate linkages. In embodiments, the hybrid oligonucleotide comprises 2, 3, 4, 5, 6, 7, 8, 9, 10, 11, 12, 13, 14, 15, 16, 17, 18, 19, or 20 consecutive P(V) morpholino nucleotide analogs linked through phosphorodiamidate linkages. In embodiments, any P(V) morpholino nucleotide analog is linked to any downstream nucleotide that is not a P(V) morpholino nucleotide analog through a phosphorothioamidate linkage.

In embodiments, the hybrid oligonucleotide comprises at least one (1) 2′-modified P(MI) ribonucleotide. In embodiments, the hybrid oligonucleotide comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(III) DNA analog, a reverse P(III) DNA analog, a P(III) RNA analog, a reverse P(III) RNA analog, or a combination thereof. In embodiments, the first nucleotide sequence comprises a 2′ modified ribonucleotide analog selected from: 2′O-methyl (2′-OMe) P(III) RNA, reverse 2′-O-methyl (2′-OMe) P(III) RNA, 2′-O-methoxyethyl (2′-OMOE) P(III) RNA, reverse 2′-O-methoxyethyl (2′-OMOE) P(III) RNA, 2′-fluoro P(III) RNA, reverse 2′-fluoro P(III) RNA, or combinations thereof.

In embodiments, the hybrid oligonucleotide comprises at least one internucleotidic linkage selected from: phosphodiester, phosphotriester, methylphosphonate, phosphoramidate, phosphorodiamidate, phosphorothioamidate, or phosphorothioate. In embodiments, the hybrid oligonucleotide comprises at least one internucleotidic linkage selected from methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). In embodiments, the hybrid oligonucleotide comprises an internucleotidic linkage selected from: phosphorodiamidate, phosphorothioamidate, phosphothioate (PS) and phosphoryl guanidine linkages. In embodiments, the hybrid oligonucleotide comprises at least one phosphorthioate linkage.

In embodiments, the hybrid oligonucleotide comprises: (i) a first nucleotide sequence comprising a first 5′ or 6′ end and a first 3′ end, wherein the terminal 3′ nucleic acid residue of the first nucleotide sequence is a morpholino nucleotide analog; (ii) a second nucleotide sequence comprising a second 5′ or 6′ end and a second 3′ end, wherein the terminal 5′ nucleic acid residue of the second nucleotide sequence is a deoxyribonucleotide or analog thereof, or a ribonucleotide or analog thereof; and (iii) at least one phosphrothioamidate linkage that links the terminal 3′ nucleic acid residue of the first nucleotide sequence and the terminal 5′ nucleic acid residue of the second nucleotide sequence. In embodiments, two or more deoxyribonucleotides or analogs, two or more ribonucleotides or analogs thereof, or combinations thereof are each linked through an internucleotidic linkage.

In embodiments, the hybrid oligonucleotide comprises from 10 to 50 nucleotides, from 15 to 30 nucleotides, or from 20 to 30 nucleotides. In embodiments, the hybrid oligonucleotide comprises 15, 16, 17, 18, 19, 20, 21, 22, 23, 24, 25, 26, 27, 28, 29, or 30 nucleotides.

In embodiments, the first nucleotide sequence comprises two or more morpholino nucleotide analogs. In embodiments, the first nucleotide sequence comprises from 2 to 20, 3 to 10, or 4 to 6 morpholino nucleotide analogs. In embodiments, the first nucleotide sequence comprises 2, 3, 4, 5, 6, 7, 8, 9, 10, 11, 12, 13, 14, 15, 16, 17, 18, 19, or 20 morpholino nucleotide analogs. In embodiments, the first nucleotide sequence comprises 2 morpholino nucleotide analogs. In embodiments, the first nucleotide sequence comprises 3 morpholino nucleotide analogs. In embodiments, the first nucleotide sequence comprises 4 morpholino nucleotide analogs. In embodiments, the first nucleotide sequence comprises 5 morpholino nucleotide analogs.

In embodiments, the morpholino nucleotide analog is a P(III) PMO or a P(V) PMO. In embodiments, the morpholino nucleotide analog is a P(III) PMO. In embodiments, the P(III) PMO is selected from: P(III) PMO-C, P(III) PMO-G, P(III) PMO-A, P(III) PMO-T, or a P(II) PMO with a non-naturally occurring nucleobase. In embodiments, the morpholino nucleotide analog is a P(V) PMO. In embodiments, the (PV) PMO comprises: P(V) PMO-C, P(V) PMO-G, P(V) PMO-A, P(V) PMO-T, or a P(V) PMO with a non-naturally occurring nucleobase.

In embodiments, 2 or more consecutive morpholino oligonucleotide analogs in the first nucleotide sequence are linked through a phosphordiamidate linkage. In embodiments, from 2 to 20, 3 to 10 or 4 to 6 consecutive morpholino oligonucleotide analogs in the first nucleotide sequence are linked through a phosphordiamidate linkage. In embodiments, 2, 3, 4, 5, 6, 7, 8, 9, 10, 11, 12, 13, 14, 15, 16, 17, 18, 19 or 20 consecutive morpholino oligonucleotide analogs in the first nucleotide sequence are linked through a phosphorodiamidate linkage. In embodiments, the terminal 3′ nucleic acid residue of the first nucleic acid sequence is a P(III) morpholino nucleotide analog. In embodiments, the terminal 3′ nucleic acid residue of the first nucleic acid sequence is a P(V) morpholino nucleotide analog. In embodiments, the terminal 3′ nucleic acid residue of the first nucleic acid sequence is bound to a solid substrate.

In embodiments, the first nucleotide sequence of the hybrid oligonucleotide comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(III) DNA analog, a reverse P(IIII) DNA analog, a P(III) RNA analog, a reverse P(III) RNA analog, or a combination thereof. In embodiments, the first nucleotide sequence of the hybrid oligonucleotide comprises at least one (1) 2′-modified P(III) ribonucleotide. In embodiments, the first oligonucleotide sequence of the hybrid oligonucleotide comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(III) DNA analog, a reverse P(IIII) DNA analog, a P(III) RNA analog, a reverse P(III) RNA analog, or a combination thereof. In embodiments, the first nucleotide sequence comprises a 2′ modified ribonucleotide analog selected from: 2′-O-methyl (2′-OMe) P(III) RNA, reverse 2′-O-methyl (2′-OMe) P(III) RNA, 2′-O-methoxyethyl (2′-OMOE) P(III) RNA, reverse 2′-O-methoxyethyl (2′-MOE) P(III) RNA, 2′-fluoro P(III) RNA, reverse 2′-fluoro P(III) RNA, or combinations thereof.

In embodiments, the first oligonucleotide sequence of the hybrid oligonucleotide comprises at least one internucleotidic linkage selected from: phosphodiester, phosphotriester, methylphosphonate, phosphoramidate, phosphorodiamidate, phosphorothioamidate, or phosphorothioate. In embodiments, the first nucleotide sequence of the hybrid oligonucleotide comprises at least one internucleotidic linkage selected from methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). In embodiments, the first oligonucleotide sequence of the hybrid oligonucleotide comprises an internucleotidic linkage selected from: phosphorodiamidate, phosphorothioamidate, phosphothioate (PS) and phosphoryl guanidine linkages. In embodiments, the first oligonucleotide sequence of the hybrid oligonucleotide comprises at least one phosphorthioate linkage.

In embodiments, the second nucleotide sequence of the hybrid oligonucleotide comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(IIII) DNA analog, a reverse P(IIII) DNA analog, a P(III) RNA analog, a reverse P(III) RNA analog, or a combination thereof. In embodiments, the second nucleotide sequence of the hybrid oligonucleotide comprises at least one (1) 2′-modified P(III) ribonucleotide. In embodiments, the second oligonucleotide sequence of the hybrid oligonucleotide comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(III) DNA analog, a reverse P(III) DNA analog, a P(III) RNA analog, a reverse P(III) RNA analog, or a combination thereof. In embodiments, the first nucleotide sequence comprises a 2′ modified ribonucleotide analog selected from: 2′-O-methyl (2′-OMe) P(III) RNA, reverse 2′-O-methyl (2′-OMe) P(III) RNA, 2′-O-methoxyethyl (2′-OMOE) P(III) RNA, reverse 2′-O-methoxyethyl (2′-MOE) P(III) RNA, 2′-fluoro P(III) RNA, reverse 2′-fluoro P(III) RNA, or combinations thereof.

In embodiments, the second nucleotide sequence comprises one or more morpholino nucleotide analogs. In embodiments, the morpholino nucleotide analog is a P(III) PMO. In embodiments, the P(III) PMO is selected from: P(III) PMO-C, P(III) PMO-G. P(III) PMO-A, P(III) PMO-T, or a P(III) PMO with a non-naturally occurring nucleobase. In embodiments, the morpholino nucleotide analog is a P(V) PMO. In embodiments, the (PV) PMO comprises: P(V) PMO-C, P(V) PMO-G, P(V) PMO-A, P(V) PMO-T, or a P(V) PMO with a non-naturally occurring nucleobase

In embodiments, the second oligonucleotide sequence of the hybrid oligonucleotide comprises at least one internucleotidic linkage selected from: phosphodiester, phosphotriester, methylphosphonate, phosphoramidate, phosphorodiamidate, phosphorothioamidate, or phosphorothioate. In embodiments, the second nucleotide sequence of the hybrid oligonucleotide comprises at least one internucleotidic linkage selected from methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). In embodiments, the second oligonucleotide sequence of the hybrid oligonucleotide comprises an internucleotidic linkage selected from: phosphorodiamidate, phosphorothioamidate, phosphorothioate (PS) and phosphoryl guanidine linkages. In embodiments, the second oligonucleotide sequence of the hybrid oligonucleotide comprises at least one phosphortohioate linkage.

In embodiments, less than 75%, less than 50%, less than 40%, less than 30%, less than 25%, less than 20%, less than 15% or less than 10% of the internucleotidic linkages in the hybrid oligonucleotide have a negative charge. In embodiments, less than less than 50%, less than 40%, less than 30%, less than 25%, less than 20%, less than 15%, less than 109% or less than 5% of the internucleotidic linkages in the first oligonucleotide sequence of the hybrid oligonucleotide have a negative charge. In embodiments, less than 75%, less than 50%, less than 40%, less than 30%, less than 25%, less than 20%, less than 15% or less than 10% of the internucleotidic linkages in the second oligonucleotide sequence of the hybrid oligonucleotide have a negative charge. In embodiments, the hybrid oligonucleotide comprises less than 20, less than 19, less than 18, less than 17, less than 16, less that 15, less than 14, less than 13, less than 12, less than 11, less than 10, less than 9, less than 8, less than 7, less than 6, or less than 5 charged internucleotidic linkages.

### Hybrid Gapmer Design

In embodiments, the hybrid oligonucleotide is a hybrid gapmer oligonucleotide. In embodiments, the hybrid gapmer oligonucleotide comprises: (i) a first nucleotide sequence comprising a 5′ flank; (ii) a second oligonucleotide sequence comprising a gap oligonucleotide sequence; and (iii) a 3′ flank.

In embodiments, the hybrid gapmer oligonucleotide comprises at least two consecutive morpholino nucleotide analogs which are linked via a neutral phosphorodiamidate linkage. Each consecutive morpholino nucleotide analog in the hybrid gapmer oligonucleotide introduces n-1 neutral linkages (wherein n=number of nucleotides in the hybrid gapmer oligonucleotide). In embodiments, the 5′ and 3′ flank each comprise at least 2 or 3 morpholino nucleotide analogs. While not wishing to be bound by theory, it is believed that the morpholino nucleotide analogs of the flanking regions improve oligonucleotide stability and binding affinity to the target nucleic acid sequence (e.g., target RNA).

In embodiments, the 5′ flank comprises from 2 to 20 nucleotides. In embodiments, the 5′ flank comprises at least one P(V) morpholino nucleotide analog. In embodiments, the 5′ flank comprises at least one P(III) ribonucleotide. It is noted that the 5′ flank can also be referred to as the 6′ flank to account for the 6 membered morpholino ring.

In embodiments, the 5′ flank comprises from 2 to 20, from 2 to 10, or from 2 to 5 morpholino nucleotide analogs. In embodiments, the 5′ flank comprises from 2 to 20, from 2 to 10, or from 2 to 5 consecutive morpholino nucleotide analogs. In embodiments, the 5′ flank comprises 2, 3, 4, 5, 6, 7, 8, 9, 10, 11, 12, 13, 14, 15, 16, 17, 18, 19 or 20 morpholino nucleotide analogs. In embodiments, the 5′ flank comprises 2, 3, 4, 5, 6, 7, 8, 9, 10, 11, 12, 13, 14, 15, 16, 17, 18, 19 or 20 consecutive morpholino nucleotide analogs.

In embodiments, 2 or more morpholino nucleotide analogs of the 5′ flank are linked through phosphorodiamidate linkages. In embodiments, from 2 to 20, from 2 to 10, or from 2 to 5 morpholino nucleotide analogs of the 5′ flank are linked through phosphorodiamidate linkages. In embodiments, 2, 3, 4, 5, 6, 7, 8, 9, 10, 11, 12, 13, 14, 15, 16, 17, 18, 19 or 20 morpholino nucleotide analogs of the 5′ flank are linked through phosphorodiamidate linkages.

In embodiments, the 5′ flank of the hybrid gapmer oligonucleotide comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(III) DNA analog, a reverse P(IIII) DNA analog, a P(III) RNA analog, a reverse P(III) RNA analog, or a combination thereof. In embodiments, the 5′ flank of the hybrid gapmer oligonucleotide comprises at least one (1) 2′-modified P(III) ribonucleotide. In embodiments, the 5′ flank comprises a 2′ modified ribonucleotide analog selected from: 2′-O-methyl (2′-OMe) P(III) RNA, reverse 2′-O-methyl (2′-OMe) P(III) RNA, 2′-O-methoxyethyl (2′-OMOE) P(III) RNA, reverse 2′-O-methoxyethyl (2′-MOE) P(III) RNA, 2′-fluoro P(III) RNA, reverse 2′-fluoro P(III) RNA, or combinations thereof.

In embodiments, the 5′ flank of the hybrid gapmer oligonucleotide comprises at least one internucleotidic linkage selected from: phosphodiester, phosphotriester, methylphosphonate, phosphoramidate, phosphorodiamidate, phosphorothioamidate, or phosphorothioate. In embodiments, the 5′ flank of the hybrid gapmer oligonucleotide comprises at least one internucleotidic linkage selected from methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). In embodiments, the 5′ flank of the hybrid gapmer oligonucleotide comprises an internucleotidic linkage selected from: phosphorodiamidate, phosphorothioamidate, phosphothioate (PS) and phosphoryl guanidine linkages.

In embodiments, the hybrid gapmer oligonucleotide comprises a 5′ flank comprising morpholino nucleotide analogs linked by phosphorodiamidate linkages, a central gap region comprising DNA nucleotides or DNA nucleotide analogs linked by phosphorothioate linkages and a 3′ flank comprising morpholino nucleotide analogs linked by phosphorodiamidate linkages (6′-PMO-DNA-PMO-3′). In embodiments, the hybrid gapmer oligonucleotide comprises a structure with a 5′ flank comprising morpholino nucleotide analogs linked by phosphorodiamidate linkages, a central gap region comprising DNA nucleotides or DNA nucleotide analogs linked by phosphorothioate linkages and a 3′ flank comprising RNA nucleotides or RNA nucleotide analogs, including, for example, 2′ modified RNA nucleotide analogs (6′-PMO-DNA-RNA-3′). In embodiments, the hybrid gapmer oligonucleotide comprises a 3′ flank comprising RNA nucleotides or RNA nucleotide analogs, including, for example, 2′ modified RNA nucleotide analogs, a central gap region comprising DNA nucleotides or DNA nucleotide analogs linked by phosphorothioate linkages and a 5′ flank comprising morpholino nucleotide analogs linked by phosphorodiamidate linkages (5′-RNA-DNA-PMO-3′).

In embodiments, the hybrid gapmer oligonucleotide comprises a thiophosphoramidate linkage between the 5′ flank and the central gap oligonucleotide. In embodiments, the 5′ or 3′ flank can include a variety of nucleotide analogs, including but not limited to morpholino nucleotide analogs, 2′-O-methyl (2′-OMe) nucleotide analogs, 2′-O-methoxyethyl (2′-OMOE) nucleotide analogs, locked nucleic acids (LNA), or combinations thereof.

In embodiments, the gap oligonucleotide sequence includes at least 8 deoxyribonucleotide or deoxyribonucleotide analogs. In embodiments, the gap oligonucleotide sequence includes at least 8 consecutive deoxyribonucleotide or deoxyribonucleotide analogs. While not wishing to be bound by theory, it is believed that a gap size of at least 8 deoxyribonucleotides or deoxyribonucleotide analogs is desirable for RNAse H activity.

In embodiments, less than 75%, less than 50%, less than 40%, less than 30%, less than 25%, less than 20%, less than 15% or less than 10% of the internucleotidic linkages in the hybrid gapmer oligonucleotide have a negative charge. In embodiments, less than less than 50%, less than 40%, less than 30%, less than 25%, less than 20%, less than 15%, less than 10% or less than 5% of the internucleotidic linkages in the 5′ flank of the hybrid gapmer oligonucleotide have a negative charge. In embodiments, less than 75%, less than 50%, less than 40%, less than 30%, less than 25%, less than 20%, less than 15% or less than 10% of the internucleotidic linkages in the gap oligonucleotide sequence of the hybrid gapmer oligonucleotide have a negative charge. In embodiments, less than less than 50%, less than 40%, less than 30%, less than 25%, less than 20%, less than 15%, less than 10% or less than 5% of the internucleotidic linkages in the 3′ flank of the hybrid gapmer oligonucleotide have a negative charge. In embodiments, the hybrid gapmer oligonucleotide comprises less than 20, less than 19, less than 18, less than 17, less than 16, less that 15, less than 14, less than 13, less than 12, less than 11, less than 10, less than 9, less than 8, less than 7, less than 6, or less than 5 charged internucleotidic linkages.

In embodiments, the terminal residue at the first 5′ (or 6′) end of the 5′ (or 6′) flank comprises a PMO 6′-phosphoramidite P(III).

In embodiments, the gap oligonucleotide sequence comprises from 8 to 20 deoxyribonucleotide or ribonucleotide analogs. In embodiments, the gap oligonucleotide sequence comprises at least 8 consecutive deoxribonucleotides or deoxyribonucleotide analogs. In embodiments, the gap oligonucleotide sequence comprises from 8 to 20 consecutive deoxyribonucleotide or ribonucleotide analogs.

In embodiments, the gap sequence of the hybrid gapmer oligonucleotide comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(III) DNA analog, a reverse P(III) DNA analog, a P(III) RNA analog, a reverse P(III) RNA analog, or a combination thereof. In embodiments, the gap sequence of the hybrid gapmer oligonucleotide comprises at least one (1) 2′-modified P(MII) ribonucleotide. In embodiments, the gap sequence comprises a 2′ modified ribonucleotide analog selected from: 2′-O-methyl (2′-OMe) P(III) RNA, reverse 2′-O-methyl (2′-OMe) P(III) RNA, 2′-O-methoxyethyl (2′-OMOE) P(III) RNA, reverse 2′-O-Methoxyethyl (2′-OMOE) P(III) RNA, 2′-fluoro P(III) RNA, reverse 2′-fluoro P(III) RNA, or combinations thereof.

In embodiments, the gap sequence of the hybrid gapmer oligonucleotide comprises at least one internucleotidic linkage selected from: phosphodiester, phosphotriester, methylphosphonate, phosphoramidate, phosphorodiamidate, phosphorothioamidate, or phosphorothioate. In embodiments, the gap sequence of the hybrid gapmer oligonucleotide comprises at least one internucleotidic linkage selected from methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). In embodiments, the gap sequence of the hybrid gapmer oligonucleotide comprises an internucleotidic linkage selected from: phosphorodiamidate, phosphorothioamidate, phosphothioate (PS) and phosphoryl guanidine linkages. In embodiments, at least 50%, at least 60%, at least 70%, at least 80%, or at least 90% of the deoxyribonucleotide analogs of the gap oligonucleotide sequence are linked through phosphothioate linkages. In embodiments, the gap sequence comprises at least two 2′ modified nucleotides linked by a phosphorthioate linkage.

In embodiments, the 3′ flank comprises from 2 to 20, from 2 to 10, or from 2 to 5 morpholino nucleotide analogs. In embodiments, the 3′ flank comprises from 2 to 20, from 2 to 10, or from 2 to 5 consecutive morpholino nucleotide analogs. In embodiments, the 3′ flank comprises 2, 3, 4, 5, 6, 7, 8, 9, 10, 11, 12, 13, 14, 15, 16, 17, 18, 19 or 20 morpholino nucleotide analogs. In embodiments, the 3′ flank comprises 2, 3, 4, 5, 6, 7, 8, 9, 10, 11, 12, 13, 14, 15, 16, 17, 18, 19 or 20 consecutive morpholino nucleotide analogs.

In embodiments, 2 or more morpholino nucleotide analogs of the 3′ flank are linked through phosphorodiamidate linkages. In embodiments, from 2 to 20, from 2 to 10, or from 2 to 5 morpholino nucleotide analogs of the 3′ flank are linked through phosphorodiamidate linkages. In embodiments, 2, 3, 4, 5, 6, 7, 8, 9, 10, 11, 12, 13, 14, 15, 16, 17, 18, 19 or 20 morpholino nucleotide analogs of the 3′ flank are linked through phosphorodiamidate linkages.

In embodiments, the 3′ flank of the hybrid gapmer oligonucleotide comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(III) DNA analog, a reverse P(IIII) DNA analog, a P(III) RNA analog, a reverse P(III) RNA analog, or a combination thereof. In embodiments, the 3′ flank of the hybrid gapmer oligonucleotide comprises at least one (1) 2′-modified P(III) ribonucleotide. In embodiments, the 3′ flank comprises a 2′ modified ribonucleotide analog selected from: 2′-O-methyl (2′-OMe) P(III) RNA, reverse 2′-O-methyl (2′-OMe) P(III) RNA, 2′-O-methoxyethyl (2′-MOE) P(III) RNA, reverse 2′-O-methoxyethyl (2′-OMOE) P(III) RNA, 2′-fluoro P(III) RNA, reverse 2′-fluoro P(III) RNA, or combinations thereof.

In embodiments, the 3′ flank of the hybrid gapmer oligonucleotide comprises at least one internucleotidic linkage selected from: phosphodiester, phosphotriester, methylphosphonate, phosphoramidate, phosphorodiamidate, phosphorothioamidate, or phosphorothioate. In embodiments, the 3′ flank of the hybrid gapmer oligonucleotide comprises at least one internucleotidic linkage selected from methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). In embodiments, the 3′ flank of the hybrid gapmer oligonucleotide comprises an internucleotidic linkage selected from: phosphorodiamidate, phosphorothioamidate, phosphothioate (PS) and phosphoryl guanidine linkages.

In embodiments, the terminal residue at the second 5′ or 6′ end of the 3′ flank comprises a PMO 6′-phosphoramidite P(III).

In embodiments, the gap oligonucleotide sequence is linked to the 3′ flank through a phosphorothioate linkage.

In embodiments, hybrid gapmer oligonucleotide comprises 10 or fewer morpholino nucleotide analogs. In embodiments, hybrid gapmer oligonucleotide comprises 7 or fewer morpholino nucleotide analogs. In embodiments, hybrid gapmer oligonucleotide comprises from 2 to 10, or 3 to 7 morpholino nucleotide analogs.

### Steric Block and Splice Switching Oligonucleotides

In embodiments, the hybrid oligonucleotide is a steric block oligonucleotide. In embodiments, the steric block oligonucleotide is a splice switching oligonucleotide.

While not wishing to be bound by theory, it is believed that PMO sequences in a steric block oligonucleotide can modulate the binding energy of the oligonucleotide to the target nucleic acid sequence. It is also believed that PMO sequence in a steric block oligonucleotide may reduce the impact of the oligonucleotide on the innate immune system, or change biodistribution, pharmacological properties, or cellular distribution of the oligonucleotide.

In embodiments, the steric block oligonucleotide comprises from 10 to 50, 15 to 40 or 20 to 30 nucleotides in length. In embodiments, the steric block oligonucleotide comprises 15, 16, 17, 18, 19, 20, 21, 22, 23, 24, 25, 26, 27, 28, 29 or 30 nucleotides in length. In embodiments, the steric block oligonucleotide comprises at least about 10%, at least about 20%, at least about 30%, at least about 40%, at least about 50%, at least about 60%, at least about 70%, at least about 80% or at least about 90% morpholino nucleotide analogs. In embodiments, the steric block oligonucleotide comprises at least about 10%, at least about 20%/o, at least about 30%, at least about 40%, at least about 50%, at least about 60%, at least about 70%, at least about 80% or at least about 90% 2′-modified RNA nucleotides.

In embodiments, the steric block oligonucleotide comprises up to 25% morpholino nucleotide analogs and up to 75% 2′-modified RNA nucleotides. In embodiments, the steric block oligonucleotide comprises up to 75% morpholino nucleotide analogs and up to 25% 2′-modified RNA nucleotides. In embodiments, the steric block oligonucleotide comprises up to 30% morpholino nucleotide analogs and up to 70% 2′-modified RNA nucleotides. In embodiments, the steric block oligonucleotide comprises up to 70% morpholino nucleotide analogs and up to 30% 2′-modified RNA nucleotides. In embodiments, the steric block oligonucleotide comprises up to 40% morpholino nucleotide analogs and up to 60% 2′-modified RNA nucleotides. In embodiments, the steric block oligonucleotide comprises up to 60% morpholino nucleotide analogs and up to 40% 2′-modified RNA nucleotides. In embodiments, the steric block oligonucleotide comprises up to 50% morpholino nucleotide analogs and up to 50% 2′-modified RNA nucleotides.

In embodiments, the steric block oligonucleotide comprises at least two consecutive morpholino nucleotide analogs which are linked via a neutral phosphorodiamidate linkage. In embodiments, the steric block oligonucleotide comprises at least 2 or at least 3 morpholino nucleotide analogs. In embodiments, the steric block oligonucleotide comprises at least one thiophosphoramidate linkage between an upstream P(V) morpholino nucleotide analog and a downstream nucleotide that is not a P(V) morpholino nucleotide analog.

In embodiments, the steric block oligonucleotide comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(III) DNA analog, a reverse P(III) DNA analog, a P(III) RNA analog, a reverse P(III) RNA analog, or a combination thereof. In embodiments, the steric block oligonucleotide comprises at least one (1) 2′-modified P(III) ribonucleotide. In embodiments, the steric block oligonucleotide comprises a 2′ modified ribonucleotide analog selected from: [0238]2′-O-methyl (2′-OMe) P(III) RNA, reverse 2′-O-methyl (2′-OMe) P(III) RNA, 2′-O-methoxyethyl (2′-OMOE) P(III) RNA, reverse 2′-O-methoxyethyl (2′-MOE) P(III) RNA, 2′-fluoro P(III) RNA, reverse 2′-fluoro P(III) RNA, or combinations thereof.

In embodiments, the steric block oligonucleotide comprises at least one internucleotidic linkage selected from: phosphodiester, phosphotriester, methylphosphonate, phosphoramidate, phosphorodiamidate, phosphorothioamidate, or phosphorothioate. In embodiments, the steric block oligonucleotide comprises at least one internucleotidic linkage selected from methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). In embodiments, the 5′ flank of the hybrid gapmer oligonucleotide comprises an internucleotidic linkage selected from: phosphorodiamidate, phosphorothioamidate, phosphothioate (PS) and phosphoryl guanidine linkages

In embodiments, the steric block oligonucleotide comprises: (i) at least one P(III) morpholino nucleotide analog; (ii) at least one P(V) morpholino nucleotide analog; (iii) at least one P(III) ribonucleotide, at least one P(III) deoxyribonucleotide analog, or a combination thereof; (iv) at least one phosphorodiamidate linkage; and (iv) at least one phosphorothioamidite linkage. In embodiments, each upstream nucleotide adjacent to each P(V) morpholino nucleotide analog is a P(III) or P(V) morpholino nucleotide analog. In embodiments, the steric block oligonucleotide comprises 2 or more consecutive P(V) morpholino nucleotide analogs linked through phosphorodiamidate linkages. In embodiments, the steric block oligonucleotide comprises from 2 to 20, 3 to 10 or 4 to 6 consecutive P(V) morpholino nucleotide analogs linked through phosphorodiamidate linkages. In embodiments, the steric block oligonucleotide comprises 2, 3, 4, 5, 6, 7, 8, 9, 10, 11, 12, 13, 14, 15, 16, 17, 18, 19, or 20 consecutive P(V) morpholino nucleotide analogs linked through phosphorodiamidate linkages. In embodiments, any P(V) morpholino nucleotide analog is linked to any downstream nucleotide that is not a P(V) morpholino nucleotide analog through a phosphorothioamidate linkage.

Short Interfering RNA (iRNA)

In embodiments, the hybrid oligonucleotide forms the sense strand, antisense strand, or both strands of a short interfering RNA (siRNA). While not wishing to be bound by theory, it is believed that the presence of PMO oligonucleotide sequences in the siRNA, particularly at the 5′ end or 3′end of the sense or antisense strand, reduces enzymatic degradation of the siRNA oligonucleotides. Again, while not wishing to be bound by theory, it is believed that PMO in the sense strand and not the antisense strand can introduce chemical asymmetry into siRNA, allowing differentiation between the antisense (guide) and sense (passenger) strands and can prevent entry of the sense strand into the RNA-induced silencing complex (RISC). It is also believed that including PMO oligonucleotide sequence in the antisense strand strand can reduce toxicity of the siRNA. PMO oligonucleotide sequences in the sense and antisense strands may also improve they pharmacologic profile of the siRNA, or change cellular distribution.

In embodiments, a siRNA is provided in which the antisense strand is a hybrid antisense oligonucleotide. In embodiments, the antisense strand is from about 10 to about 50, about 15 to about 30, or about 20 to about 30 nucleotides in length. In embodiments, the hybrid antisense oligonucleotide is about 20, 21, 22, 23, 24, 25, 26, 27, 28, 29 or 30 nucleotides in length. In embodiments, the hybrid antisense oligonucleotide of an siRNA is about 21 nucleotides in length.

In embodiments, a siRNA is provided in which the sense strand is a hybrid sense oligonucleotide. In embodiments, the sense strand is from about 10 to about 50, about 15 to about 30, or about 20 to about 30 nucleotides in length. In embodiments, the hybrid sense oligonucleotide is about 20, 21, 22, 23, 24, 25, 26, 27, 28, 29 or 30 nucleotides in length. In embodiments, the hybrid antisense oligonucleotide of an siRNA is about 21 nucleotides in length.

In embodiments, the double stranded region of the siRNA duplex includes from about 10 to about 50, about 15 to about 30, or about 20 to about 30 nucleotides. In embodiments, the double stranded region of the siRNA duplex is about 20, 21, 22, 23, 24, 25, 26, 27, 28, 29 or 30 nucleotides in length. In embodiments, the double stranded region of the siRNA duplex is about 21 nucleotides in length.

In embodiments, the double-stranded siRNA duplex includes a single strand or unpaired region at one or both ends of the molecule. In embodiments, the sense strand of the double-stranded siRNA duplex includes a 5′ overhang, a 3′ overhang, or both. In embodiments, the antisense strand of the double-stranded siRNA duplex includes a 5′ overhang, a 3′ overhang, or both. In embodiments, the overhang(s) are each independently from about 1 to about 5 nucleotides in length, or about 1 to about 3 nucleotides in length, or about 1, 2, 3, 4, or 5 nucleotides in length. The overhangs can be the result of one strand being longer than the other, or the result of two strands of the same length being staggered. In embodiments, the siRNA duplex includes at least one 3′ overhang. In embodiments, the siRNA duplex includes at least one 5′ overhang. In embodiment, both the sense and antisense strands of the siRNA duplex have a 3′ overhang. In embodiment, both the sense and antisense strands of the siRNA duplex have a 5′ overhang.

In embodiments, the sense strand of the siRNA is the same length as the or shorter than the antisense strand, e.g., 15-16 mer. In embodiments, morpholino nucleotide analogs are included in the antisense strand, the sense strand, or both. In embodiments, morpholino nucleotide analogs are included in only in one of the strands of the siRNA duplex. In embodiments, morpholino nucleotide analogs are included in the sense strand of the siRNA duplex. In embodiments, morpholino nucleotide analogs are included in the antisense strand of the siRNA duplex. In embodiments, morpholino nucleotide analogs are included only in the sense strand of the siRNA duplex.

In embodiments, the sense strand includes 2′ modified nucleotides, such as 2′F, 2′O-MOE or 2′ OMe modifications at one or both the 5′ and the 3′ end of the sense strand. In embodiments, the sense strand includes 2′ modified nucleotides at one or more of positions 1, 2 or 3 from the end 5′ or 3′ end of the sense strand. In embodiments, the siRNA sense strand has from 1 to 5 2′ modified nucleotides.

In embodiments, the antisense strand includes 2′ modified nucleotides, such as 2′F, 2′O-MOE or 2′ OMe modifications at one or both the 5′ and the 3′ end of the antisense strand. In embodiments, the antisense strand includes 2′ modified nucleotides at one or more of positions 1, 2 or 3 from the end 5′ or 3′ end of the antisense strand. In embodiments, the siRNA antisense strand has from 1 to 5 2′ modified nucleotides.

In embodiments, the sense strand comprises at least two consecutive morpholino nucleotide analogs which are linked via a neutral phosphorodiamidate linkage. In embodiments, the sense strand comprises at least 2 or at least 3 morpholino nucleotide analogs. In embodiments, the sense strand comprises at lease one thiophosphoramidate linkage between an upstream P(V) morpholino nucleotide analog and a downstream nucleotide that is not a P(V) morpholino nucleotide analog.

In embodiments, the sense strand comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(III) DNA analog, a reverse P(III) DNA analog, a P(III) RNA analog, a reverse P(III) RNA analog, or a combination thereof. In embodiments, the sense strand comprises at least one (1) 2′-modified P(III) ribonucleotide. In embodiments, the sense strand comprises a 2′ modified ribonucleotide analog selected from:

2′-O-methyl (2′-OMe) P(II) RNA, reverse 2′-O-methyl (2′-OMe) P(III) RNA, 2′-O-methoxyethyl (2′-OMOE) P(III) RNA, reverse 2′-O-methoxyethyl (2′-MOE) P(III) RNA, 2′-fluoro P(III) RNA, reverse 2′-fluoro P(III) RNA, or combinations thereof.

In embodiments, the sense strand comprises at least one internucleotidic linkage selected from: phosphodiester, phosphotriester, methylphosphonate, phosphoramidate, phosphorodiamidate, phosphorothioamidate, or phosphorothioate. In embodiments, sense strand comprises at least one internucleotidic linkage selected from methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). In embodiments, the 5′ flank of the hybrid gapmer oligonucleotide comprises an internucleotidic linkage selected from: phosphorodiamidate, phosphorothioamidate, phosphothioate (PS) and phosphoryl guanidine linkages

In embodiments, the sense strand comprises: (i) at least one P(III) morpholino nucleotide analog; (ii) at least one P(V) morpholino nucleotide analog; (iii) at least one P(III) ribonucleotide, at least one P(III) deoxyribonucleotide analog, or a combination thereof; (iv) at least one phosphorodiamidate linkage; and (iv) at least one phosphorothioamidite linkage. In embodiments, each upstream nucleotide adjacent to each P(V) morpholino nucleotide analog is a P(III) or P(V) morpholino nucleotide analog. In embodiments, the sense strand comprises 2 or more consecutive P(V) morpholino nucleotide analogs linked through phosphorodiamidate linkages. In embodiments, the sense strand comprises from 2 to 20, 3 to 10 or 4 to 6 consecutive P(V) morpholino nucleotide analogs linked through phosphorodiamidate linkages. In embodiments, the sense strand comprises 2, 3, 4, 5, 6, 7, 8, 9, 10, 11, 12, 13, 14, 15, 16, 17, 18, 19, or 20 consecutive P(V) morpholino nucleotide analogs linked through phosphorodiamidate linkages. In embodiments, any P(V) morpholino nucleotide analog is linked to any downstream nucleotide that is not a P(V) morpholino nucleotide analog through a phosphorothioamidate linkage.

In embodiments, the antisense strand comprises at least two consecutive morpholino nucleotide analogs which are linked via a neutral phosphorodiamidate linkage. In embodiments, the antisense strand comprises at least 2 or at least 3 morpholino nucleotide analogs. In embodiments, the antisense strand comprises at lease one thiophosphoramidate linkage between an upstream P(V) morpholino nucleotide analog and a downstream nucleotide that is not a P(V) morpholino nucleotide analog.

In embodiments, the antisense strand comprises one or more nucleotide residues comprising a deoxyribonucleotide or analog thereof, or a ribonucleotide or analog thereof, or a combination thereof. In embodiments, the deoxyribonucleotide analog or ribonucleotide analog is selected from: a P(III) DNA analog, a reverse P(III) DNA analog, a P(III) RNA analog, a reverse P(III) RNA analog, or a combination thereof. In embodiments, the antisense strand comprises at least one (1) 2′-modified P(I) ribonucleotide. In embodiments, the antisense strand comprises a 2′ modified ribonucleotide analog selected from: 2′-O-methyl (2′-OMe) P(IIII) RNA, reverse 2′O-methyl (2′-OMe) P(III) RNA, 2′-O-methoxyethyl (2′-OMOE) P(III) RNA, reverse 2′-O-methoxyethyl (2′-OMOE) P(III) RNA, 2′-fluoro P(III) RNA, reverse 2′-fluoro P(III) RNA, or combinations thereof.

In embodiments, the antisense strand comprises at least one internucleotidic linkage selected from: phosphodiester, phosphotriester, methylphosphonate, phosphoramidate, phosphorodiamidate, phosphorothioamidate, or phosphorothioate. In embodiments, antisense strand comprises at least one internucleotidic linkage selected from methylenemethylimino (—CH₂—N(CH₃)—O—CH₂—), thiodiester (—O—C(O)—S—), thionocarbamate (—O—C(O)(NH)—S—), siloxane (—O—Si(H)₂—O—); and N,N′-dimethylhydrazine (—CH₂—N(CH₃)—N(CH₃)—). In embodiments, the 5′ flank of the hybrid gapmer oligonucleotide comprises an internucleotidic linkage selected from: phosphorodiamidate, phosphorothioamidate, phosphothioate (PS) and phosphoryl guanidine linkages

In embodiments, the antisense strand comprises: (i) at least one P(III) morpholino nucleotide analog; (ii) at least one P(V) morpholino nucleotide analog; (iii) at least one P(III) ribonucleotide, at least one P(III) deoxyribonucleotide analog, or a combination thereof; (iv) at least one phosphorodiamidate linkage; and (iv) at least one phosphorothioamidite linkage. In embodiments, each upstream nucleotide adjacent to each P(V) morpholino nucleotide analog is a P(III) or P(V) morpholino nucleotide analog. In embodiments, the antisense strand comprises 2 or more consecutive P(V) morpholino nucleotide analogs linked through phosphorodiamidate linkages. In embodiments, the antisense strand comprises from 2 to 20, 3 to 10 or 4 to 6 consecutive P(V) morpholino nucleotide analogs linked through phosphorodiamidate linkages. In embodiments, the antisense strand comprises 2, 3, 4, 5, 6, 7, 8, 9, 10, 11, 12, 13, 14, 15, 16, 17, 18, 19, or 20 consecutive P(V) morpholino nucleotide analogs linked through phosphorodiamidate linkages. In embodiments, any P(V) morpholino nucleotide analog is linked to any downstream nucleotide that is not a P(V) morpholino nucleotide analog through a phosphorothioamidate linkage.

### Endosomal Escape Vehicles (EEVs)

An endosomal escape vehicle (EEV) is provided herein that can be used to transport a cargo across a cellular membrane, for example, to deliver the cargo to the cytosol or nucleus of a cell. Cargo can include a therapeutic moiety (TM), for example, a hybrid oligonucleotide as described herein. The EEV can comprise a cell penetrating peptide (CPP), for example, a cyclic cell penetrating peptide (cCPP), which is conjugated to an exocyclic peptide (EP). The EP can be referred to interchangeably as a modulatory peptide (MP).

The EP can comprise a sequence of a nuclear localization signal (NLS). The EP can be coupled to the cargo. The EP can be coupled to the cCPP. The EP can be coupled to the cargo and the cCPP. Coupling between the EP, cargo, cCPP, or combinations thereof, may be non-covalent or covalent. The EP can be attached through a peptide bond to the N-terminus of the cCPP. The EP can be attached through a peptide bond to the C-terminus of the cCPP. The EP can be attached to the cCPP through a side chain of an amino acid in the cCPP. The EP can be attached to the cCPP through a side chain of a lysine which can be conjugated to the side chain of a glutamine in the cCPP. The EP can be conjugated to the 5′ or 3′ end of an oligonucleotide cargo. The EP can be coupled to a linker. The exocyclic peptide can be conjugated to an amino group of the linker. The EP can be coupled to a linker via the C-terminus of an EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to attach the cCPP, the C- or N-terminus may be attached to a linker on the cargo.

### Exocyclic Peptides

In embodiments, a compound is provided that includes a hybrid oligonucleotide, a cyclic cell penetrating peptide (cCCP) and an exocyclic peptide (EP). The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, alloisoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amino acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2 lysine residues. The EP can comprise 3 lysine residues. The EP can comprise 4 lysine residues. The amino group on the side chain of each lysine residue can be substituted with a protecting group, including, for example, trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH, KHKK, KKHK, KKKH, KHKH, HKHK, KKKK, KKRK, KRKK, KRRK, RKKR, RRRR, KGKK, KKGK, HBHBH, HBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH, RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG, wherein B is beta-alanine. The amino acids in the EP can have D or L stereochemistry.

The EP can comprise KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can comprise PKKKRKV, RR, RRR, RHR, RBR, RBRBR, RBHBR, or HBRBH, wherein B is beta-alanine. The amino acids in the EP can have D or L stereochemistry.

The EP can consist of KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can consist of PKKKRKV, RR, RRR, RHR, RBR, RBRBR, RBHBR, or HBRBH, wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV. The EP can consist of an NLS comprising the amino acid sequence PKKKRKV. The EP can comprise an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK. The EP can consist of an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK

All exocyclic sequences can also contain an N-terminal acetyl group. Hence, for example, the EP can have the structure: Ac-PKKKRKV.

### Cell Penetrating Peptides (CPP)

The cell penetrating peptide (CPP) can comprise 6 to 20 amino acid residues. The cell penetrating peptide can be a cyclic cell penetrating peptide (cCPP). The cCPP is capable of penetrating a cell membrane. An exocyclic peptide (EP) can be conjugated to the cCPP, and the resulting construct can be referred to as an endosomal escape vehicle (EEV). The cCPP can direct a cargo (e.g., a therapeutic moiety (TM), for example, a hybrid oligonucleotide as described herein) to penetrate the membrane of a cell. The cCPP can deliver the cargo to the cytosol of the cell. The cCPP can deliver the cargo to a cellular location where a target (e.g., pre-mRNA) is located. To conjugate the cCPP to a cargo (e.g., a hybrid oligonucleotide as described herein), at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP disclosed herein can comprise 6 to 10 amino acids. By way of example, cCPP comprising 6-10 amino acid residues can have a structure according to any of Formula I-A to I-E:

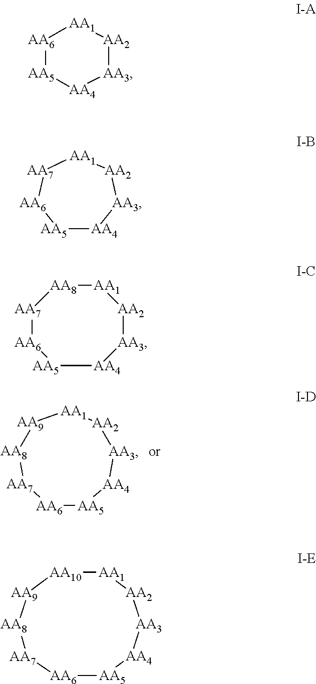

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein.

TABLE 1
Amino Acid Abbreviations

| | Abbrevi- | Abbrevi- |
|---|---|---|
| | ations* | ations* |
| Amino Acid | L-amino acid | D-amino acid |
| Alanine | Ala (A) | ala (a) |
| Allo-isoleucine | Aile | Aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Citrulline | Cit | cit |
| Cyclohexylalanine | Cha | cha |
| 2,3-diaminopropionic acid | Dap | dap |
| 4-fluorophenylalanine | Fpa (Σ) | pfa |
| Glutamic acid | Glu (E) | glu (e) |
| Glutamine | Gln (Q) | gln (q) |
| Glycine | Gly (G) | gly (g) |
| Histidine | His (H) | his (h) |
| Homoproline (aka pipecolic acid) | Pip (Θ) | pip (θ) |
| Isoleucine | Ile (I) | ile (i) |
| Leucine | Leu (L) | leu (l) |
| Lysine | Lys (K) | lys (k) |
| Methionine | Met (M) | met (m) |
| 3-(2-naphthyl)-alanine | Nal (Φ) | nal (ϕ) |
| 3-(1-naphthyl)-alanine | 1-Nal | 1-nal |
| Norleucine | Nle (Ω) | nle |
| Phenylalanine | Phe (F) | phe (f) |
| Phenylglycine | Phg (Ψ) | phg |
| 4- | F₂Pmp (∧) | f₂pmp |
| (phosphonodifluoromethyl)phenylalanine | | |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |

*single letter abbreviations: when shown in capital letters herein it indicates the L-amino acid form, when shown in lower case herein it indicates the D-amino acid form.

The cCPP can comprise 6 to 20 amino acids, wherein: (i) at least one amino acid has a side chain comprising a guanidine group, or a protonated form thereof; (ii) at least one amino acid has no side chain or a side chain comprising

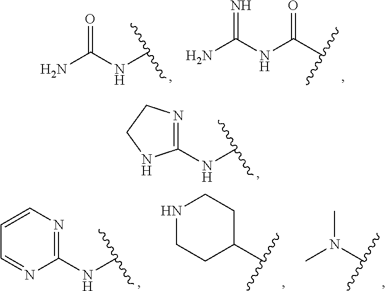

or a protonated form thereof; and (iii) at least two amino acids independently have a side chain comprising an aromatic or heteroaromatic group.

At least two amino acids can have no side chain or a side chain comprising

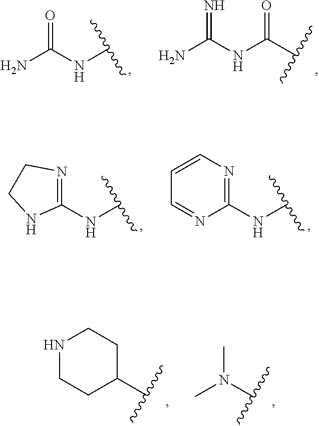

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or β-alanine.

The cCPP can comprise from 6 to 20 amino acid residues which form the cCPP, wherein: (i) at least one amino acid can be glycine, β-alanine, or 4-aminobutyric acid residues; (ii) at least one amino acid can have a side chain comprising an aryl or heteroaryl group; and (iii) at least one amino acid has a side chain comprising a guanidine group,

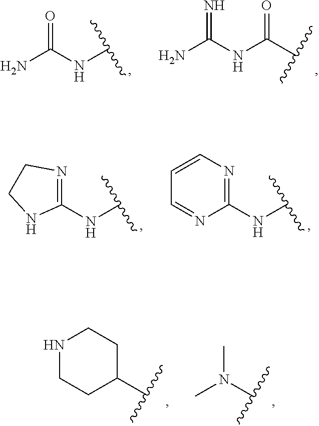

or a protonated form thereof.

The cCPP can comprise from 6 to 20 amino acid residues which form the cCPP, wherein: (i) at least two amino acid can independently be glycine, β-alanine, or 4-aminobutyric acid residues; (ii) at least one amino acid can have a side chain comprising an aryl or heteroaryl group; and (iii) at least one amino acid has a side chain comprising a guanidine group,

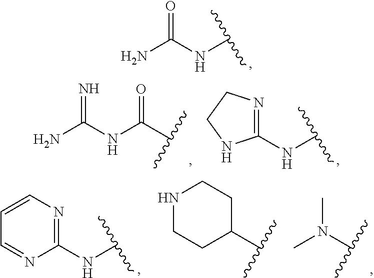

or a protonated form thereof.

The cCPP can comprise from 6 to 20 amino acid residues which form the cCPP, wherein: (i) at least three amino acids can independently be glycine, β-alanine, or 4-aminobutyric acid residues; (ii) at least one amino acid can have a side chain comprising an aromatic or heteroaromatic group; and (iii) at least one amino acid can have a side chain comprising a guanidine group,

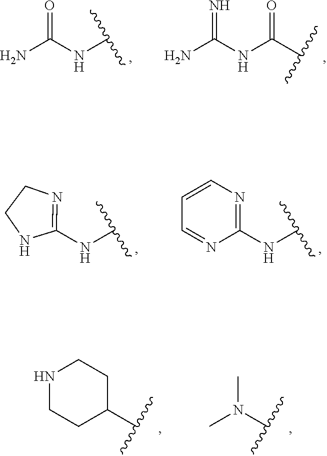

a protonated form thereof.

### Glycine and Related Amino Acid Residues

The cCPP can comprise (i) 1, 2, 3, 4, 5, or 6 glycine, β-alanine, 4-aminobutyric acid residues, or combinations thereof. The cCPP can comprise (i) 2 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (i) 3, 4, 5, or 6 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, pi-alanine, 4-aminobutyric acid residues, or combinations thereof. The cCPP can comprise (i) 3, 4, or 5 glycine, pi-alanine, 4-aminobutyric acid residues, or combinations thereof. The cCPP can comprise (i) 3 or 4 glycine, β-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues

In embodiments, none of the glycine, β-alanine, or 4-aminobutyric acid residues in the cCPP are contiguous. Two or three glycine, β-alanine, 4- or aminobutyric acid residues can be contiguous. Two glycine, β-alanine, or 4-aminobutyric acid residues can be contiguous.

In embodiments, none of the glycine residues in the cCPP are contiguous. Each glycine residues in the cCPP can be separated by an amino acid residue that cannot be glycine. Two or three glycine residues can be contiguous. Two glycine residues can be contiguous

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group. The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

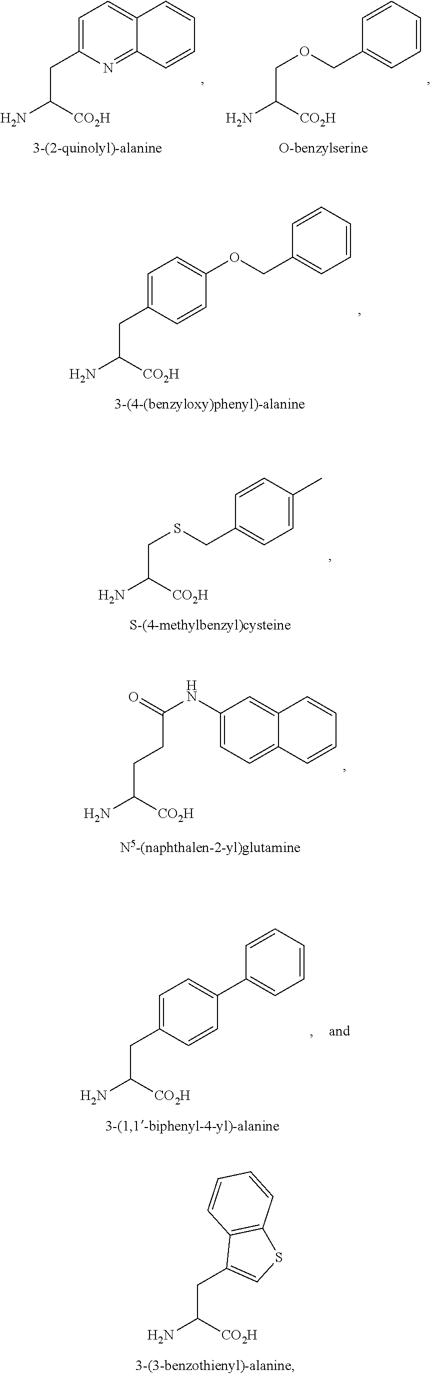

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, 0-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

While not wishing to be bound by theory, it is believed that amino acids having an aromatic or heteroaromatic group having higher hydrophobicity values (i.e., amino acids having side chains comprising aromatic or heteroaromatic groups) can improve cytosolic delivery efficiency of a cCPP relative to amino acids having a lower hydrophobicity value. Each hydrophobic amino acid can independently have a hydrophobicity value greater than that of glycine. Each hydrophobic amino acid can independently be a hydrophobic amino acid having a hydrophobicity value greater than that of alanine. Each hydrophobic amino acid can independently have a hydrophobicity value greater or equal to phenylalanine. Hydrophobicity may be measured using hydrophobicity scales known in the art. Table B lists hydrophobicity values for various amino acids as reported by Eisenberg and Weiss (Proc. Natl. Acad. Sci. U.S.A. 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A. 1981; 78(6):3824-3828), and Janin (Nature. 1979; 277(5696):491-492), the entirety of each of which is herein incorporated by reference. Hydrophobicity can be measured using the hydrophobicity scale reported in Engleman, et al.

TABLE B
Amino Acid Hydrophobicity

| Amino | | Eisenberg | Engleman | Kyrie and | Hoop and | |
|---|---|---|---|---|---|---|
| Acid | Group | and Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

### Amino Acid Residues Having a Side Chain Comprising a Guanidine Group, Guanidine Replacement Group, or Protonated Form Thereof

As used herein, guanidine refers to the structure:

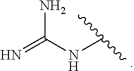 [SMILES: *NC(=N)N]

As used herein, a protonated form of guanidine refers to the structure.

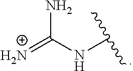 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

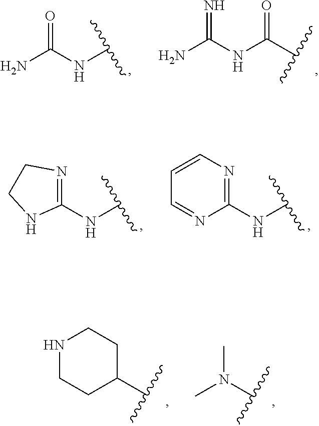

or a protonated form thereof.

The disclosure relates to a cCPP comprising from 4 to 20 amino acids residues, wherein: (i) at least one amino acid has a side chain comprising a guanidine group, or a protonated form thereof; (ii) at least one amino acid residue has no side chain or a side chain comprising

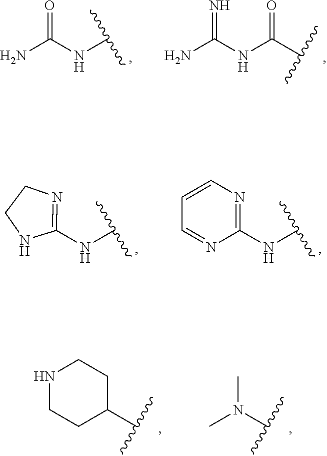

or a protonated form thereof, and (iii) at least two amino acids residues independently have a side chain comprising an aromatic or heteroaromatic group.

At least two amino acids residues can have no side chain or a side chain comprising

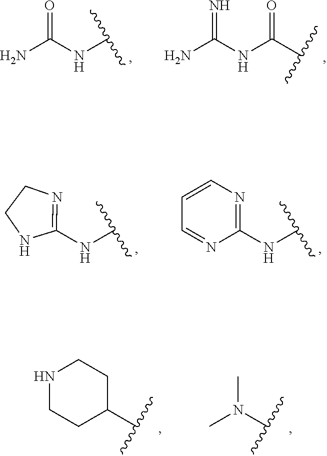

or a protonated form thereof. As used herein, when no side chain is present, the amino acid residue have two hydrogen atoms on the carbon atom(s) (e.g., —CH₂—) linking the amine and carboxylic acid.

The cCPP can comprise at least one amino acid having a side chain comprising one of the following moieties:

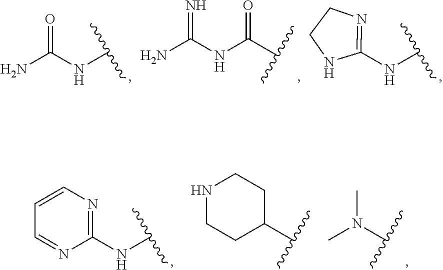

or a protonated form thereof.

The cCPP can comprise at least two amino acids each independently having one of the following moieties

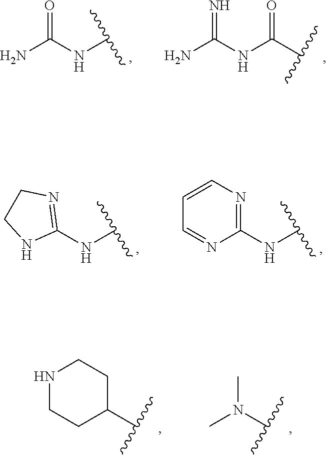

or a protonated form thereof. At least two amino acids can have a side chain comprising the same moiety selected from:

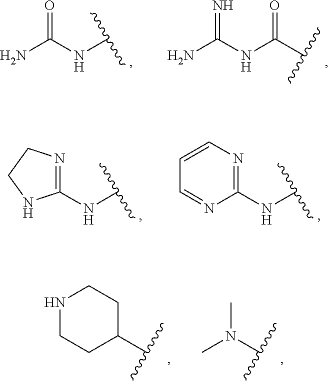

or a protonated form thereof. At least one amino acid can have a side chain comprising

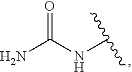 [SMILES: *NC(N)=O]

or a protonated form thereof. At least two amino acids can have a side chain comprising

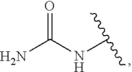 [SMILES: *NC(N)=O]

or a protonated form thereof. One, two, three, or four amino acids can have a side chain comprising

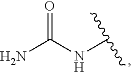 [SMILES: *NC(N)=O]

or a protonated form thereof. One amino acid can have a side chain comprising

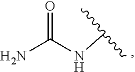 [SMILES: *NC(N)=O]

or a protonated form thereof. Two amino acids can have a side chain comprising

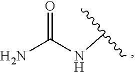 [SMILES: *NC(N)=O]

or a protonated form thereof.

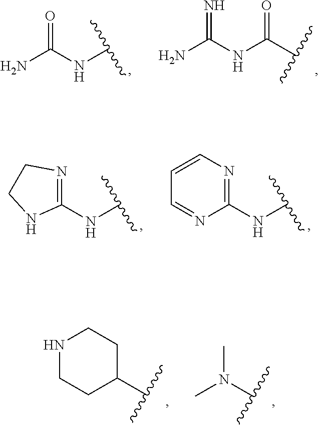

a protonated form thereof, can be attached to the terminus of the amino acid side chain.

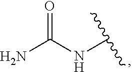 [SMILES: *NC(N)=O]

can be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently be

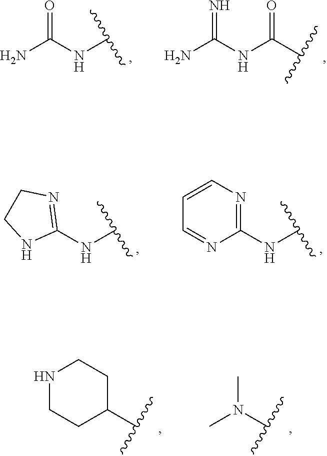

or a protonated form thereof.

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutamine acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids. The amino acid residues that form the cCPP can all be D-amino acids.

At least two of the amino acids can have a different chirality. At least two amino acids having a different chirality can be adjacent to each other. At least three amino acids can have different chirality relative to an adjacent amino acid. At least four amino acids can have different chirality relative to an adjacent amino acid. At least two amino acids have the same chirality and at least two amino acids have a different chirality. One or more amino acid residues that form the cCPP can be achiral. The cCPP can comprise a motif of 3, 4, or 5 amino acids, wherein two amino acids having the same chirality can be separated by an achiral amino acid. The cCPPs can comprise the following sequences: D-X-D; D-X-D-X; D-X-D-X-D; L-X-L; L-X-L-X; or L-X-L-X-L, wherein X is an achiral amino acid. The achiral amino acid can be glycine.

An amino acid having a side chain comprising:

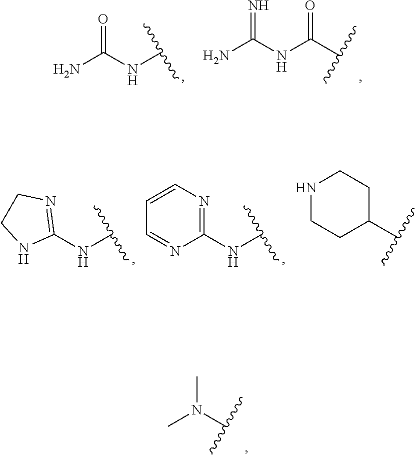

or a protonated form thereof, can be adjacent to an amino acid having a side chain comprising an aromatic or heteroaromatic group. An amino acid having a side chain comprising:

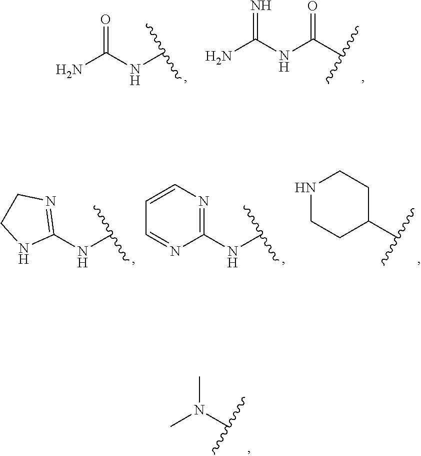

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

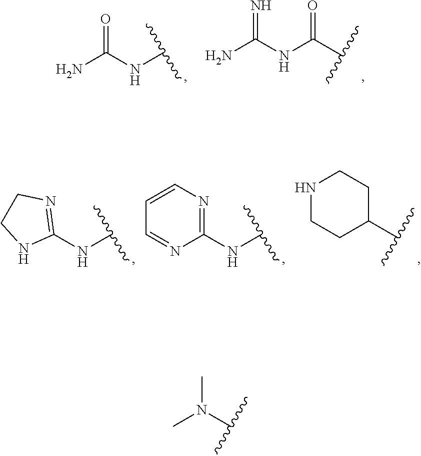

or protonated forms there, can be adjacent to each other. Two amino acids having a side chain comprising a guanidine or protonated form thereof are adjacent to each other. The cCPPs can comprise at least two contiguous amino acids having a side chain can comprise an aromatic or heteroaromatic group and at least two non-adjacent amino acids having a side chain comprising:

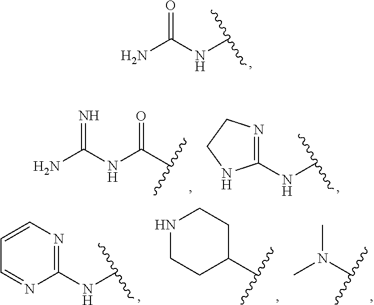

or a protonated form thereof. The cCPPs can comprise at least two contiguous amino acids having a side chain comprising an aromatic or heteroaromatic group and at least two non-adjacent amino acids having a side chain comprising

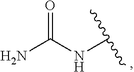 [SMILES: *NC(N)=O]

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

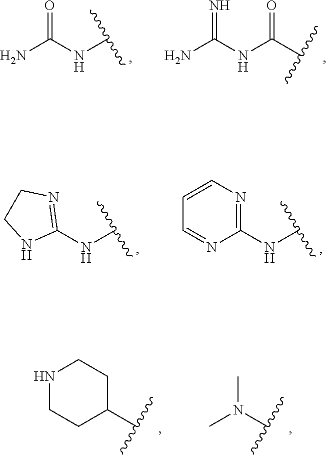

or a protonated form thereof, are alternating with at least two amino acids having a side chain comprising a guanidine group or protonated form thereof.

The cCPP can comprise the structure of Formula (A):

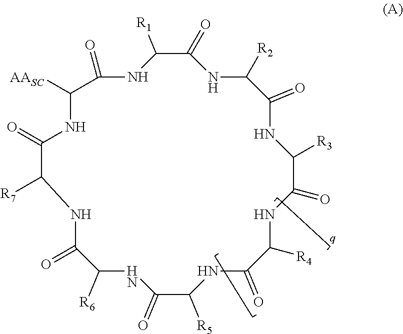

or a protonated form thereof,
wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;
AA_{SC} is an amino acid side chain; and
q is 1, 2, 3 or 4.

In embodiments, at least one of R₄, R₅, R₆, R₇ are independently an uncharged, non-aromatic side chain of an amino acid. In embodiments, at least one of R₄, R₅, R₆, R₇ are independently H or a side chain of citrulline.

In embodiments, q is 1. In embodiments, q is 2. In embodiments, q is 3. In embodiments, q is 4.

In embodiments, compounds are provided that include a cyclic peptide having 6 to 12 amino acids, wherein at least two amino acids of the cyclic peptide are charged amino acids, at least two amino acids of the cyclic peptide are aromatic hydrophobic amino acids and at least two amino acids of the cyclic peptide are uncharged, non-aromatic amino acids. In embodiments, at least two charged amino acids of the cyclic peptide are arginine. In embodiments, at least two aromatic, hydrophobic amino acids of the cyclic peptide are phenylalanine, naphtha alanine (3-Naphth-2-yl-alanine) or a combination thereof. In embodiments, at least two uncharged, non-aromatic amino acids of the cyclic peptide are citrulline, glycine or a combination thereof. In embodiments, the compound is a cyclic peptide having 6 to 12 amino acids wherein two amino acids of the cyclic peptide are arginine, at least two amino acids are aromatic, hydrophobic amino acids selected from phenylalanine, naphtha alanine and combinations thereof, and at least two amino acids are uncharged, non-aromatic amino acids selected from citrulline, glycine and combinations thereof.

The cCPP can comprise the structure of Formula (I):

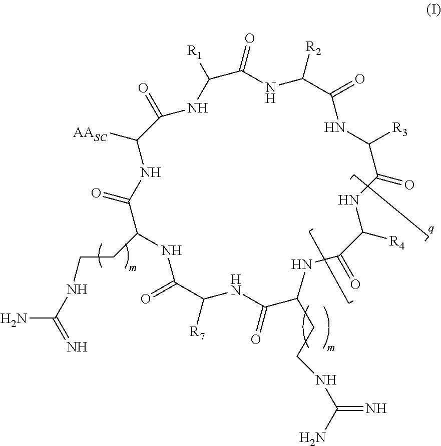

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₇ are independently H or an amino acid side chain;
AA_{SC} is an amino acid side chain;
q is 1, 2, 3 or 4; and
each m is independently an integer 0, 1, 2, or 3.

R₁, R₂, and R₃ can each independently be H, -alkylene-aryl, or -alkylene-heteroaryl. R₁, R₂, and R₁ can each independently be H, —C₁₋₃alkylene-aryl, or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂ and R₃ can each independently be H, —CH₂Ph, or —CH₂Naphthyl. R₁, R₂, and R₃ can each independently be H or —CH₂Ph.

R₁, R₂, and Rz can each independently be the side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ can be the side chain of tyrosine. R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of 3-benzothienylalanine. R₁ can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of β-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

R₂ can be the side chain of tyrosine. R₂ can be the side chain of phenylalanine. R₂ can be the side chain of 1-naphthylalanine. R₁ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of β-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

R₃ can be the side chain of tyrosine. R₁ can be the side chain of phenylalanine. R₃ can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. R₃ can be the side chain of 4-trifluoromethylphenylalanine. R₃ can be the side chain of 2,3,4,5,6-pentafluorophenylalanine. R₃ can be the side chain of homophenylalanine. R₃ can be the side chain of β-homophenylalanine. R₁ can be the side chain of 4-tert-butyl-phenylalanine. R₃ can be the side chain of 4-pyridinylalanine. R₃ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

R₄ can be H, -alkylene-aryl, -alkylene-heteroaryl. R₄ can be H, —C₁₋₃alkylene-aryl, or —C₁₋₃alkylene-heteroaryl. R₄ can be H or -alkylene-aryl. R₄ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₄ can be H, —C₁₋₃alkylene-Ph or —C₁₋₃alkylene-Naphthyl. R₄ can be H or the side chain of an amino acid in Table A or Table C. R₄ can be H or an amino acid residue having a side chain comprising an aromatic group. R₄ can be H, —CH₂Ph, or —CH₂Naphthyl. R₄ can be H or —CH₂Ph.

R₅ can be H, -alkylene-aryl, -alkylene-heteroaryl. R₅ can be H, —C₁₋₃alkylene-aryl, or —C₁₋₃ alkylene-heteroaryl. R₅ can be H or -alkylene-aryl. R₅ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₅ can be H, —C₁₋₃alkylene-Ph or —C₁₋₃alkylene-Naphthyl. R₅ can be H or the side chain of an amino acid in Table 1 or Table 3. R₄ can be H or an amino acid residue having a side chain comprising an aromatic group. R₅ can be H, —CH₂Ph, or -CH₂Naphthyl. R₄ can be H or —CH₂Ph.

R can be H, -alkylene-aryl, -alkylene-heteroaryl. R₆ can be H, —C₁₋₃alkylene-aryl, or —C₁₋₃ alkylene-heteroaryl. R₆ can be H or -alkylene-aryl. R₆ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₆ can be H, —C₁₋₃alkylene-Ph or —C₁₋₃alkylene-Naphthyl. R₆ can be H or the side chain of an amino acid in Table A or Table C. R₆ can be H or an amino acid residue having a side chain comprising an aromatic group. R₆ can be H, —CH₂Ph, or —CH₂Naphthyl. R₆ can be H or —CH₂Ph.

R₇ can be H, -alkylene-aryl, -alkylene-heteroaryl. R₇ can be H, —C₁₋₃alkylene-aryl, or —C₁₋₃ alkylene-heteroaryl. R₇ can be H or -alkylene-aryl. R₇ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₇ can be H, —C₁₋₃alkylene-Ph or —C₁₋₃alkylene-Naphthyl. R₇ can be H or the side chain of an amino acid in Table A or Table C. R₇ can be H or an amino acid residue having a side chain comprising an aromatic group. R₇ can be H, —CH₂Ph, or —CH₂Naphthyl. R₇ can be H or —CH₂Ph.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₄, R₅, R₆, and R₇ can be —CH₂Ph. Three of R₁, R₂, R₃, R₄, R₅, R₆, and R₇ can be —CH₂Ph. At least one of R₁, R₂, R₃, R₄, R₅, R₆, and R₇ can be —CH₂Ph. No more than four of R₁, R₂, R₃, R₄, R₅, R₆, and R₇ can be —CH₂Ph.

One, two or three of R₁, R₂, R₃, and R₄ are —CH₂Ph. One of R₁, R₂, R₃, and R₄ is —CH₂Ph. Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₁, R₆, and R₇ can be H. One of R₁, R₂, R₃, R₁, R₁, R₆, and R₇ can be H. Two of R₁, R₂, R₃, R₄, R₅, R₆, and R₇ are H. Three of R₁, R₂, R₃, R₅, R₆, and R₇ can be H. At least one of R₁, R₂, R₃, R₄, R₅, R₆, and R can be H. No more than three of R₁, R₂, R₃, R₄, R₅, R₆, and R₇ can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

At least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid. At least one of R₄, R₅, R₆, and R₇ can be side chain of arginine. At least one of R₄, R₅, R₆, and R₇ can be side chain of homoarginine. At least one of R₄, R₅, R₆, and R₇ can be side chain of N-methylarginine. At least one of R₄, R₅, R₆, and R₇ can be side chain of N,N-dimethylarginine. At least one of R₄, R₅, R₆, and R₇ can be side chain of 2,3-diaminopropionic acid. At least one of R₄, R₅, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R₇ can be side chain of N,N-dimethyllysine, D-homoarginine. At least one of R₄, R₅, R₆, and R₇ can be side chain of 3-(1-piperidinyl)alanine.

At least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid. At least two of R₄, R₅, R₆, and R₇ can be side chain of arginine. At least two of R₄, R₅, R₆, and R₇, can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₅, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, β-homoarginine. At least two of R₁, R₅, R₆, and R₇ can be side chain of 3-(1-piperidinyl)alanine.

At least three of R₄, R₅, R₆, and R can be side chain of 3-guanidino-2-aminopropionic acid. At least three of R₄, R₅, R₆, and R₇ can be side chain of 4-guanidino-2-aminobutanoic acid. At least three of R₄, R₅, R₆, and R₇ can be side chain of arginine. At least three of R₁, R₅, R₆, and R₇ can be side chain of homoarginine. At least three of R₄, R₅, R₆, and R₇ can be side chain of N-methylarginine. At least three of R₄, R₅, R₆, and R₇ can be side chain of N,N-dimethylarginine. At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R₇ can be side chain of N-ethyllysine. At least three of R₄, R₅, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N,N-dimethyllysine, β-homoarginine. At least three of R₄, R₅, R₆, and R₇ can be side chain of 3-(1-piperidinyl)alanine.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.
q can be 1, 2, or 3, q can 1 or 2, q can be 1, q can be 2, q can be 3, q can be 4.
m can be 1-3, m can be 1 or 2, m can be 0, m can be 1, m can be 2, m can be 3.

The cCPP of Formula (A) can comprise the structure of Formula (I)

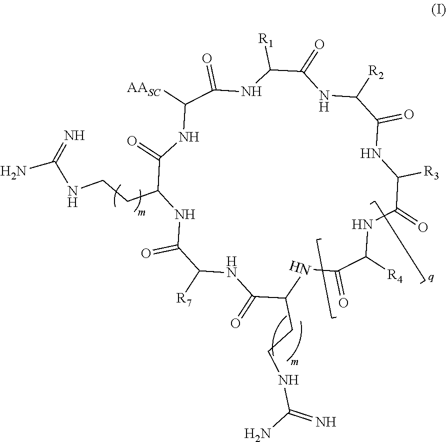

or protonated form thereof, wherein AA_{SC}, R₁, R₂, R₃, R₄, R₇, m and q are as defined herein

The cCPP of Formula (A) can comprise the structure of Formula (I-a) or Formula (I-b):

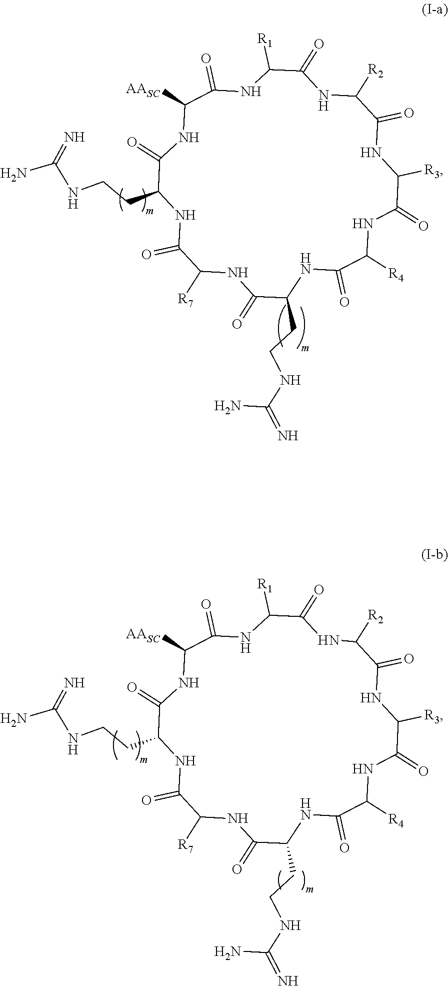

or protonated form thereof, wherein AA_{SC}, R₁, R₂, R₃, R₄, and m are as defined herein.

The cCPP of Formula (A) can comprise the structure of Formula (I-1), (I-2), (I-3) or (I-4):

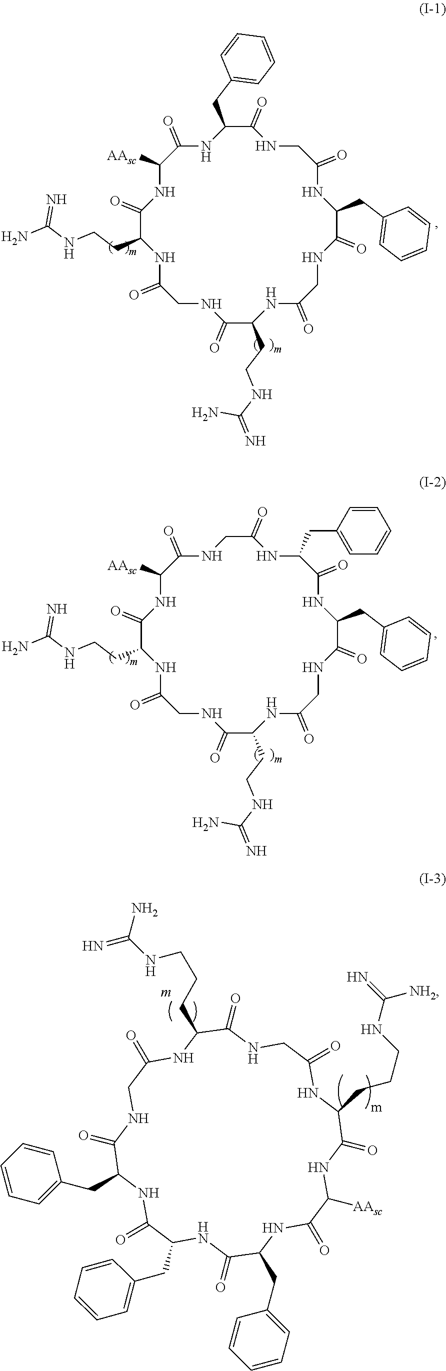

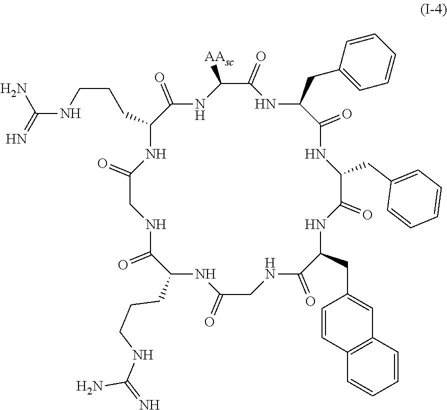

or protonated form thereof, wherein AA_{SC} and m are as defined herein.

The cCPP of Formula (A) can comprise the structure of Formula (I-5) or (I-6):

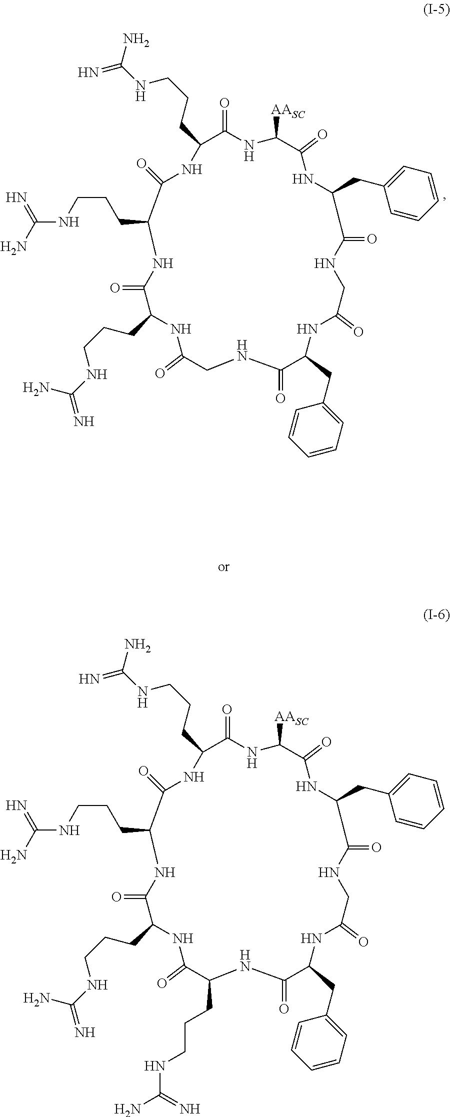

or protonated form thereof, wherein AA_{SC} is as defined herein.

The cCPP can comprise one of the following sequences: FGFGRGR; GfFGrGr, FfΦGRGR; FfFGRGR; or FfΦGrGr. The cCPP can have one of the following sequences: FGFGRGRQ; GfFGrGrQ, FfΦGRGRQ; FfFGRGRQ; or FfΦGrGrQ.

The disclosure also relates to a cCPP having the structure of Formula (II):

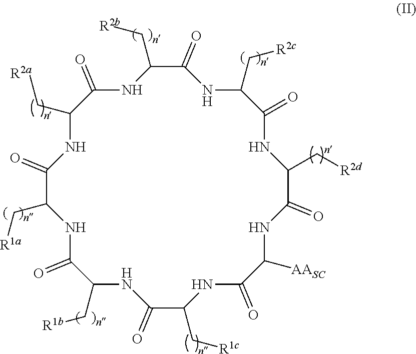

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is H

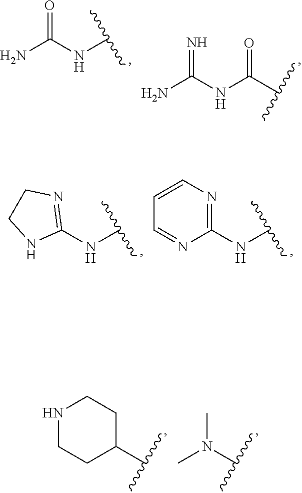

or a protonated form thereof;
at least one of R^{2a}, R^{2b}, R^{2c} and R^{2d} is guanidine or a protonated form thereof;
each n″ is independently an integer 0, 1, 2, 3, 4, or 5;
each n′ is independently an integer from 0, 1, 2, or 3; and
if n′ is 0 then R^{2a}, R^{2b}, R^{2c} or R^{2d} is absent.

At least two of R^{2a}, R^{2b}, R^{2c} and R^{2d} can be

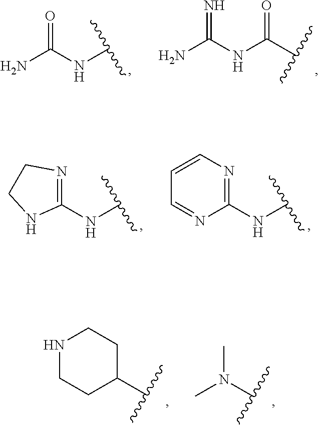

or a protonated form thereof. Two or three of R^{2a}, R^{2b}, R^{2c} and R^{2d} can be

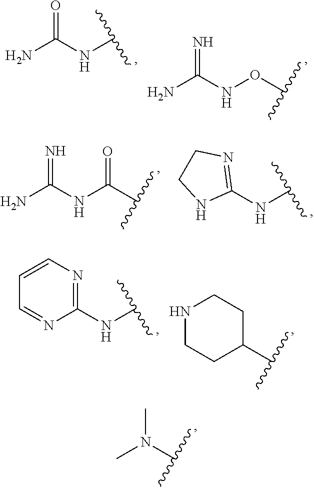

or a protonated form thereof. One or R^{2a}, R^{2b}, R^{2c} and R^{2d} can be

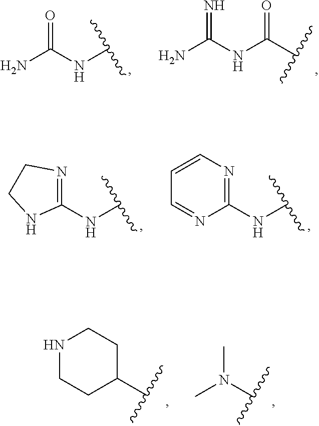

or a protonated form thereof. At least one or R^{2a}, R^{2b}, R^{2c} and R^{2d} can be

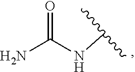 [SMILES: *NC(N)=O]

or a protonated form thereof, and the remaining of R^{2a}, R^{2b}, R^{2c} and R^{2d} can be guanidine or a protonated form thereof. At least two of R^{2a}, R^{2b}, R^{2c} and R^{2d} can be

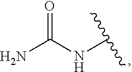 [SMILES: *NC(N)=O]

or a protonated form thereof, and the remaining of R^{2a}, R^{2b}, R^{2c} and R^{2d} can be guanidine, or a protonated form thereof.

All of R^{2a}, R^{2b}, R^{2c} and R^{2d} can be

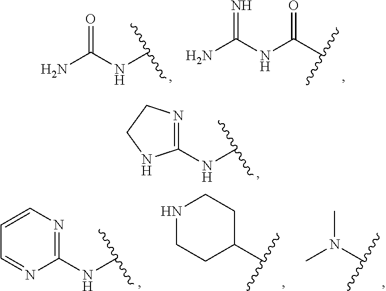

or a protonated form thereof. At least of R^{2a}, R^{2b}, R^{2c} and R^{2d} can be

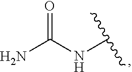 [SMILES: *NC(N)=O]

or a protonated form thereof, and the remaining of R^{2a}, R^{2b}, R^{2c} and R^{2d} can be guanidine or a protonated form thereof. At least two R^{2a}, R^{2b}, R^{2c} and R^{2d} groups can be

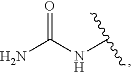 [SMILES: *NC(N)=O]

or a protonated form thereof, and the remaining of R^{2a}, R^{2b}, R^{2c} and R^{2d} are guanidine, or a protonated form thereof.

Each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

AA_{SC} can be

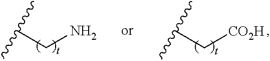

wherein t can be an integer from 0 to 5. AA_{SC} can be

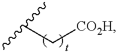

wherein t can be an integer from 0 to 5. t can be 1 to 5. t is 2 or 3. t can be 2. t can be 3.

R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

The cCPP of Formula (II) can have the structure of Formula (II-1):

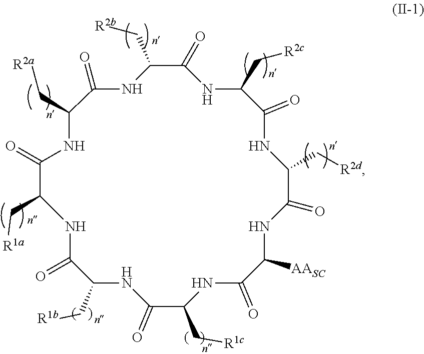

wherein R^{1a}, R^{1b}, R^{1c}, R^{2a}, R^{2b}, R^{2c}, R^{2d}, AA_{SC}, n′ and n″ are as defined herein.

The cCPP of Formula (II) can have the structure of Formula (IIa):

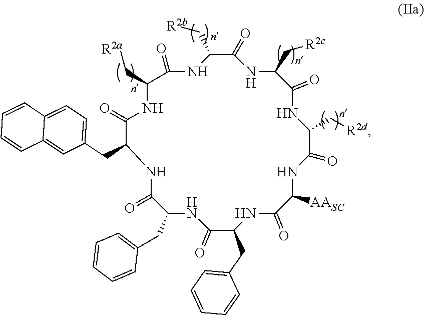

wherein R^{1a}, R^{1b}, R^{1c}, R^{2d}, R^{2b}, R^{2c}, R^{2d}, AA_{SC} and n′ are as defined herein.

The cCPP of formula (II) can have the structure of Formula (IIb):

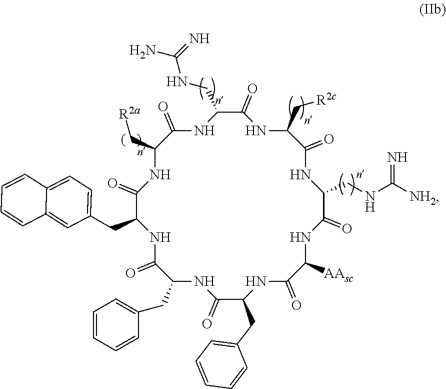

wherein R^{2a}, R^{2b}, AA_{SC}, and n′ are as defined herein.

The cCPP can have the structure of Formula (IIc):

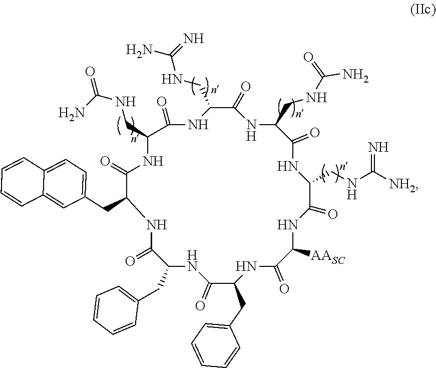

or a protonated form thereof,
wherein:
AA_{SC} and n′ are as defined herein.

The cCPP can have the structure of Formula (III):

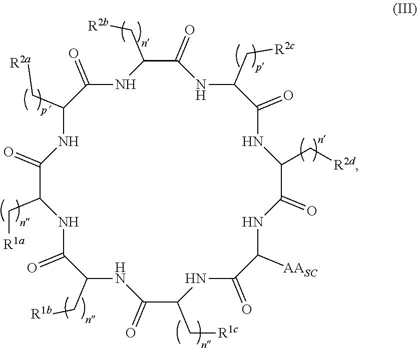

wherein:
AA_{SC} is an amino acid side chain;
R^{1a}, R^{1b}, and R^{1c} are each independently a 6- to 14-membered aryl or a 6- to 14-membered heteroaryl;
R^{2a} and R^{2c} are each independently H,

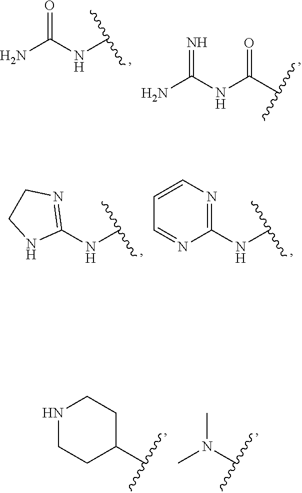

or a protonated form thereof;
R^{2b} and R^{2d} are each independently guanidine or a protonated form thereof;
each n″ is independently an integer from 1 to 3;
each n′ is independently an integer from 1 to 5; and
each p′ is independently an integer from 0 to 5.

The cCPP of Formula (III) can have the structure of Formula (III-1):

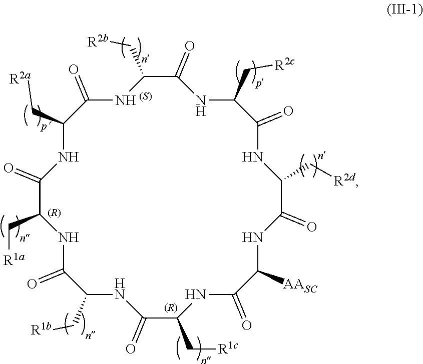

wherein:
AA_{SC}, R^{1a}, R^{1b}, R^{1c}, R^{2a}, R^{2c}, R^{2b}, R^{2d} n′, n″, and p′ are as defined herein.

The cCPP of Formula (III) can have the structure of Formula (IIIa):

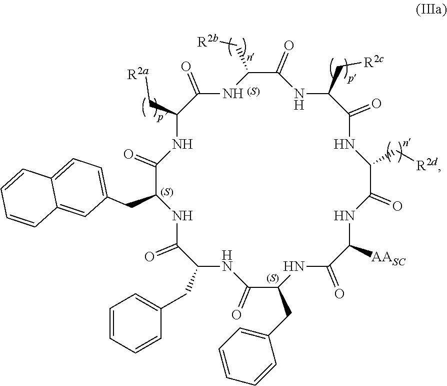

wherein:
AA_{SC}, R^{2a}, R^{2c}, R^{2b}, R^{2d} n′, n″, and p′ are as defined herein.

In Formulas (III), (III-1), and (IIa), R^{a} and R^{c} can be H. R^{a} and R^{c} can be H and R^{b} and R^{d} can each independently be guanidine or protonated form thereof. R^{a} can be H. R^{b} can be H. p′ can be 0. R^{a} and R^{c} can be H and each p′ can be 0.

In Formulas (III), (III-1), and (IIIa), R^{a} and R^{c} can be H, R^{b} and R^{d} can each independently be guanidine or protonated form thereof, n″ can be 2 or 3, and each p′ can be 0.

p′ can 0. p′ can 1. p′ can 2. p′ can 3. p′ can 4. p′ can be 5.

The cCPP can have the structure:

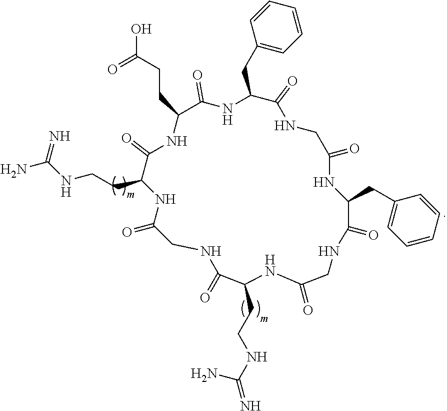

The cCPP of Formula (A) can be selected from:

CPP Sequence

(FfΦRrRrQ)
(FfΦCit-r-Cit-rQ)
(FfΦGrGrQ)
(FfFGRGRQ)
(FGFGRGRQ)
(GfFGrGrQ)
(FGFGRRRQ)
(FGFRRRRQ)

The cCPP of Formula (A) can be selected from:

CPP Sequence

FΦRRRRQ
fΦRrRrQ
FfΦRrRrQ
FfΦCit-r-Cit-rQ
FfΦGrGrQ
FfΦRGRGQ
FfFGRGRQ
FGFGRGRQ
GfFGrGrQ
FGFGRRRQ
FGFRRRRQ

In embodiments, the cCPP is selected from.

| CPP sequence | CPP sequence | CPP sequence |
|---|---|---|
| FΦRRRQ | FRRRRΦQ | CRRRRFWQ |
| FΦRRRC | rRFRΦRQ | FΦRRRRQK |
| FΦRRRU | RRΦFRRQ | FΦRRRRQC |
| RRRΦFQ | CRRRRFWQ | fΦRrRrRQ |
| RRRRΦF | FfΦRrRrQ | FØRRRRRQ |
| FΦRRRR | FFΦRRRRQ | RRRRΦFDΩC |
| FϕrRrRq | RFRFRΦRQ | FΦRRR |
| FϕrRrRQ | URRRRFWQ | FWRRR |
| FΦRRRRQ | RRFRΦRQ | RRRΦF |
| fΦRrRrQ | FRRRRΦQ | RRRWF |
| RRFRΦRQ | | |

Φ = L-naphthylalanine; ϕ = D-naphthylalanine; Ω = L-norleucine

The cCPP can comprise the structure of Formula (D)

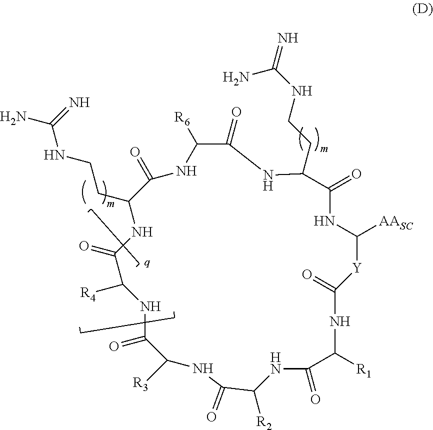

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
Y is

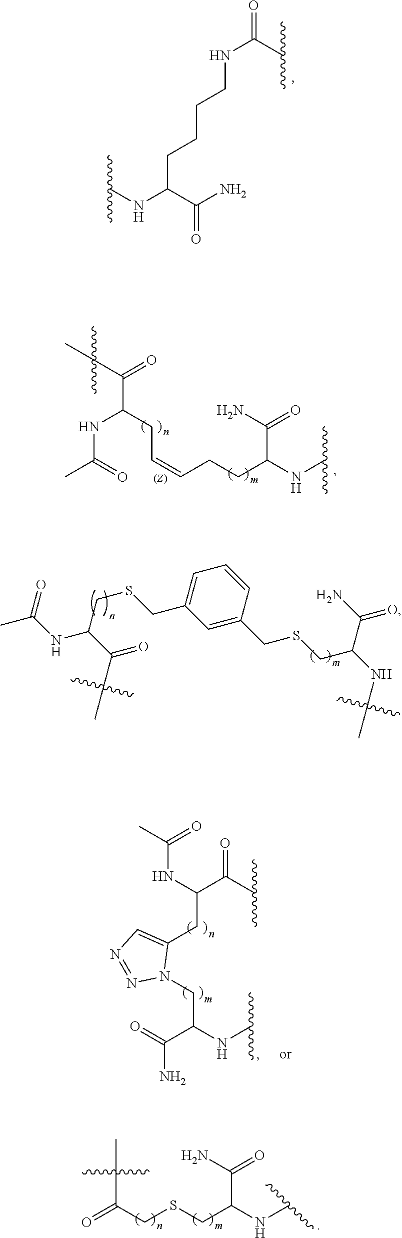

q is 1, 2, 3 or 4;
each m is independently an integer 0, 1, 2, or 3, and
each n is independently an integer 0, 1, 2, or 3.

The cCPP of Formula (D), wherein Y is

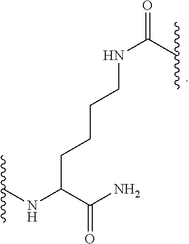 [SMILES: *NC(CCCCNC(*)=O)C(N)=O]

The cCPP of Formula (D), wherein Y is:

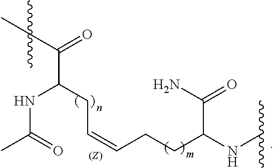

The cCPP of Formula (D), wherein Y is:

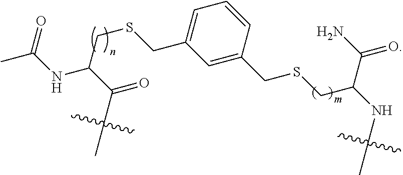

The cCPP of Formula (D), wherein Y is:

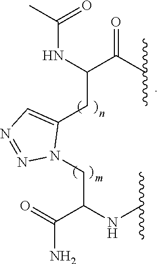

The cCPP of Formula (D), wherein Y is:

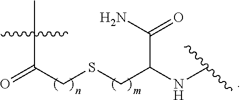

In embodiments. AA_{SC} can be conjugated to a linker.

### Linker

The cCPP of the disclosure can be conjugated to a linker. The linker can link a cargo to the cCPP. The linker can be attached to the side chain of an amino acid of the cCPP, and the cargo can be attached at a suitable position on linker.

The linker can be any appropriate moiety which can conjugate a cCPP to one or more additional moieties, e.g., an exocyclic peptide (EP) and/or a cargo. Prior to conjugation to the cCPP and one or more additional moieties, the linker has two or more functional groups, each of which are independently capable of forming a covalent bond to the cCPP and one or more additional moieties. If the cargo is an oligonucleotide, the linker can be covalently bound to the 5′ end of the cargo or the 3′ end of the cargo. The linker can be covalently bound to the 5′ end of the cargo. The linker can be covalently bound to the 3′ end of the cargo. If the cargo is a peptide, the linker can be covalently bound to the N-terminus or the C-terminus of the cargo. The linker can be covalently bound to the backbone of the oligonucleotide or peptide cargo. The linker can be any appropriate moiety which conjugates a cCPP described herein to a cargo such as an oligonucleotide, peptide or small molecule.

The linker can comprise hydrocarbon linker.

The linker can comprise a cleavage site. The cleavage site can be a disulfide, or caspase-cleavage site (e.g., Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹—R²)z″- subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) —(R¹-J)z″- or -(J-R¹)z″-, wherein each of R¹, at each instance, is independently alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or -(R¹J-R²)z″-, wherein each of R¹ and R², at each instance, are independently alkylene, each J is independently C, NR³, —NR³C(O)—, S, and O, wherein R is independently selected from H and alkyl, and z″ is an integer from 1 to 50; or combinations thereof.

The linker can comprise a —(OCH₂CH₂)_{z}— (e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂) z′ can also be referred to as polyethylene glycol (PEG).

The linker can comprise one or more amino acids. The linker can comprise a peptide. The linker can comprise a —(OCH₂CH₂)_{z′}—, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the cargo is conjugated to the linker.

The linker can comprises (i) a β alanine residue and lysine residue; (ii) -(J-R¹)z″; or (iii) a combination thereof. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of β-alanine, glycine, lysine, 4-aminobutyric acid, 5-aminopentanoic acid, 6-aminohexanoic acid or combinations thereof; and (ii) —(R¹J)z″- or -(J-R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be 0. The linker can comprise glycine, beta-alanine, 4-aminobutyric acid, 5-aminopentanoic acid, 6-aminohexanoic acid, or a combination thereof.

The linker can be a trivalent linker. The linker can have the structure:

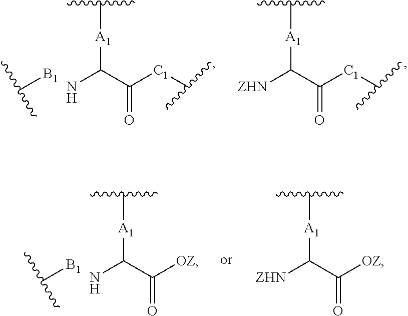

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)˜-COOH], or caspase-cleavage site (Val-Cit-PABC).

The hydrocarbon can be a residue of glycine or beta-alanine.

The linker can be bivalent and link the cCPP to a cargo. The linker can be bivalent and link the cCPP to an exocyclic peptide (EP).

The linker can be trivalent and link the cCPP to a cargo and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The linker can have the structure:

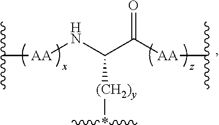

wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

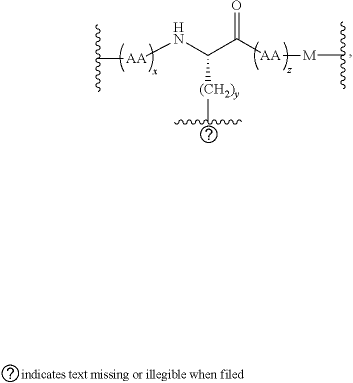

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

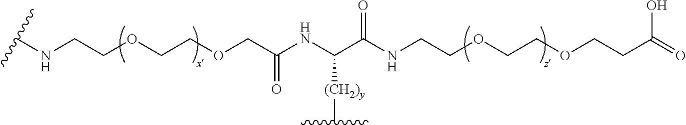

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

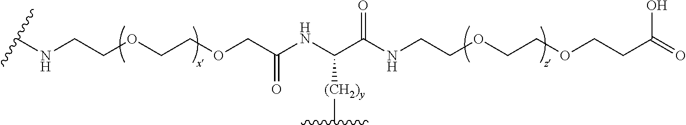

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

x can be an integer from 1-10, e.g., 1, 2, 3, 4, 5, 6, 7, 8, 9, or 10, inclusive of all ranges and subranges therebetween.

x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.

y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.

z can be an integer from 1-10, e.g., 1, 2, 3, 4, 5, 6, 7, 8, 9, or 10, inclusive of all ranges and subranges therebetween.

z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is part of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can be bound to the side chain of aspartic acid, glutamic acid, glutamine, asparagine, or lysine, or a modified side chain of glutamine or asparagine (e.g., a reduced side chain having an amino group), on a peptide cargo. The linker can be bound to the side chain of lysine on the peptide cargo.

The linker can have a structure:

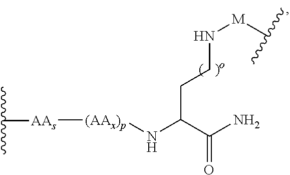

wherein
M is a group that conjugates L to a cargo, for example, an oligonucleotide;
AAₛ is a side chain or terminus of an amino acid on the cCPP;
each AAₓ is independently an amino acid residue;
o is an integer from 0 to 10; and
p is an integer from 0 to 5.

The linker can have a structure:

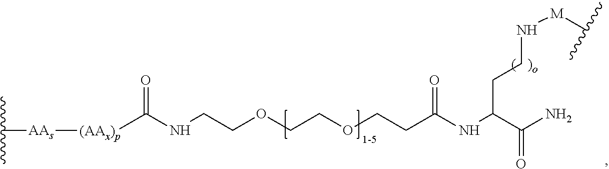

wherein
M is a group that conjugates L to a cargo, for example, an oligonucleotide;
AAₛ is a side chain or terminus of an amino acid on the cCPP;
each AAₓ is independently an amino acid residue;
is an integer from 0 to 10; and
p is an integer from 0 to 5.

M can comprise an alkylene, alkenylene, alkynylene, carbocyclyl, or heterocyclyl, each of which is optionally substituted. M can be selected from:

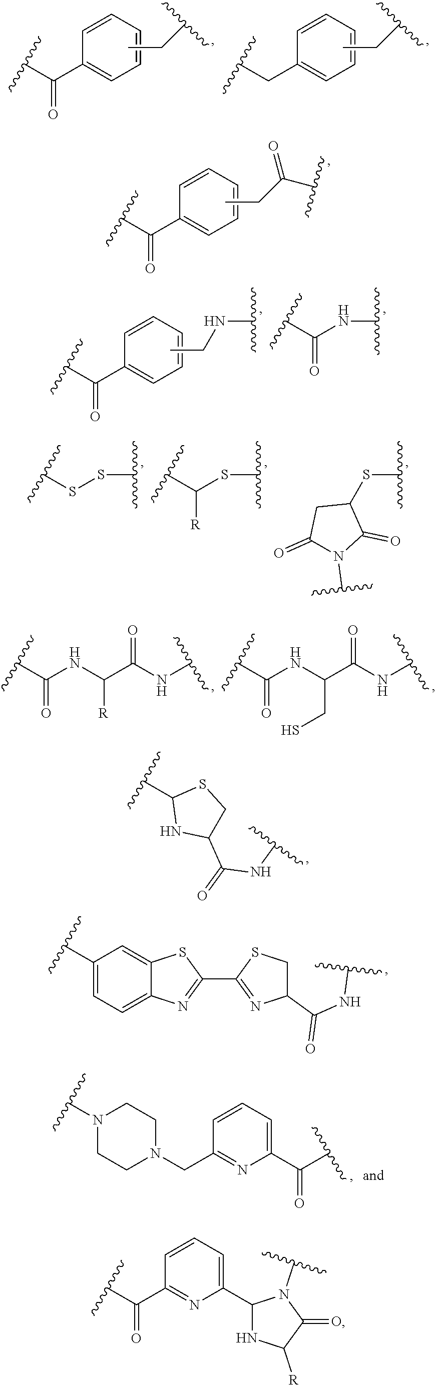

wherein R is alkyl, alkenyl, alkynyl, carbocyclyl, or heterocyclyl.

M can be selected from:

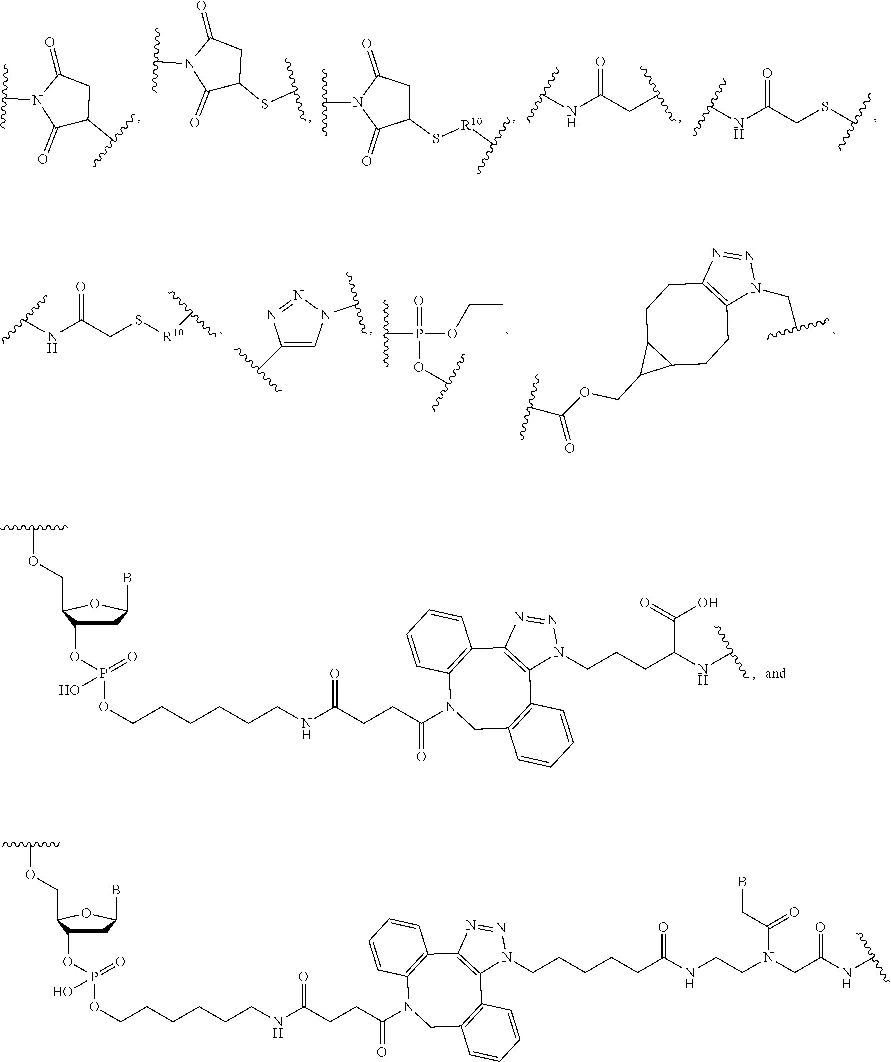

wherein: R¹⁰ is alkylene, cycloalkyl, or

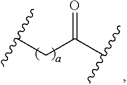

wherein a is 0 to 10.

M can be

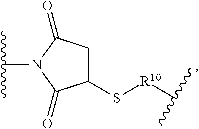

### R¹⁰ can be

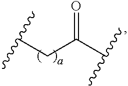

### and a is 0 to 10. M can be

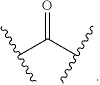 [SMILES: *C(*)=O]

M can be a heterobifunctional crosslinker, e.g.,

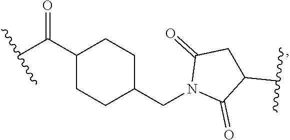 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The β-amino acid can be β-alanine.

o can be an integer from 0 to 10, e.g., 0, 1, 2, 3, 4, 5, 6, 7, 8, 9, and 10. o can be 0, 1, 2, or 3. o can be 0. o can be 1. o can be 2. o can be 3.

p can be 0 to 5, e.g., 0, 1, 2, 3, 4, or 5. p can be 0. p can be 1. p can be 2. p can be 3. p can be 4. p can be 5.

The linker can have the structure:

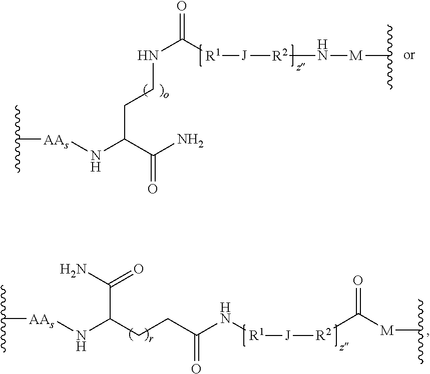

wherein M, AAₛ, each —(R¹-J-R²)z″—, o and z″ are defined herein; r can be 0 or 1.

r can be 0. r can be 1.

The linker can have the structure:

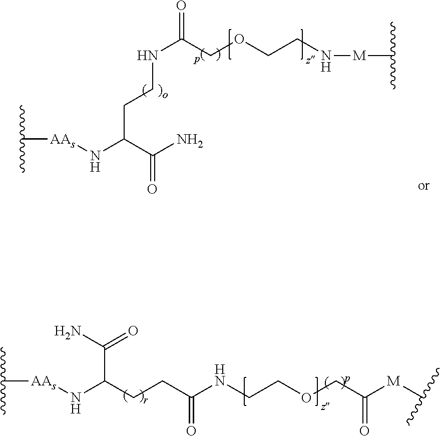

wherein each of M, AAₛ, o, p, q, r and z″ can be as defined herein.

z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

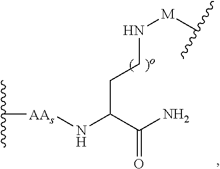

wherein:
M, AAₛ and o are as defined herein.

Other non-limiting examples of suitable linkers include:

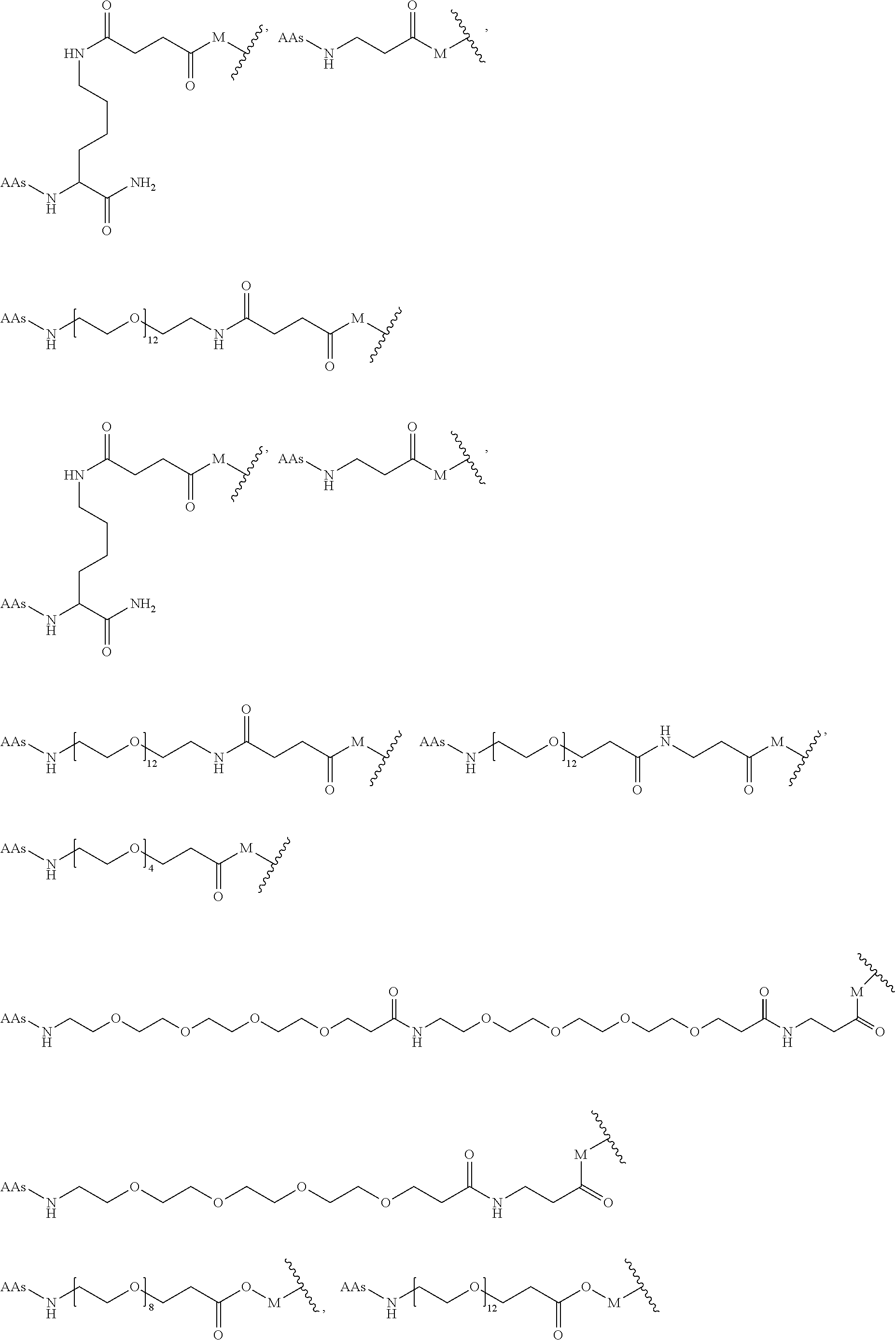

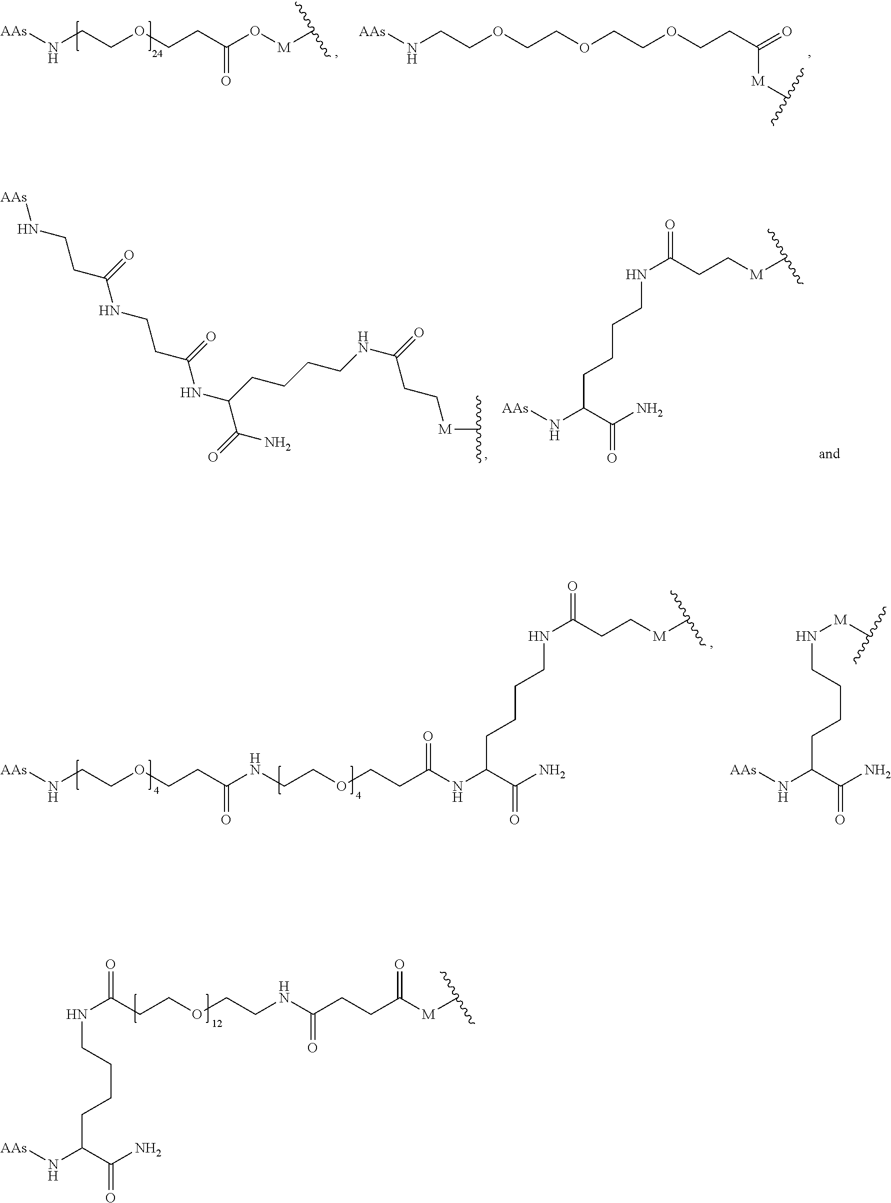

wherein M and AA; are as defined herein.

Provided herein is a compound comprising a cCPP and a cargo that comprises a hybrid oligonucleotide, for example, an antisense oligonucleotide, that is complementary to a target in a pre-mRNA sequence, wherein the compound further comprises L, wherein the linker is conjugated to the hybrid oligonucleotide through a bonding group (M), wherein M is selected from:

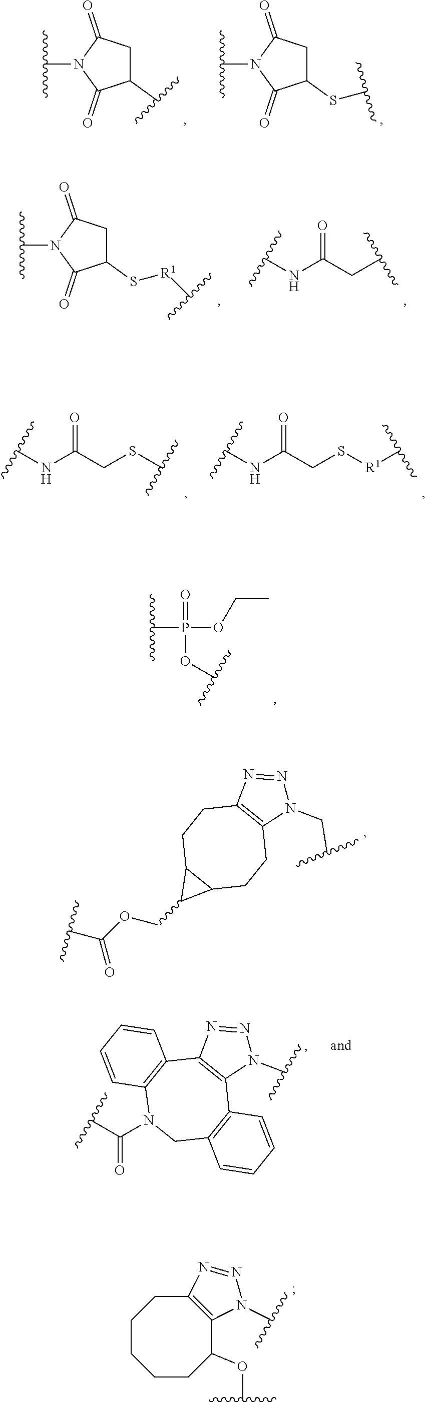

wherein: R¹ is alkylene, cycloalkyl, or

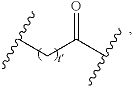

wherein t′ is 0 to 10 wherein each R is independently an alkyl, alkenyl, alkynyl, carbocyclyl, or heterocyclyl, wherein R¹ is

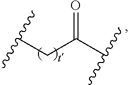

and t′ is 2.

The linker can have the structure:

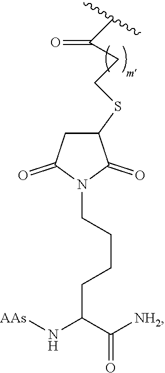

wherein AAₛ is as defined herein, and m′ is 0-10.

The linker can be of the formula:

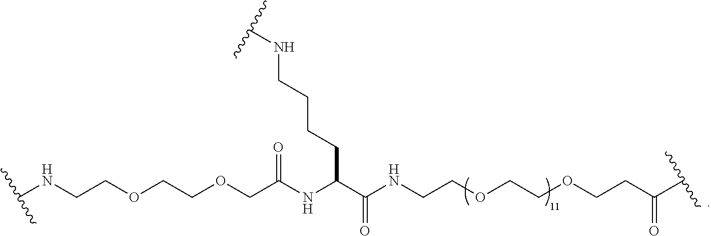

The linker can be of the formula:

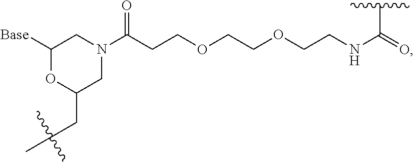

wherein “base” corresponds to a nucleobase at the 3′ end of a cargo phosphorodiamidate morpholino oligomer.

The linker can be of the formula:

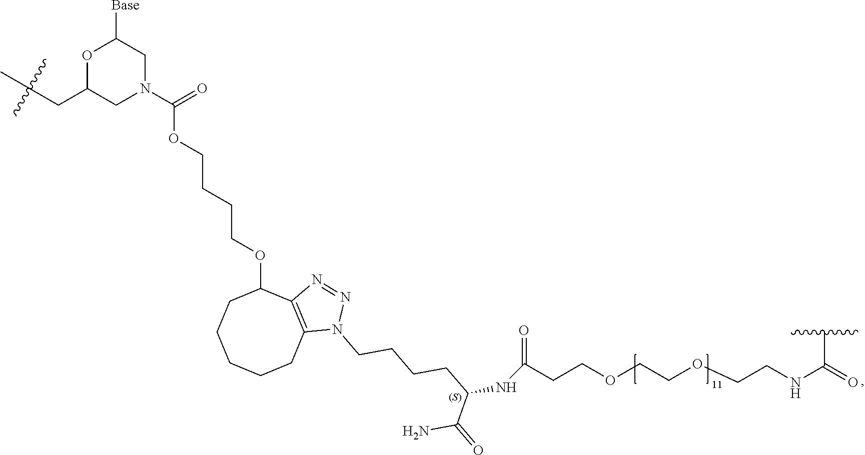

wherein “base” corresponds to a nucleobase at the 3′ end of a cargo phosphorodiamidate morpholino oligomer.

The linker can be of the formula:

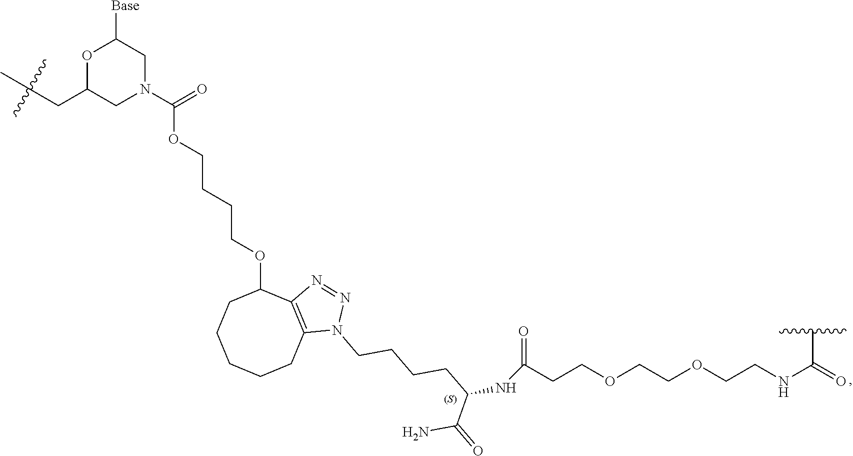

wherein “base” corresponds to a nucleobase at the 3′ end of a cargo phosphorodiamidate morpholino oligomer.

The linker can be of the formula:

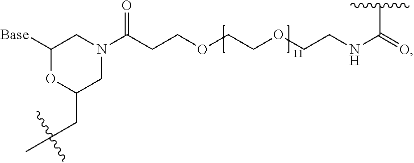

wherein “base” corresponds to a nucleobase at the 3′ end of a cargo phosphorodiamidate morpholino oligomer.

The linker can be of the formula:

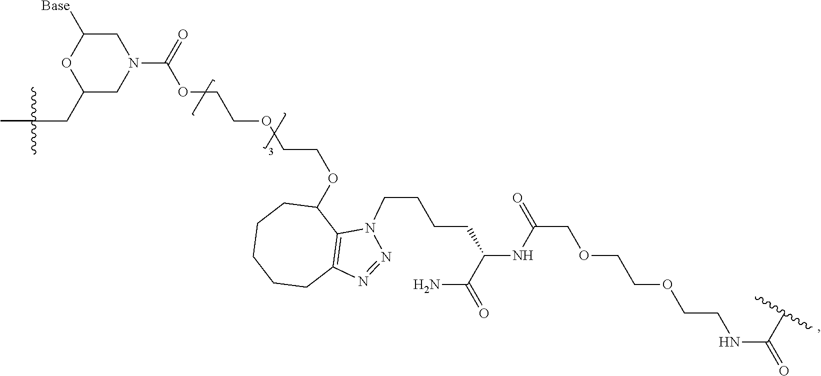

The linker can be covalently bound to a cargo at any suitable location on the cargo. The linker is covalently bound to the 3′ end of cargo or the 5′ end of an oligonucleotide cargo The linker can be covalently bound to the backbone of a cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

cCPP-Linker Conjugates

The cCPP can be conjugated to a linker defined herein. The linker can be conjugated to an AA_{SC} of the cCPP as defined herein.

The linker can comprise a —(OCH₂CH₂)_{z′}— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z′} is also referred to as PEG. The cCPP-linker conjugate can have a structure selected from Table D:

TABLE D
cCPP-linker conjugates

cyclo(FfΦ-4gp-r-4gp-rQ)-PEG₄-K-NH₂
cyclo(FfΦ-Cit-r-Cit-rQ)-PEG₄-K-NH₂
cyclo(FfΦ-Pia-r-Pia-rQ)-PEG₄-K-NH₂
cyclo(FfΦ-Dml-r-Dml -rQ)-PEG₄-K-NH₂
cyclo(FfΦ-Cit-r-Cit-rQ)-PEG₁₂-OH
cyclo(fΦR-Cit-R-Cit-Q)-PEG₁₂-OH

The linker can comprise a —(OCH₂CH₂)_{z′}— subunit, wherein z′ is an integer from 1 to 23, and a peptide subunit. The peptide subunit can comprise from 2 to 10 amino acids. The cCPP-linker conjugate can have a structure selected from Table E:

TABLE E
cCPP-linker conjugate

Ac-PKKKRKV-Lys(cyclo[FfΦ-R-r-Cit-rQ])-PEG₁₂-K(N₃)-NH₂
Ac-PKKKRKV-Lys(cyclo[FfΦ-Cit-r-R-rQ])-PEG₁₂-K(N₃)-NH₂
Ac-PKKKRKV-K(cyclo(FfΦR-cit-R-cit-Q))-PEG₁₂-K(N₃)-NH₂
Ac-PKKKRKV-PEG2-Lys(cyclo[FfΦ-Cit-r-Cit-rQ])-B-k(N₃)-NH₂
Ac-PKKKRKV-PEG2-Lys(cyclo[FfΦ-Cit-r-Cit-rQ])-PEG2-k(N₃)-NH₂
Ac-PKKKRKV-PEG2-Lys(cyclo[FfΦ-Cit-r-Cit-rQ])-PEG4-k(N₃)-NH₂
Ac-PKKKRKV-Lys(cyclo[FfΦ-Cit-r-Cit-rQ])-PEG12-k(N₃)-NH₂
Ac-pkkkrkv-PEG2-Lys(cyclo[FfΦ-Cit-r-Cit-rQ])-PEG12-k(N₃)-NH₂
Ac-rrv-PEG2-Lys(cyclo[FfΦ-Cit-r-Cit-rQ])-PEG12-OH
Ac-PKKKRKV-PEG2-Lys(cyclo[FfΦ-Cit-r-Cit-r-Q])-PEG12-k(N₃)-NH₂
Ac-PKKK-Cit-KV-PEG2-Lys(cyclo[FfΦ-Cit-r-Cit-r-Q])-
PEG12-k(N₃)-NH₂
Ac-PKKKRKV-PEG2-Lys(cyclo[FfΦ-Cit-r-Cit-r-Q]-PEG12-K(N₃)-NH₂

EEVs comprising a cyclic cell penetrating peptide (cCPP), linker and exocyclic peptide (EP) are provided. An EEV can comprise the structure of Formula (B):

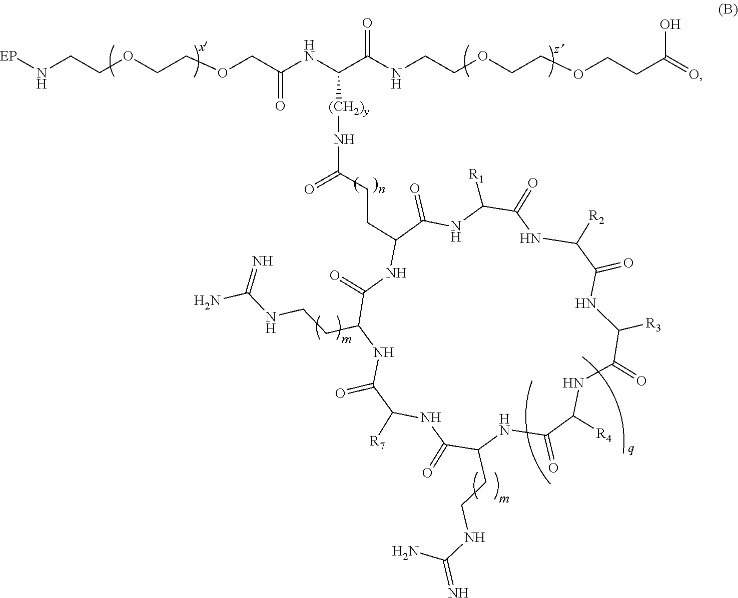

or a protonated form thereof, wherein:
R₁, R₂, and R₃ are each independently H or an aromatic or heteroaromatic side chain of an amino acid;
R₄ and R₇ are independently H or an amino acid side chain;
EP is an exocyclic peptide as defined herein;
each m is independently an integer from 0-3;
n is an integer from 0-2:
x′ is an integer from 1-20;
y is an integer from 1-5;
q is 1-4; and
z′ is an integer from 1-23.

R₁, R₂, R₃, R₄, R₇, EP, m, q, y, x′, z′ are as described herein.

n can be 0. n can be 1. n can be 2.

The EEV can comprise the structure of Formula (B-a) or (B-b):

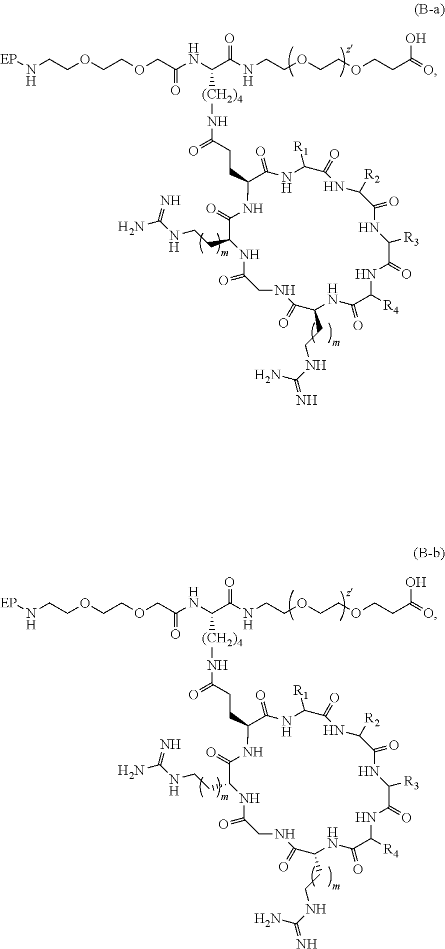

or a protonated form thereof, wherein EP, R¹, R², R³, R⁴, m and z′ are as defined above in Formula (B).

The EEV can comprises the structure of Formula (B-c):

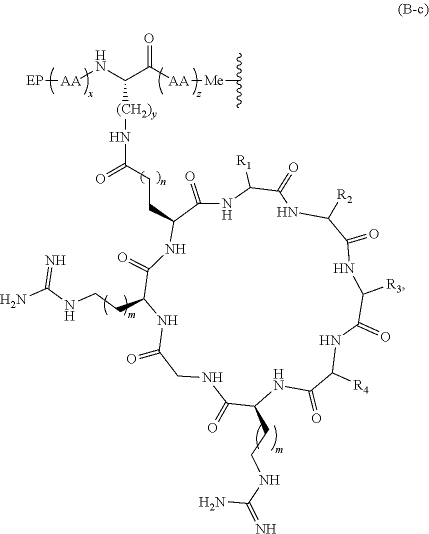

or a protonated form thereof, wherein EP, R¹, R², R³, R₄, and m are as defined above in Formula (B); AA is an amino acid as defined herein; M is as defined herein; n is an integer from 0-2; x is an integer from 1-10; y is an integer from 1-5; and z is an integer from 1-10.

The EEV can have the structure of Formula (B-1), (B-2), (B-3), or (B-4):

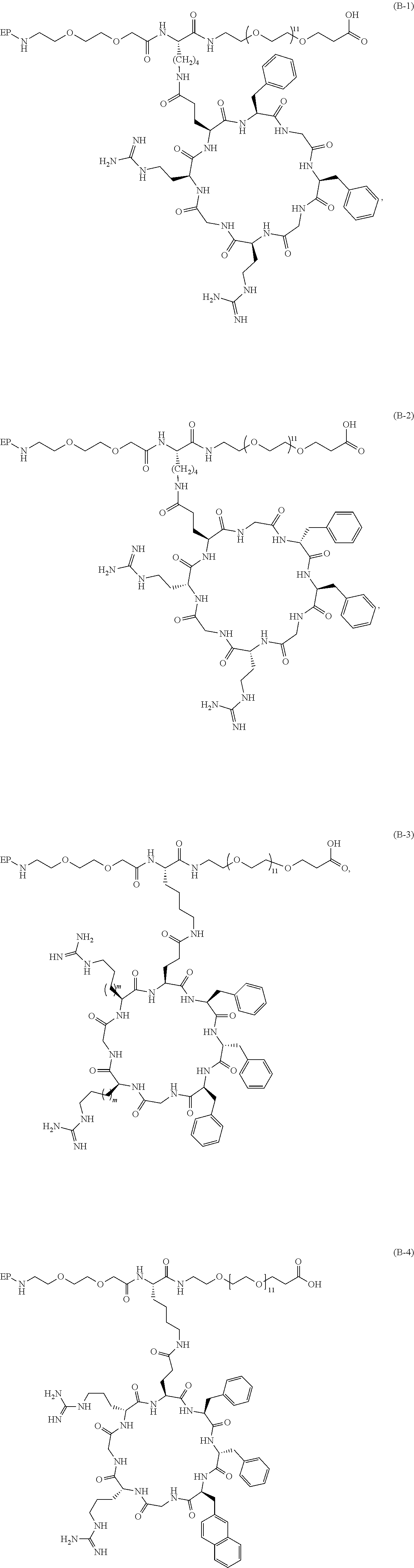

or a protonated form thereof, wherein EP is as defined above in Formula (B).

The EEV can comprise Formula (B) and can have the structure: Ac-PKKKRKV-AEEA—K(cyclo[FGFGRGRQ])-PEG₁₂—OH or Ac-PKKKRKV-AEEA—K(cyclo[GfFGrGrQ])-PEG₁₂—OH.

The EEV can comprise a cCPP of formula:

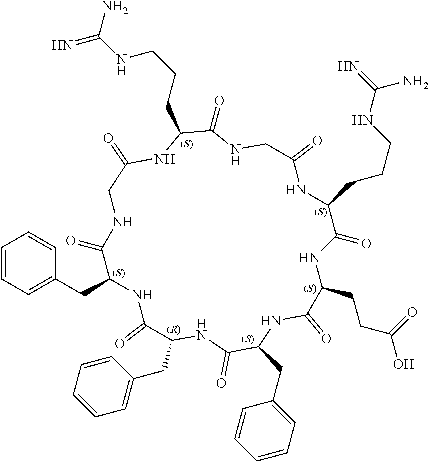

The EEV can comprise formula: Ac-PKKKRKV-miniPEG2-Lys(cyclo(FfFGRGRQ)-miniPEG2—K(N3).

The EEV can be Ac-P—K(Tfa)—K(Tfa)—K(Tfa)-R—K(Tfa)-V-AEEA—K-(cyclo[FGFGRGRQ])-PEG12—OH.

The EEV can be:

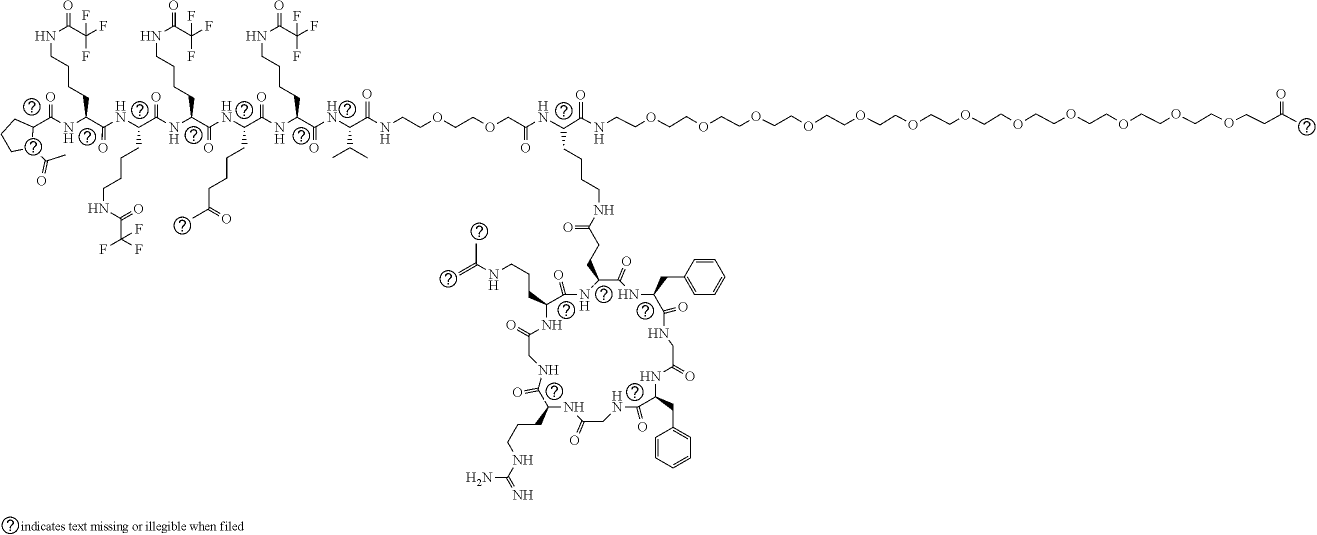

The EEV can be Ac-PKKKRKV-AEEA-Lys-(cyclo[FGFGRGRQ])-PEG12—OH. The EEV can be:

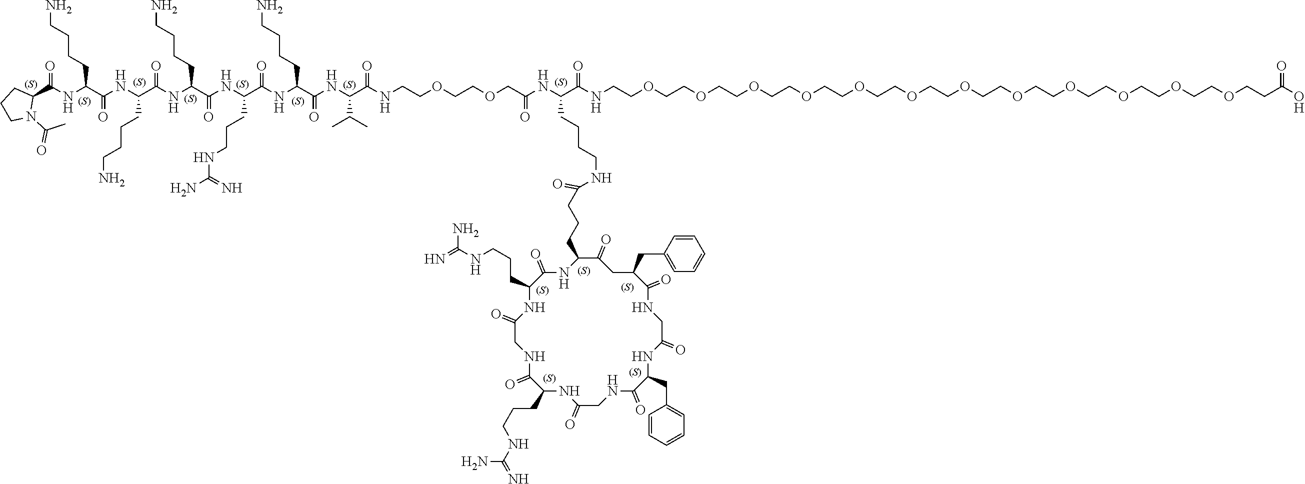

The EEV can be selected from
Ac-rr-miniPEG₂-Dap[cyclo(FfΦ-Cit-r-Cit-rQ)]-PEG₁₂—OH
Ac-frr-PEG2-Dap(cyclo(FfΦ-Cit-r-Cit-rQ))-PEG₁₂—OH
Ac-rfr-PEG2-Dap(cyclo(FfΦ-Cit-r-Cit-rQ))-PEG₁₂—OH
Ac-rbfbr-PEG2-Dap(cyclo(FfΦ-Cit-r-Cit-rQ))-PEG₁₂—OH
Ac-rrr-PEG2-Dap(cyclo(FfΦ-Cit-r-Cit-rQ))-PEG₁₂—OH
Ac-rbr-PEG2-Dap(cyclo(FfΦ-Cit-r-Cit-rQ))-PEG₁₂—OH
Ac-rbrbr-PEG2-Dap(cyclo(FfΦ-Cit-r-Cit-rQ))-PEG₁₂—OH
Ac-hh-PEG2-Dap(cyclo(FfΦ-Cit-r-Cit-rQ))-PEG₁₂—OH
Ac-hbh-PEG2-Dap(cyclo(FfΦ-Cit-r-Cit-rQ))-PEG₁₂—OH
Ac-hbhbh-PEG2-Dap(cyclo(FfΦ-Cit-r-Cit-rQ))-PEG₁₂—OH
Ac-rbhbh-PEG2-Dap(cyclo(FfΦ-Cit-r-Cit-rQ))-PEG₁₂—OH
Ac-hbrbh-PEG2-Dap(cyclo(FfΦ-Cit-r-Cit-rQ))-PEG₁₂—OH
Ac-rr-Dap(cyclo(FfΦ-Cit-r-Cit-rQ))-b—OH
Ac-frr-Dap(cyclo(FfΦ-Cit-r-Cit-rQ))-b—OH
Ac-rfr-Dap(cyclo(FfΦ-Cit-r-Cit-rQ))-b—OH
Ac-rbfbr-Dap(cyclo(FfΦ-Cit-r-Cit-rQ))-b—OH
Ac-rrr-Dap(cyclo(FfΦ-Cit-r-Cit-rQ))-b—OH
Ac-rbr-Dap(cyclo(FfΦ)-Cit-r-Cit-rQ))-b—OH
Ac-rbrbr-Dap(cyclo(FfΦ-Cit-r-Cit-rQ))-b—OH
Ac-hh-Dap(cyclo(FfΦ-Cit-r-Cit-rQ))-b—OH

The EEV can be selected from:
Ac-hbh-Dap(cyclo(FfΦ-Cit-r-Cit-rQ))-b—OH
Ac-hbhbh-Dap(cyclo(FfΦ-Cit-r-Cit-rQ))-b—OH
Ac-rbhbh-Dap(cyclo(FfΦ-Cit-r-Cit-rQ))-b—OH
Ac-hbrbh-Dap(cyclo(FfΦ-Cit-r-Cit-rQ))-b—OH
Ac—KKKK-miniPEG2-Lys(cyclo(Ff-Nal-GrGrQ))-miniPEG₂—K(N₃)-NH₂
Ac—KGKK-miniPEG2-Lys(cyclo(Ff-Nal-GrGrQ))-miniPEG₂—K(N₃)-NH₂
Ac—KKGK-miniPEG2-Lys(cyclo(Ff-Nal-GrGrQ))-miniPEG₂—K(N₃)-NH₂
Ac—KKK-miniPEG2-Lys(cyclo(Ff-Nal-GrGrQ))-miniPEG₂—K(N₃)-NH₂
Ac—KK-miniPEG2-Lys(cyclo(Ff-Nal-GrGrQ))-miniPEG₂—K(N₃)-NH₂
Ac—KGK-miniPEG2-Lys(cyclo(Ff-Nal-GrGrQ))-miniPEG—K(N₃)-NH₂
Ac—KBK-miniPEG2-Lys(cyclo(Ff-Nal-GrGrQ))-miniPEG—K(N₃)-NH₂
Ac—KBKBK-miniPEG2-Lys(cyclo(Ff-Nal-GrGrQ))-miniPEG—K(N₃)-NH₂
Ac—KR-miniPEG2-Lys(cyclo(Ff-Nal-GrGrQ))-miniPEG—K(N₃)-NH₂
Ac—KBR-miniPEG2-Lys(cyclo(Ff-Nal-GrGrQ))-miniPEG₂—K(N₃)-NH₂
Ac-PKKKRKV-miniPEG2-Lys(cyclo(Ff-Nal-GrGrQ))-miniPEG₂—K(N₃)-NH₂
Ac-PKKKRKV-miniPEG2-Lys(cyclo(Ff-Nal-GrGrQ))-miniPEG₂—K(N₃)-NH₂
Ac-PGKKRKV-miniPEG2-Lys(cyclo(Ff-Nal-GrGrQ))-miniPEG₂—K(N₃)-NH₂
Ac-PKGKRKV-miniPEG2-Lys(cyclo(Ff-Nal-GrGrQ))-miniPEG₂—K(N₃)-NH₂
Ac-PKKGRKV-miniPEG2-Lys(cyclo(Ff-Nal-GrGrQ))-miniPEG₂—K(N₃)-NH₂
Ac-PKKKGKV-miniPEG2-Lys(cyclo(Ff-Nal-GrGrQ))-miniPEG₂—K(N₃)-NH₂
Ac-PKKKRGV-miniPEG2-Lys(cyclo(Ff-Nal-GrGrQ))-miniPEG₂—K(N₃)-NH₂
Ac-PKKKRKG-miniPEG2-Lys(cyclo(Ff-Nal-GrGrQ))-miniPEG₂—K(N₃)-NH₂
Ac—KKKRK-miniPEG2-Lys(cyclo(Ff-Nal-GrGrQ))-miniPEG₂—K(N₃)-NH₂
Ac—KKRK-miniPEG2-Lys(cyclo(Ff-Nal-GrGrQ))-miniPEG₂—K(N₃)-NH₂ and
Ac—KRK-miniPEG2-Lys(cyclo(Ff-Nal-GrGrQ))-miniPEG₂—K(N₃)-NH₂. [0453]The EEV can be selected from:
Ac-PKKKRKV-Lys(cyclo[FfΦGrGrQ])-PEG_{12—K(N}₃)-NH₂
Ac-PKKKRKV-miniPEG₂-Lys(cyclo[FfΦGrGrQ])-miniPEG₂—K(N₃)-NH₂
Ac-PKKKRKV-miniPEG₂-Lys(cyclo[FGFGRGRQ])-miniPEG₂—K(N₃)-NH₂
Ac—KR-PEG₂—K(cyclo[FGFGRGRQ])-PEG₂—K(N₃)-NH

The EEV can be selected from:
Ac-PKKKGKV-PEG₂—K(cyclo[FGFGRGRQ])-PEG₂—K(N₃)-NH2
Ac-PKKKRKG-PEG₂—K(cyclo[FGFGRGRQ])-PEG₂—K(N₃)-NH2
Ac—KKKRK-PEG₂—K(cyclo[FGFGRGRQ])-PEG₂—K(N₃)-NH2
Ac-PKKKRKV-miniPEG₂-Lys(cyclo[FFΦGRGRQ])-miniPEG₂—K(N₃)-NH2
Ac-PKKKRKV-miniPEG₂-Lys(cyclo[BhFfΦGrGrQ])-miniPEG₂—K(N₃)-NH2 and
Ac-PKKKRKV-miniPEG₂-Lys(cyclo[FfΦSrSrQ])-miniPEG₂—K(N₃)-NH2.

The EEV can be selected from:
Ac-PKKKRKV-miniPEG₂-Lys(cyclo(GfFGrGrQ])-PEG₁₂—OH
Ac-PKKKRKV-miniPEG₂-Lys(cyclo[FGFKRKRQ])-PEG₁₂—OH
Ac-PKKKRKV-miniPEG₂-Lys(cyclo[FGFRGRGQ])-PEG₁₂—OH
Ac-PKKKRKV-miniPEG₂-Lys(cyclo[FGFGRGRGRQ])-PEG₁₂—OH
Ac-PKKKRKV-miniPEG₂-Lys(cyclo[FGFGRrRQ])-PEG₁₂—OH
Ac-PKKKRKV-miniPEG₂-Lys(cyclo[FGFGRRRQ])-PEG₁₂—OH and
Ac-PKKKRKV-miniPEG₂-Lys(cyclo[FGFRRRRQ])-PEG₁₂—OH.

The EEV can be selected from:
Ac—K—K—K—R—K_13 G-miniPEG₂—K(cyclo[FGFGRGRQ])-PEG₁₂—OH
Ac—K—K—K—R—K-miniPEG₂—K(cyclo[FGFGRGRQ])-PEG₁₂—OH
Ac—K—K-R—K—K-PEG4—K(cyclo[FGFGRGRQ])-PEG₁₂—OH
Ac—K-R—K—K—K-PEGa—K(cyclo[FGFGRGRQ])-PEG₁₂—OH
Ac—K—K—K—K-R-PEG4—K(cyclo[FGFGRGRQ])-PEG₁₂—OH
Ac-R—K—K—K—K-PEGa—K(cyclo[FGFGRGRQ])-PEG₁₂—OH and
Ac—K—K—K-R—K-PEG4—K(cyclo[FGFGRGRQ])-PEG₁₂—OH. [0456]The EEV can be selected from:
Ac-PKKKRKV-PEG₂—K(cyclo[FGFGRGRQ])-PEG₂—K(N₃)-NH2
Ac-PKKKRKV-PEG₂—K(cyclo[FGFGRGRQ])-PEG₁₂—OH
Ac-PKKKRKV-PEG₂—K(cyclo[GfFGrGrQ])-PEG₂—K(N₃)-NH2 and
Ac- PKKKRKV-PEG₂—K(cyclo[GfFGrGrQ])-PEG₁₂—OH.

The cargo can be an AC and the EEV can be selected from:
Ac-PKKKRKV-PEG₂—K(cyclo[FfΦGrGrQ])-PEG₁₂—OH
Ac-PKKKRKV-PEG₂—K(cyclo[FfΦCit-r-Cit-rQ])-PEG₁₂—OH
Ac-PKKKRKV-PEG₂—K(cyclo[FfFGRGRQ])-PEG₁₂—OH
Ac-PKKKRKV-PEG₂—K(cyclo[FGFGRGRQ])-PEG₁₂—OH
Ac-PKKKRKV-PEG₂—K(cyclo[GfFGrGrQ])-PEG₁₂—OH
Ac-PKKKRKV-PEG₂—K(cyclo[FGFGRRRQ])-PEG₁₂—OH
Ac-PKKKRKV-PEG₂—K(cyclo[FGFRRRRQ])-PEG₁₂—OH
Ac-rr-PEG₂—K(cyclo[FfΦGrGrQ])-PEG₁₂—OH
Ac-rr-PEG₂—K(cyclo[FfΦCit-r-Cit-rQ])-PEG₁₂—OH
Ac-rr-PEG₂—K(cyclo[FfF-GRGRQ])-PEG₁₂—OH
Ac-rr-PEG₂—K(cyclo[FGFGRGRQ])-PEG₁₂—OH
Ac-rr-PEG₂—K(cyclo[GfFGrGrQ])-PEG₁₂—OH
Ac-rr-PEG₂—K(cyclo[FGFGRRRQ])-PEG₁₂—OH
Ac-rr-PEG₂—K(cyclo[FGFRRRRQ])-PEG₁₂—OH
Ac-rrr-PEG₂—K(cyclo[FfΦGrGrQ])-PEG₁₂—OH
Ac-rrr-PEG₂—K(cyclo[FfΦCit-r-Cit-rQ])-PEG₁₂—OH
Ac-rrr-PEG₂—K(cyclo[FfFGRGRQ])-PEG₁₂—OH
Ac-rrr-PEG₂—K(cyclo[FGFGRGRQ])-PEG₁₂—OH
Ac-rrr-PEG₂—K(cyclo[GfFGrGrQ])-PEG₁₂—OH
Ac-rrr-PEG₂—K(cyclo[FGFGRRRQ])-PEG₁₂—OH
Ac-rrr-PEG₂—K(cyclo[FGFRRRRQ])-PEG₁₂—OH
Ac-rhr-PEG₂—K(cyclo[FfΦGrGrQ])-PEG₁₂—OH
Ac-rhr-PEG₂—K(cyclo[FfΦCit-r-Cit-rQ])-PEG₁₂—OH
Ac-rhr-PEG₂—K(cyclo[FfFGRGRQ])-PEG₁₂—OH
Ac-rhr-PEG₂—K(cyclo[FGFGRGRQ])-PEG₁₂—OH
Ac-rhr-PEG₂—K(cyclo[GfFGrGrQ])-PEG₁₂—OH
Ac-rhr-PEG₂—K(cyclo[FGFGRRRQ])-PEG₁₂—OH
Ac-rhr-PEG₂—K(cyclo[FGFRRRRQ])-PEG₁₂—OH
Ac-rbr-PEG₂—K(cyclo[FfΦGrGrQ])-PEG₁₂—OH
Ac-rbr-PEG₂—K(cyclo[FfΦCit-r-Cit-rQ])-PEG₁₂—OH
Ac-rbr-PEG₂—K(cyclo[FfFGRGRQ])-PEG₁₂—OH
wherein b is beta-alanine, and the exocyclic sequence can be D or L stereochemistry.

In embodiments, the cCPP can be
Ac-rbr-PEG₂—K(cyclo[FGFGRGRQ])-PEG₁₂—OH
Ac-rbr-PEG₂—K(cyclo[GfFGrGrQ])-PEG₁₂—OH
Ac-rbr-PEG₂—K(cyclo[FGFGRRRQ])-PEG₁₂—OH
Ac-rbr-PEG₂—K(cyclo[FGFRRRRQ])-PEG₁₂—OH
Ac-rbrbr-PEG₂—K(cyclo[FfΦGrGrQ])-PEG₁₂—OH
Ac-rbrbr-PEG₂—K(cyclo[FfΦCit-r-Cit-rQ])-PEG₁₂—OH
Ac-rbrbr-PEG₂—K(cyclo[FfFGRGRQ])-PEG₁₂—OH
Ac-rbrbr-PEG₂—K(cyclo[FGFGRGRQ])-PEG₁₂—OH
Ac-rbrbr-PEG₂—K(cyclo[GfFGrGrQ])-PEG₁₂—OH
Ac-rbrbr-PEG₂—K(cyclo[FGFGRRRQ])-PEG₁₂—OH
Ac-rbrbr-PEG₂—K(cyclo[FGFRRRRQ])-PEG₁₂—OH
Ac-rbhbr-PEG₂—K(cyclo[FfΦGrGrQ])-PEG₁₂—OH
Ac-rbhbr-PEG₂—K(cyclo[FfΦCit-r-Cit-rQ])-PEG₁₂—OH
Ac-rbhbr-PEG₂—K(cyclo[FfFGRGRQ])-PEG₁₂—OH
Ac-rbhbr-PEG₂—K(cyclo[FGFGRGRQ])-PEG₁₂—OH
Ac-rbhbr-PEG₂—K(cyclo[GfFGrGrQ])-PEG₁₂—OH
Ac-rbhbr-PEG₂—K(cyclo[FGFGRRRQ])-PEG₁₂—OH
Ac-rbhbr-PEG₂—K(cyclo[FGFRRRRQ])-PEG₁₂—OH
Ac-hbrbh-PEG₂—K(cyclo[FfΦGrGrQ])-PEG₁₂—OH
Ac-hbrbh-PEG₂—K(cyclo[FfΦCit-r-Cit-rQ])-PEG₁₂—OH
Ac-hbrbh-PEG₂—K(cyclo[FfFGRGRQ])-PEG₁₂—OH
Ac-hbrbh-PEG₂—K(cyclo[FGFGRGRQ])-PEG₁₂—OH
Ac-hbrbh-PEG₂—K(cyclo[GfFGrGrQ])-PEG₁₂—OH
Ac-hbrbh-PEG₂—K(cyclo[FGFGRRRQ])-PEG₁₂—OH and
Ac- hbrbh -PEG₂—K(cyclo[FGFRRRRQ])-PEG₁₂—OH,

Structure

| | | |
|---|---|---|
| cyclo(fΦRrRQ) | cyclo(FΦFRRRQ) | cyclo(hF-fΦRrRrQ) |
| cyclo(fΦrRrQ) | cyclo(fFfrRrQ) | cyclo(Sta-fΦRrRrQ) |
| cyclo(FΦrRrQ) | cyclo(fFfRrRQ) | cyclo(Tba-fΦRrRrQ) |
| cyclo(FΦRrRQ) | cyclo(fFϕrRrQ) | cyclo(Ff-1 Nal-RrRrQ) |
| cyclo(FϕRrRQ) | cyclo(fΦfrRrQ) | cyclo(Ff-Bpa-RrRrQ) |
| cyclo(FϕrRrQ) | cyclo(ϕFfrRrQ) | cyclo(Ff-3aa-RrRrQ) |
| cyclo(f-G[decyl]rRrQ) | cyclo(FFFRRRRQ) | cyclo(Ff-1aa-RrRrQ) |
| cyclo(f- | cyclo(RFRFRFRQ) | cyclo(FfΦ-Agp-r-Agp- |
| G[dodecyl]rRrQ) | | rQ) |
| cyclo(f- | cyclo(FfFRrRrQ) | cyclo(FfΦ-Agb-r-Agb- |
| D[dodecenamide]rRrQ) | | rQ) |
| cyclo(f- | cyclo(fFfrRrRQ) | cyclo(FfΦ-hR-r-hR-rQ) |
| D[tetradecanamide]rRrQ) | | |
| cyclo(f- | cyclo(fffrrrrq) | cyclo(FfΦ-4gp-r-4gp- |
| D[hexadecanamide]rRrQ) | | rQ) |
| cyclo(f-Dbg-rRrQ) | cyclo(FfΦRrRaQ) | cyclo(FfΦ-Cit-r-Cit-rQ) |
| cyclo(f-Bta-rRrQ) | cyclo(FfΦRrArQ) | cyclo(Tic-fΦRrRrQ) |
| cyclo(f-Bpa-rRrQ) | cyclo(FfΦRaRrQ) | cyclo(F-tic-ΦRrRrQ) |
| cyclo(fFrRrQ) | cyclo(FfΦArRrQ) | cyclo(NMeF-FΦ-NMeR-R- |
| | | NMeR-RQ) |
| cyclo(fΦrRrQ)-F | cyclo(FfARrRrQ) | cyclo(FfΦRrRrQ)-F |
| cyclo(fΦrRrQ)-f | cyclo(FaΦRrRrQ) | cyclo(FfΦRrRrQ)-f |
| cyclo(fΦrRrQ)-Φ | cyclo(AfΦRrRrQ) | cyclo(FfΦRrRrQ)-Φ |
| cyclo(fΦrRrQ)-ϕ | cyclo(Cpa-fΦRrRrQ) | cyclo(FfΦRrRrQ)-ϕ |
| cyclo(fΦrRrQ)-R | cyclo(Cha-fΦRrRrQ) | cyclo(FfΦRrRrQ)-R |
| cyclo(fΦrRrQ)-r | cyclo(3,4DiF- | cyclo(FfΦRrRrQ)-r |
| | fΦRrRrQ) | |
| cyclo(FFFRRRQ) | cyclo(Tfm-fΦRrRrQ) | cyclo(FfΦRrRrN)-F |
| cyclo(FRFRFRQ) | cyclo(Pfp-fΦRrRrQ) | cyclo(FfΦRrRr-hQ)-F |
| cyclo(FFΦRRRQ) | cyclo(tBuF-fΦRrRrQ) | cyclo(FfΦRrRrq)-F |

The cargo can be a protein and the EEV can be selected from:
Ac-PKKKRKV-PEG₂—K(cyclo[Ff-Nal-GrGrQ])-PEG₁₂—OH
Ac-PKKKRKV-PEG₂—K(cyclo[Ff-Nal-Cit-r-Cit-rQ])-PEG₁₂—OH
Ac-PKKKRKV-PEG₂—K(cyclo[FfF-GRGRQ])-PEG₁₂—OH
Ac-PKKKRKV-PEG₂—K(cyclo[FGFGRGRQ])-PEG₁₂—OH
Ac-PKKKRKV-PEG₂—K(cyclo[GfFGrGrQ])-PEG₁₂—OH
Ac-PKKKRKV-PEG₂—K(cyclo[FGFGRRRQ])-PEG₁₂—OH
Ac-PKKKRKV-PEG₂—K(cyclo[FGFRRRRQ])-PEG₁₂—OH
Ac-rr-PEG₂—K(cyclo[Ff-Nal-GrGrQ])-PEG₁₂—OH
Ac-rr-PEG₂—K(cyclo[Ff-Nal-Cit-r-Cit-rQ])-PEG₁₂—OH
Ac-rr-PEG₂—K(cyclo[FfF-GRGRQ])-PEG₁₂—OH
Ac-rr-PEG₂—K(cyclo[FGFGRGRQ])-PEG₁₂—OH
Ac-rr-PEG₂—K(cyclo[GfFGrGrQ])-PEG₁₂—OH
Ac-rr-PEG₂—K(cyclo[FGFGRRRQ])-PEG₁₂—OH
Ac-rr-PEG₂—K(cyclo[FGFRRRRQ])-PEG₁₂—OH
Ac-rrr-PEG₂—K(cyclo[Ff-Nal-GrGrQ])-PEG₁₂—OH
Ac-rrr-PEG₂—K(cyclo[Ff-Nal-Cit-r-Cit-rQ])-PEG₁₂—OH
Ac-rrr-PEG₂—K(cyclo[FfF-GRGRQ])-PEG₁₂—OH
Ac-rrr-PEG₂—K(cyclo[FGFGRGRQ])-PEG₁₂—OH
Ac-rrr-PEG₂—K(cyclo[GfFGrGrQ])-PEG₁₂—OH
Ac-rrr-PEG₂—K(cyclo[FGFGRRRQ])-PEG₁₂—OH
Ac-rrr-PEG₂—K(cyclo[FGFRRRRQ])-PEG₁₂—OH
Ac-rhr-PEG₂—K(cyclo[Ff-Nal-GrGrQ])-PEG₁₂—OH
Ac-rhr-PEG₂—K(cyclo[Ff-Nal-Cit-r-Cit-rQ])-PEG₁₂—OH
Ac-rhr-PEG₂—K(cyclo[FfF-GRGRQ])-PEG₁₂—OH
Ac-rhr-PEG₂—K(cyclo[FGFGRGRQ])-PEG₁₂—OH
Ac-rhr-PEG₂—K(cyclo[GfFGrGrQ])-PEG₁₂—OH
Ac-rhr-PEG₂—K(cyclo[FGFGRRRQ])-PEG₁₂—OH
Ac-rhr-PEG₂—K(cyclo[FGFRRRRQ])-PEG₁₂—OH
Ac-rbr-PEG₂—K(cyclo[Ff-Nal-GrGrQ])-PEG₁₂—OH
Ac-rbr-PEG₂—K(cyclo[Ff-Nal-Cit-r-Cit-rQ])-PEG₁₂—OH
Ac-rbr-PEG₂—K(cyclo[FfF-GRGRQ])-PEG₁₂—OH
Ac-rbr-PEG₂—K(cyclo[FGFGRGRQ])-PEG₁₂—OH
Ac-rbr-PEG₂—K(cyclo[GfFGrGrQ])-PEG₁₂—OH
Ac-rbr-PEG₂—K(cyclo[FGFGRRRQ])-PEG₁₂—OH
Ac-rbr-PEG₂—K(cyclo[FGFRRRRQ])-PEG₁₂—OH
Ac-rbrbr-PEG₂—K(cyclo[Ff-Nal-GrGrQ])-PEG₁₂—OH
Ac-rbrbr-PEG₂—K(cyclo[Ff-Nal-Cit-r-Cit-rQ])-PEG₁₂—OH
Ac-rbrbr-PEG₂—K(cyclo[FfF-GRGRQ])-PEG₁₂—OH
Ac-rbrbr-PEG₂—K(cyclo[FGFGRGRQ])-PEG₁₂—OH
Ac-rbrbr-PEG₂—K(cyclo[GfFGrGrQ])-PEG₁₂—OH
Ac-rbrbr-PEG₂—K(cyclo[FGFGRRRQ])-PEG₁₂—OH
Ac-rbrbr-PEG₂—K(cyclo[FGFRRRRQ])-PEG₁₂—OH
Ac-rbhbr-PEG₂—K(cyclo[Ff-Nal-GrGrQ])-PEG₁₂—OH
Ac-rbhbr-PEG₂—K(cyclo[Ff-Nal-Cit-r-Cit-rQ])-PEG₁₂—OH
Ac-rbhbr-PEG₂—K(cyclo[FfF-GRGRQ])-PEG₁₂—OH
Ac-rbhbr-PEG₂—K(cyclo[FGFGRGRQ])-PEG₁₂—OH
Ac-rbhbr-PEG₂—K(cyclo[GfFGrGrQ])-PEG₁₂—OH
Ac-rbhbr-PEG₂—K(cyclo[FGFGRRRQ])-PEG₁₂—OH
Ac-rbhbr-PEG₂—K(cyclo[FGFRRRRQ])-PEG₁₂—OH
Ac-hbrbh-PEG₂—K(cyclo[Ff-Nal-GrGrQ])-PEG₁₂—OH
Ac-hbrbh-PEG₂—K(cyclo[Ff-Nal-Cit-r-Cit-rQ])-PEG₁₂—OH
Ac-hbrbh-PEG₂—K(cyclo[FfF-GRGRQ])-PEG₁₂—OH
Ac-hbrbh-PEG₂—K(cyclo[FGFGRGRQ])-PEG₁₂—OH
Ac-hbrbh-PEG₂—K(cyclo[GfFGrGrQ])-PEG₁₂—OH
Ac-hbrbh-PEG₂—K(cyclo[FGFGRRRQ])-PEG₁₂—OH
Ac-hbrbh-PEG₂—K(cyclo[FGFRRRRQ])-PEG₁₂—OH
wherein b is beta-alanine, and the exocyclic sequence can be D or L stereochemistry.

### Cargo

The cell penetrating peptide (CPP), such as a cyclic cell penetrating peptide (e.g., cCPP), can be conjugated to a cargo. The cargo can be a therapeutic moiety. The cargo can be conjugated to a terminal carbonyl group of a linker. At least one atom of the cyclic peptide can be replaced by a cargo or at least one lone pair can form a bond to a cargo. The cargo can be conjugated to the cCPP by a linker. The cargo can be conjugated to an AA_{SC} by a linker. At least one atom of the cCPP can be replaced by a therapeutic moiety or at least one lone pair of the cCPP forms a bond to a therapeutic moiety. A hydroxyl group on an amino acid side chain of the cCPP can be replaced by a bond to the cargo. A hydroxyl group on a glutamine side chain of the cCPP can be replaced by a bond to the cargo. The cargo can be conjugated to the cCPP by a linker. The cargo can be conjugated to an AA_{SC} by a linker.

The cargo can comprise one or more detectable moieties, one or more therapeutic moieties, one or more targeting moieties, or any combination thereof. The cargo can be a hybrid oligonucleotide as described herein.

Cyclic Cell Penetrating Peptides (cCPPs) Conjugated to a Cargo Moiety

The cyclic cell penetrating peptide (cCPP) can be conjugated to a cargo moiety.

The cargo moiety can be conjugated to cCPP through a linker. The cargo moiety can comprise therapeutic moiety. The therapeutic moiety can comprise an oligonucleotide. The oligonucleotide can comprise a hybrid oligonucleotide as described herein. The cargo moiety can be conjugated to the linker at the terminal carbonyl group to provide the following structure:

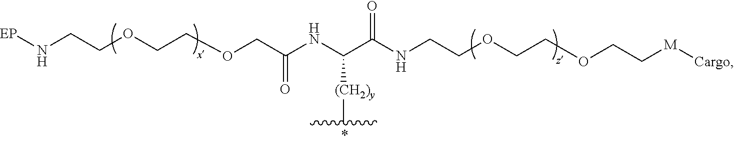

wherein:
EP is an exocyclic peptide and M, AA_{SC}, Cargo, x′, y, and z′ are as defined above, * is the point of attachment to the AA_{SC}. x′ can be 1. y can be 4. z′ can be 11. —(OCH₂CH₂)_{x′}— and/or —(OCH₂CH₂)_{z′}— can be independently replaced with one or more amino acids, including, for example, glycine, beta-alanine, 4-aminobutyric acid, 5-aminopentanoic acid, 6-aminohexanoic acid, or combinations thereof.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C):

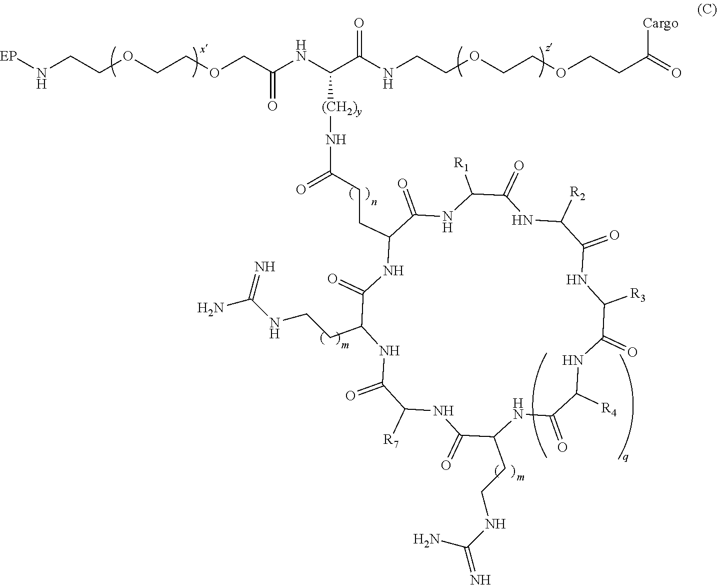

or a protonated form thereof,
wherein:
   R₁, R₂, and R₃ can each independently be H or an amino acid residue having a side chain comprising an aromatic group;
R₄ is H or an amino acid side chain;
EP is an exocyclic peptide as defined herein;
Cargo is a moiety as defined herein;
each m is independently an integer from 0-3;
n is an integer from 0-2;
x′ is an integer from 2-20;
y is an integer from 1-5;
q is an integer from 1-4; and
z′ is an integer from 2-20.

R₁, R₂, R₃, R₄, EP, cargo, m, n, x′, y, q, and z′ are as defined herein.

The EEV can be conjugated to a cargo and the EEV-conjugate can comprise the structure of Formula (C-a) or (C-b):

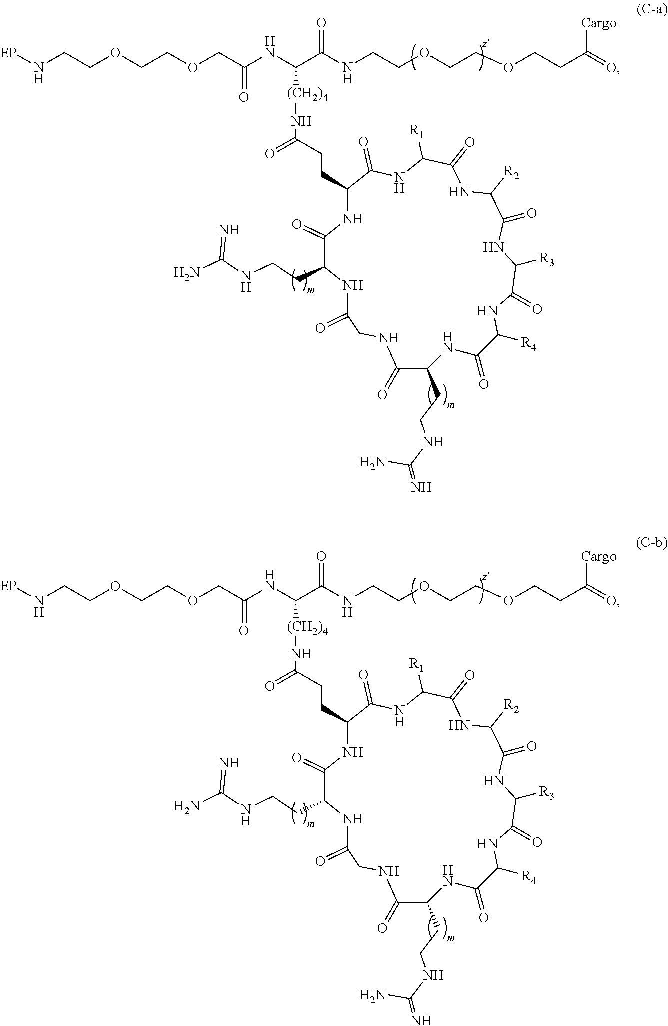

or a protonated form thereof, wherein EP, m and z are as defined above in Formula (C).

The EEV can be conjugated to a cargo and the EEV-conjugate can comprise the structure of Formula (C-c):

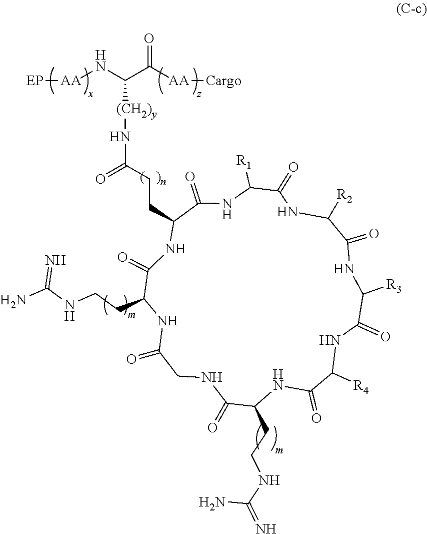

or a protonated form thereof,
wherein EP, R¹, R², R³, R⁴, and m are as defined above in Formula (III); AA can be an amino acid as defined herein; n can be an integer from 0-2; x can be an integer from 1-10; y can be an integer from 1-5; and z can be an integer from 1-10.

The EEV can be conjugated to an oligonucleotide cargo and the EEV-oligonucleotide conjugate can comprises a structure of Formula (C-1), (C-2), (C-3), or (C-4):

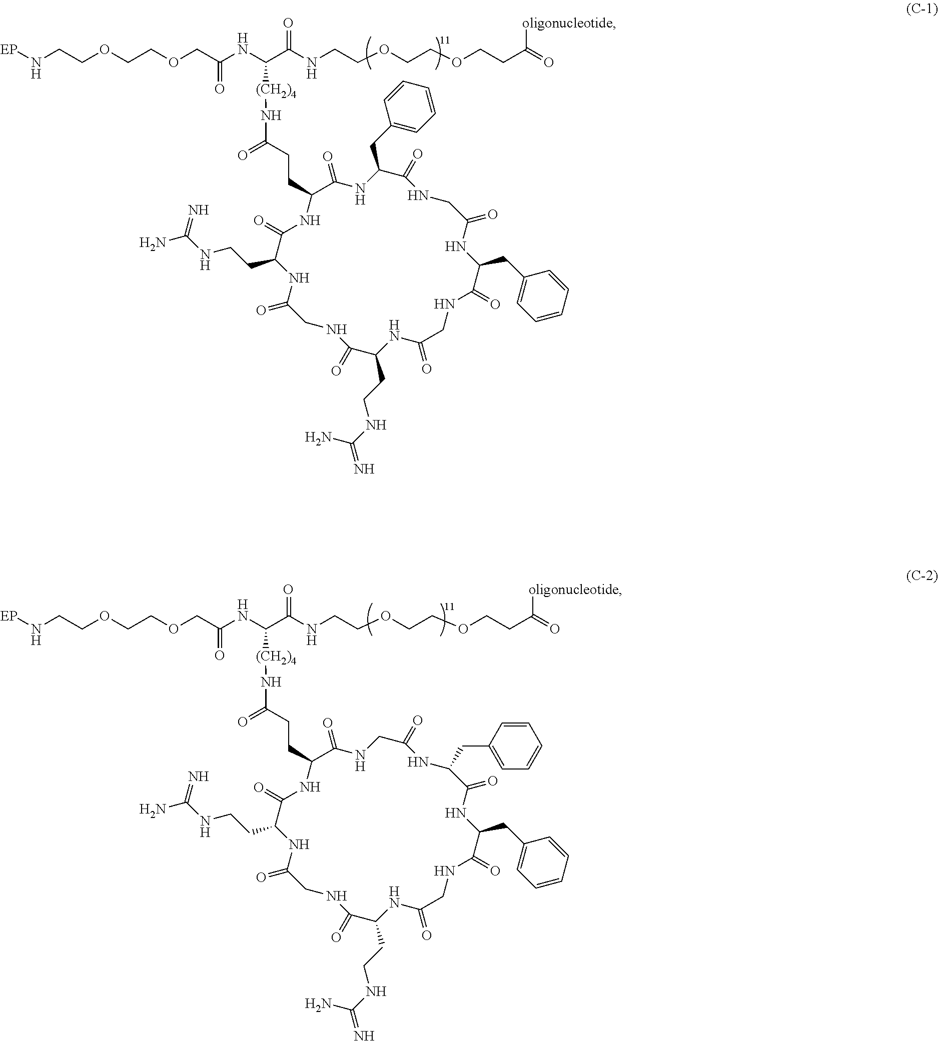

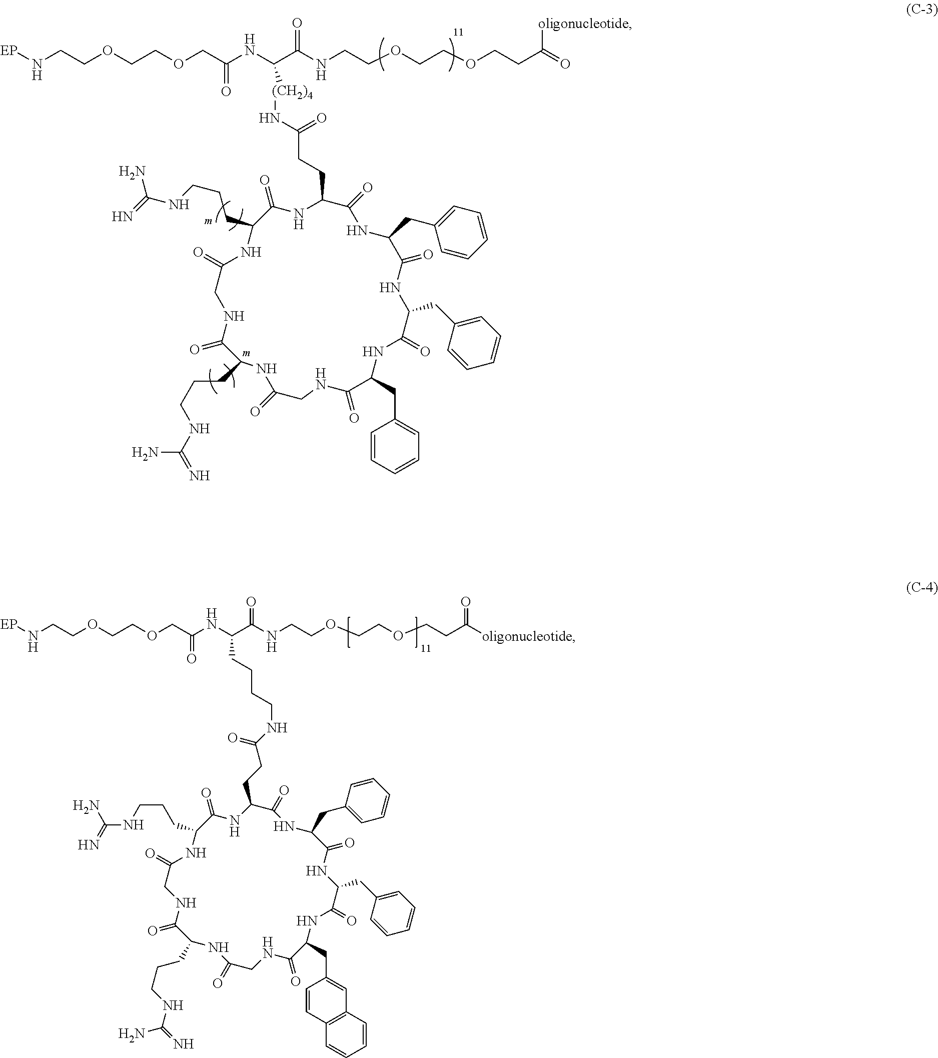

### Diseases

In embodiments, the disease is a genetic disease or disorder. In embodiments, the disease is an inflammatory disease. In embodiments, the disease is an autoimmune disease. In embodiments, the disease is cancer. In embodiments, the disease is a neurological disease. In embodiments, the disease is a cardiovascular disease. In embodiments, the disease is a metabolic disease. In embodiments, the disease is an infectious disease. In embodiments, the disease is a hematological disease. In embodiments, the disease is a musculo-skeletal disease. In embodiments, the disease is a degenerative nerve disease. In embodiments, the disease is a respiratory disease. In embodiments, the disease is a gastrointestinal disease. In embodiments, the disease is an endocrinological disease. In embodiments, the disease is an ophthalmological disease. In embodiments, the disease is a nephrological disease.

In embodiments, the disease is Duchenne muscular dystrophy.

In embodiments, the disease is Huntington's disease (HD), Huntington disease-like 2 (HDL2), myotonic dystrophy type 1 (DM1), myotonic dystrophy type 2 (DM2), spinocerebellar ataxia (e.g., spinal cerebellar ataxia type 1 (SCA1), spinal cerebellar ataxia type 2 (SCA2), or spinal cerebellar ataxia type 3 (SCA3)), spinal and bulbar muscular atrophy (SBMA), dentatorubral-pallidoluysian atrophy (DRPLA), amyotrophic lateral sclerosis, frontotemporal dementia, Fragile X syndrome, fragile X mental retardation 1 (FMR1), fragile X mental retardation 2 (FMR2), Fragile XE mental retardation (FRAXE). Friedreich's ataxia (FRDA), fragile X-associated tremor/ataxia syndrome (FXTAS), myoclonic epilepsy, oculopharyngeal muscular dystrophy (OPMD), or syndromic or non-syndromic X-linked mental retardation.

In embodiments, the disease is Friedreich's ataxia (FRDA). In embodiments, the target gene is FXN, which encodes for frataxin. In embodiments, the compounds provided herein comprise an antisense oligonucleotide that targets FXN.

### Compositions and Methods of Administration

The compounds of the present disclosure may be formulated into compositions suitable for in vivo applications. The compounds and/or compositions may be administered to a patient that has, or is suspected of having, a genetic disease or disorder.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable composition and administered by any suitable route known in the art including, for example, oral and parenteral routes of administration. As used herein, the term parenteral includes subcutaneous, intradermal, intravenous, intramuscular, intraperitoneal, intrasternal, and intrathecal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow-release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated into pharmaceutical compositions according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The form depends on the intended mode of administration and therapeutic application. The compositions also include conventional pharmaceutically acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors, poly[bis(p-carboxyphenoxy) propane:sebacic acid] in a 20:80 molar ratio (as used in GLIADEL); chondroitin; chitin; and chitosan.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, isotonic agents may be included, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, methods of preparation include vacuum drying and the freeze-drying techniques, which yield a powder of the active ingredient plus any additional desired ingredient present in the previously sterile-filtered solutions.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound are disclosed herein. The dose administered to a patient, particularly a human, should be sufficient to achieve a therapeutic response in the patient over a reasonable time frame, without lethal toxicity, and causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein and/or pharmaceutical compositions containing the same, in one or more containers. The disclosed kits can optionally include pharmaceutically acceptable carriers and/or diluents. In one embodiment, a kit includes one or more other components, adjuncts, or adjuvants as described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### Methods of Treatment

The present disclosure provides a method of treating disease in a subject in need thereof, comprising administering a compound and/or composition containing the compound disclosed herein. In embodiments, the disease is a genetic disease. As used herein, “genetic disease” refers to any disease or disorder that is caused, in whole or in part, by a change in the genomic DNA sequence from a wild-type sequence. In embodiments, the compound disclosed herein targets a gene or gene transcript associated with a genetic disease. Treatment of the disease and/or symptoms of the disease may occur through a variety of molecular mechanisms such as those described herein.

In embodiments, the patient is identified as having, or at risk of having, any disease as described herein.

In embodiments, treatment refers to partial or complete alleviation, amelioration, relief, inhibition, delaying onset, reducing severity and/or incidence of one or more symptoms in a subject.

In embodiments, a method is provided for altering the expression, activity or a combination thereof of a target gene in a subject in need thereof, comprising administering a compound disclosed herein. In embodiments, the treatment results in reduced expression of a target protein from a target transcript. In embodiments, treatment results in reduced levels of a target transcript. In embodiments, treatment results in the modulation of splicing of downstream gene transcripts that are regulated by the target transcript and/or proteins that bind to the target transcript. In embodiments, modulation of splicing of downstream gene transcripts results in an increase in downstream transcripts and/or downstream proteins isoforms that are associated with healthy phenotypes. In embodiments, the alternative splicing results in a decrease in downstream transcripts and/or downstream proteins isoforms that are associated with disease phenotypes.

In embodiments, treatment results in a decreased level of the target transcript and/or expression of the target transcript as compared to the average level of the target transcript or target protein in the subject before the treatment or compared to one or more control individuals with similar disease without treatment.

In embodiments, treatment results in a decreased level of downstream transcript and/or expression of a downstream gene product that is associated with a disease phenotype as compared to the average level of the protein in the subject before the treatment or compared to one or more control individuals with similar disease without treatment.

In embodiments, treatment results in an increased level of downstream transcript and/or expression of a downstream gene product that is associated with a healthy phenotype as compared to the average level of the protein in the subject before the treatment or compared one or more control individuals with similar disease without treatment.

In embodiments, treatment results in decreased expression of a protein in a subject's tissue of interest as compared to the average level of the protein in the subject's tissue of interest before the treatment or compared to one or more control individuals with similar disease without treatment.

In embodiments, treatment results in increased expression of an alternately spliced downstream protein in a subject's tissue of interest as compared to the average level of the downstream protein in the subject's tissue of interest before treatment or compared to one or more control individuals with similar disease without treatment.

In embodiments, treatment results in increased or decreased expression of a wild-type downstream protein isomer in a subject's tissue of interest as compared to the average level of the downstream protein in the subject's tissue of interest before the treatment or compared to one or more control individuals with similar disease without treatment.

The terms, “improve,” “increase,” “reduce,” “decrease,” and the like, as used herein, indicate values that are relative to a control. In embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with the same disease, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having a disease or having the potential to develop a disease. The individual may have a disease mediated by aberrant gene expression or aberrant gene splicing. In embodiments, the individual having the disease may have downstream protein expression or activity levels that are less than about 1% to less than about 99% of normal wild type protein expression or activity levels in an individual not afflicted with the disease. In embodiments, the individual may have downstream protein expression or activity levels that are more than about 10% or more than about 500% greater than normal wild type target protein expression or activity levels in an individual not afflicted with the disease.

### Nucleotide Repeat Expansion

In embodiments, a method is provided for treating a disease associated with a nucleotide repeat expansion. Diseases can be associated with tri-, tetra-, penta-, hexa- and dodeca-nucleotide repeat expansions and include, but are not limited to:

| Nucleotide Repeat | |
|---|---|
| Expansion | Disease |
| CAG | Huntington disease, spinal and bulbar muscular atrophy, dentatorubral- |
| | pallidoluysian atrophy and spinocerebellar ataxia (SCA) |
| GGG | Fragile X, Fragile X tremor ataxia syndrome |
| CTG | Myotonic Dystrophy type 1 (DM1), Huntington′s disease-like 2, |
| | spinocerebellar ataxia type 8 (SCA8), Fuch Corneal Dystrophy |
| GAA | Friedrich′s Ataxia |
| GCC | FRAXE mental retardation |
| GCG | Oculopharyngeal muscular dystrophy |
| CCTG | Myotonic Dystrophy type 1 (DM1) |
| ATTCT | Spinocerebellar ataxia type 10 (SCA10) |
| TGGAA | Spinocerebellar ataxia type 31 (SCA31) |
| GGCCTG | Spinocerebellar ataxia type 36 (SCA36) |
| GGGGCC | C90RF72 frontotemporal dementia/amyotrophic lateral sclerosis |
| CCCCGCCCCGCG | Myoclonic epilepsy (EPM1) |

See, Paulson, H. (2018) “Repeat expansion disease,” Handb. Clin. Neurol. 147:105-123.

In embodiments, a method is provided for treating a disease associated with a trinucleotide repeat expansion. In embodiments, the trinucleotide repeat expansion is in a 3′ untranslated region of a gene/transcript. In embodiments, the trinucleotide repeat expansion is a CTG CUG expansion. In embodiments, a method is provided for treating myotonic dystrophy (DM1).

In embodiments, a method is provided for treating myotonic dystrophy type 1 (DM1) by reducing sequestration of at least one RNA-binding protein to a pre-mRNA comprising at least one expanded CUG repeat. In embodiments, a method is provided for treating DM1 by reducing accumulation of a pre-mRNA comprising at least one expanded CUG repeat. In embodiments, a method is provided for treating DM1 by correcting splicing defects of downstream gene transcripts

In embodiments, treatment results in a decreased number of CUG repeat RNA nuclear foci of a target gene as compared to the average level foci in the subject before the treatment or compared to one or more control individuals with similar disease without treatment.

All publications, patents and patent applications mentioned in the specification are indicative of the level of skill of those skilled in the art to which this invention pertains. All publications, patents and patent applications are herein incorporated by reference to the same extent as if each individual publication or patent application was specifically and individually indicated to be incorporated by reference.

### EXAMPLES

### Example 1: Synthetic Method

### Methods

Syntheses of the oligonucleotides were carried out on a 10 μmol scale on a Nitto UnyLinker support 195 μmol/g. Three sets of ACGT monomers were used. PMO 6′-amidites were purchased from Granlen Inc. PMO 6′-phosphoroamidates were purchased from Hongene Biotech Corporation. Reversed DNA or RNA amidites were purchased from ChemGenes Corporation or Hongene Biotech Corporation. For sequences starting with PMO the first monomer was PMO amidite. All solid phase syntheses were carried out using a Mermade 6 automated synthesizer starting from the 5′ end. The PMO fragments were synthesized in a 11 mL glass vial, or 25 mL glass peptide synthesizer with a frit on a larger scale.

Addition of the first morpholino monomer to the solid support: 1.0 g of UnyLinker 195 μmol/g loading support was placed in a glass peptide synthesis vessel having a fritted glass support. Following detritylation and acetonitrile washing steps, a solution of an A-PMO amidite (600 mg, 0.77 mmol, 4 eq) monomer in acetonitrile (0.1M, 7 mL) and solid 5-ethylthio-1H-tetrazol (ETT) (200 mg, 1.53 mmol) were added. The vessel was capped and agitated for 30 minutes at room temperature. After drainage of the solvent, the coupling step was repeated. Oxidation was carried out using 0.05M iodine-water (10%)-pyridine (90%) solution until discoloration no longer occurred. Acetonitrile wash removed traces of the oxidizing solution.

Detritylation solution (CYTFA): 100 mM 4-cyanopyridine, 100 mM TFA in dichloromethane:trifluoroethanol:ethanol (80:20:1).

Neutralization Solution (N): 5% DIPEA in dichloromethane:isopropanol (3:1).

Coupling solution (C: 1.6 g of PMO monomer. 9.0 mL of DMI, 1.0 mL of NEM.

Wash solution (W): dichloromethane.

Storage solution (S): 30% trifluoroethanol in dichloromethane.

TABLE 1
Conditions for the manual PMO synthesis.

| Step | Reaction | Reagent | Time [min] | Temperature [° C.] |
|---|---|---|---|---|
| 1 | Wash | W | 3 × 1 | rt |
| 2 | Detritylation | CYTFA | 6 × 10 | rt |
| 3 | Wash | W | 3 × 1 | rt |
| 4 | Neutralization | N | 4 × 5 | rt |
| 5 | Wash | W | 3 × 1 | rt |
| 6 | Coupling | C | 1 × 120 | 50 |
| 7 | Wash | W | 3 × 1 | rt |

### Addition of DNA Monomers

Addition of reversed DNA amidites to terminal PMO monomer: The support (100 mg) was weighed in a 4 mL fritted Mermade 6 column and placed in a vacuum manifold for solid phase extraction. The trityl group was removed using CYTFA (6×10 min) and Neutralization solutions (4×5 min). Column was transferred to Mermade 6 to perform coupling with the first amidite (3×10 min) followed by sulfurization with 3-((Dimethylamino-methylidene)amino)-3H-1,2,4-dithiazole-3-thione (DDTT) solution (2×3 minutes). The synthesis of ASO fragment was continued in an automated manner and terminated with a dimethoxytrityl (DMT) protecting group.

ASO synthesis on 10-15 μmol scale(

Wash: Acetonitrile

Deblock: 3% trichloroacetic acid in DCM

Amidite solution is 0.1 M in Acetonitrile

Activator solution is 0.2 M benzyl-S-tetrazole in Acetonitrile

Oxidation reagent: 0.05 M iodine, 10% water, 90% pyridine

Sulfurizing reagent: 0.1 M DDTT in pyridine

Cap A solution: 10% lutidine, 10% iso-Butyric anhydride, 80% ACN

Cap B solution: 16% 1-Methylimidazole, 85% THF

### Automated Synthesis

Initialization:

ACN wash 2×1.5 mL

Run steps:

Deblock 4×1.2 mL

ACN wash 4×1.5 mL

Coupling 2×800 μL of amidite solution+800 μL of the activator (3 minutes for DNA, 10 minutes for RNA, reverse DNA, LNA)

ACN wash 1.5 mL

Oxidition 2×1000 μL of oxidizer solution or 1000 μL of sulfurizing solution

ACN wash 1.5 mL

Cap 2×(A 400 uL+Cap B 400 uL)

ACN wash 2×1.5 mL

Finalization:

Deblock 4×1.2 mL

ACN wash 4×1.5 mL

### Deprotection and Purification

For simplicity of IEX purification trityl group was kept on. A suspension of solid support in 15 mL of 1 M 1,8-Diazabicyclo[5.4.0]undec-7-ene (DBU) solution in dry acetonitrile (ACN) was shaken at RT for 1.5 hours. The clear solution was decanted, and the process was repeated twice with a fresh DBU solution. The support was rinsed with dry ACN, dried and placed in a 30 mL flask with 15 ml of conc. ammonia. The flask was heated overnight at 50° C. The solution was filtered and analyzed by MALDI and IEX-HPLC. Purification yielded 3′-Trityl protected oligonucleotide. Sample was desalted using a Sephadex column, collected in a 50 mL Falcon tube and dried in vacuo.

The solid was dissolved in water (2 mL), followed by addition of 20% H₃PO₄ (40 uL). The resulting suspension was placed in a heater-shaker at 50° C. for 30 minutes. Concentrated ammonia (1 mL) was added, and the suspension was filtered before purification on IEX-HPLC. Fractions containing deprotected product were pooled and desalted on Sephadex. Filtration and evaporation provided fully deprotected PMO-DNA gapmer.

Diethylamine (20%) solution in ACN was passed through the support (8 mL) over 2 minutes. Support was air dried and placed in a 30 mL flask for ammonia deprotection. Concentrated ammonia (9 mL) was added, and the suspension was placed at 50° C. overnight. The flask was cooled to −20° C., then aqueous methylamine (9 mL, 40%) was added. The flask was heated for 6 more hour at 50° C. Analysis by HPLC and MALDI was followed by IEX-HPLC purification. Fractions containing deprotected product were pooled and desalted on Sephadex. Filtration and evaporation provided fully deprotected PMO-DNA gapmer.

### Results

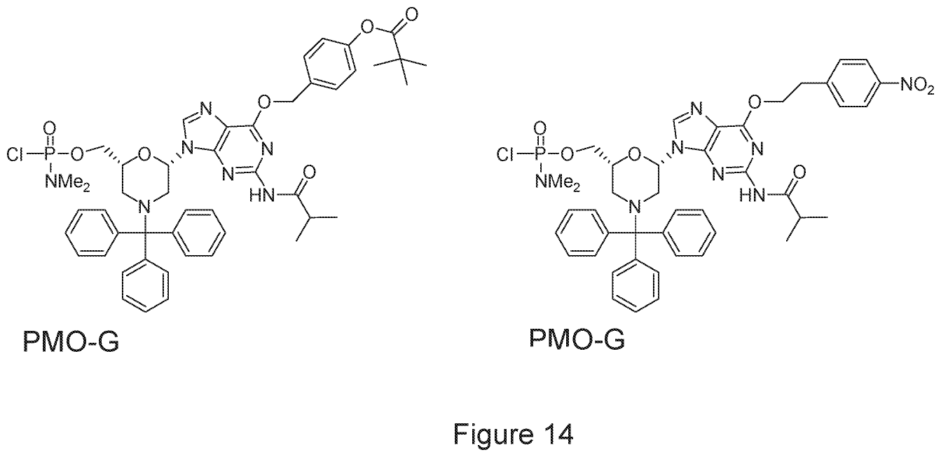

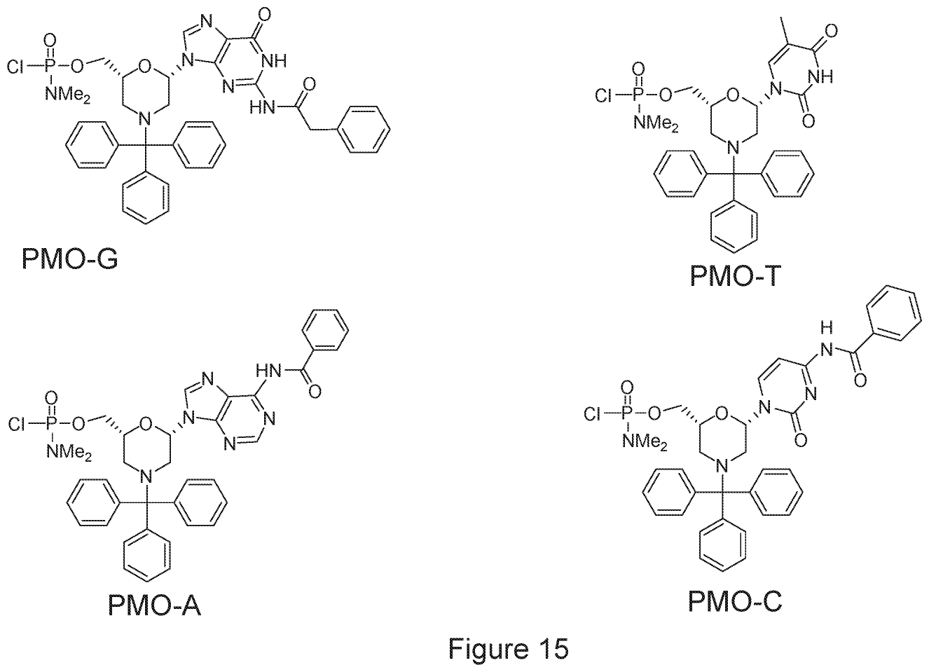

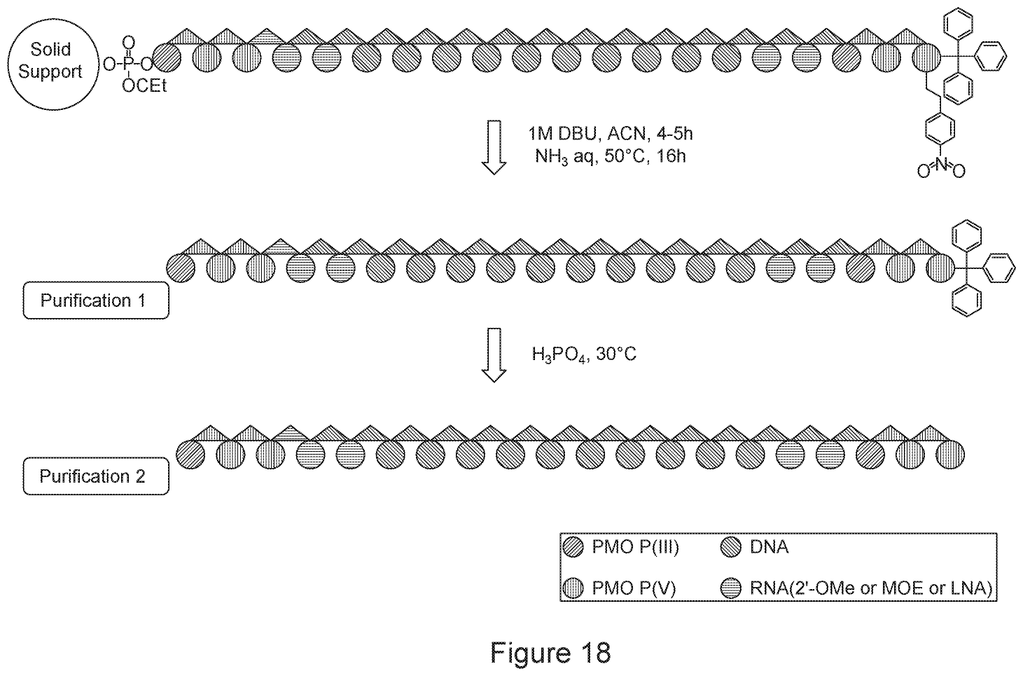

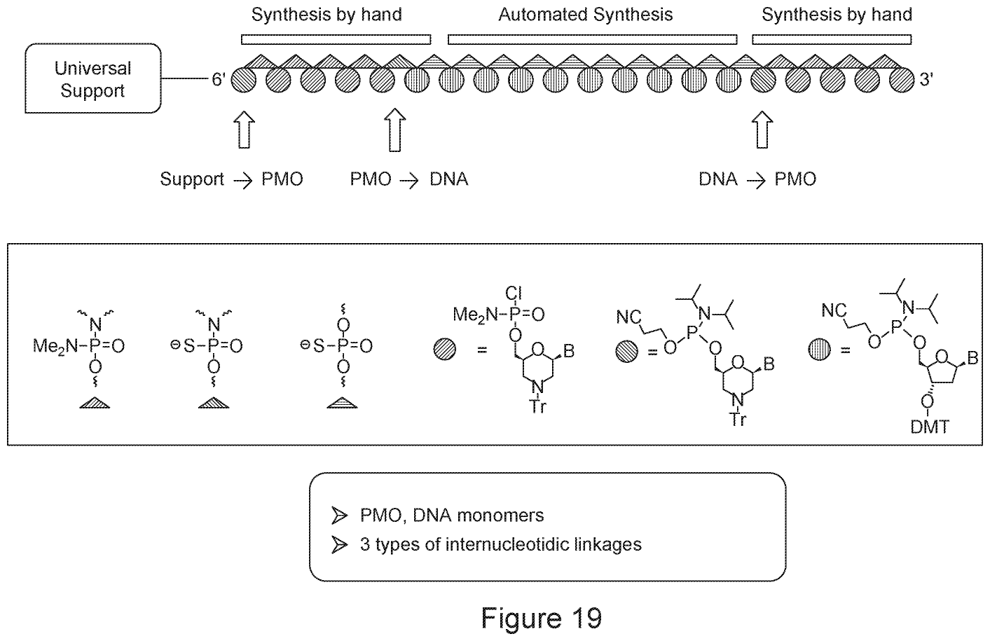

A common misconception is that synthetic oligonucleotides can be made only in a 3′ to 5′ direction. Although not widely used, reversed ribose 5′-phosphoramidites are commercially available and have been employed before by others (Bhagat et al. J. Med. Chem. 2011, 54, 3027-3036, which is incorporated by reference as if fully set forth herein). Reversed DNA amidites as well as their 2′-F, 2′-OMe, 2′-OMOE congeners are available. The compatibility of PMO and phosphoramidite (PS) chemistries was tested using a 20 nucleotide (nt) long human DMPK sequence 5′-ACAGACAATAAATACCGAGG-3′ as a template. That length allows for comparative studies with 5-10-5 gapmers, 20 nt long PMO and for testing different chimeric designs. Synthesis of PMO 6′-flanking domain was accomplished by manual addition of PMO monomers to the solid support B (FIGS. 18 and 19). The 3′-terminal secondary amino group was acid deprotected and neutralized to prepare ground for the addition of 5′-cyanophosphoramidites. Coupling of first DNA monomer to the secondary amino group of the morpholino ring resulted in formation of stable thiophosphoramidate linkage. The DNA-PS was then extended in an automated fashion using standard DNA synthesis protocol to build the gap sequence. The standard deprotection conditions (3% TCA in DCM) did not seem to affect the integrity of the phosphordiamidate linkage even after multiple additions of DNA monomers. After DNA gap completion, the second PMO wing (flank) was added. For a smooth transition between DNA-PS domain and the PMO 3′-flank, PMO 6′-phosphoramidite P(III) was employed as the “lynchpin” or the first PMO nucleotide. Surprisingly, the PMO synthesis was not successful, N-acetyl truncated oligonucleotide was detected instead of the full construct. Apparent migration of acetyl protecting and capping groups to a more nucleophilic secondary amino group likely had occurred. The cytidine N-acetyl protecting group was replaced with N-benzoyl. To slow down the O-acyl group migration to secondary amine, its steric bulk was increased by replacing the acetic anhydride in Cap A solution with iso-butyric anhydride. A similar strategy was reported previously to prevent N-acetylation of “fast deprotecting” nucleobases (Q. Zhu, M. O. Delaney, M. Geenberg Bioorganic & Medicinal Chemistry Letters 11, 2001, 1105-1107, which is incorporated by reference as if fully set forth herein). Thus, simple cap A modification allowed for completion of PMO 3′-flank synthesis. The commercially available P(III) PMO-G monomer displayed low coupling efficiency. Addition of molecular sieves improved the yield, as well as freshly prepared cyanoethyl phosphoroamidite. Addition of P(V) PMO-G monomers also resulted in lower coupling yield. Notably 1^{st} and 2^{nd} gen PMO-G (FIG. 14) feature two nucleobase protecting groups contributing to steric hindrance. The alternative solutions might include monoprotected PMO-G (FIG. 15) as well as allyl, methyl or benzyl groups as a replacement for cyanoethyl phosphoramidites. For testing, a mixed design gapmers 6′-PMO-DNA-RNA-3′, were employed, meaning that the 3′-flank would be composed of 2′-OMOE or 2′-OMe RNA modifications typical of 2^{nd} generation ASO design.

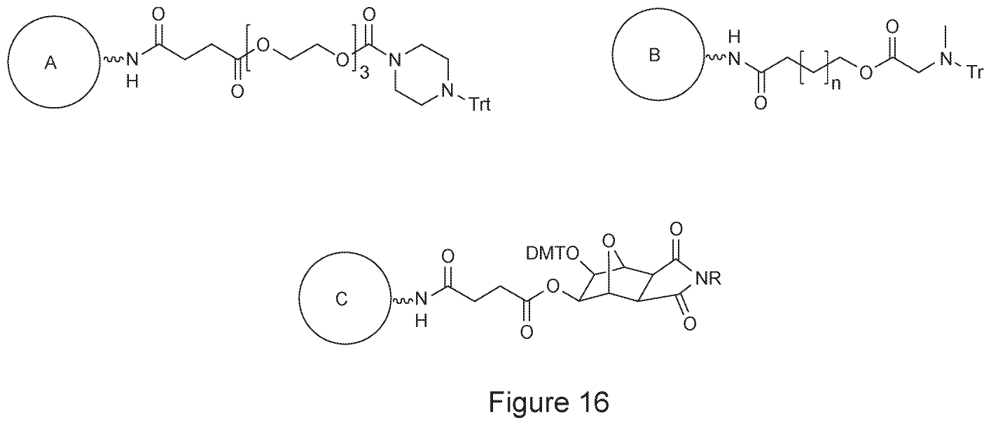

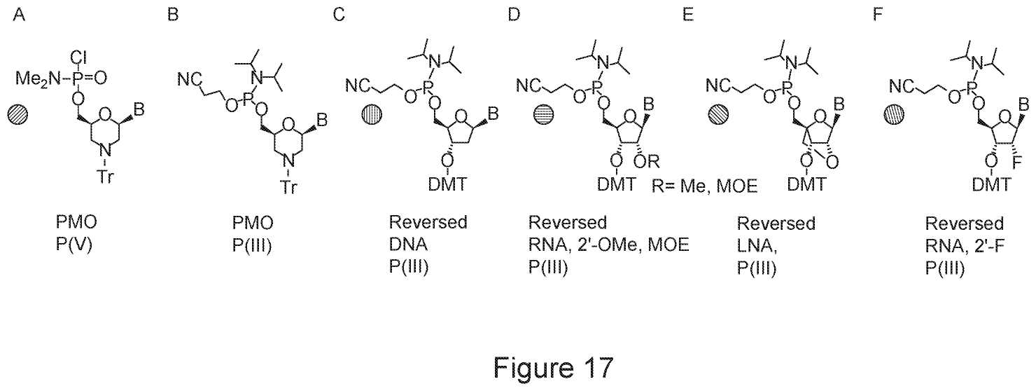

In addition to introducing PMO (III) monomers in the first position of the 3′-flank similar substitution in the first base of the 5′-flank permitted the use of the Universal solid support C typical of conventional DNA, RNA synthesis (FIG. 16). The solid supports used in trityl-based PMO synthesis employ secondary amine anchor decorated with PEG3 (Sarepta) A or Sarcosine (Genetools) B (FIG. 16). Fragments of those anchors are still present in the 6′ terminus of the final product after ammonia deprotection. A switch to P(III) chemistry allows for synthesis of “clean” 6′—OH terminated oligonucleotides.

Access to 6′-conjugation chemistries is available via conventional phosphoramidite approach.

### Example 2: Condition Screening

| Step | WO2022/125987 | Conditions |
|---|---|---|
| Trityl deblock | 3% trichloroacetic acid in | 100 mM 4-cyanopyridine, 100 mM |
| | DCM | TFA in DCM:TFE:Ethanol (80:20:1) |
| | 40 s | |
| Cap A (DNA part) | 10% Ac2O in THF | 10% (iPrCO)2O, 10% 2,6-Lutidine, |
| Cap B | 10% N-Me-imidazole, 10% | 80% THF |
| | pyridine, 80% THF | 16% N-Me-imidazole, 84% THF |
| Cap A (PMO part) | 10% Ac2O, 10% 2,6- | No Capping |
| Cap B | Lutidine, 80% THF | |
| | 16% N-Me-imidazole, | |
| | 84% THF | |
| Coupling step | Monomer in DMI (0.1M) | Monomer in DMI (0.1M) |
| | Pentamethylpiperidine | N-Ethylmorpholine, |
| | Room temperature, 8 h | 50° C., 2 h |

### Example 3: Oligonucleotide Libraries

### Methods

Syntheses of the oligonucleotides were carried out on a 10 mol scale on a Nitto UnyLinker support 195 μmol/g. Three sets of ACGT monomers were used. PMO 6′-phosphoramidites were purchased from Granlen Inc. PMO 6′-phosphoramidates were sourced from Hongene Biotech Corporation or Wuxi Corporation. Reversed β-cyanoethylamidites were purchased from ChemGenes Corporation or Hongene Biotech Corporation. 0.1 M DDTT was used as a sulfurizing agent to oxidize trialkylphosphite intermediates. For sequences starting with PMO the first monomer was PMO 6′-phosphomamidite. All solid phase syntheses were carried out using a Mermade 6 automated synthesizer starting from the 5′ end. The PMO fragments were synthesized manually in a glass peptide reaction vessel with a frit or in a 4 mL polyethylene Mermade 6 column. 1^{st} and 2^{nd} generation double protected PMO-G were used for synthesis of libraries 1 and 2. After the synthesis, oligonucleotides were deprotected, IEX-HPLC purified on Åkta™ Avant 25 (Source15Q) and desalted on Sephadex column (2.5×25 cm). The oligonucleotides were lyophilized and resuspended in sterile water and the concentrations were determined by measuring the UV absorbance at 260 nm. All oligonucleotides were characterized by HPLC (Agilent Technologies 1260 Infinity 11), MALDI-ToF (Bruker Autoflex Max) and LCMS for molecular mass. LCMA characterization was performed by Novatia, LLC.

### HPLC Methods:

Analytical IEX injections were carried out on DNAPac 200 oligonucleotide HPLC column on Agilent 1260 HPLC using and increasing gradient of 1M sodium perchloride in a 20% acetonitrile solution in water, TRIS buffer pH 8.0.

### Oligonucleotide Purification:

Preparative purification of Trityl ON oligonucleotides was carried out by anion exchange chromatography (IEX-HPLC) using Source 15Q column i.d. 10 mm×200 mm on Åkta′ Avant 25 HPLC, eluting at 8 mL/min using an increasing gradient of 2M NaCl in 20% acetonitrile-water Tris buffer, pH 8.0. Fractions were analyzed by HPLC, pooled, desalted on a Sephadex column and evaporated in a Genevac Elite 2.3 evaporator. Trityl-Off oligonucleotides we generated by adding 60 μL of 20% H₃PO₄ to a 2 mL water solution of Tr ON oligonucleotide and 30 min incubation in a heater shaker at 50° C. The suspension was treated with 1 mL of 28% ammonia in water, syringe filtered and loaded into a Source 15Q column. Gradient elution provided fully deprotected oligonucleotide.

Addition of the first morpholino monomer to the solid support: 1.0 g of UnyLinker 195 μmol/g loading support was placed in a glass peptide synthesis vessel having a fritted glass support support. Following a detritylation and acetonitrile wash steps, a solution of the A-PMO amidite (600 mg, 0.77 mmol, 4 eq) monomer in acetonitrile (0.1M, 7 mL) and solid ETT (200 mg, 1.53 mmol) were added. The vessel was capped and agitated for 30 min at room temperature. After drainage of the solvent, the coupling step was repeated. Oxidation was carried out using 0.05M iodine-water (10%)-pyridine (90%) solution until discoloration no longer occurred. Acetonitrile wash removed reminders of the oxidizing solution.

FIG. 18 shows an example of processing and purification of a library.

### Addition of PMO (V) Monomers:

Detritylation solution (CYTFA): 100 mM 4-cyanopyridine, 100 mM TFA in dichloromethane:trifluoroethanol:ethanol (80:20:1).

Neutralization Solution (N): 5% DIPEA in dichloromethane:isopropanol (3:1).

Coupling solution (C): 1.6 g of PMO monomer, 9.0 mL of DMI, 1.0 mL of NEM.

Wash solution (W): dichloromethane.

Storage solution (S): 30% trifluoroethanol in dichloromethane.

TABLE 2
Conditions for the manual PMO synthesis.

| | | | | Temperature |
|---|---|---|---|---|
| Step | Step | Reagent | Time [min] | [° C.] |
| 1 | Wash | W | 3 × 1 | rt |
| 2 | Detritylation | CYTFA | 6 × 10 | rt |
| 3 | Wash | W | 3 × 1 | rt |
| 4 | Neutralization | N | 4 × 5 | rt |
| 5 | Wash | W | 3 × 1 | rt |
| 6 | Coupling | C | 1 × 120 | 50 |
| 7 | Wash | W | 3 × 1 | rt |

Addition of conventional amidites to terminal PMO monomer: The support (100-160 mg) was weighed in a 4 mL fritted Mermade 6 column and placed in a vacuum manifold for solid phase extraction. The trityl was removed using CYTFA (6×10 min) and Neutralization solutions (4×5 min). Column was transferred to Mermade 6 to perform coupling with the first deoxyribose phosphoramidite (2×15 min) followed by sulfurization with DDTT solution (2×3 min). The synthesis of DNA-PS gap was continued in an automated manner and terminated with a DMT-ON.

### Deprotection and Purification of the First Library

For simplicity of IEX purification, the trityl group was kept on. A suspension of solid support in 15 mL of 1 M DBU solution in dry ACN was shaken at RT for 1.5 hours. The clear solution was decanted, and the process was repeated twice with a fresh DBU solution. The support was rinsed with dry ACN, dried and placed in a 30 mL flask with 15 ml of concentrated aqueous ammonia. The flask was then heated overnight at 50° C. The solution was filtered and analyzed by MALDI and IEX-HPLC. Purification yielded 3′-N-trityl protected oligonucleotide. Sample was desalted on Sephadex, collected in a 50 mL Falcon tube and dried in vacuo. The resulting solid was dissolved in water (2 mL), followed by addition of 20% H₃PO₄ (40 uL). The resulting suspension was placed in a heater-shaker at 50° C. for 30 min. Concentrated aqueous ammonia (1 mL) was added and the suspension was syringe filtered before purification on IEX-HPLC. Fractions containing deprotected product were pooled and desalted on Sephadex. Filtration and evaporation provided fully deprotected PMO-DNA gapmer.

### Deprotection and Purification of the Second Library

Diethylamine (20%, 8 mL) solution in ACN was passed through the DMT-OFF support over 2 minutes. Support was air dried and placed in a 30 mL flask for ammonia deprotection. Concentrated aqueous ammonia (9 mL) was added, and the suspension was placed at 50° C. overnight. The flask was briefly cooled to −20° C., then aqueous methylamine (9 mL, 40%) was added. The flask was heated for 6 more hours at 50° C. Analysis by HPLC and MALDI was followed by IEX-HPLC purification. Fractions containing deprotected product were pooled and desalted on Sephadex. Filtration and evaporation provided fully deprotected PMO-DNA gapmer.

TABLE 3
Library 1 of 20 nt chimeric oligonucleotides tested in Hela cells.

| | | | Flank | | | Mw |
|---|---|---|---|---|---|---|
| Sequence # | Sequence (6′-3′)^{a} | Pattern | size | KD | Tm | Calcd./Found |
| Oligo 1 | 6′-ACAGACAATAAATACCGAGG-3′ | 5M-10-5 | 10 | 76 | 57.2 | 6823.6/6822.2 |
| Oligo 2 | 5′-ACAGACAATAAATACCGAGG-3′ | 5-2M-8-5 | 12 | 52 | — | 6765.6/6765.7 |
| Oligo 3 | 5′-ACAGACAATAAATACCGAGG-3′ | 5-10-3-2M | 10 | 29 | 53.4 | 6706.5/6704.4 |
| Oligo 4 | 6′-ACAGACAATAAATACCGAGG-3′ | 2M-16-2M | 4 | 12 | 52.4 | 6475.3/6473.7 |
| Oligo 5 | 6′-ACAGACAATAAATACCGAGG-3′ | 3M-14-3M | 6 | 35 | 51.9 | 6495.4/6494.1 |
| Oligo 6 | 6′-ACAGACAATAAATACCGAGG-3′ | 3M-15-2M | 5 | 42 | 52.4 | 6485.4/6484.4 |
| Oligo 7 | 6′-ACAGACAATAAATACCGAGG-3′ | 4M-14-2M | 6 | 4 | 51.9 | 6495.4/6494.1 |
| Oligo 8 | 6′-ACAGACAATAAATACCGAGG-3′ | 5M-13-2M | 7 | 58 | 53.2 | 6505.4/6504.4 |
| Oligo 9 | 6′-ACAGACAATAAATACCGAGG-3′ | 6M-12-2M | 8 | 78 | 54.2 | 6515.4/6514.3 |
| Oligo 10 | 6′-ACAGACAATAAATACCGAGG-3′ | 7M-11-2M | 9 | 78 | 53.2 | 6525.5/6523.8 |
| Oligo 11 | 6′-ACAGACAATAAATACCGAGG-3′ | 8M-10-2M | 10 | 84 | 53.0 | 6535.5/6534.1 |
| Oligo 12 | 6′-ACAGACAATAAATACCGAGG-3′ | 9M-9-2M | 11 | 85 | 55.2 | 6545.5/6544.2 |
| Oligo 13 | 6′-ACAGACAATAAATACCGAGG-3′ | 10M-8-2M | 12 | 76 | 56.2 | 6555.5/6554.0 |
| Oligo 14 | 5′-ACAGACAATAAATACCGAGG-3′ | 5-10-5 | 10 | 56 | 60.9 | 7226.1/7229.7 |
| Oligo 15 | 5′-TAGTGCGGACCTACCCACGA-3′ | 5-10-5 | 10 | 3 | 50.4 | 7160.4/7160.0 |

^{a}ATCG indicate PMO, ATCG indicates DNA, underlined 2′-OMe RNA, bold underlined 2′-OMOE RNA.

TABLE 4
Library 2 of 20 nt long chimeric oligonucleotides tested in Hela cells.

| | | | | KD% | | |
|---|---|---|---|---|---|---|
| | | | Flank | Nucleo- | | |
| Sequence # | Sequence (6′-3′)ª | Pattern | size | fection | Tm | Mw Calcd./Found |
| Oligo 1 | 6′-ACAGACAATAAATACCGAGG-3′ | 5M-10-5 | 10 | 50 | 57.2 | 6823.6/6822.2 |
| Oligo 16 | 6′-ACAGACAATAAATACCGAGG-3′ | 6M-9-5 | 11 | 63 | 58.3 | 6889.8/6890.0 |
| Oligo 17 | 6′-ACAGACAATAAATACCGAGG-3′ | 7M-8-5 | 11 | 72 | 58.3 | 6899.9/6898.9 |
| Oligo 18 | 6′-ACAGACAATAAATACCGAGG-3′ | 8M-8-4 | 12 | 67 | 55.0 | 6821.2/6821.8 |
| Oligo 19 | 6′-ACAGACAATAAATACCGAGG-3′ | 9M-8-3 | 12 | 67 | 56.0 | 6757.4/6758.7 |
| Oligo 20 | 6′-ACAGACAATAAATACCGAGG-3′ | 9M-9-2 | 11 | 49 | 53.3 | 6596.5/6594.0 |
| Oligo 21 | 6′-ACAGACAATAAATACCGAGG-3′ | 10M-8-2 | 12 | 69 | 54.3 | 6694.7/6693.3 |
| Oligo 22 | 6′-ACAGACAATAAATACCGAGG-3′ | 11M-7-2 | 13 | 46 | 53.3 | 6704.7/6703.6 |
| Oligo 23 | 6′-ACAGACAATAAATACCGAGG-3′ | 12M-6-2 | 14 | 39 | 54.8 | 6714.7/6713.5 |
| Oligo 24 | 6′-ACAGACAATAAATACCGAGG-3′ | 14M-4-2 | 15 | 16 | 54.9 | 6724.7/6723.8 |
| Oligo 25 | 6′-ACAGACAATAAATACCGAGG-3′ | 15M-3-2 | 16 | 11 | 56.4 | 6733.8/6733.8 |
| Oligo 26 | 6′-ACAGACAATAAATACCGAGG-3′ | 20M | 20 | 0 | 61.7 | 6823.9/6821.3 |
| Oligo 55 | 6′-ACAGACAATAAATACCGAGG-3′ | 5M-10-5 | 10 | 84 | 56.5 | 6880.80/6881 |

^{a}ATCG indicate PMO, ATCG indicates DNA, underlined 2′-OMe RNA, bold underlined 2′-OMOE RNA.

TABLE S
Library of 25 nt long chimeric oligonucleotides tested in Hela cells.

| | | | Flank | | | |
|---|---|---|---|---|---|---|
| Sequence # | Sequence (6′-3′)ª | Pattern | size | KD | Tm | Mw Calcd./Found |
| Oligo 27 | 6′-ACAGACAATAAATACCGAGGAATGT-3′ | 10M-10-5 | 15 | 70 | 58.4 | 8561.2/8559.1 |
| Oligo 28 | 6′-ACAGACAATAAATACCGAGGAATGT-3′ | 11M-10-4 | 15 | 69 | 57.6 | 8497.2/8495.3 |
| Oligo 29 | 6′-ACAGACAATAAATACCGAGGAATGT-3′ | 12M-10-3 | 15 | 59 | 56.4 | 8433.1/8430.9 |
| Oligo 30 | 6′-ACAGACAATAAATACCGAGGAATGT-3′ | 13M-10-2 | 15 | 48 | 56.4 | 8369.1/8366.8 |
| Oligo 31 | 6′-ACAGACAATAAATACCGAGGAATGT-3′ | 14M-9-2 | 16 | 52 | 57.2 | 8379.1/8377.4 |
| Oligo 32 | 6′-ACAGACAATAAATACCGAGGAATGT-3′ | 15M-8-2 | 17 | 58 | 57.8 | 8389.1/8387.3 |

ªATCG indicate PMO, ATCG indicates DNA, underlined 2′-OMe RNA, bold underlined 2′-OMOE RNA.

### Results

### Determining the Optimum Size of PMO Flanks and DNA Gap by in Vino KD in HeLa Cells.

Two positive controls were synthesized: sequence Oligo 33 (5′-ACAGACAATAAATACCGAGG-3′), a (2′-OMe) 5-10-5 gapmer and, a (2′-OMOE) 5-10-5 gapmer control sequence (Oligo 14). They both nucleofected the HeLa cells over span of 24 h. Oligo 14 showed 56% DMPK knockdown as opposed to sequence 33 (43% KD) and thus was selected for future experiments as a positive reference. Scrambled sequence (Oligo 15) was selected as the negative control. The first library was of 20 nt long PMO-DNA-PMO gapmers prepared by gradually increasing the number of 6′-PMO wing (flank) from 2 to 10 nucleotides. Conversely, the size of 3′-PMO flank was initially limited to two nucleotides, as it was deemed sufficient to provide nuclease protection. The PMO-DNA gapmers were initially delivered at 15 μM by electroporation to HeLa cells to eliminate uptake rate differences due to different PS content. Oligo 1 and Oligos 9, 10, 11, 12 and 13, which featured high PMO content in one or both flanks showed increased knockdown over the 56% of the positive control gapmer sequence (Oligo 14) (Table 3). Oligo 2, which differed from control Oligo 33 by additional two PMO in left wing showed similar knockdown. Replacement of last two 3′ flank nucleotides by PMO (Oligo 3) resulted in diminished activity (29%) in comparison to Oligo 14. Similarly low PMO content and wing size in Oligos 4, 5, 6, and 7 proved insufficient to knockdown the DMPK.

The requirement for two step purification of the first library prompted the use of 2′-O-MOE nucleotides in a 3′-flank. The bulk of neutral PMOs was placed in a 6′-flank, so replacing two nucleotides in the 3′-flank would have a negligible impact on the overall oligonucleotide charge. The role of the second library (Table 4) was to probe the effect of gradual increasing the 6′-flank from 6 to 15 PMO nucleotides (ONs and gradual decreasing the 3′-flank from five to two 2′-O-MOE nucleotides. The size of the DNA-PS gap would shrink from 10 in Oligo 1 to 3 nucleotides in Oligo 25. RNAse H activity improved with a gap of at least 7 charged nucleotides. Indeed, Oligos 22, 23, 24, and 25 featuring short gaps 7-3 nt long displayed knockdown diminishing from 46 to 11%. Conversely, Oligos16, 17, 18, 19, 20, and 21 that had gap size 8 or 10 provided highest KD equal or better than control Oligo 14.

A library of 25 nt long gapmers was prepared (Table 5). The gap size was maintained at the level mentioned above for RNAse H activity, e.g., 8-10 nt, while the PMO flank size varied between 10 and 15 nt.

Lipofectamine delivery of PMO-DNA hybrids—DMPK knockdown in Hek293 cells.

### Thermal Denaturation Studies

The binding affinity of an oligonucleotide may play an indicative role in determining its therapeutic potential. Factors such as electrical charge, presence of hydrophobic groups, hydrogen bonds, sugar pucker can affect the energy of binding. Conformationally restricted nucleotides have been developed to improve the binding affinity of ONs by entropic gain. Some synthetic ONs can increase the binding affinity while stereorandom phosphorothioates decrease it. In addition to backbone type, the number of modifications and their position within a sequence seem to play a crucial role in determining binding affinity. Morpholino phosphoramidites as well as isoelectronic N3′—P5′ phosphoramidates and sulfur analogues showed increased binding with complementary RNA. Thiophosphoroamidate morpholino oligonucleotides (TMOs) showed depressed binding with DNA (−12.6° C.) and increased binding with RNA target (+10° C.). In contrast, alternating morpholino thiophosphoroamidate and DNA-PS subunits increase binding affinity towards both DNA and RNA (6-10° C.). It was hypothesized that alternation of subunits can result in increased flexibility to an otherwise rigid ON permitting efficient binding with the complementary strand. Encouraged by these findings, duplex formation properties of PMO-DNA chimeras towards complementary RNA were investigated (Tables 3-5). The data presented in Table 3 show that the increase in thermal stability is correlated with the size of 6′ PMO wing. Increase in number of PMO modifications from 2 to 10 (Oligos 4-13) resulted in modest Tm increase (+2.8° C., or +0.35° C. per base). For Oligos 20, 21, 22, 23, 24, and 25 the increase in PMO numbers from 9 to 15 (Table 4) changed Tm by +4.9° C., or +0.82° C. per base. For isosequential 25 nt long gapmers (Oligos 30, 31, 32) (Table 5) the change from 13 to 15 PMO increased Tm by +1.4° C., or +0.7° C. per base. In contrast, decreasing the size of 2′-MOE 3′ wing from 5 to 2 in Oligos 16, 17, 18, 19, and 21 decreased Tm by −4.0° C., or −1.33° C. per base even despite increasing the size of 6′ PMO wing by the same number of nucleotides. The same decrease of 2′-MOE size in a series of 25 nt long gapmers (Oligos 27, 28, 29, and 30) resulted in a more modest decrease of −2.0° C. or −0.67° C. per base. Fully PMO modified Oligo 26 shows similar Tm of 61.7° C. to a positive control (5-10-5) 2^{nd} gen gapmer sequence 126 (60.9° C.). These results indicate that introduction of PMO in a block fashion does not alter the RNA binding properties of PMO-DNA chimeras.

### RNAse H Activation by PMO-DNA(PS) Hybrids

The mechanisms by which an oligonucleotide can exert biological activity are complex. In contrast to a steric blocker, RNase H dependent oligonucleotides can inhibit protein expression by targeting practically any region of the mRNA. RNase H degradation is catalytic and single ASO can direct degradation of multiple copies of target mRNA. Conversely, steric blocking oligonucleotides can bind and target only single RNA molecule. To verify if the PMO-DNA hybrids can direct RNAse H, heteroduplexes were prepared with synthesized 5′-fluorescein labeled RNA and annealed at room temperature with PMO-ASO oligonucleotides or DNA control sequence (Oligo 33). RNAse H buffer (1 mM MgCl₂, 50 mM KCl, 10 mM Tris-HCl pH 8.5) was prepared. Heteroduplexes at concentration 100 nM in RNase H buffer and U RNase H1 (Sigma Aldrich, MI) were mixed and incubated at 37° C. for various time points. The enzymatic reaction was terminated by heat inactivation at 65° C. for 20 minutes and then snap frozen in dry ice. Gel electrophoresis was used to determine RNase H activity, with commercially available Invitrogen EX E-Gels (4% Agarose, Sybrgold).

### 3′-Exonuclease (Snake Venom Phosphodiesterase L, SVPDE) Susceptibility Assay

To determine exonuclease stability of hybrid oligonucleotides in situ, Snake Venom Phosphodiesterase I (Crotalus Adamanteus) was used to demonstrate cleavage of phosphodiester bonds in a sequential manner starting at 3′ position. Phosphodiester oligonucleotides, such as DNA containing PO linkages are digested by SVDPE. Conversely, control sequence (Oligo 33), by design with PS linkages should only occur little to no cleavage by SVPDE. To perform enzymatic hydrolysis, (13.3 mM) oligonucleotide hybrids were incubated at 37° C. in a reaction mixture containing 100 mM Tris-HCl buffer (pH 8.5), 14 mM MgCl₂, 72 mM NaCl and SVPDE enzyme (0.1 U/mL). Aliquots (45 mL) of time points were heat inactivated (95° C.), and flash frozen in dry ice until analyzed by IEX-HPLC. Detectable peak broadening and degradation occurred for 2^{nd} gen gapmer (Oligo 33) over a period of 23 h. The major oligonucleotide peak at 14 min retention time was degraded to minor peaks over time after being subjected to exonuclease conditions. SVPDE is Rp selective which explains why degradation of sequence (Oligo 33) slows down after hydrolysis of each nucleotide. No peak broadening was observed for (Oligo 3) although some detectable degradation is likely due to presence of impurities. A combination of morpholino units and phosphorothioates resulted in exceptional nuclease stability. Replacement of a terminal phosphorothioate in PMO-ASO hybrid (Oligo 6) with non-ionic phosphorodiamidate represents one step further towards increasing the nuclease stability.

### CONCLUSIONS

PMO-DNA-RNA hybrids can be synthesized using solid phase methods and commercially available nucleotides. Structurally diverse libraries have been made and evaluated in vitro and in vivo. These gapmers offer stability, lead to increased knockdown of mRNA targets and support RNAse H activity. PMO-DNA gapmers maintain or surpass the activity of 2^{nd} gen ASOs. Reducing the overall charge improves not only pharmacological properties but at the same time allow for reduced aggregation of conjugates with positively charged peptides. PMO chimeras may be employed in other platforms such as DNAzymes, siRNA or steric block oligonucleotides to name a few.

### Example 4: Testing of Exemplary Libraries

### Methods

Synthesis of the libraries disclosed in this Example were prepared as described above. In vitro DMPK knockdown using the ASO/PMO hybrids was tested in vino using HeLa cells with a 15 uM dose. The cells were nucleofected with the gapmers using Lonza nucleofection kits and incubated for 24 hrs. Oligo 1 (Table 6), which showed significant knockdown, was used as reference.

TABLE 6

| | | Flank | | | Tm | |
|---|---|---|---|---|---|---|
| Sequence # | | size | Pattern | KD | [° C.] | Mw Calcd/Found |
| Oligo 1 | 5′-ACAGACAATAAATACCGAGG-3′ | 10 | 5M-10-5 | 76 | 57.2 | 6823.6/6822.2 |
| Oligo 2 | 5′-ACAGACAATAAATACCGAGG-3′ | 12 | 5-2M-8-5 | 52 | | 6765.6/6765.7 |
| Oligo 3 | 5′-ACAGACAATAAATACCGAGG-3′ | 10 | 5-10-3-2M | 29 | 53.4 | 6706.5/6704.4 |
| Oligo 4 | 5′-ACAGACAATAAATACCGAGG-3′ | 4 | 2M-16-2M | 12 | 52.4 | 6475.3/6473.7 |
| Oligo 5 | 5′-ACAGACAATAAATACCGAGG-3′ | 6 | 3M-14-3M | 35 | 51.9 | 6495.4/6494.1 |
| Oligo 7 | 5′-ACAGACAATAAATACCGAGG-3′ | 5 | 3M-15-2M | 14 | 52.4 | 6485.4/6484.4 |
| Oligo 6 | 5′-ACAGACAATAAATACCGAGG-3′ | 6 | 4M-14-2M | 42 | 51.9 | 6495.4/6494.1 |
| Oligo 8 | 5′-ACAGACAATAAATACCGAGG-3′ | 7 | 5M-13-2M | 58 | 53.2 | 6505.4/6504.4 |
| Oligo 9 | 5′-ACAGACAATAAATACCGAGG-3′ | 8 | 6M-12-2M | 78 | 54.2 | 6515.4/6514.3 |
| Oligo 10 | 5′-ACAGACAATAAATACCGAGG-3′ | 9 | 7M-11-2M | 78 | 53.2 | 6525.5/6523.8 |
| Oligo 11 | 5′-ACAGACAATAAATACCGAGG-3′ | 10 | 8M-10-2M | 84 | 53.0 | 6535.5/6534.1 |
| Oligo 12 | 5′-ACAGACAATAAATACCGAGG-3′ | 11 | 9M-9-2M | 85 | 55.2 | 6545.5/6544.2 |
| Oligo 13 | 5′-ACAGACAATAAATACCGAGG-3′ | 12 | 10M-8-2M | 76 | 56.2 | 6555.5/6554.0 |
| Oligo 14 | 5′-ACAGACAATAAATACCGAGG-3′ | 10 | 5-10-5 | 56 | 60.9 | 7226.1/7229.7 |
| Oligo 15 | 5′-TAGTGCGGACCTACCCACGA-3′ | 10 | 5-10-5 | 3 | 50.4 | 7160.4/7160.0 |

ATCG indicate PMO, ATCG indicates DNA, underlined 2′-OMe RNA, bold underlined 2′-OMOE RNA.

TABLE 7

| Sequence# | | Pattern |
|---|---|---|
| Oligo 1 | 5′-ACAGACAATAAATACCGAGG-3′ | 5M-10-5 |
| Oligo 16 | 5′-ACAGACAATAAATACCGAGG-3′ | 6M-9-5 |
| Oligo 17 | 5′-ACAGACAATAAATACCGAGG-3′ | 7M-8-5 |
| Oligo 18 | 5′-ACAGACAATAAATACCGAGG-3′ | 8M-8-4 |
| Oligo 19 | 5′-ACAGACAATAAATACCGAGG-3′ | 9M-8-3 |
| Oligo 21 | 5′-ACAGACAATAAATACCGAGG-3′ | 10M-8-2 |
| Oligo 22 | 5′-ACAGACAATAAATACCGAGG-3′ | 11M-7-2 |
| Oligo 34 | 5′-ACAGACAATAAATACCGAGG-3′ | 12M-6-2 |
| Oligo 23 | 5′-ACAGACAATAAATACCGAGG-3′ | 13M-5-2 |
| Oligo 35 | 5′-ACAGACAATAAATACCGAGG-3′ | 14M-4-2 |
| Oligo 25 | 5′-ACAGACAATAAATACCGAGG-3′ | 15M-3-2 |
| Oligo 36 | Sarco-5′-ACAGACAATAAATACCGAGG-3′ | 20M-0-0 |
| Oligo 20 | 5′-ACAGACAATAAATACCGAGG-3′ | 9M-9-2 |
| Oligo 14 (Positive) | 5′-ACAGACAATAAATACCGAGG-3′ | 5-10-5 |
| Oligo 15 (Negative) | 5′-TAGTGCGGGACCTACCCACGA-3′ | 5-10-5 |

ATCG indicate PMO, ATCG indicates DNA, underlined 2′-OMe RNA, bold underlined 2′-OMOE RNA.

TABLE 8

| | | | | | KD% | KD% |
|---|---|---|---|---|---|---|
| | | | | Tm | Free | Lipofect- |
| Sequence # | | Pattern | Mw Calcd/Found | [° C.] | uptake | amine |
| Oligo 1 | 5′-ACAGACAATAAATACCGAGG-3′ | 5M-10-5 | 6823.6/6822.2 | 57.2 | 55.0 | 67.4 |
| Oligo 16 | 5′-ACAGACAATAAATACCGAGG-3′ | 6M-9-5 | 6889.8/6890.0 | 58.3 | 61.6 | 59.1 |
| Oligo 17 | 5′-ACAGACAATAAATACCGAGG-3′ | 7M-8-5 | 6899.9/6898.9 | 58.3 | 61.8 | 33.8 |
| Oligo 18 | 5′-ACAGACAATAAATACCGAGG-3′ | 8M-8-4 | 6821.2/6821.8 | 55.0 | 32.2 | 56.4 |
| Oligo 19 | 5′-ACAGACAATAAATACCGAGG-3′ | 9M-8-3 | 6757.4/6758.7 | 56.0 | 32.0 | 97 |
| Oligo 21 | 5′-ACAGACAATAAATACCGAGG-3′ | 10M-8-2 | 6694.7/6693.3 | 54.3 | 35.7 | 93.3 |
| Oligo 22 | 5′-ACAGACAATAAATACCGAGG-3′ | 11M-7-2 | 6704.7/6703.6 | 53.3 | NA | 94 |
| Oligo 34 | 5′-ACAGACAATAAATACCGAGG-3′ | 12M-6-2 | 6714.7/6713.5 | 54.8 | NA | 46.0 |
| Oligo 23 | 5′-ACAGACAATAAATACCGAGG-3′ | 13M-5-2 | 6724.7/6723.8 | 54,9 | NA | 9.2 |
| Oligo 35 | 5′-ACAGACAATAAATACCGAGG-3′ | 14M-4-2 | 6733.8/6733.8 | 56.4 | NA | 5.4 |
| Oligo 25 | 5′-ACAGACAATAAATACCGAGG-3′ | 15M-3-2 | 6744.8/6743.5 | 58.2 | 19.3 | 14.6 |
| Oligo 36 | Sarco-5′-ACAGACAATAAATACCGAGG-3′ | 20M-0-0 | 6823.9/6821.3 | 61.7 | NA | 28.3 |
| Oligo 20 | 5′-ACAGACAATAAATACCGAGG-3′ | 9M-9-2 | 6596.5/6594.0 | 53.3 | 63.3 | 88.2 |

ATCG indicate PMO, ATCG indicates DNA, underlined 2′-OMe RNA, bold underlined 2′-OMOE RNA.

TABLE 9

| | | | | | KD% | | |
|---|---|---|---|---|---|---|---|
| | | | Mw | | Free | KD% | KD% |
| | Sequence | | Calcd/ | Tm | up- | Nucle | Lipofect- |
| | # | Pattern | Found | [° C.] | take | of. | amine |
| 5′-ACAGACAATAAATACCGAGG-3′ | Oligo 1 | SM-10-5 | 6823.6/ | 57.2 | 55.0 | 50 | 67.4 |
| | | | 6822.2 | | | | |
| 5′-ACAGACAATAAATACCGAGG-3′ | Oligo 16 | 6M-9-5 | 6889.8/ | 58.3 | 61.6 | 63 | 59.1 |
| | | | 6890.0 | | | | |
| 5′-ACAGACAATAAATACCGAGG-3′ | Oligo 17 | 7M-8-5 | 6899.9/ | 58.3 | 61.8 | 72 | 33.8 |
| | | | 6898.9 | | | | |
| 5′-ACAGACAATAAATACCGAGG-3′ | Oligo 18 | 8M-8-4 | 6821.2/ | 55.0 | 32.2 | 67 | 56.4 |
| | | | 6821.8 | | | | |
| 5′-ACAGACAATAAATACCGAGG-3′ | Oligo 19 | 9M-8-3 | 6757.4/ | 56.0 | 32.0 | 67 | 97.0 |
| | | | 6758.7 | | | | |
| 5′-ACAGACAATAAATACCGAGG-3′ | Oligo 21 | 10M-8-2 | 6694.7/ | 54.3 | 35.7 | 69 | 93.3 |
| | | | 6693.3 | | | | |
| 5′-ACAGACAATAAATACCGAGG-3′ | Oligo 22 | 11M-7-2 | 6704.7/ | 53.3 | NA | 46 | 94.0 |
| | | | 6703.6 | | | | |
| 5′-ACAGACAATAAATACCGAGG-3′ | Oligo 34 | 12M-6-2 | 6714.7/ | 54.8 | NA | 39 | 46.0 |
| | | | 6713.5 | | | | |
| 5′-ACAGACAATAAATACCGAGG-3′ | Oligo 23 | 13M-5-2 | 6724.7/ | 54.9 | NA | 16 | 9.2 |
| | | | 6723.8 | | | | |
| 5′-ACAGACAATAAATACCGAGG-3′ | Oligo 35 | 14M-4-2 | 6733.8/ | 56.4 | NA | 11 | 5.4 |
| | | | 6733.8 | | | | |
| 5′-ACAGACAATAAATACCGAGG-3′ | Oligo 25 | 15M-3-2 | 6744.8/ | 58.2 | 19.3 | 6 | 14.6 |
| | | | 6743.5 | | | | |
| Sarco-5′-ACAGACAATAAATACCGAGG-3′ | Oligo 36 | 20M-0-0 | 6823.9/ | 61.7 | NA | 0 | 28.3 |
| | | | 6821.3 | | | | |
| 5′-ACAGACAATAAATACCGAGG-3′ | Oligo 20 | 9M-9-2 | 6596.5/ | 53.3 | 63.3 | 49 | 88.2 |
| | | | 6594.0 | | | | |
| 5′-ACAGACAATAAATACCGAGG-3′ | Oligo 14 | 5-10-5 | 7226.1/ | 60.9 | 59 | 80 | 96 |
| | | | 7229.7 | | | | |
| 6′-ACAGACAATAAATACCGAGG-3′ | Oligo 55 | 5M-10-5 | 6880.80/ | 56.50 | 43 | 84 | 87 |
| | | | 6881 | | | | |

ATCG indicate PMO, ATCG indicates DNA, underlined 2′-OMe RNA, bold underlined 2′-OMOE RNA. ACTG = 2′-MOE RNA ACTG = PMO NA = At NT level

TABLE 10

| | | | Mw | | KD% | KD% | KD% |
|---|---|---|---|---|---|---|---|
| | Sequence | | Calcd/ | Tm | Free | Nucle | Lipofect- |
| | # | Pattern | Found | [° C.] | uptake | of. | amine |
| 5′-ACAGACAATAAATACCGAGGAATGT-3′ | Oligo 27 | 10M-10-5 | 8561.2/ | 58.4 | NA | | 70.6 |
| | | | 8559.1 | | | | |
| 5′-ACAGACAATAAATACCGAGGAATGT-3′ | Oligo 28 | 11M-10-4 | 8497.2/ | 57.6 | NA | | 55.8/70.7 |
| | | | 8495.3 | | | | |
| 5′-ACAGACAATAAATACCGAGGAATGT-3′ | Oligo 29 | 12M-10-3 | 8433.1/ | 56.4 | 15.7 | | 71.9 |
| | | | 8430.9 | | | | |
| 5′-ACAGACAATAAATACCGAGGAATGT-3′ | Oligo 30 | 13M-10-2 | 8369.1/ | 56.4 | 24.1 | | 77.7 |
| | | | 8366.8 | | | | |
| 5′-ACAGACAATAAATACCGAGGAATGT-3′ | Oligo 31 | 14M-9-2 | 8379.1/ | 57.2 | 18.6 | | 43.8/98.1 |
| | | | 8377.4 | | | | |
| 5′-ACAGACAATAAATACCGAGGAATGT-3′ | Oligo 32 | 15M-8-2 | 8389.1/ | 57.8 | 13.4 | | 95.8 |
| | | | 8387.3 | | | | |
| 5′-ACAGACAATAAATACCGAGG-3′, | Oligo 38 | | 6152/ | | | | |
| all PO | | | 6149 | | | | |
| FAM-5′-CCUCGGUAUUUAUUGUCUGU*-3′ | Oligo 39 | | | | | | |
| | (RNA*) | | | | | | |
| 5′-ACAGACAATAAATACCGAGG-3′ | Oligo 33 | 5-10-5 | | | | | |
| 5′-ACAGACAATAAATACCGAGG-3′, | Oligo 37 | | | | | | |
| all PS | | | | | | | |

### Results

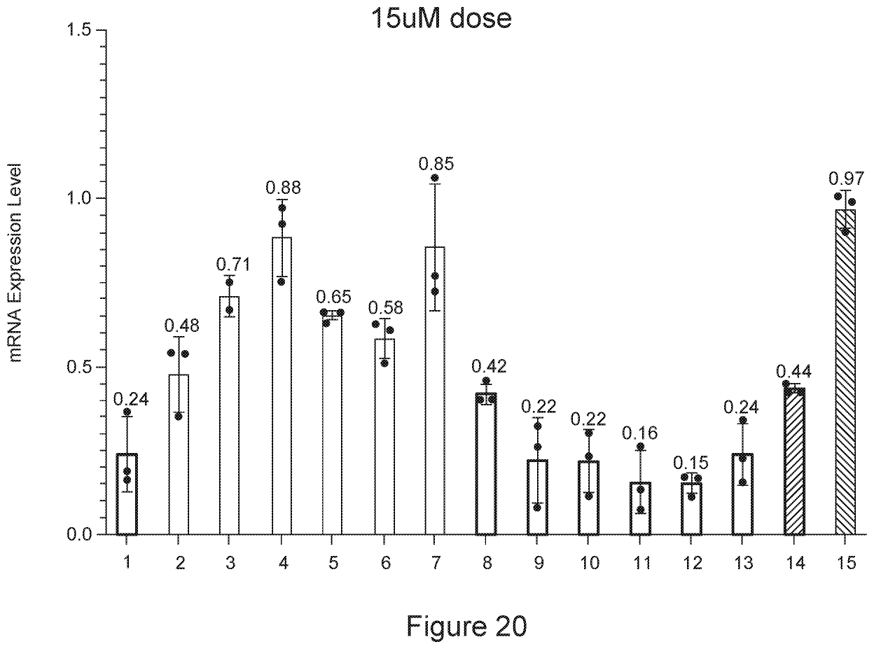

FIG. 20 shows results for mRNA expression after electroporation of 15 μM of 20-mer oligonucleotides from Table 6.

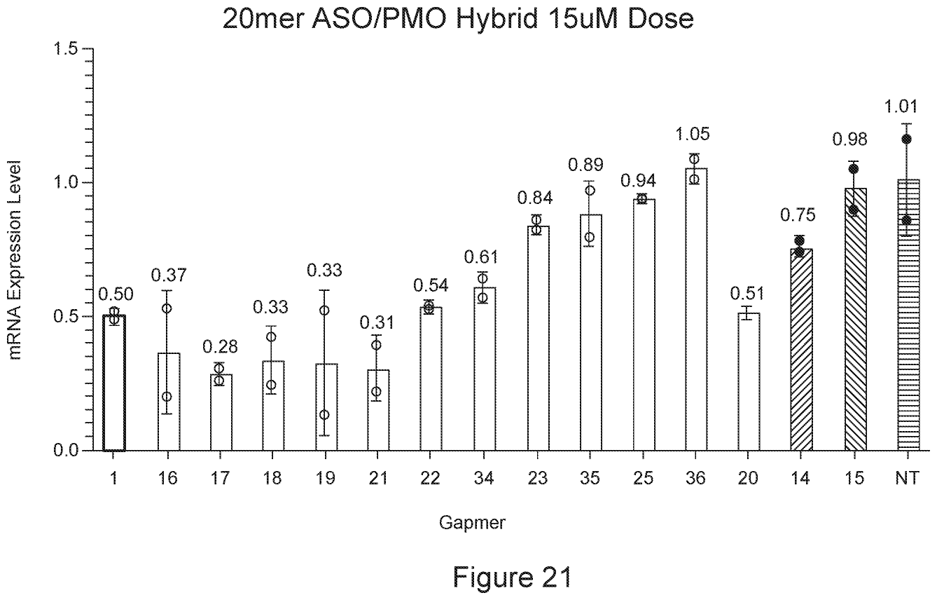

FIG. 21 shows qPCR results for mRNA expression after electroporation of 15 μM of oligonucleotides from Table 7.

Tables 8-10 show percent uptake of hybrid oligonucleotides with and without lipofectamine.

The mRNA expression level is lowered as the PMO length increases until it reaches a 10M-8-2 pattern.

### Example 5: DMPK Knockdown

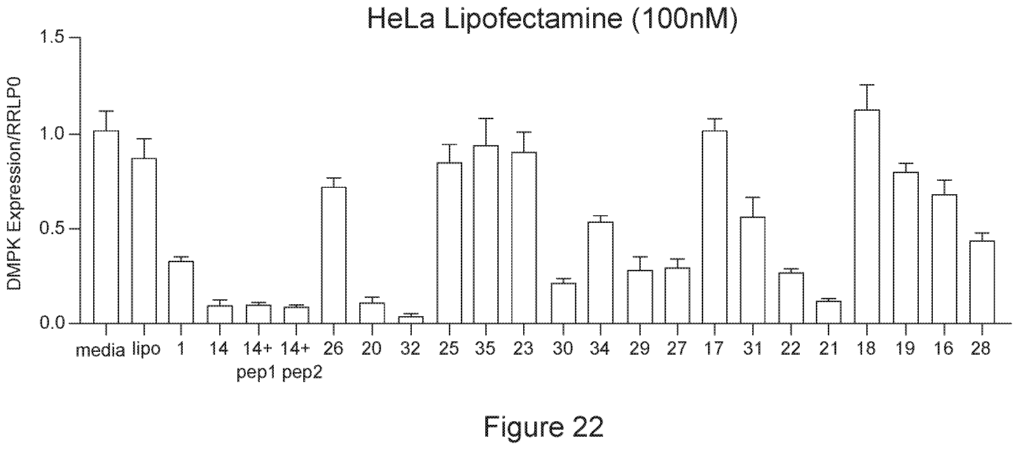

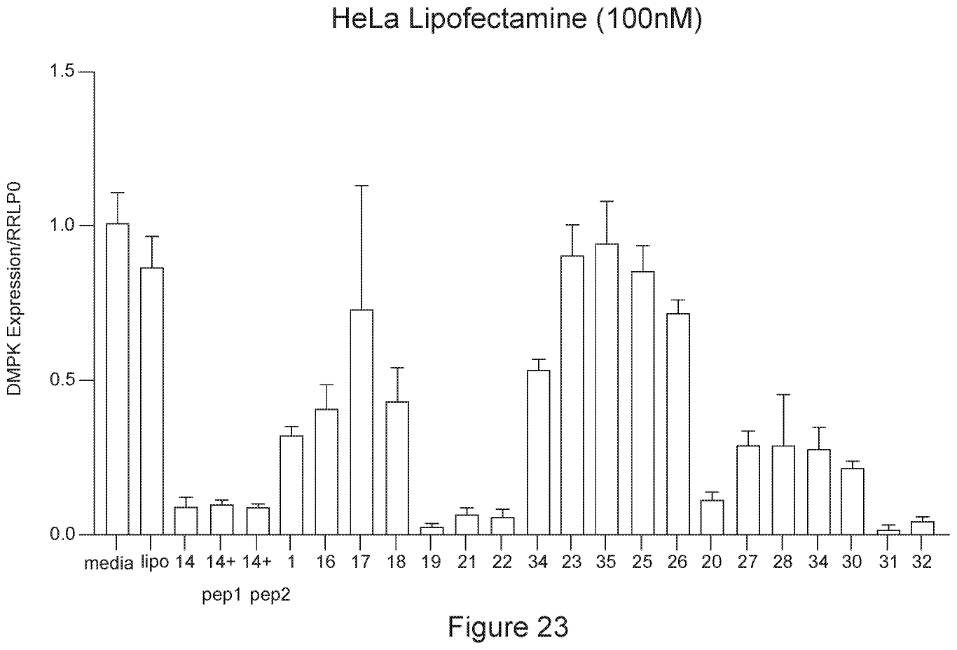

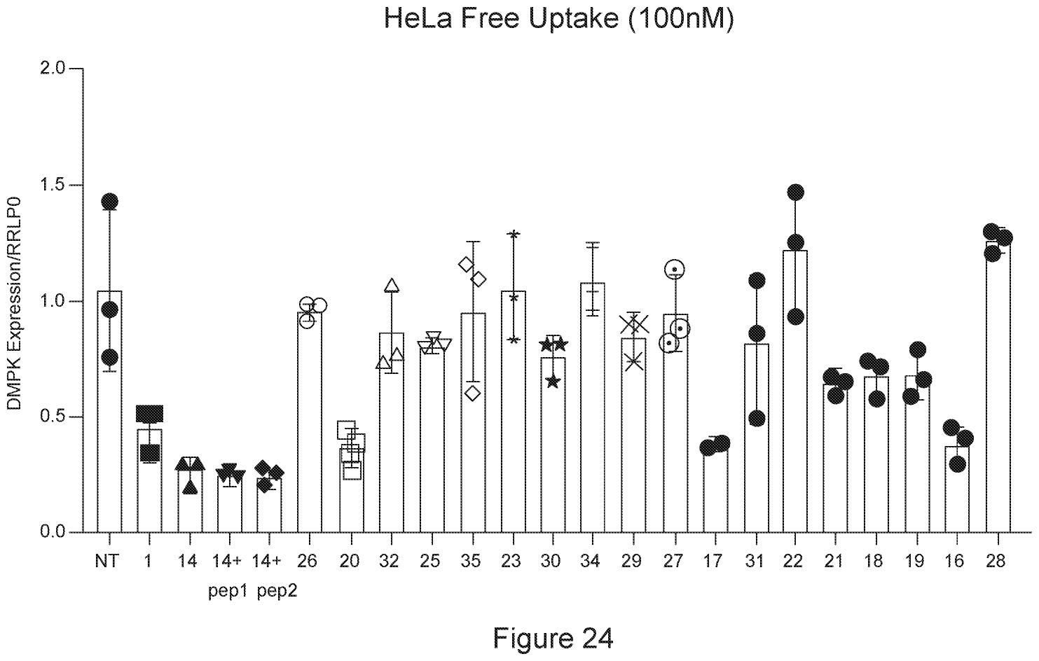

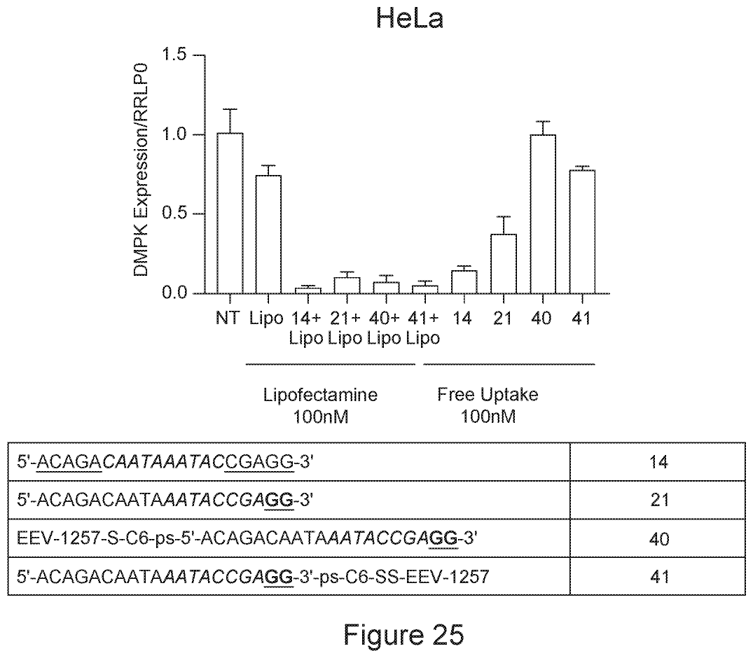

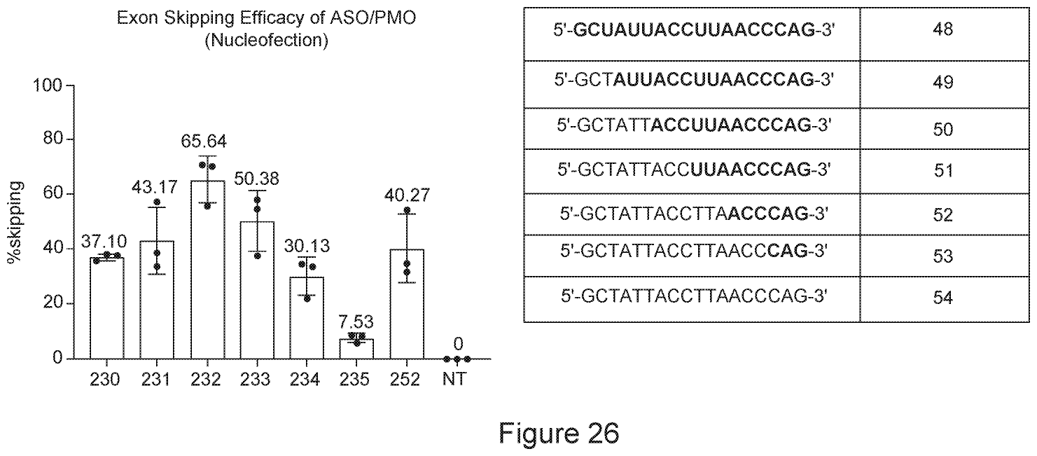

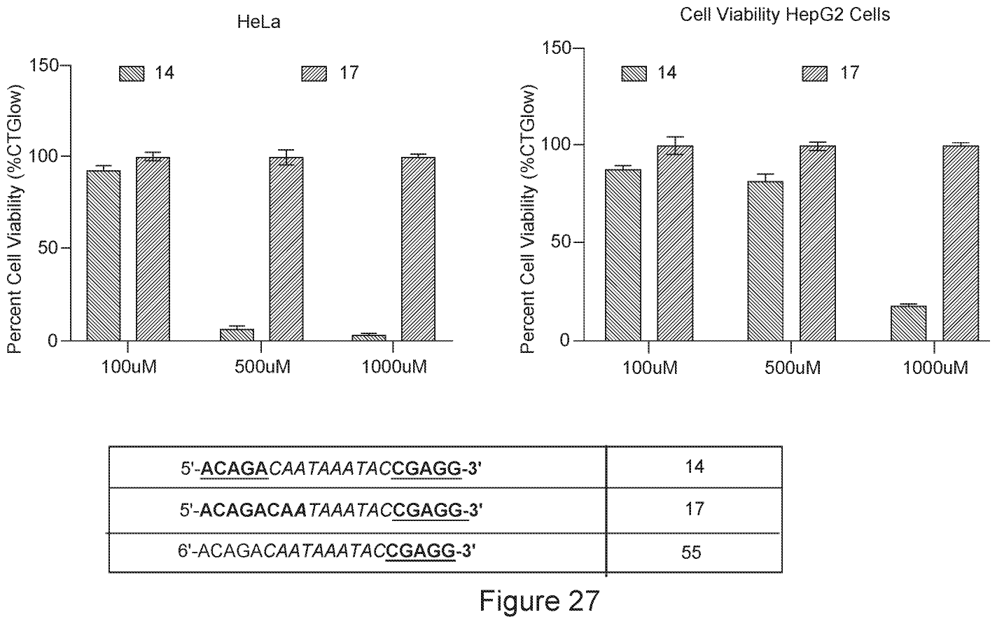

FIG. 22, FIG. 23, FIG. 24 and FIG. 25 show DMPK knockdown results for oligonucleotides shown in Tables 6-Table 11 in HeLa cells (free or lipofectamine delivery). Briefly, HeLa cells in a 24-well format were treated with PMO/ASO hybrid in OPTI-MEM media for 48 hours. No significant toxicity observed except for peptide control at 100 μM (data not included). With lipofectamine transfection, all PMO/ASO hybrid and ASO gapmers show target K.D. For free uptake, ASO and PMO/ASO hybrid gapmers shows K.D., but not EEV-conjugated gapmers (FIG. 25).

TABLE 11

| | Sequence # | Target | Mw Calcd. |
|---|---|---|---|
| 5′-ACAGACAATAAATACCGAGG-3′ (control) | Oligo 21 | DMPK | 6695 |
| EEV1-S-C₆H₁₂-ps-5′-ACAGACAATAAATACCGAGG-3′ | Oligo 40 | DMPK | 9014 |
| 5′-ACAGACAATAAATACCGAGG-3′-ps-C₆H₁₂-SS-EEV2 | Oligo 41 | DMPK | 9026 |
| 5′-ACAGACAATAAATACCGAGG-3′ (control) | Oligo 33 | DMPK | 7226 |
| Peptide (control) | peptide-1 | — | 1600 |

EEV1: CPP12-Q-PEG12-K(Mal)
EEV2: Ac-CPEG12-K(cyclo(FfΦRrRrQ))-NH₂
Peptide 1: Cyclo(FfΦRrRrQ)-PEG8- OH
ATCG indicate PMO, ATCG indicates DNA, underlined indicates 2′-OMe modified RNA, bold underlined indicates 2′- OMOE modified RNA.

### Example 6: Examples of siRNA

TABLE 12

| | | | Mw |
|---|---|---|---|
| Sequence | Strand | # | Calcd/Found |
| 3′-UuUuAgAgUuUuAgUaGgG-5′ | SS | Oligo 42 | 6304/6308 |
| 3′-UuUuAgAgUuUuAgUaGGG-5′ | SS | Oligo 43 | 6265/6281 |
| 3′-TTUuAgAgUuUuAgUaGgG-5′ | SS | Oligo 44 | 6293/6289 |
| 3′-TTUuAgAgUuUuAgUaGGG-5′ | SS | Oligo 45 | 6268/6277 |
| 5′-aAaAuCuCaAaAuCAuCcCu-3′ | AS | Oligo 46 | 6483/6479 |
| 5′-aAaAuCuCaAaAuCAuCcCT-3′ | AS | Oligo 47 | 6463/6460 |

ACUG = 2′-OMe RNA; ACTG = PMO; actg = 2′-F RNA; SS = sense strand; AS = antisense strand

Pairings for siRNA Duplexes are:
Oligo 42 and Oligo 47
Oligo 44 and Oligo 46
Oligo 43 and Oligo46
Oligo 42 and Oligo 46
Oligo 45 and Oligo 46

### Example 7: ASO-PMO/RNA Duplex is a Substrate for RNase H

### Method

RNAse H 1, 5, 8, 10, 20, or 40 U activity (Buffer 10 mM Tris-HCl, 1 mM MgCl2, 50 mM KCl)

5.6 ug, diluted to 65 uL or 85 uL

Incubated at 37° C. for 1 hour, 8 minutes or 16 minutes

Heat shock protein at 65° C., then 10 uL per reaction on e-gel, 4% agarose 15 minutes

The ASO/PMO is Oligo 11 (5′-ACAGACAATAAATACCGAGG-3′):

### Results

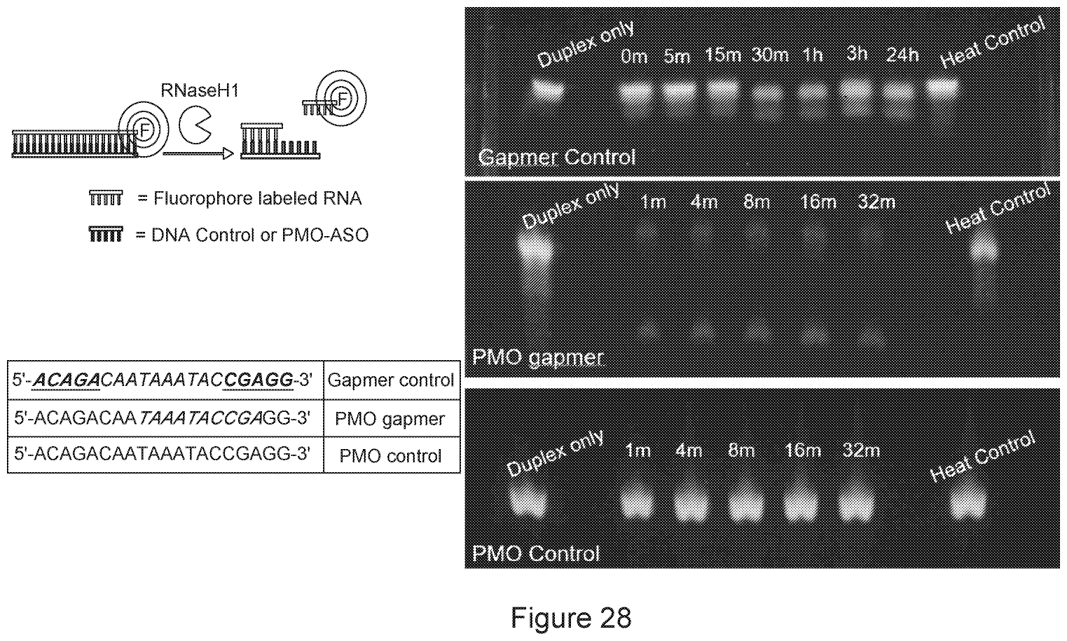

FIG. 28 shows that RNase H is active with PMO-ASO gapmer duplex but not the DNA/RNA control or PMO/RNA control.

FIG. 29 is a demonstration of RNase H cleavage of PMO-ASO but not a DNA/RNA control duplex with increasing RNase units.

FIG. 30 is a demonstration of RNase H cleavage of control of experimental duplexes. RNA/PMO-ASO duplexes were made with fluorescent RNA (25mer) and annealed with PMO-ASO sequences. Duplexes are incubated in 8 U RNase H buffer, at various time points, aliquots removed, and heat shocked (65° C.) to deactivate RNase H protein. The products are visualized using gel electrophoresis (E-gel, 4% agarose). A duplex with a short gap shows little to no degradation (Oligo 25 and 23).

### Example 8: Stability with Snake Venom Phosphodiesterase Digestion

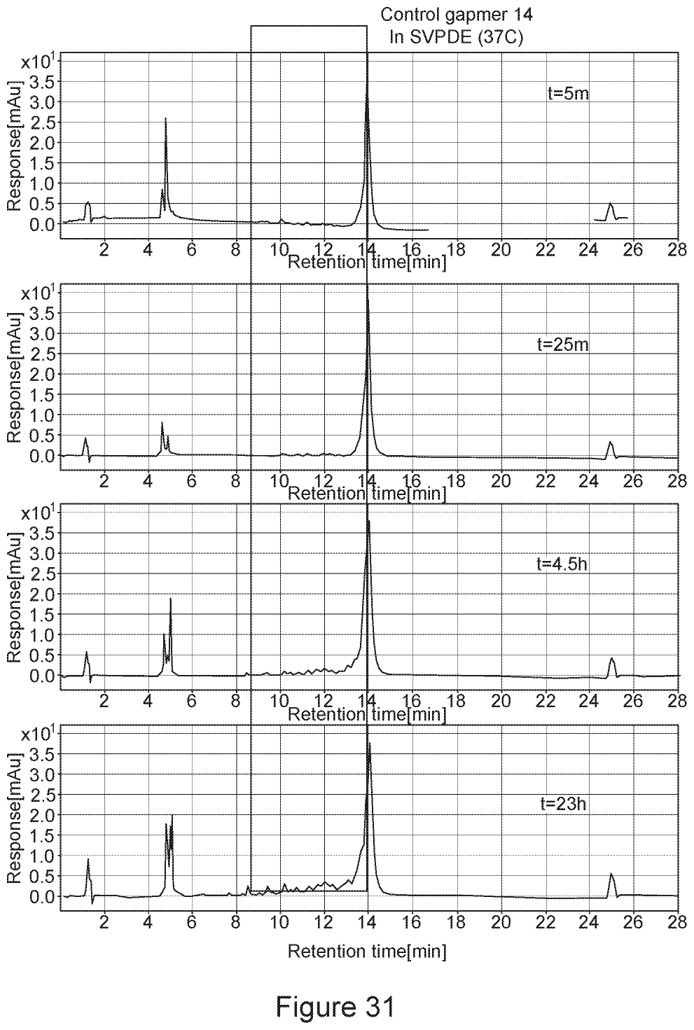

FIG. 31 shows that Snake Venom Phosphodiesterase digests oligonucleotides from 3′ end. Snake Venom Phosphodiesterase (SVPDE Crotalus adamanteus), a hydrolytic enzyme, metabolizes extracellular nucleotides typically involved with intercellular signaling. Hydrolysis occurs at 3′ end and releases 5′ nucleotides.

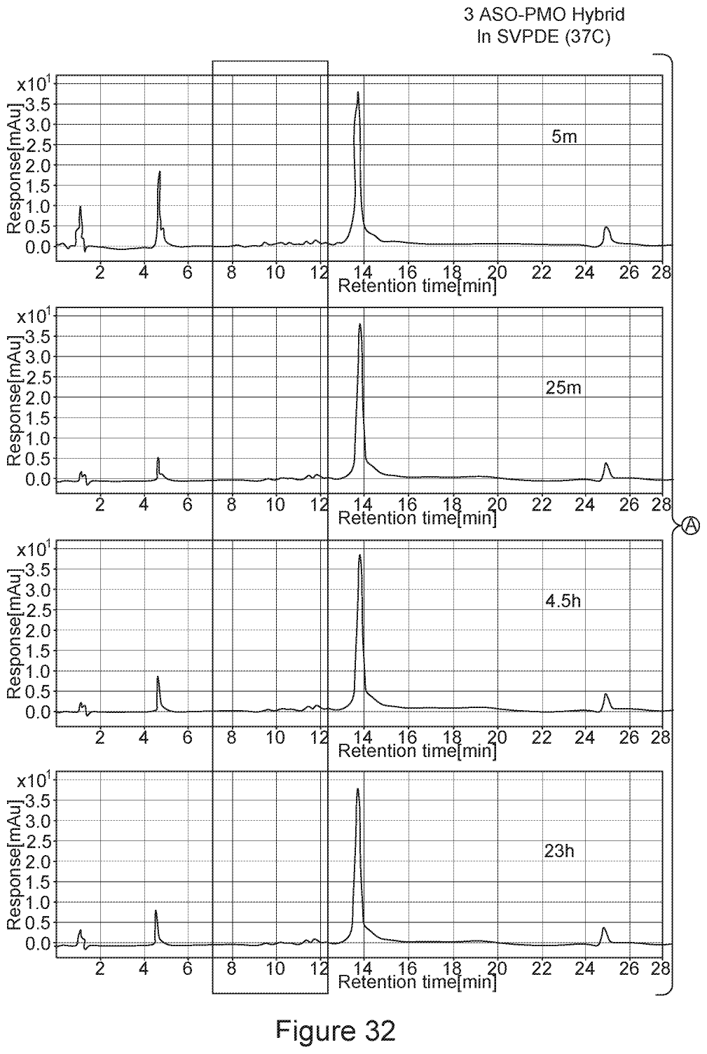

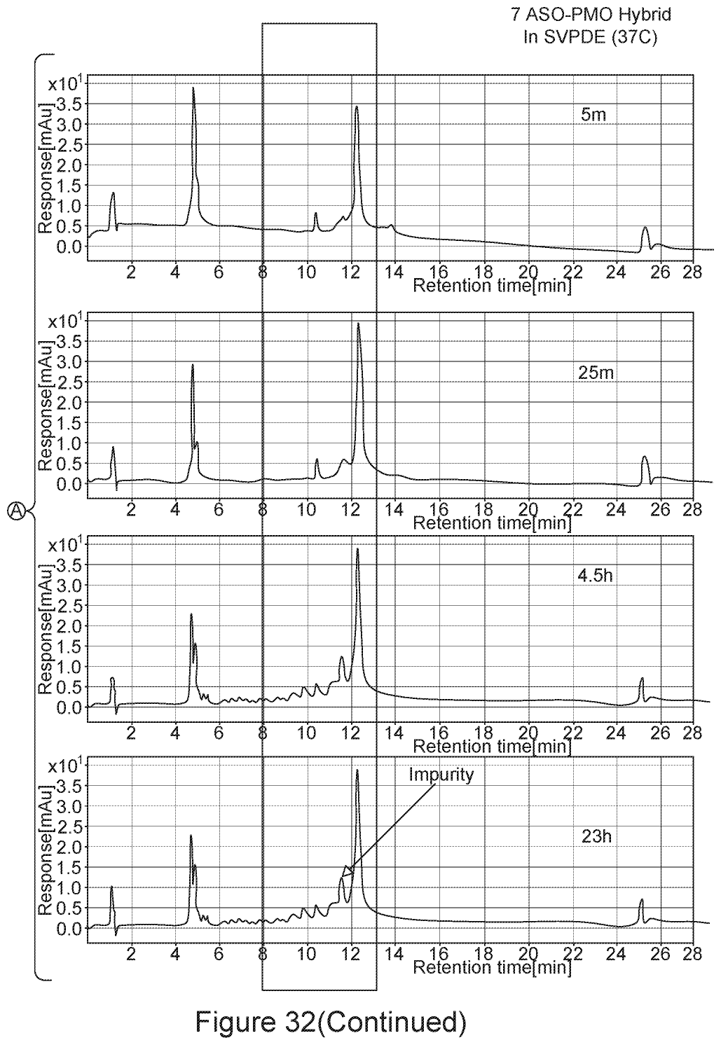

FIG. 32 indicates that ASO-PMO hybrids are stable in the presence of SVDPE.

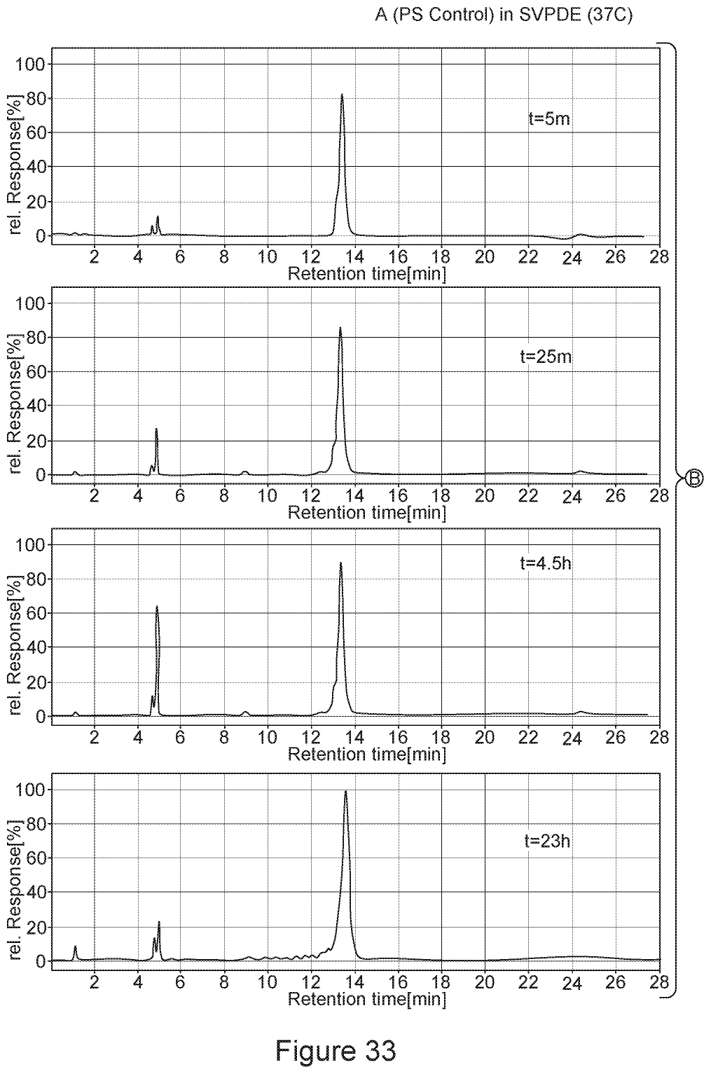

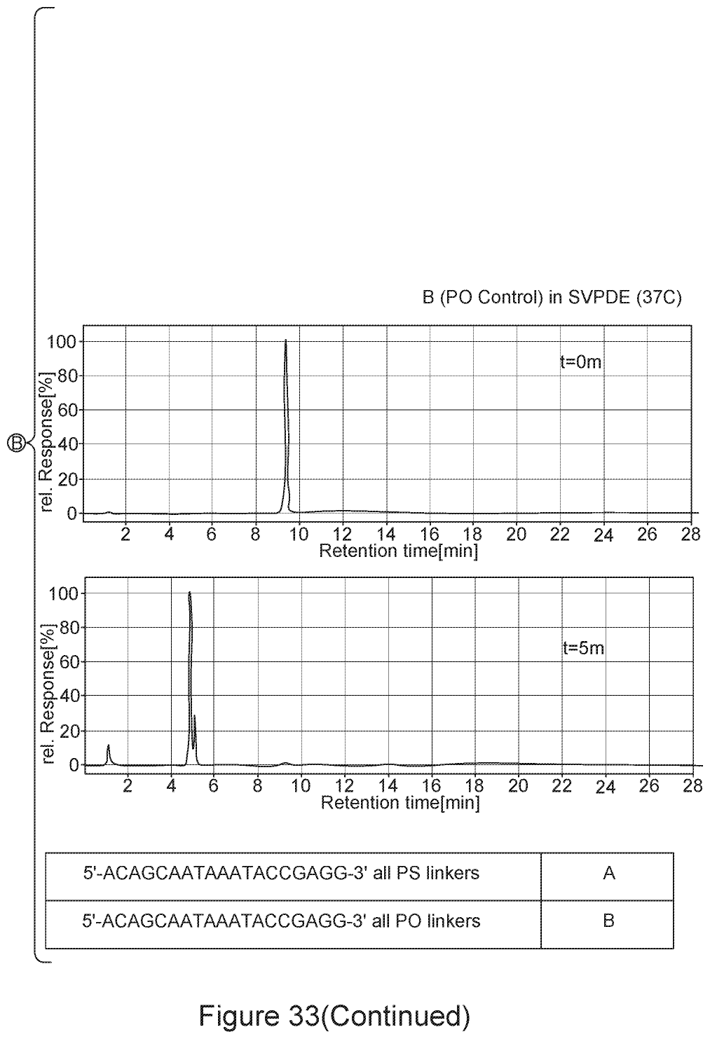

FIG. 33 shows LEX-HPLC data for a SVDPE assay using a control gapmer with a phosphorothiote (PS) backbone (Oligo 37) and phosphodiester (Oligo 38).

### Example 9: Stability with Phosphodiesterase 11

### Methods

Phosphodiesterase II (5′-exonucleoase) bovine spleen phosphodiesterase (Sigma, 10U). Digestion was performed with:

1.4 nmol oligonucleotide (Oligo 21:5′-ACAGACAATAAATACCGAGG-3′)

100 mM ammonium acetate

1 mM EDTA

1 mM TWEEN 80, pH 6-7

Final enzyme concentration 10 mU/μL

37° C., time points were collected, heat inactivated at 65° C. and snap frozen.

### Results

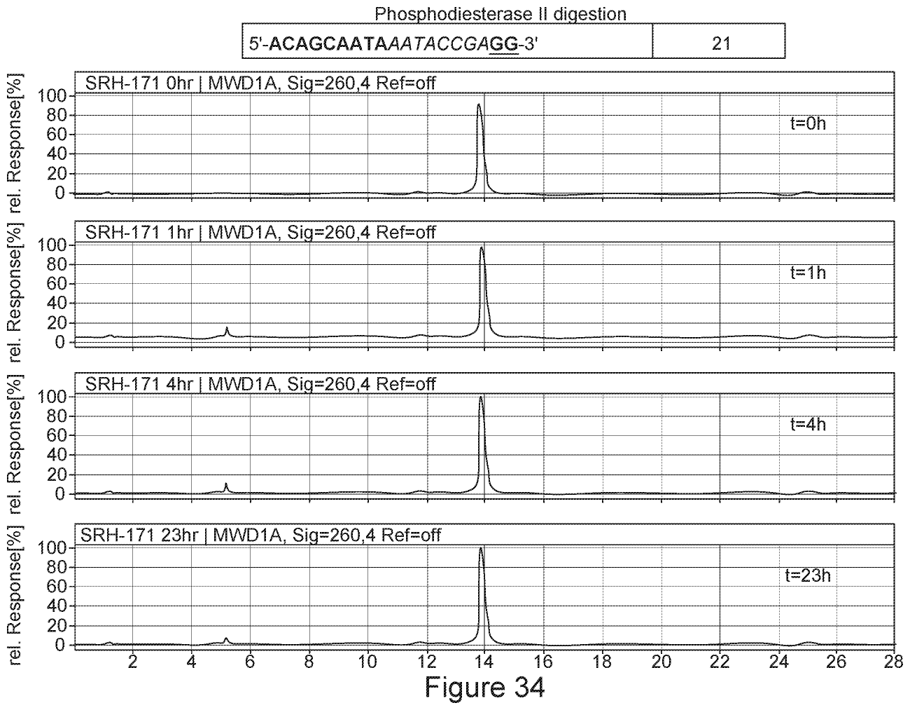

FIG. 34 provides data showing that PMO-ASO (Oligo 21) is stable during Phosphodiesterase II digestion.

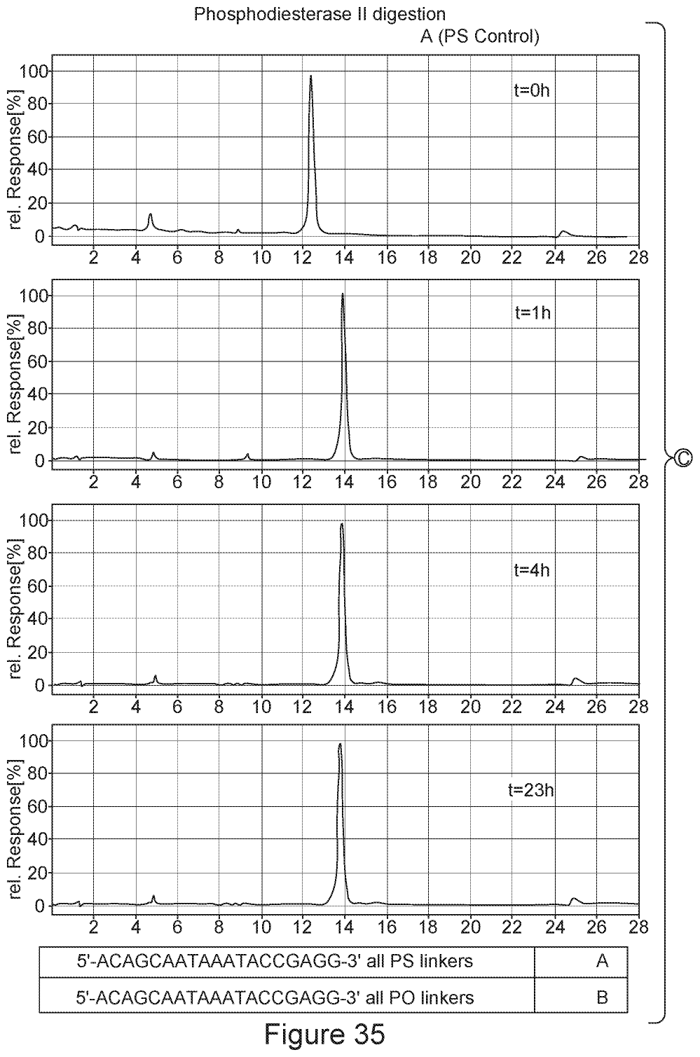

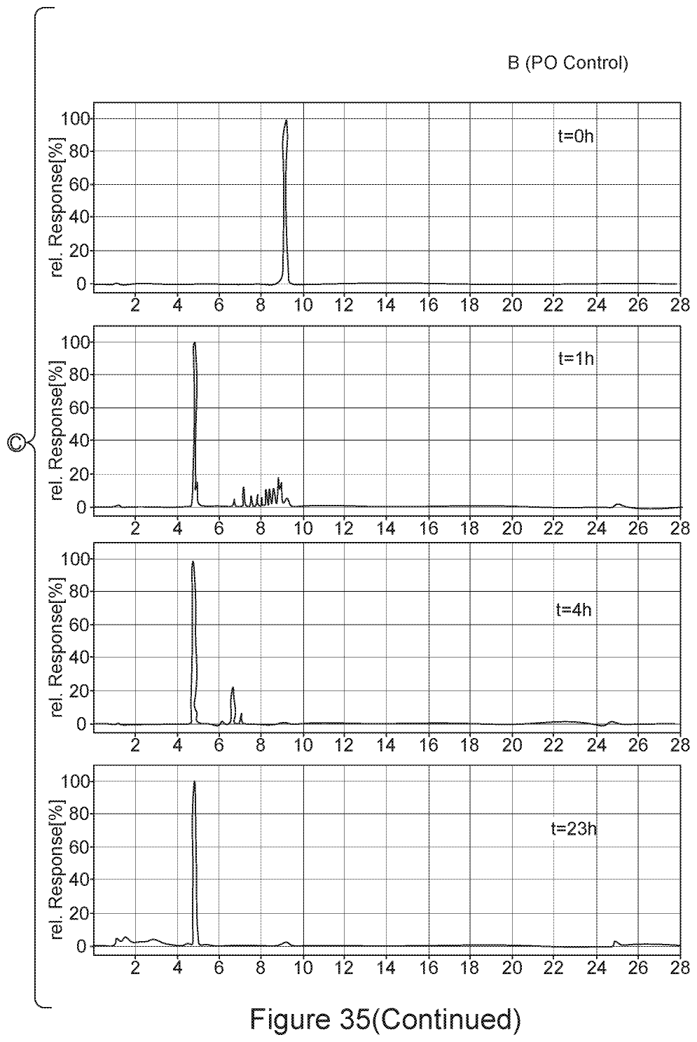

FIG. 35 shows show data for PO and PS Controls.

### Example 10: Other Hybrids

Oligo 48
5′-GCU AUU ACC UUA ACC CAG-3′
Oligo 49
5′-GCT AUU ACC UUA ACC CAG-3′
Oligo 50
5′-GCT ATT ACC UUA ACC CAG-3′
Oligo 51
5′-GCT ATT ACC UUA ACC CAG-3′
Oligo 52
5′-GCT ATT ACC TTA ACC CAG-3′
Oligo 53
5′-GCT ATT ACC TTA ACC CAG-3′
Oligo 54
5′-GCT ATT ACC TTA ACC CAG-3
ACUG = 2′-OMe RNA; ACTG = PMO
5′-ACAGACAATAAATACCGAGG-3
EEV1-po-5′-ACAGACAATAAATACCGAGG-3
5′-ACAGACAATAAATACCGAGG-3′-ps-EEV1
Control: 5′-ACAGACAATAAATACCGAGG-3′
ACUG = 2′-OMe RNA; ACTG = PMO; ACTG = 2′-OMOE RNA; ATCG = DNA

### Example 11: Examples of Oligonucleotides, Linkage Groups and Nucleosides

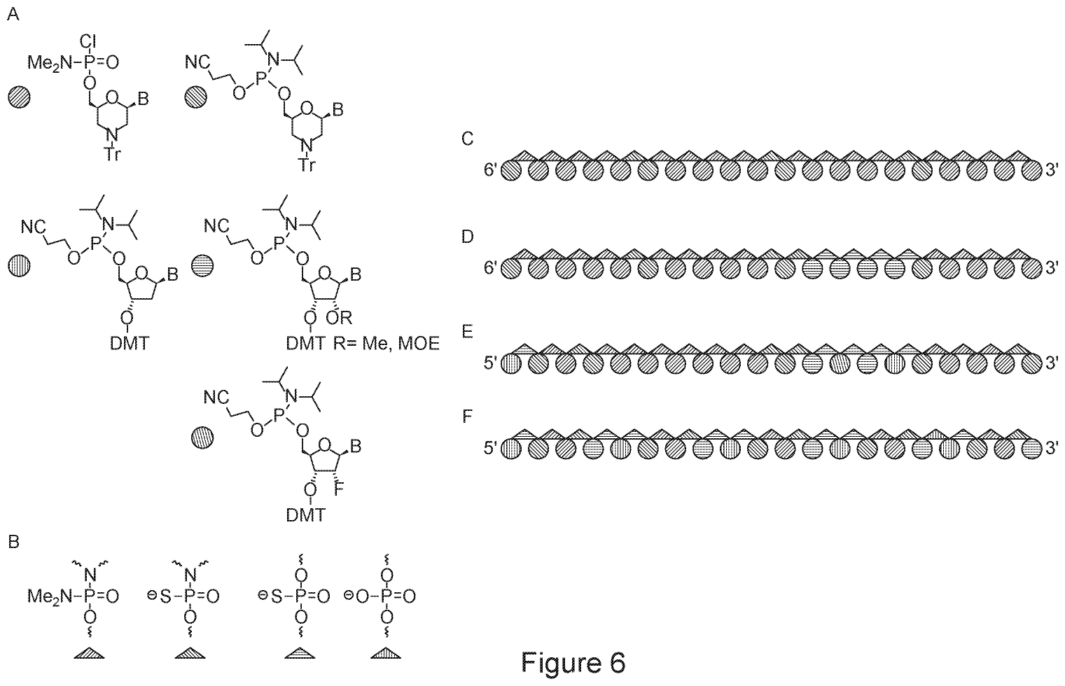

FIGS. 5 and 6 depicts sample of hybrid gapmer oligonucleotides.

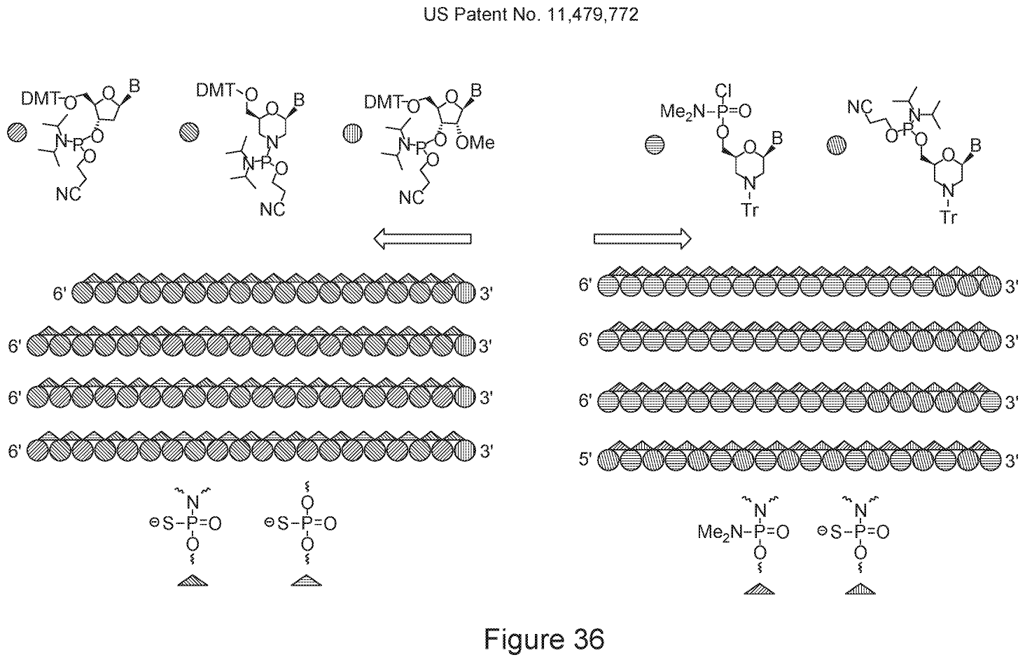

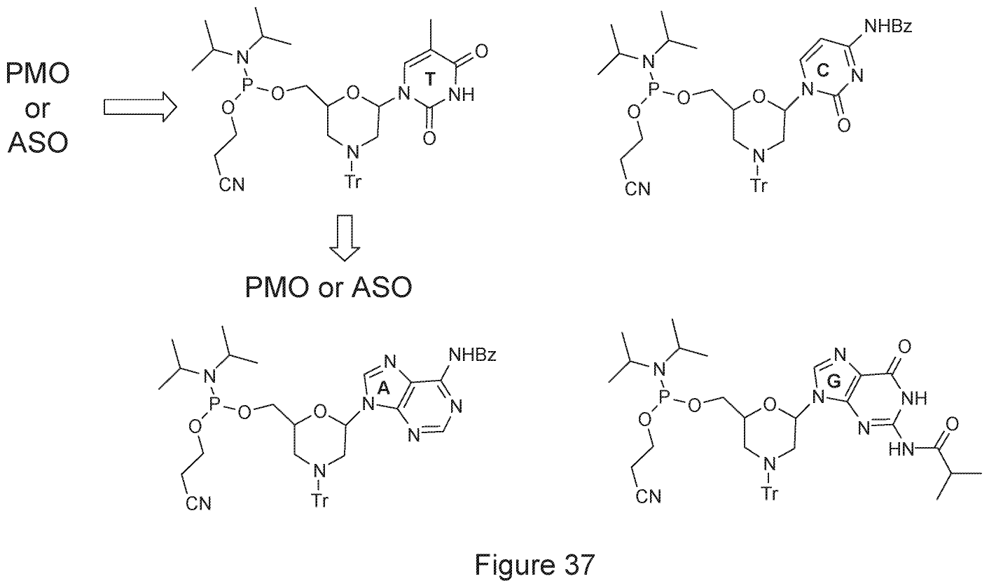

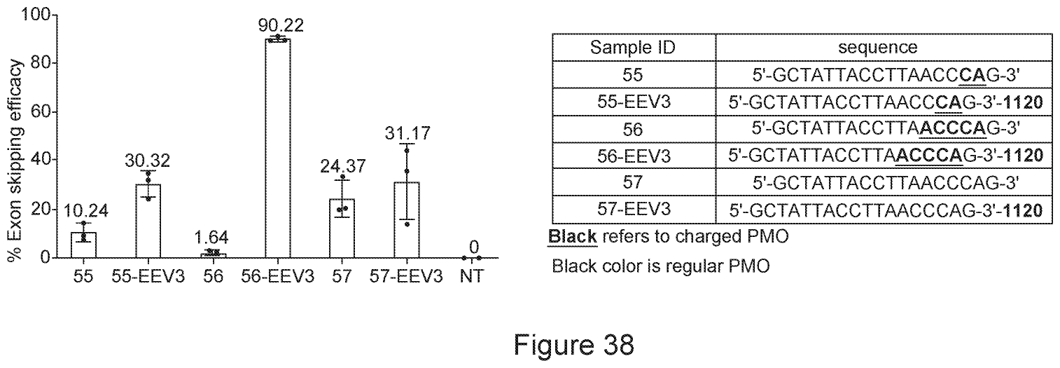

FIG. 36 depicts s a hybrid gapmer oligonucleotide with 5′ and 3′ flanks linked by a phosphorothioamidate linkage and a central gap region comprising DNA P(III) nucleotides linked by phosphorothioate linkages.

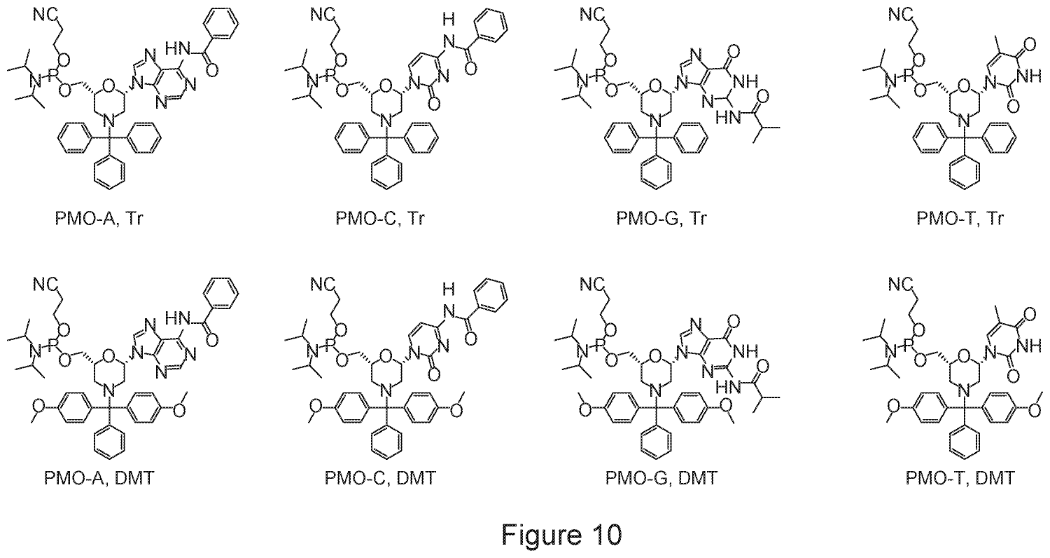

FIG. 10 shows Tr or DMT protected P(III) morpholine analogs.

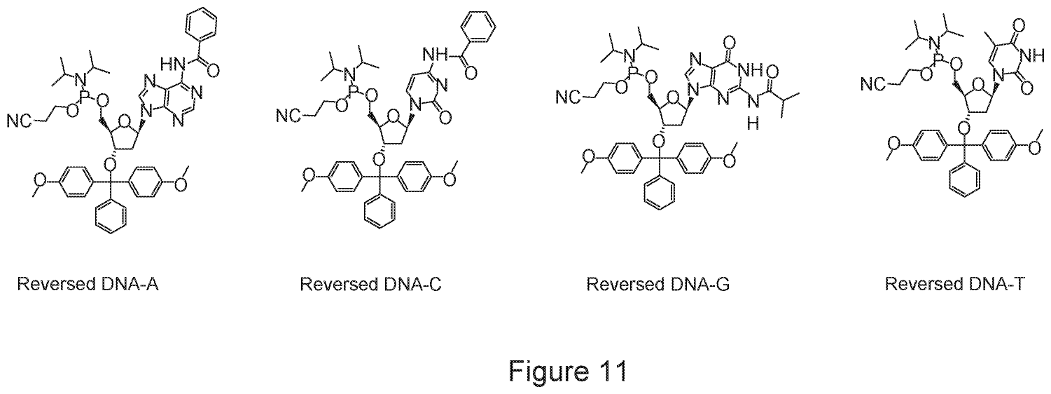

FIG. 11 shows DMT protected reverse DNA P(III) nucleotide analogs.

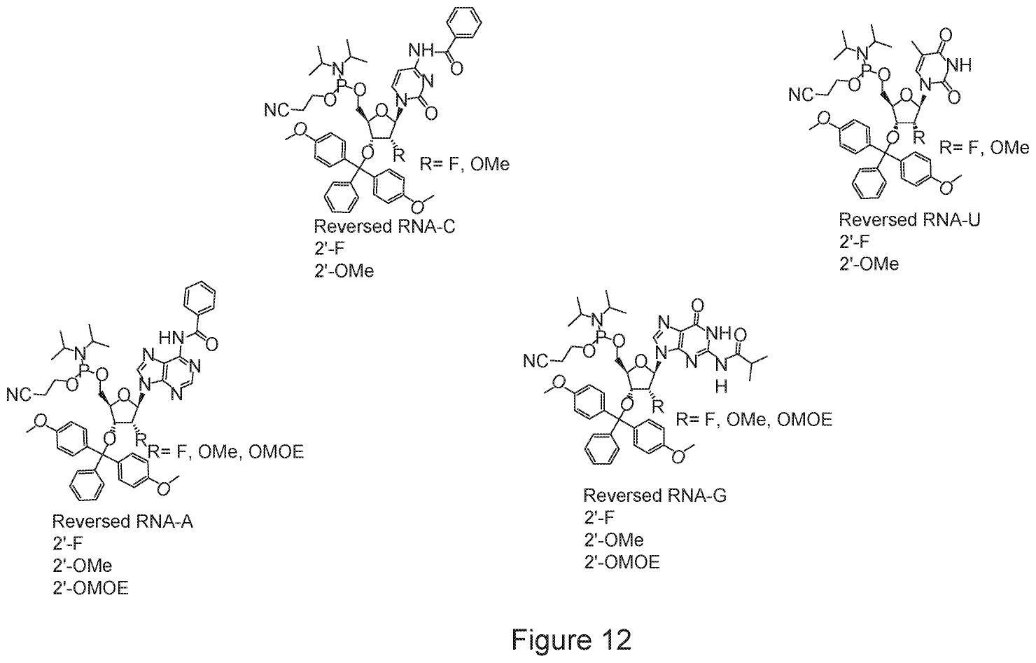

FIG. 12 shows DMT protected 2′ modified reverse RNA P(III) nucleotide analogs.

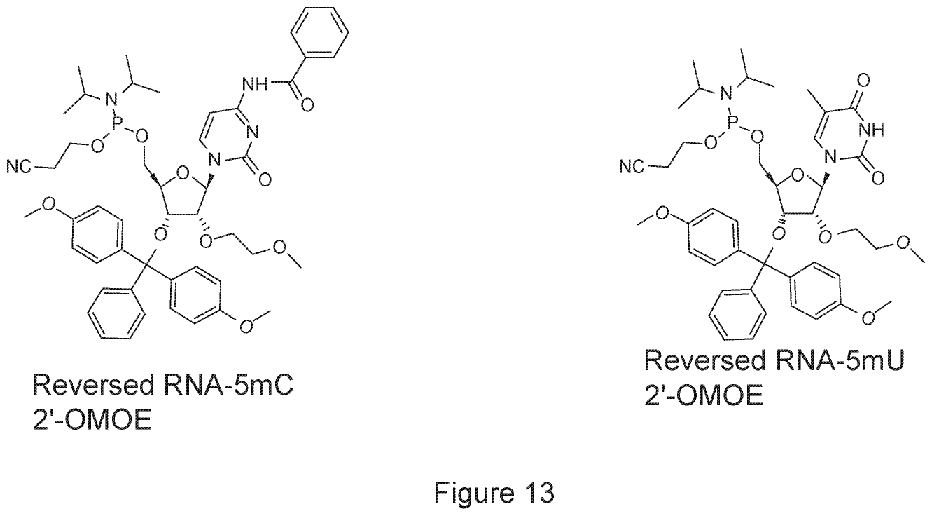

FIG. 13 shows DMT protected 2′ modified reverse RNA P(III) nucleotide analogs with methylated C and U bases.

FIG. 14 shows Tr protected P(V) morpolino nucleobase analogs with double protected G bases.

FIG. 15 depicts Tr protected P(V) morpholino nucleobase analogs.

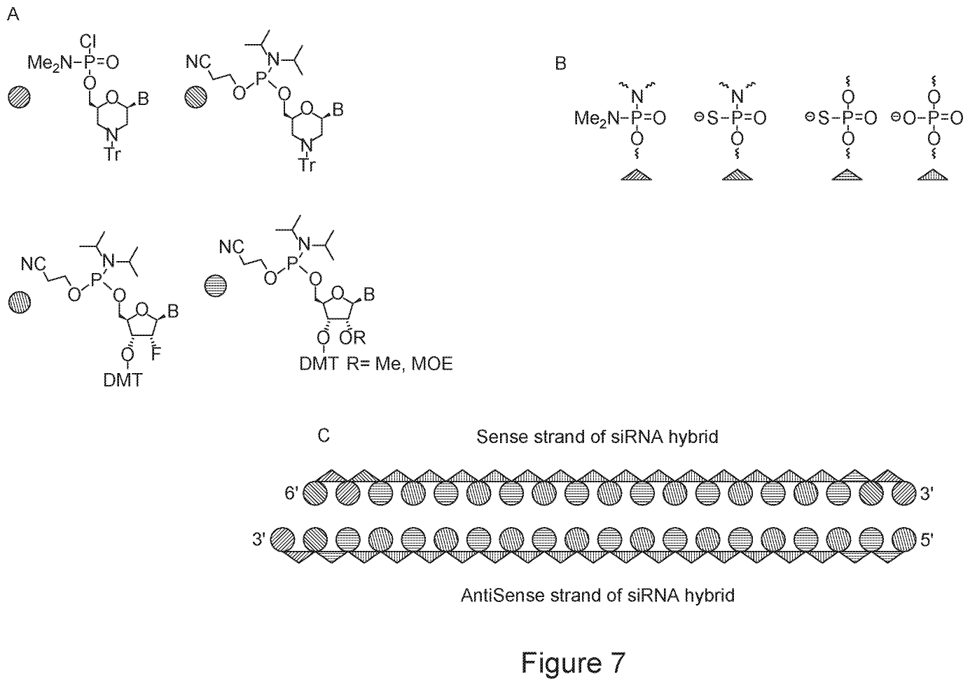

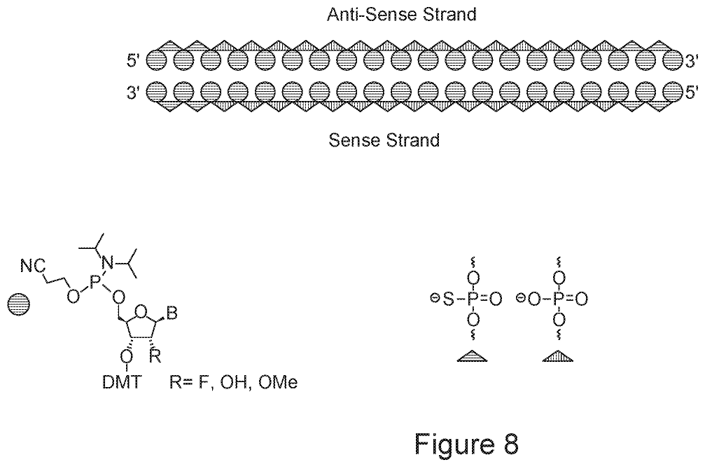

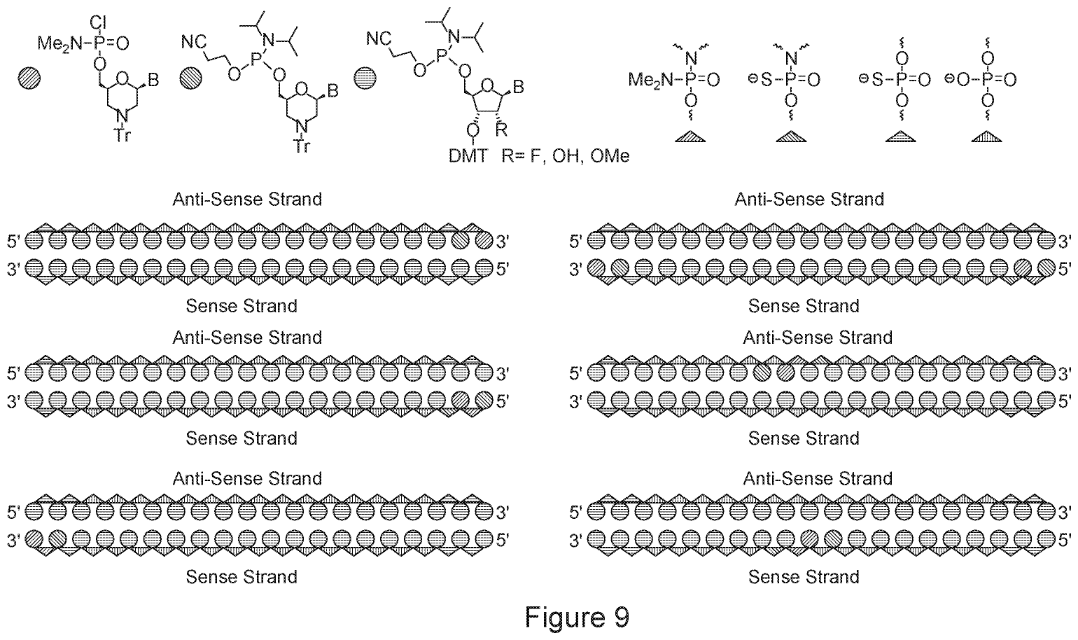

FIG. 7 depicts a hybrid sense and antisense strands of siRNA and the nucleotides and linkages therein. Any combination of the nucleotides and linkages can be employed to prepare a hybrid PMO oligonucleotide.

### Example 12: Examples of Guide and Passenger RNAs

PMOs can be the end of the guide (antisense) or passenger (sense) strand or in the middle of the guide or passenger strand (ACUG=2′-OMe RNA; ACTG=PMO; actg=2′-F RNA).

passenger
3′-TTUuAgAgUuUuAgUaGgG-5′
guide
5′-aAaAuCuCaAaAuCAuCcCu-3′
passenger
3′-TTUuAgAgUuUuAgUaGgG-5′
guide
5′-aAaAuCuCaAaAuCAuCcCU-3′
passenger
3′-TtUuAgAgUUUuAgUaGgG-5′
guide
5′-aAaAuCuCaAaAuCAuCcCu-3′
passenger
3′-TtUuAgAgUuUuAgUaGgG-5′
guide
5′-aAaUcUcAaAaUCAuCcCu-3′
passenger
3′-TtUuAgAgUuUuAgUaGGG-5′
guide
5′-aAaUcUcAaAaUCAuCcCu-3′

1-94. (canceled)

95. A hybrid oligonucleotide comprising:
(i) a first nucleotide sequence consisting of a series of two or more morpholino nucleotide analogs linked by phophorodiamidate linkages and comprising a 6′ end and a 3′ end;
(ii) a second nucleotide sequence consisting of a series of two or more deoxyribonucleotides or analogs thereof and/or ribonucleotides or analogs thereof and comprising a 5′ end and a 3′ end; and
(iii) a phosphorothioamidate linkage that links the terminal 3′ nucleic acid residue of the first nucleotide sequence and the terminal 5′ nucleic acid residue of the second nucleotide sequence.

96. The hybrid oligonucleotide of claim 95, wherein the series of deoxyribonucleotides or analogs thereof and/or ribonucleotides or analogs thereof comprises a series of 2′-modified ribonucleotide analogs.

97. The hybrid oligonucleotide of claim 95, wherein the series of 2′-modified ribonucleotide analogs is a series of 2′-OMe ribonucleotide analogs.

98. The hybrid oligonucleotide of claim 95, wherein the hybrid oligonucleotide comprises at least about 10% and up to 50% morpholino nucleotide analogs.

99. The hybrid oligonucleotide of claim 95, wherein the hybrid oligonucleotide comprises from up to 25% morpholino nucleotide analogs to up to 50% morpholino nucleotide analogs.

100. The hybrid oligonucleotide of claim 95, wherein the hybrid oligonucleotide comprises from up to 25% morpholino nucleotide analogs to up to 40% morpholino nucleotide analogs.

101. The hybrid oligonucleotide of claim 95, wherein the hybrid oligonucleotide is a steric block oligonucleotide.

102. The hybrid oligonucleotide of claim 101, wherein the steric block oligonucleotide is configured to induce exon skipping.

103. The hybrid oligonucleotide of claim 95, wherein each morpholino nucleotide analog that is linked to an adjacent morpholino nucleotide analog is linked to the adjacent morpholino nucleotide analog via a phosphorodiamidate linkage.

104. The hybrid oligonucleotide of claim 95, wherein each RNA or DNA nucleotide or nucleotide analog that is linked to an adjacent RNA or DNA nucleotide or nucleotide analog is linked to the adjacent RNA or DNA nucleotide or nucleotide analog via a phosphorothioate linkage.

105. A method of making a hybrid oligonucleotide comprising assembling P(III) and P(V) nucleotides in a 6′ to 3′ or 5′ to 3′ direction on a support and employing PMO 6′-phosphoramidite P(III) or phosphoramidate P(V) as the first nucleotide from the support, wherein at least one of the assembled P(III) and P(V) nucleotides is a first morpholino nucleotide analog and at least one of the P(III) and P(V) assembled nucleotides is a first reverse RNA or a first reverse DNA nucleotide or nucleotide analog, and the method comprises connecting the first morpholino nucleotide analog to the first reverse RNA or the first reverse DNA nucleotide or nucleotide analog via a phosphorothioamidate linkage.

106. The method of claim 105, wherein PMO 6′-phosphoramidite P(III) is the first nucleotide from the support.

107. The method of claim 105, wherein the hybrid oligonucleotide comprises:
(a) at least one phosphorothioamidate linkage; and
(b) at least one phosphorothioester linkage, phosphorodiester linkage, phosphorodiamidate linkage or a combination thereof.

108. The method of claim 105, wherein the method comprises coupling a reverse amidite to a secondary amino group of a morpholino ring to form a phosphorothioamidate linkage.

109. The method of claim 105, wherein each morphino analog of the hybrid oligonucleotide that is coupled to a RNA or a reverse DNA nucleotide or nucleotide analog of the hybrid oligonucleotide is coupled to the reverse RNA or the first reverse DNA nucleotide or nucleotide analog via a phosphorothioamidate linkage.

110. A method of making a hybrid oligonucleotide comprising assembling P(III) and P(V) nucleotides in a 6′ to 3′ or 5′ to 3′ direction on a support and employing PMO 6′-phosphoramidite P(III) as the first nucleotide from the support.

111. The method of claim 110, wherein the hybrid oligonucleotide comprises:
(a) at least one phosphorothioamidate linkage; and
(b) at least one phosphorothioester linkage, phosphorodiester linkage, phosphorodiamidate linkage or a combination thereof.

112. The method of claim 110, wherein each morphino analog of the hybrid oligonucleotide that is coupled to a RNA or a reverse DNA nucleotide or nucleotide analog of the hybrid oligonucleotide is coupled to the reverse RNA or the first reverse DNA nucleotide or nucleotide analog via a phosphorothioamidate linkage.

113. A hybrid oligonucleotide comprising a plurality of morpholino nucleotide analogs and a plurality of RNA or DNA nucleotides or nucleotide analogs, wherein each morpholino nucleotide analog that is linked to an adjacent RNA or DNA nucleotide or nucleotide analog is linked to the adjacent RNA or DNA nucleotide or nucleotide analog via a phosphorothioamidate linkage.


SEQUENCE LISTING (196 sequences)

SEQ ID NO: 1 - DNA, 20 bp, synthetic construct
  source 1..20: mol_type = other DNA
acagacaataaataccgagg

SEQ ID NO: 2 - DNA, 18 bp, synthetic construct
  source 1..18: mol_type = other DNA
gctattaccttaacccag

SEQ ID NO: 3 - RNA, 18 nt, synthetic construct
  source 1..18: mol_type = other RNA
gctattaccttaacccag

SEQ ID NO: 4 - skipped

SEQ ID NO: 5 - protein, 7 aa, synthetic construct
PKKKRKV

SEQ ID NO: 6 - protein, 9 aa, synthetic construct
KFGFGRGRQ

SEQ ID NO: 7 - protein, 4 aa, synthetic construct
HHHH

SEQ ID NO: 8 - protein, 4 aa, synthetic construct
KHKK

SEQ ID NO: 9 - protein, 4 aa, synthetic construct
KKHK

SEQ ID NO: 10 - protein, 4 aa, synthetic construct
KKKH

SEQ ID NO: 11 - protein, 4 aa, synthetic construct
KHKH

SEQ ID NO: 12 - protein, 4 aa, synthetic construct
HKHK

SEQ ID NO: 13 - protein, 4 aa, synthetic construct
KKKK

SEQ ID NO: 14 - protein, 4 aa, synthetic construct
KKRK

SEQ ID NO: 15 - protein, 4 aa, synthetic construct
KRKK

SEQ ID NO: 16 - protein, 4 aa, synthetic construct
KRRK

SEQ ID NO: 17 - protein, 4 aa, synthetic construct
RKKR

SEQ ID NO: 18 - protein, 4 aa, synthetic construct
RRRR

SEQ ID NO: 19 - protein, 4 aa, synthetic construct
KGKK

SEQ ID NO: 20 - protein, 4 aa, synthetic construct
KKGK

SEQ ID NO: 21 - protein, 5 aa, synthetic construct
HAHAH

SEQ ID NO: 22 - protein, 5 aa, synthetic construct
HAKAH

SEQ ID NO: 23 - protein, 5 aa, synthetic construct
RRRRR

SEQ ID NO: 24 - protein, 5 aa, synthetic construct
KKKKK

SEQ ID NO: 25 - protein, 5 aa, synthetic construct
KKKRK

SEQ ID NO: 26 - protein, 5 aa, synthetic construct
RKKKK

SEQ ID NO: 27 - protein, 5 aa, synthetic construct
KRKKK

SEQ ID NO: 28 - protein, 5 aa, synthetic construct
KKRKK

SEQ ID NO: 29 - protein, 5 aa, synthetic construct
KKKKR

SEQ ID NO: 30 - protein, 5 aa, synthetic construct
KAKAK

SEQ ID NO: 31 - protein, 6 aa, synthetic construct
RKKKKG

SEQ ID NO: 32 - protein, 6 aa, synthetic construct
KRKKKG

SEQ ID NO: 33 - protein, 6 aa, synthetic construct
KKRKKG

SEQ ID NO: 34 - protein, 6 aa, synthetic construct
KKKKRG

SEQ ID NO: 35 - protein, 6 aa, synthetic construct
RKKKKA

SEQ ID NO: 36 - protein, 6 aa, synthetic construct
KRKKKA

SEQ ID NO: 37 - protein, 6 aa, synthetic construct
KKRKKA

SEQ ID NO: 38 - protein, 6 aa, synthetic construct
KKKKRA

SEQ ID NO: 39 - protein, 6 aa, synthetic construct
KKKRKV

SEQ ID NO: 40 - protein, 6 aa, synthetic construct
RRRRRR

SEQ ID NO: 41 - protein, 6 aa, synthetic construct
HHHHHH

SEQ ID NO: 42 - protein, 6 aa, synthetic construct
RHRHRH

SEQ ID NO: 43 - protein, 6 aa, synthetic construct
HRHRHR

SEQ ID NO: 44 - protein, 6 aa, synthetic construct
KRKRKR

SEQ ID NO: 45 - protein, 6 aa, synthetic construct
RKRKRK

SEQ ID NO: 46 - protein, 6 aa, synthetic construct
RARARA

SEQ ID NO: 47 - protein, 6 aa, synthetic construct
KAKAKA

SEQ ID NO: 48 - protein, 7 aa, synthetic construct
PKKKRKV

SEQ ID NO: 49 - protein, 7 aa, synthetic construct
PGKKRKV

SEQ ID NO: 50 - protein, 7 aa, synthetic construct
PKGKRKV

SEQ ID NO: 51 - protein, 7 aa, synthetic construct
PKKGRKV

SEQ ID NO: 52 - protein, 7 aa, synthetic construct
PKKKGKV

SEQ ID NO: 53 - protein, 7 aa, synthetic construct
PKKKRGV

SEQ ID NO: 54 - protein, 7 aa, synthetic construct
PKKKRKG

SEQ ID NO: 55 - protein, 5 aa, synthetic construct
RARBR

SEQ ID NO: 56 - protein, 5 aa, synthetic construct
RAHAR

SEQ ID NO: 57 - protein, 5 aa, synthetic construct
HARAH

SEQ ID NO: 58 - protein, 19 aa, synthetic construct
NLSKRPAAIKKAGQAKKKK

SEQ ID NO: 59 - protein, 9 aa, synthetic construct
PAAKRVKLD

SEQ ID NO: 60 - protein, 11 aa, synthetic construct
RQRRNELKRSF

SEQ ID NO: 61 - protein, 27 aa, synthetic construct
RMRKFKNKGKDTAELRRRRVEVSVELR

SEQ ID NO: 62 - protein, 14 aa, synthetic construct
KAKKDEQILKRRNV

SEQ ID NO: 63 - protein, 8 aa, synthetic construct
VSRKRPRP

SEQ ID NO: 64 - protein, 8 aa, synthetic construct
PPKKARED

SEQ ID NO: 65 - protein, 8 aa, synthetic construct
PQPKKKPL

SEQ ID NO: 66 - protein, 12 aa, synthetic construct
SALIKKKKKMAP

SEQ ID NO: 67 - protein, 5 aa, synthetic construct
DRLRR

SEQ ID NO: 68 - protein, 7 aa, synthetic construct
PKQKKRK

SEQ ID NO: 69 - protein, 10 aa, synthetic construct
RKLKKKIKKL

SEQ ID NO: 70 - protein, 10 aa, synthetic construct
REKKKFLKRR

SEQ ID NO: 71 - protein, 20 aa, synthetic construct
KRKGDEVDGVDEVAKKKSKK

SEQ ID NO: 72 - protein, 17 aa, synthetic construct
RKCLQAGMNLEARKTKK

SEQ ID NO: 73 - protein, 7 aa, synthetic construct
FGFGRGR

SEQ ID NO: 74 - protein, 7 aa, synthetic construct
GFFGRGR

SEQ ID NO: 75 - protein, 7 aa, synthetic construct
  VARIANT 3: note = 3-(2-naphthyl)-alanine
FFXGRGR

SEQ ID NO: 76 - protein, 7 aa, synthetic construct
FFFGRGR

SEQ ID NO: 77 - protein, 7 aa, synthetic construct
  VARIANT 3: note = 3-(2-naphthyl)-alanine
FFXGRGR

SEQ ID NO: 78 - protein, 8 aa, synthetic construct
FGFGRGRQ

SEQ ID NO: 79 - protein, 8 aa, synthetic construct
GFFGRGRQ

SEQ ID NO: 80 - protein, 8 aa, synthetic construct
  VARIANT 3: note = 3-(2-naphthyl)-alanine
FFXGRGRQ

SEQ ID NO: 81 - protein, 8 aa, synthetic construct
FFFGRGRQ

SEQ ID NO: 82 - protein, 8 aa, synthetic construct
  VARIANT 3: note = 3-(2-naphthyl)-alanine
FFXGRGRQ

SEQ ID NO: 83 - protein, 8 aa, synthetic construct
  VARIANT 3: note = 3-(2-naphthyl)-alanine
FFXRRRRQ

SEQ ID NO: 84 - protein, 8 aa, synthetic construct
  VARIANT 3: note = 3-(2-naphthyl)-alanine
  VARIANT 4: note = Citrulline
  VARIANT 6: note = Citrulline
FFXXRXRQ

SEQ ID NO: 85 - protein, 8 aa, synthetic construct
  VARIANT 3: note = 3-(2-naphthyl)-alanine
FFXGRGRQ

SEQ ID NO: 86 - protein, 8 aa, synthetic construct
FFFGRGRQ

SEQ ID NO: 87 - protein, 8 aa, synthetic construct
FGFGRGRQ

SEQ ID NO: 88 - protein, 8 aa, synthetic construct
GFFGRGRQ

SEQ ID NO: 89 - protein, 8 aa, synthetic construct
FGFGRRRQ

SEQ ID NO: 90 - protein, 8 aa, synthetic construct
FGFRRRRQ

SEQ ID NO: 91 - protein, 7 aa, synthetic construct
  VARIANT 2: note = 3-(2-naphthyl)-alanine
FXRRRRQ

SEQ ID NO: 92 - protein, 7 aa, synthetic construct
  VARIANT 2: note = 3-(2-naphthyl)-alanine
FXRRRRQ

SEQ ID NO: 93 - protein, 8 aa, synthetic construct
  VARIANT 3: note = 3-(2-naphthyl)-alanine
FFXRRRRQ

SEQ ID NO: 94 - protein, 8 aa, synthetic construct
  VARIANT 3: note = 3-(2-naphthyl)-alanine
  VARIANT 4: note = Citrulline
  VARIANT 6: note = Citrulline
FFXXRXRQ

SEQ ID NO: 95 - protein, 8 aa, synthetic construct
  VARIANT 3: note = 3-(2-naphthyl)-alanine
FFXGRGRQ

SEQ ID NO: 96 - protein, 8 aa, synthetic construct
  VARIANT 3: note = 3-(2-naphthyl)-alanine
FFXRGRGQ

SEQ ID NO: 97 - protein, 8 aa, synthetic construct
FFFGRGRQ

SEQ ID NO: 98 - protein, 8 aa, synthetic construct
FGFGRGRQ

SEQ ID NO: 99 - protein, 8 aa, synthetic construct
GFFGRGRQ

SEQ ID NO: 100 - protein, 8 aa, synthetic construct
FGFGRRRQ

SEQ ID NO: 101 - protein, 8 aa, synthetic construct
FGFRRRRQ

SEQ ID NO: 102 - protein, 6 aa, synthetic construct
  VARIANT 2: note = 3-(2-naphthyl)-alanine
FXRRRQ

SEQ ID NO: 103 - protein, 6 aa, synthetic construct
  VARIANT 2: note = 3-(2-naphthyl)-alanine
FXRRRC

SEQ ID NO: 104 - protein, 6 aa, synthetic construct
  VARIANT 2: note = L-naphthylalanine
FXRRRU

SEQ ID NO: 105 - protein, 6 aa, synthetic construct
  VARIANT 4: note = L-naphthylalanine
RRRXFQ

SEQ ID NO: 106 - protein, 6 aa, synthetic construct
  VARIANT 5: note = L-naphthylalanine
RRRRXF

SEQ ID NO: 107 - protein, 6 aa, synthetic construct
  VARIANT 2: note = L-naphthylalanine
FXRRRR

SEQ ID NO: 108 - protein, 7 aa, synthetic construct
  VARIANT 2: note = D-naphthylalanine
FXRRRRQ

SEQ ID NO: 109 - protein, 7 aa, synthetic construct
  VARIANT 2: note = D-naphthylalanine
FXRRRRQ

SEQ ID NO: 110 - protein, 7 aa, synthetic construct
  VARIANT 2: note = L-naphthylalanine
FXRRRRQ

SEQ ID NO: 111 - protein, 7 aa, synthetic construct
  VARIANT 2: note = L-naphthylalanine
FXRRRRQ

SEQ ID NO: 112 - protein, 7 aa, synthetic construct
  VARIANT 5: note = L-naphthylalanine
RRFRXRQ

SEQ ID NO: 113 - protein, 7 aa, synthetic construct
  VARIANT 6: note = L-naphthylalanine
FRRRRXQ

SEQ ID NO: 114 - protein, 7 aa, synthetic construct
  VARIANT 5: note = L-naphthylalanine
RRFRXRQ

SEQ ID NO: 115 - protein, 7 aa, synthetic construct
  VARIANT 3: note = L-naphthylalanine
RRXFRRQ

SEQ ID NO: 116 - protein, 8 aa, synthetic construct
CRRRRFWQ

SEQ ID NO: 117 - protein, 8 aa, synthetic construct
  VARIANT 3: note = L-naphthylalanine
FFXRRRRQ

SEQ ID NO: 118 - protein, 8 aa, synthetic construct
  VARIANT 3: note = L-naphthylalanine
FFXRRRRQ

SEQ ID NO: 119 - protein, 8 aa, synthetic construct
  VARIANT 6: note = L-naphthylalanine
RFRFRXRQ

SEQ ID NO: 120 - protein, 8 aa, synthetic construct
URRRRFWQ

SEQ ID NO: 121 - protein, 7 aa, synthetic construct
  VARIANT 5: note = L-naphthylalanine
RRFRXRQ

SEQ ID NO: 122 - protein, 7 aa, synthetic construct
  VARIANT 6: note = L-naphthylalanine
FRRRRXQ

SEQ ID NO: 123 - protein, 8 aa, synthetic construct
CRRRRFWQ

SEQ ID NO: 124 - protein, 8 aa, synthetic construct
  VARIANT 2: note = L-naphthylalanine
FXRRRRQK

SEQ ID NO: 125 - protein, 8 aa, synthetic construct
  VARIANT 2: note = L-naphthylalanine
FXRRRRQC

SEQ ID NO: 126 - protein, 8 aa, synthetic construct
  VARIANT 2: note = L-naphthylalanine
FXRRRRRQ

SEQ ID NO: 127 - protein, 8 aa, synthetic construct
  VARIANT 2: note = L-naphthylalanine
FXRRRRRQ

SEQ ID NO: 128 - protein, 9 aa, synthetic construct
  VARIANT 5: note = L-naphthylalanine
  VARIANT 8: note = L-norleucine
RRRRXFDXC

SEQ ID NO: 129 - protein, 5 aa, synthetic construct
  VARIANT 2: note = L-naphthylalanine
FXRRR

SEQ ID NO: 130 - protein, 5 aa, synthetic construct
FWRRR

SEQ ID NO: 131 - protein, 5 aa, synthetic construct
  VARIANT 4: note = L-naphthylalanine
RRRXF

SEQ ID NO: 132 - protein, 5 aa, synthetic construct
RRRWF

SEQ ID NO: 133 - protein, 7 aa, synthetic construct
  VARIANT 2: note = L-naphthylalanine
  VARIANT 4: note = Citrulline
  VARIANT 6: note = Citrulline
FXRXRXQ

SEQ ID NO: 134 - protein, 8 aa, synthetic construct
PKKKRKVK

SEQ ID NO: 135 - protein, 8 aa, synthetic construct
  VARIANT 3: note = L-naphthylalanine
  VARIANT 6: note = Citrulline
FFXRRXRQ

SEQ ID NO: 136 - protein, 8 aa, synthetic construct
  VARIANT 3: note = L-naphthylalanine
  VARIANT 4: note = Citrulline
FFXXRRRQ

SEQ ID NO: 137 - protein, 8 aa, synthetic construct
  VARIANT 3: note = L-naphthylalanine
  VARIANT 5: note = Citrulline
  VARIANT 7: note = Citrulline
FFXRXRXQ

SEQ ID NO: 138 - protein, 7 aa, synthetic construct
  VARIANT 5: note = Citrulline
PKKKXKV

SEQ ID NO: 139 - protein, 12 aa, synthetic construct
PKKKRKVAEEAK

SEQ ID NO: 140 - protein, 5 aa, synthetic construct
RAFAR

SEQ ID NO: 141 - protein, 5 aa, synthetic construct
RAHAH

SEQ ID NO: 142 - protein, 5 aa, synthetic construct
HARAH

SEQ ID NO: 143 - protein, 8 aa, synthetic construct
  VARIANT 3: note = L-naphthylalanine
FFXSRSRQ

SEQ ID NO: 144 - protein, 8 aa, synthetic construct
FGFKRKRQ

SEQ ID NO: 145 - protein, 8 aa, synthetic construct
FGFRGRGQ

SEQ ID NO: 146 - protein, 10 aa, synthetic construct
FGFGRGRGRQ

SEQ ID NO: 147 - protein, 8 aa, synthetic construct
FGFGRRRQ

SEQ ID NO: 148 - DNA, 12 bp, Homo sapiens
  source 1..12: mol_type = other DNA
ccccgccccgcg

SEQ ID NO: 149 - DNA, 20 bp, synthetic construct
  source 1..20: mol_type = other DNA
tagtgcggacctacccacga

SEQ ID NO: 150 - DNA, 25 bp, synthetic construct
  source 1..25: mol_type = other DNA
acagacaataaataccgaggaatgt

SEQ ID NO: 151 - RNA, 20 nt, synthetic construct
  source 1..20: mol_type = other RNA
cctcggtatttattgtctgt

SEQ ID NO: 152 - RNA, 19 nt, synthetic construct
  source 1..19: mol_type = other RNA
ttttagagttttagtaggg

SEQ ID NO: 153 - RNA, 19 nt, synthetic construct
  source 1..19: mol_type = other RNA
ttttagagttttagtaggg

SEQ ID NO: 154 - RNA, 19 nt, synthetic construct
  source 1..19: mol_type = other RNA
ttttagagttttagtaggg

SEQ ID NO: 155 - RNA, 19 nt, synthetic construct
  source 1..19: mol_type = other RNA
ttttagagttttagtaggg

SEQ ID NO: 156 - RNA, 20 nt, synthetic construct
  source 1..20: mol_type = other RNA
aaaatctcaaaatcatccct

SEQ ID NO: 157 - RNA, 20 nt, synthetic construct
  source 1..20: mol_type = other RNA
aaaatctcaaaatcatccct

SEQ ID NO: 158 - RNA, 18 nt, synthetic construct
  source 1..18: mol_type = other RNA
gctattaccttaacccag

SEQ ID NO: 159 - RNA, 18 nt, synthetic construct
  source 1..18: mol_type = other RNA
gctattaccttaacccag

SEQ ID NO: 160 - RNA, 18 nt, synthetic construct
  source 1..18: mol_type = other RNA
gctattaccttaacccag

SEQ ID NO: 161 - RNA, 18 nt, synthetic construct
  source 1..18: mol_type = other RNA
gctattaccttaacccag

SEQ ID NO: 162 - DNA, 18 bp, synthetic construct
  source 1..18: mol_type = other DNA
gctattaccttaacccag

SEQ ID NO: 163 - DNA, 18 bp, synthetic construct
  source 1..18: mol_type = other DNA
gctattaccttaacccag

SEQ ID NO: 164 - DNA, 18 bp, synthetic construct
  source 1..18: mol_type = other DNA
gctattaccttaacccag

SEQ ID NO: 165 - protein, 6 aa, synthetic construct
FGRRRQ

SEQ ID NO: 166 - protein, 6 aa, synthetic construct
FDRRRQ

SEQ ID NO: 167 - protein, 6 aa, synthetic construct
FFRRRQ

SEQ ID NO: 168 - protein, 7 aa, synthetic construct
  VARIANT 2: note = L-naphthylalanine
FXRRRQF

SEQ ID NO: 169 - protein, 7 aa, synthetic construct
  VARIANT 2: note = 3-(2-naphthyl)-alanine
  VARIANT 7: note = 3-(2-naphthyl)-alanine
FXRRRQX

SEQ ID NO: 170 - protein, 7 aa, synthetic construct
  VARIANT 2: note = 3-(2-naphthyl)-alanine
FXRRRQR

SEQ ID NO: 171 - protein, 7 aa, synthetic construct
FFFRRRQ

SEQ ID NO: 172 - protein, 7 aa, synthetic construct
FRFRFRQ

SEQ ID NO: 173 - protein, 7 aa, synthetic construct
  VARIANT 3: note = 3-(2-naphthyl)-alanine
FXFRRRQ

SEQ ID NO: 174 - protein, 7 aa, synthetic construct
FFFRRRQ

SEQ ID NO: 175 - protein, 7 aa, synthetic construct
  VARIANT 3: note = 3-[2-naphthyl]-alanine
FFXRRRQ

SEQ ID NO: 176 - protein, 7 aa, synthetic construct
  VARIANT 1: note = 3-[2-naphthyl]-alanine
XFFRRRQ

SEQ ID NO: 177 - protein, 8 aa, synthetic construct
FFFRRRRQ

SEQ ID NO: 178 - protein, 8 aa, synthetic construct
RFRFRFRQ

SEQ ID NO: 179 - protein, 8 aa, synthetic construct
FFFRRRRQ

SEQ ID NO: 180 - protein, 8 aa, synthetic construct
  VARIANT 3: note = 3-[2-naphthyl]-alanine
FFXRRRAQ

SEQ ID NO: 181 - protein, 8 aa, synthetic construct
  VARIANT 3: note = 3-[2-naphthyl]-alanine
FFXRRARQ

SEQ ID NO: 182 - protein, 8 aa, synthetic construct
  VARIANT 3: note = 3-[2-naphthyl]-alanine
FFXRARRQ

SEQ ID NO: 183 - protein, 8 aa, synthetic construct
  VARIANT 3: note = 3-[2-naphthyl]-alanine
FFXARRRQ

SEQ ID NO: 184 - protein, 8 aa, synthetic construct
FFARRRRQ

SEQ ID NO: 185 - protein, 8 aa, synthetic construct
  VARIANT 3: note = 3-[2-naphthyl]-alanine
FAXRRRRQ

SEQ ID NO: 186 - protein, 8 aa, synthetic construct
  VARIANT 3: note = 3-[2-naphthyl]-alanine
AFXRRRRQ

SEQ ID NO: 187 - protein, 7 aa, synthetic construct
  VARIANT 2: note = 3-[2-naphthyl]-alanine
FXRRRRQ

SEQ ID NO: 188 - protein, 8 aa, synthetic construct
  VARIANT 3: note = 3-[2-naphthyl]-alanine
FFXRRRRQ

SEQ ID NO: 189 - protein, 8 aa, synthetic construct
  VARIANT 3: note = 3-(1-naphthyl)-alanine
FFXRRRRQ

SEQ ID NO: 190 - protein, 9 aa, synthetic construct
  VARIANT 3: note = 3-[2-naphthyl]-alanine
FFXRRRRQF

SEQ ID NO: 191 - protein, 9 aa, synthetic construct
  VARIANT 3: note = 3-[2-naphthyl]-alanine
  VARIANT 9: note = 3-[2-naphthyl]-alanine
FFXRRRRQX

SEQ ID NO: 192 - protein, 9 aa, synthetic construct
  VARIANT 3: note = 3-[2-naphthyl]-alanine
FFXRRRRQR

SEQ ID NO: 193 - protein, 9 aa, synthetic construct
  VARIANT 3: note = 3-[2-naphthyl]-alanine
FFXRRRRNF

SEQ ID NO: 194 - protein, 10 aa, synthetic construct
  VARIANT 3: note = 3-[2-naphthyl]-alanine
FFXRRRRHQF

SEQ ID NO: 195 - protein, 9 aa, synthetic construct
  VARIANT 3: note = 3-[2-naphthyl]-alanine
FFXRRRRQF

SEQ ID NO: 196 - protein, 4 aa, synthetic construct
RRRQ